(19) United States
(12) Patent Application Publication
(10) Pub. No.: US 2022/0040281 A1
(43) Pub. Date: Feb. 10, 2022

(54) RNA FOR MALARIA VACCINES

(71) Applicant: CureVac AG, Tübingen (DE)

(72) Inventors: Kim Ellen SCHWENDT, Tübingen (DE); Benjamin PETSCH, Tübingen (DE); Nicole ROTH, Tübingen (DE)

(73) Assignee: CureVac AG, Tübingen (DE)

(21) Appl. No.: 17/416,731

(22) PCT Filed: Dec. 20, 2019
(86) PCT No.: PCT/EP2019/086777

§ 371 Date: Jun. 21, 2021

(30) Foreign Application Priority Data
Dec. 21, 2018 (EP) PCT/EP2018/086797

(51) Int. Cl. A61K 39/015; A61P 33/06

(52) U.S. Cl. CPC A61K 39/015; A61K 2039/53; A61P 33/06

(57) ABSTRACT

The present invention is directed to a coding RNA for a Malaria vaccine. The coding RNA comprises at least one heterologous untranslated region (UTR), preferably a 3′-UTR and/or a 5′-UTR, and a coding region encoding at least one antigenic peptide or protein derived from a Malaria parasite, in particular at least one antigenic protein derived from circumsporozoite protein (CSP) of a Malaria parasite (e.g. Plasmodium falciparum). The present invention is also directed to compositions and vaccines comprising said coding RNA in association with a polymeric carrier, a polycationic protein or peptide, or a lipid nanoparticle (LNP). Further, the invention concerns a kit, particularly a kit of parts comprising the coding RNA, or the composition, or the vaccine. The invention is also directed to a method of treating or preventing Malaria, and the first and second medical uses of the coding RNA, the composition, the vaccine, and the kit.

### INTRODUCTION

The present invention is directed to a coding RNA for a Malaria vaccine. The coding RNA comprises at least one heterologous untranslated region (UTR), preferably a 3′-UTR and/or a 5′-UTR, and a coding region encoding at least one antigenic peptide or protein derived from a Malaria parasite, in particular at least one antigenic protein derived from circumsporozoite protein (CSP) of a Malaria parasite (e.g. Plasmodium falciparum). The present invention is also directed to compositions and vaccines comprising said coding RNA in association with a polymeric carrier, a polycationic protein or peptide, or a lipid nanoparticle (LNP). Further, the invention concerns a kit, particularly a kit of parts comprising the coding RNA, or the composition, or the vaccine. The invention is also directed to a method of treating or preventing Malaria, and the first and second medical uses of the coding RNA, the composition, the vaccine, and the kit.

Malaria infections cause about 200 million clinical cases, and about 500,000 to 600,000 deaths annually.

Malaria is a mosquito-borne infectious disease caused by protozoan parasites from the genus Plasmodium. Anopheles mosquitoes transmit malaria, and they must have been infected through a previous blood meal taken from an infected person. When a mosquito bites an infected person, a small amount of blood is taken in and contains malaria parasites. There are four main types of malaria parasites which infect humans: Plasmodium falciparum, Plasmodium vivax, Plasmodium malariae, and Plasmodium ovale. Of those, Falciparum Malaria is the deadliest type.

Many malaria parasites are now immune to the most common drugs used to treat the disease. According to the Malaria Eradication Research Agenda initiative, malaria eradication will be only achievable through effective vaccination. However, the most advanced malaria vaccine candidate, RTS,S, has presented modest results in extend and duration of protection during phase 3 clinical trials (RTS,S Clinical Trials Partnership, 2015). RTS,S contains a formulated virus-like particle that encompasses the central and carboxyl-terminal domains of the circumsporozoite protein (CSP) fused to a hepatitis B virus surface antigen. RTS,S protects approximately 30% to 50% of children from clinical disease for a limited duration. Multiple studies have shown that RTS,S induces protective antibody and CD4+ T-cell responses, but only negligible CD8+ T cell responses. However, as CD8+ T cells are a major protective immune mechanism against intracellular infections caused by Malaria parasites, an effective Malaria vaccine should induce strong CD8+ T cells responses. RTS,S was further developed with the aim to enhance vaccine efficacy by generating a more immunogenic CSP-based particle vaccine (this next-generation RTS,S like vaccine is called R21) (Collins, Katharine A., et al. “Enhancing protective immunity to malaria with a highly immunogenic virus-like particle vaccine.” Scientific reports 7 (2017): 46621). The major improvement is that in contrast to RTS,S, R21 particles are formed from a single CSP-hepatitis B surface antigen (HBsAg) fusion protein, and this leads to a vaccine composed of a much higher proportion of CSP than in RTS,S. Preclinical studies required adjuvants (Abisco-100 and Matrix-M) or TRAP-based viral vectors to induce effective and protective immune responses, especially for the induction of CSP-specific CD8+ T-cells. Adjuvants often induce tissue reactions or other unwanted side effects. Phase I assessment of first-in-human administration of the novel malaria anti-sporozoite vaccine candidate, R21 in matrix-M adjuvant, in UK and Burkinabe volunteers shows comparable immunogenicity to RTS,S/AS01B, even when administered at a five-fold lower 10 μg dose in UK and African populations.

Accordingly, using a more full-length CSP as an antigen might induce broader humoral and cellular antibody responses compared to the truncated RTS,S vaccine. Moreover, a more full-length CSP may provide additional T cell epitopes, leading to increased cellular immunity, which could potentially enhance protection against Malaria. Furthermore antibodies against a portion of the N-terminal region including R1 showed reduced risk of disease (Bongfen, Silayuv E., et al. “The N-terminal domain of Plasmodium falciparum circumsporozoite protein represents a target of protective immunity.” Vaccine 27.2 (2009): 328-335). However, the manufacturing of a Malaria vaccine that is based on a more full-length CSP is not feasible with the current state-of-the-art vaccine technologies (e.g., protein-based vaccines).

Reported problems in manufacturing of full-length protein CSP may due to unique properties of the P. falciparum parasite, which include an extremely A/T-rich genome with many lysine and arginine repeats, and proteins that contain multiple disulfide bonds. Expression of malaria proteins in bacterial systems, such as E. coli, often results in insoluble expression that requires purification from inclusion bodies and steps to refold the protein. Noe et al developed a full-length, recombinant CSP (rCSP)-based vaccine candidate against P. falciparum malaria suitable for current Good Manufacturing Practice (cGMP) production, utilizing a novel high-throughput Pseudomonas expression platform (Noe, Amy R., et al. “A full-length Plasmodium falciparum recombinant circumsporozoite protein expressed by Pseudomonas fluorescens platform as a malaria vaccine candidate.” PloS one 9.9 (2014): e107764). The rCSP, when formulated with various adjuvants, induced antigen-specific antibody responses), as well as CD4+ T-cell responses and conferred protection in mice Furthermore, heterologous prime/boost regimens with adjuvanted rCSP and an adenovirus type 35-vectored CSP (Ad35CS) showed modest improvements in eliciting CSP-specific T-cell responses and anti-malarial protection. Adjuvants often induce tissue reactions or other unwanted side effects.

Summarizing the above, the provision of an effective Malaria vaccine remains an unmet medical need of major importance for global health.

An effective Malaria vaccine should not only induces strong humoral immune responses, but also induce CD8+ T-cell responses. Therefore, an effective Malaria vaccine should ideally provide a more full-length CSP to cover also the T-cell epitopes in the N-terminal region for a strong induction of a CD8+ T-cell response. Such a Malaria vaccine should ideally be manufactured in an efficient, reliable, and scalable manner to ensure global supply. Moreover, the new vaccine should allow cost-effective production. Furthermore, the malaria vaccine should be well tolerated without possible side-effects and preferably without the use of adjuvants.

The objects outlined above are solved by the claimed subject matter, that is, inter alia, by the provision of coding RNA for a Malaria vaccine.

Notably, an RNA-based Malaria vaccine has some superior advantages over e.g. DNA-based vaccines. As generally known in the art, transfection of DNA may lead to serious problems. E.g. application of DNA bears the risk of integration into the host genome which can influence expression of the host genes, or can trigger expression of an oncogene via e.g. destruction of a tumor suppressor gene. In addition, a DNA vaccine would have to cross several membrane barriers to reach the nucleus, whereas an RNA-based vaccine does not have to cross the barrier to the nucleus and is directly translated in the cytoplasm.

Advantageously, RNA can be manufactured in a large-scale fashion, and enables the production a Malaria vaccine based on RNA encoding, for example, a more full-length CSP.

Further, it would be desirable that such an RNA-based composition or vaccine has at least some of the following advantageous features:
Improved translation of coding RNA constructs at the site of injection (e.g. muscle);
Very efficient induction of antigen-specific immune responses against the encoded CSP protein at a very low dosages and dosing regimen;
Suitability for maternal immunization;
Suitability for vaccination of infants and/or newborns;
Suitability for intramuscular administration;
Induction of specific and functional humoral immune response against Malaria (e.g. CSP of a malaria parasite);
Induction of broad, functional cellular T-cell responses against Malaria (e.g. CSP of a malaria parasite);
Induction specific B-cell memory against Malaria (e.g. CSP of a malaria parasite);
Fast onset of immune protection against Malaria (e.g. CSP of a malaria parasite);
Longevity of the induced immune responses against Malaria (e.g. CSP of a malaria parasite);
No excessive induction of systemic cytokine or chemokine response after application of the Malaria vaccine; which could lead to an undesired high reactogenicity upon vaccination
Well tolerability, no side-effects, non toxicity of the Malaria vaccine;
No enhancement of a Malaria infection due to vaccination;
Advantageous stability characteristics of the RNA-based Malaria vaccine;
Speed, adaptability, simplicity and scalability of Malaria vaccine production.
Advantageous vaccination regimen that only requires one or two vaccination for sufficient protection.

### Definitions

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as percentages by weight (wt.-%).

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells). In the context of the invention, the antigen is provided by the RNA coding sequence encoding at least one antigenic peptide or protein (e.g. CSP).

Antigen: The term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins derived from e.g. CSP comprising at least one epitope are understood as antigens in the context of the invention. In the context of the present invention, an antigen may be the product of translation of a provided coding RNA as specified herein.

Antigenic peptide or protein: The term “antigenic peptide or protein” or “immunogenic peptide or protein” will be recognized and understood by the person of ordinary skill in the art, and is e.g. intended to refer to a peptide, protein (or polyprotein) derived from a (antigenic or immunogenic) protein/polyprotein which stimulates the body's adaptive immune system to provide an adaptive immune response. Therefore an antigenic/immunogenic peptide or protein comprises at least one epitope (as defined herein) or antigen (as defined herein) of the protein it is derived from (e.g., CSP protein of a malaria parasite).

Cationic: Unless a different meaning is clear from the specific context, the term “cationic” means that the respective structure bears a positive charge, either permanently or not permanently but in response to certain conditions such as pH. Thus, the term “cationic” covers both “permanently cationic” and “cationisable”.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., in some embodiments, if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In other embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention is preferably an RNA sequence, consisting of a number of nucleotides that may be divided by three, which starts with a start codon and which preferably terminates with a stop codon.

Compound: As used herein, a “compound” means a chemical substance, which is a material consisting of molecules having essentially the same chemical structure and properties. For a small molecular compound, the molecules are typically identical with respect to their atomic composition and structural configuration. For a macromolecular or polymeric compound, the molecules of a compound are highly similar but not all of them are necessarily identical. E.g., a segment of a polymer that is designated to consist of 50 monomeric units may also contain individual molecules with e.g. 48 or 53 monomeric units.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

Thus, it is understood, if a antigenic peptides or protein is “derived from” CSP, the antigenic peptides or protein that is “derived from” said CSP may represent a variant or fragment of said respective CSP protein. Moreover, the antigenic peptides or protein that is “derived from” said CSP may differ in the amino acid sequence, sharing a certain percentage of identity as defined above.

Epitope: The term “epitope” (also called “antigen determinant” in the art) as used herein will be recognized and understood by the person of ordinary skill in the art, and is e.g. intended to refer to T cell epitopes and B cell epitopes. T cell epitopes or parts of the antigenic peptides or proteins may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 to about 20 or even more amino acids, wherein these fragments may be selected from any part of the amino acid sequence. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens, preferably having 5 to 15 amino acids, more preferably having 5 to 12 amino acids, even more preferably having 6 to 9 amino acids, which may be recognized by antibodies, i.e. in their native form. Such epitopes of proteins or peptides may furthermore be selected from any of the herein mentioned variants of such proteins or peptides. In this context antigenic determinants can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain. In the context of the present invention, an epitope may be the product of translation of a provided coding RNA as specified herein.

Fragment: The term “fragment” as used throughout the present specification in the context of a nucleic acid sequence (e.g. RNA sequence) or an amino acid sequence may typically be a shorter portion of a full-length sequence of e.g. a nucleic acid sequence or an amino acid sequence. Accordingly, a fragment, typically, consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 10%, 20%, 30%, 40%, 50%, 60%, 70%, 80%, 90%, 95% of the total (i.e. full-length) molecule from which the fragment is derived (e.g. CSP of a malaria parasite). The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide. In the context of antigens such fragment may have a length of about 6 to about 20 or even more amino acids, e.g. fragments as processed and presented by MHC class I molecules, preferably having a length of about 6 to about 12 amino acids, e.g. 6, 7, 8, 9, 10, 11, 12 amino acids, or fragments as processed and presented by MHC class II molecules, preferably having a length of about 13 or more amino acids, e.g. 11, 12, 13, 14, 15, 16, 17, 18, 19, 20 or even more amino acids, wherein these fragments may be selected from any part of the amino acid sequence. These fragments are typically recognized by T-cells in form of a complex consisting of the peptide fragment and an MHC molecule. Fragments of proteins or peptides may comprise at least one epitope of those proteins or peptides.

Heterologous: The terms “heterologous” or “heterologous sequence” as used throughout the present specification in the context of a nucleic acid sequence or an amino acid sequence refers to a sequence (e.g. DNA, RNA, amino acid) will be recognized and understood by the person of ordinary skill in the art, and is intended to refer to a sequence that is derived from another gene, from another allele, from another species. Two sequences are typically understood to be “heterologous” if they are not derivable from the same gene or in the same allele. I.e., although heterologous sequences may be derivable from the same organism, they naturally (in nature) do not occur in the same nucleic acid molecule, such as e.g. in the same RNA or protein.

Humoral immune response: The terms “humoral immunity” or “humoral immune response” will be recognized and understood by the person of ordinary skill in the art, and are e.g. intended to refer to B-cell mediated antibody production and optionally to accessory processes accompanying antibody production. A humoral immune response may be typically characterized, e.g. by Th2 activation and cytokine production, germinal center formation and isotype switching, affinity maturation and memory cell generation. Humoral immunity may also refer to the effector functions of antibodies, which include pathogen and toxin neutralization, classical complement activation, and opsonin promotion of phagocytosis and pathogen elimination.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or aa sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Immunogen, immunogenic: The terms “immunogen” or “immunogenic” will be recognized and understood by the person of ordinary skill in the art, and are e.g. intended to refer to a compound that is able to stimulate/induce an immune response. Preferably, an immunogen is a peptide, polypeptide, or protein. An immunogen in the sense of the present invention is the product of translation of a provided RNA, comprising at least one coding sequence encoding at least one antigenic peptide, protein derived from CSP as defined herein. Typically, an immunogen elicits an adaptive immune response.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Immune system: The term “immune system” will be recognized and understood by the person of ordinary skill in the art, and is e.g. intended to refer to a system of the organism that may protect the organisms from infection. If a pathogen succeeds in passing a physical barrier of an organism and enters this organism, the innate immune system provides an immediate, but non-specific response. If pathogens evade this innate response, vertebrates possess a second layer of protection, the adaptive immune system. Here, the immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts typically contains so called humoral and cellular components.

Innate immune system: The term “innate immune system” (also known as non-specific or unspecific immune system) will be recognized and understood by the person of ordinary skill in the art, and is e.g. intended to refer to a system typically comprising the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be, e.g. activated by ligands of Toll-like receptors (TLRs) or other auxiliary substances such as lipopolysaccharides, TNF-alpha, CD40 ligand, or cytokines, monokines, lymphokines, interleukins or chemokines, IL-1 to IL-33, IFN-alpha, IFN-beta, IFN-gamma, GM-CSF, G-CSF, M-CSF, LT-beta, TNF-alpha, growth factors, and hGH, a ligand of human Toll-like receptor (e.g., TLR1 to TLR10), a ligand of murine Toll-like receptor, (e.g., TLR1 to TLR13), a ligand of a NOD-like receptor, a ligand of a RIG-I like receptor, an immunostimulatory nucleic acid, an immunostimulatory RNA (isRNA), a CpG-DNA, an antibacterial agent, or an anti-viral agent.

Lipidoid compound: A lipidoid compound, also simply referred to as lipidoid, is a lipid-like compound, i.e. an amphiphilic compound with lipid-like physical properties. In the context of the present invention the term lipid is considered to encompass lipidoid compounds.

Monovalent vaccine, monovalent composition: The terms “monovalent vaccine”, “monovalent composition” “univalent vaccine” or “univalent composition” will be recognized and understood by the person of ordinary skill in the art, and are e.g. intended to refer to a composition or a vaccine comprising only one antigen from a pathogen (e.g., CSP of a Malaria parasite). Accordingly, said vaccine or composition comprises only one RNA species encoding a single antigen of a single organism. The term “monovalent vaccine” includes the immunization against a single valence. In the context of the invention, a monovalent Malaria vaccine or composition would comprise a coding RNA encoding one single antigenic peptide or protein derived from one specific Malaria parasite (e.g. CSP of a Malaria parasite).

Nucleic acid: The terms “nucleic acid” or “nucleic acid molecule” will be recognized and understood by the person of ordinary skill in the art, and are for example intended to refer to a molecule comprising, preferably consisting of nucleic acid components. The term nucleic acid molecule preferably refers to DNA or RNA molecules. It is preferably used synonymous with the term polynucleotide. Preferably, a nucleic acid or a nucleic acid molecule is a polymer comprising or consisting of nucleotide monomers, which are covalently linked to each other by phosphodiester-bonds of a sugar/phosphate-backbone. The term “nucleic acid molecule” also encompasses modified nucleic acid molecules, such as base-modified, sugar-modified or backbone-modified DNA or RNA molecules as defined herein.

Nucleic acid sequence/RNA sequence/amino acid sequence: The terms “nucleic acid sequence”, “RNA sequence” or “amino acid sequence” will be recognized and understood by the person of ordinary skill in the art, and e.g. refer to particular and individual order of the succession of its nucleotides or amino acids respectively.

Permanently cationic: The term “permanently cationic” as used herein will be recognized and understood by the person of ordinary skill in the art, and means, e.g., that the respective compound, or group or atom, is positively charged at any pH value or hydrogen ion activity of its environment. Typically, the positive charge results from the presence of a quaternary nitrogen atom. Where a compound carries a plurality of such positive charges, it may be referred to as permanently polycationic, which is a subcategory of permanently cationic.

Pharmaceutically effective amount: The terms “pharmaceutically effective amount” or “effective amount” will be recognized and understood by the person of ordinary skill in the art, and are e.g. intended to refer to an amount of a compound (e.g. the RNA of the invention) that is sufficient to induce a pharmaceutical effect, such as, in the context of the invention, an immune response against a Malaria antigen.

Polyvalent/multivalent vaccine, polyvalent/multivalent composition: The terms “polyvalent vaccine”, “polyvalent composition” “multivalent vaccine” or “multivalent composition” will be recognized and understood by the person of ordinary skill in the art, and are e.g. intended to refer to a composition or a vaccine comprising antigens from more than one strain of a Malaria parasite, or comprising different antigens of the same Malaria parasite, or any combination thereof. The terms describe that said vaccine or composition has more than one valence. In the context of the invention, a polyvalent Malaria vaccine would comprise RNA encoding antigenic peptides or proteins derived from several different Malaria parasite species or comprising RNA encoding different antigens from the same Malaria parasite species, or a combination thereof. In preferred embodiment, a polyvalent Malaria vaccine or composition comprises more than one, preferably 2, 3, 4 or even more different coding RNA species each encoding at least one peptide or protein of Malaria (e.g. CSP of Plasmodium falciparum 3D7, and CSP of Plasmodium falciparum NF54, and CSP of Plasmodium falciparum GB4). Methods to produce polyvalent RNA vaccines are disclosed in published patent application WO2017/1090134A1.

Stabilized RNA: The term “stabilized RNA” refer to an RNA molecule that is modified such, that it is more stable to disintegration or degradation, e.g., by environmental factors or enzymatic digest, such as by exo- or endonuclease degradation, than the RNA molecule without the modification. Preferably, a stabilized RNA in the context of the present invention is stabilized in a cell, such as a prokaryotic or eukaryotic cell, preferably in a mammalian cell, such as a human cell. The stabilization effect may also be exerted outside of cells, e.g. in a buffer solution etc., for example, in a manufacturing process for a pharmaceutical composition comprising the stabilized nucleic acid molecule.

T-cell responses: The terms “cellular immunity” or “cellular immune response” or “cellular T-cell responses” as used herein will be recognized and understood by the person of ordinary skill in the art, and are for example intended to refer to the activation of macrophages, natural killer cells (NK), antigen-specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface. In the context of the invention, the antigen is provided by the RNA encoding at least one antigenic peptide or protein derived from CSP. Suitably, the coding RNA, the composition, the vaccine, advantageously elicit cellular T-cell responses against the encoded Malaria antigens.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). Preferably, these fragments and/or variants have the same, or a comparable specific antigenic property (immunogenic variants, antigenic variants). “Variants” of proteins or peptides as defined herein may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, e.g., an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means, in the context of the invention, that the variant exerts essentially the same, or at least 40%, 50%, 60%, 70%, 80%, 90% of the immunogenicity as the protein it is derived from.

5′-terminal oligopyrimidine tract (TOP), TOP-UTR: The term “5′-terminal oligopyrimidine tract (TOP)” has to be understood as a stretch of pyrimidine nucleotides located in the 5′-terminal region of a nucleic acid molecule, such as the 5′-terminal region of certain RNA molecules or the 5′-terminal region of a functional entity, e.g. the transcribed region, of certain genes. The sequence starts with a cytidine, which usually corresponds to the transcriptional start site, and is followed by a stretch of usually about 3 to 30 pyrimidine nucleotides. For example, the TOP may comprise 3-30 or even more nucleotides. The pyrimidine stretch and thus the 5′-TOP ends one nucleotide 5′ to the first purine nucleotide located downstream of the TOP. Messenger RNA that contains a 5′-terminal oligopyrimidine tract is often referred to as TOP mRNA. Accordingly, genes that provide such messenger RNAs are referred to as TOP genes. The term “TOP motif” or “5′-TOP motif” has to be understood as a nucleic acid sequence which corresponds to a 5′-TOP as defined above. Thus, a TOP motif in the context of the present invention is preferably a stretch of pyrimidine nucleotides having a length of 3-30 nucleotides. Preferably, the TOP-motif consists of at least 3 pyrimidine nucleotides, preferably at least 4 pyrimidine nucleotides, preferably at least 5 pyrimidine nucleotides, more preferably at least 6 nucleotides, more preferably at least 7 nucleotides, most preferably at least 8 pyrimidine nucleotides, wherein the stretch of pyrimidine nucleotides preferably starts at its 5′-end with a cytosine nucleotide. In TOP genes and TOP mRNAs, the TOP-motif preferably starts at its 5′-end with the transcriptional start site and ends one nucleotide 5′ to the first purine residue in said gene or mRNA. A TOP motif in the sense of the present invention is preferably located at the 5′-end of a sequence which represents a 5′-UTR or at the 5′-end of a sequence which codes for a 5′-UTR. Thus, preferably, a stretch of 3 or more pyrimidine nucleotides is called “TOP motif” in the sense of the present invention if this stretch is located at the 5′-end of a respective sequence, such as the RNA, the 5′-UTR element of the RNA, or the RNA sequence which is derived from the 5′-UTR of a TOP gene as described herein. In other words, a stretch of 3 or more pyrimidine nucleotides, which is not located at the 5′-end of a 5′-UTR or a 5′-UTR element but anywhere within a 5′-UTR or a 5′-UTR element, is preferably not referred to as “TOP motif”. In some embodiments, the nucleic acid sequence of the 5′-UTR element, which is derived from a 5′-UTR of a TOP gene, terminates at its 3′-end with a nucleotide located at position 1, 2, 3, 4, 5, 6, 7, 8, 9 or 10 upstream of the start codon (e.g. A(U/T)G) of the gene or RNA it is derived from. Thus, the 5′-UTR element does not comprise any part of the protein coding sequence. Thus, preferably, the only protein coding part of the at least one nucleic acid sequence, particularly of the RNA sequence, is provided by the coding sequence.

### Short Description of the Invention

The present invention is based on the inventor's surprising finding that at least one peptide or protein derived from CSP of a Malaria parasite encoded by the RNA of the invention can efficiently be expressed in a mammalian cell. Even more unexpected, the inventors showed that the coding RNA of the invention can induce specific functional immune responses in e.g. mice (see e.g. Examples 2 and 3). Through different optimizations in CSP antigen design, the immune responses could be further improved. Heterologous elements like e.g. heterologous transmembrane domains, secretory signal peptides, T helper epitopes or antigen clustering domains resulted in improved immune responses (see e.g. Examples 7, 8, and 9). Furthermore, optimization in mRNA design (improved UTR combinations, use of cap 1 analogous, etc) could effectively improve the immune responses; mainly T-cell based immune responses (see Examples 11, 12 and 13. Advantageously, said coding RNA of the invention induces very efficient antigen-specific immune responses against the encoded CSP (humoral and cellular responses). Further, the coding RNA of the invention comprised in lipid nanoparticles (LNPs) very efficiently induces antigen-specific immune responses against CSP at a very low dosages and dosing regimen (see e.g. Example 2, 3, 6-13). Accordingly, the coding RNA, and the composition/vaccine comprising said coding RNA of the invention are suitable for eliciting an immune response against CSP of a Malaria parasite in a mammalian subject. The coding RNA and the composition/vaccine comprising said coding RNA is therefore suitable for use as a vaccine, e.g. as a human vaccine.

In a first aspect, the present invention provides a coding RNA, preferably a coding RNA for a vaccine, comprising at least one 5′ untranslated region (UTR) and/or at least one 3′ untranslated region (UTR), and at least one coding sequence operably linked to said 3′-UTR and/or 5′-UTR encoding at least one antigenic protein derived from CSP of a malaria parasite, or an immunogenic fragment or immunogenic variant thereof.

In a second aspect, the present invention provides a composition, preferably an immunogenic composition, comprising the coding RNA of the first aspect. Suitably, the composition may comprise the coding RNA of first aspect complexed with, encapsulated in, or associated with one or more lipids, thereby forming lipid nanoparticles.

In a third aspect, the present invention provides a Malaria vaccine wherein the vaccine comprises the coding RNA of the first aspect or the composition of the second aspect.

In a fourth aspect, the present invention provides a kit or kit of parts, wherein said kit or kit of parts comprises the coding RNA of the first aspect, and/or the composition of the second aspect, and/or the vaccine of the third aspect.

The invention further concerns a method of treating or preventing Malaria in a subject, first and second medical uses of the coding RNA, compositions, and vaccines. Further, the invention is directed to a kit, particularly to a kit of parts, comprising the coding RNA, compositions, and vaccines. Also provided are methods of manufacturing the coding RNA, the composition or the vaccine.

### DETAILED DESCRIPTION OF THE INVENTION

The present application is filed together with a sequence listing in electronic format, which is part of the description of the present application (WIPO standard ST.25). The information contained in the sequence listing is incorporated herein by reference in its entirety. Where reference is made herein to a “SEQ ID NO”, the corresponding nucleic acid sequence or amino acid (aa) sequence in the sequence listing having the respective identifier is referred to. For many sequences, the sequence listing also provides additional detailed information, e.g. regarding certain structural features, sequence optimizations, GenBank identifiers, or additional detailed information regarding its coding capacity. In particular, such information is provided under numeric identifier <223> in the WIPO standard ST.25 sequence listing. Accordingly, information provided under said numeric identifier <223> is explicitly included herein in its entirety and has to be understood as integral part of the description of the underlying invention.

### Coding RNA for Vaccination:

In a first aspect, the invention relates to a coding RNA, preferably a coding RNA for a vaccine, comprising
a) at least one heterologous 5′ untranslated region (5′-UTR) and/or at least one heterologous 3′ untranslated region (3′-UTR); and
b) at least one coding sequence operably linked to said 3′-UTR and/or 5′-UTR encoding at least one antigenic protein, preferably derived from circumsporozoite protein (CSP) of a Malaria parasite, or an immunogenic fragment or immunogenic variant thereof.

The terms “coding RNA” as used herein will be recognized and understood by the person of ordinary skill in the art, and are e.g. intended to refer to an RNA comprising a coding sequence (“cds”) comprising several nucleotide triplets, wherein said cds may be translated into a peptide or protein.

The term “coding RNA for a vaccine” as used herein has to be understood as a coding RNA having certain advantageous features that makes the RNA suitable for in vivo administration to a subject, e.g. a human. Moreover, a “coding RNA for a vaccine” is preferably expressed, that is translated into protein, when administered to a subject, e.g. a human. In addition, the “coding RNA for a vaccine” preferably induces a specific immune response against the encoded protein after administration to a subject, e.g. a human.

Preferably, intramuscular or intradermal administration of said “coding RNA for a vaccine” results in expression of the encoded CSP antigen in a subject.

The term “immunogenic fragment” or “immunogenic variant” has to be understood as a fragment/variant of the corresponding antigen (e.g. CSP) that is capable of raising an immune response in a subject.

In general, the RNA of the invention may be composed of a protein-coding region (also referred to as coding sequence “cds”, or “ORF”), and 5′- and/or 3′-untranslated regions (UTRs). The 3′-UTR is variable in sequence and size; it spans between the stop codon and the poly(A) tail. Importantly, the 3′-UTR sequence harbors several regulatory motifs that determine RNA turnover, stability and localization, and thus governs many aspects of post-transcriptional regulation. In medical application of RNA (e.g. immunotherapy applications, vaccination) the regulation of RNA translation into protein is of paramount importance to therapeutic safety and efficacy. The present inventors surprisingly discovered that certain RNA constructs enable the rapid and transient expression of high amounts of CSP antigenic peptides or proteins. Further, said RNA molecules induce, when administered to a subject, a balanced immune response, comprising both cellular and humoral immunity. Accordingly, the coding RNA provided herein is particularly useful and suitable for various applications in vivo, including the vaccination against Malaria parasites, and may, accordingly, be a suitable component of a vaccine for treating and/or preventing Malaria.

### Malaria Parasites:

As used herein, the term “Malaria parasite” refers to any protozoan parasite capable of causing Malaria in a subject.

Typically, Malaria is caused by parasitic protozoan species of the genus Plasmodium (NCBI Taxonomy ID: 5820), or the subgenus Plasmodium (NCBI Taxonomy ID: 418103)). Accordingly, Plasmodium species have to be understood, and will be recognized as “Malaria parasites” by the person of skill in the art. The term “Plasmodium” refers to any species in the Plasmodium genus or subgenus, and is not limited to a particular species, sub-species, strain, variant, or isolate, etc. Accordingly, the term “Plasmodium” may refer to a Plasmodium species, a Plasmodium sub-species, a Plasmodium strain, a Plasmodium variant, a Plasmodium isolate of any origin. Preferred is a “Plasmodium” that may cause a disease in humans or animals, e.g. at least mild symptoms associated with Malaria.

In preferred embodiments, the at least one antigenic protein of the invention may be derived from any one of the Malaria parasites selected from Plasmodium falciparum (Pf), Plasmodium knowlesi (Pk), Plasmodium ovale (Po), Plasmodium simiovale (Ps), and Plasmodium vivax (Pv). In preferred embodiments, the Malaria parasite is Plasmodium falciparum (Pf), Plasmodium malariae (Pm). Plasmodium ovale curtisi (Poc), Plasmodium ovale wallikeri (Pow), Plasmodium berghei (Pb).

According to various embodiments, the coding sequence of the RNA of the first aspect comprises or consists of a nucleic acid sequence encoding an antigenic protein derived from any one of the Malaria parasites provided in List 1 below. Therein, for each of the suitable Malaria parasites, in particular, for each of the suitable Plasmodium species (e.g. Plasmodium falciparum (Pf), Plasmodium knowlesi (Pk), Plasmodium ovale (Po), Plasmodium simiovale (Ps), and Plasmodium vivax (Pv)), the respective NCBI Taxonomy ID (“NCBI-ID”) is indicated.

List 1: Malaria Parasites/Plasmodium Species and Subspecies with Respective NCBI Taxonomy IDs:

Species: Plasmodium falciparum (Pf) (malaria parasite P. falciparum) (5833); Subspecies: Pf 303.1 (1245013); Pf 309.1 (1245014), Pf 311 (57265), Pf 318.1 (1245015), Pf 326.1 (1245016), Pf 327.1 (1245017), Pf 365.1 (1245018), Pf 366.1 (1245019), Pf 377.1 (1245020), Pf 383.1 (1245021), Pf 397.1 (1245022), Pf 398.1 (1245023), Pf 3D7 (36329), Pf 58.1 (1245012), Pf 7G8 (57266), Pf 803_H2 (1226433), Pf 87_239 (685969), Pf B1E4_6273_2 clone2 (1226423), Pf CAMP/Malaysia (5835), Pf CDC/Honduras (5836), Pf Cp803 (1226435), Pf D10 (478861), Pf D6 (478860), Pf Dd2 (57267), Pf FC27/Papua New Guinea (5837), Pf FcB1/Columbia (186763), Pf FCBR/Columbia (33631), Pf FCC-2/Hainan (478862), Pf FCH-5 (1036724), Pf FCH/4 (132416), Pf FCM17/Senegal (5845), Pf FCR-3/Gambia (5838), Pf Fid3/India (70152), Pf GB4 (5833), Pf HB3 (137071), Pf IGH-CR14 (580059), Pf IMR143 (57268), Pf WELLCOME (5848), Pf K1 (5839), Pf KF1916 (57269), Pf LES (5840), Pf Mad20/Papua New Guinea (5841), Pf Mad71/Papua New Guinea (70154), Pf MaliPS096_E11 (1036727), Pf ML-14 (685970), Pf MLW.2745 (1226410), Pf MLW.2749 (1226408), Pf MLW.2786 (1226411), Pf MLW.2788 (1226413), Pf MLW.2861 (1226420), Pf MLW.2927 (1226417), Pf MLW.2928 (1226415), Pf MLW.2929 (1226421), Pf MLW.2941 (1226418), Pf MLW.2953 (1226419), Pf MLW.2955 (1226412), Pf MLW.2965 (1226414), Pf MLW.2970 (1226409), Pf MLW.2979 (1226422), Pf MLW.2998 (1226416), Pf NF135/5.C10 (1036726), Pf NF54 (5843), Pf NF7/Ghana (5842), Pf Nig32/Nigeria (70150), Pf P27.02 (871297), Pf P51.02 (871296), Pf Palo Alto/Uganda (57270), Pf RAJ116 (580058), Pf RO-33 (5834), Pf Santa Lucia (478859), Pf Senegal_V34.04 (478863), Pf SenP05.02 (871286), Pf SenP08.04 (871278), Pf SenP09.04 (871279), Pf SenP11.02 (871276), Pf SenP19.04 (871277), Pf SenP26.04 (871275), Pf SenP31.01 (871284), Pf SenP60.02 (871285), Pf SenT002.09 (1107494), Pf SenT015.09.c (1226430), Pf SenT016.10.d (1226436), Pf SenT021.09 (1107495), Pf SenT021.10.d (1226437), Pf SenT029.09 (1107496), Pf SenT032.09 (1107493), Pf SenT033.09 (1107497), Pf SenT042.09.c (1226427), Pf SenT046.09.c (1226434), Pf SenT047.09.c (1226425), Pf SenT049.10.d (1226438), Pf SenT061.10.d (1226439), Pf SenT065.10.d (1226440), Pf SenT069.10.d (1226441), Pf SenT076.10.d (1226442), Pf SenT077.09.c (1226426), Pf SenT079.09.c (1226424), Pf SenT079.10.d (1226443), Pf SenT086.09 (1107498), Pf SenT090.09 (1107499), Pf SenT092.09.c (1226431), Pf SenT104.10.d (1226444), Pf SenT106.09.d (1226445), Pf SenT108.10.d (1226446), Pf SenT109.09.c (1226429), Pf SenT111.09 (1107500), Pf SenT111.10.d (1226447), Pf SenT112.09 (1107501), Pf SenT112.10.d (1226448), Pf SenT116.09.d (1226449), Pf SenT117.09.d (1226450), Pf SenT118.10.d (1226451), Pf SenT121.09.d (1226452), Pf SenT123.09 (1107502), Pf SenT125.10.d (1226453), Pf SenT126.10.d (1226454), Pf SenT127.09 (1107503), Pf SenT128.09 (1107504), Pf SenT131.10.d (1226455), Pf SenT131.11.d (1226456), Pf SenT135.09 (1107505), Pf SenT135.10.d (1226457), Pf SenT137.09 (1107506), Pf SenT139.09.d (1226458), Pf SenT142.09 (1107507), Pf SenT145.10.d (1226459), Pf SenT147.09.d (1226460), Pf SenT148.09 (1107508), Pf SenT149.09 (1107509), Pf SenT151.09 (1107510), Pf SenT153.09.c (1226428), Pf SenT155.10.d (1226461), Pf SenT161.09.d (1226462), Pf SenT161.10.d (1226726), Pf SenT162.10.d (1226463), Pf SenT165.09 (1107511), Pf SenT166.09 (1107512), Pf SenT183.10.d (1226464), Pf SenT184.10.d (1226465), Pf SenT250.08.c (1226432), Pf SenT26.04 (871281), Pf SenT28.04 (871280), Pf SenV34.04 (871283), Pf SenV35.04 (871282), Pf T4/Thailand (5846), Pf TAK 9 (57276), Pf Tanzania (2000708) (1036725), Pf Th10.04_D10 (871287), Pf Th105.07 (871292), Pf Th113.09 (871299), Pf Th130.09 (871298), Pf Th15.04 (871294), Pf Th230.08 (871290), Pf Th231.08 (871289), Pf Th232.08 (871288), Pf Th74.08 (871293), Pf THTN/Thailand (70151), Pf UGK 396.1 (1050250), Pf UGK 408.2 (1050252), Pf UGK 432.4 (1050253), Pf UGK 443.2 (1050251), Pf UGK 659.1 (1050254), Pf UGK 661.1 (1050255), Pf UGK 674.4 (1050256), Pf UGK 707.3 (1050257), Pf UGK 730.2 (1050258), Pf UGK 815.1 (1050259), Pf UGT5.1 (1237627), Pf V1 (5847), Pf V42.05 (871295), Pf V92.05 (871291), Pf Vietnam Oak-Knoll (FVO) (1036723), Pf VS/1 (478864), Pf W2mef (5833); Species: Plasmodium knowlesi (Pk) (5850); Subspecies: Pk H (5851), Pk Nuri (5852); Species: Plasmodium malariae (Pm) (5858); Species: P. cf. malariae (196059); Species: Plasmodium cf. malariae type2 (1583084); Species: Plasmodium ovale (Po) (malaria parasite P. ovale) (36330); Subspecies: P. ovale curtisi (Poc) (864141),Po Nigeria 1/CDC (573885),P. ovale wallikeri (Pow) (864142); Species: Plasmodium cf. ovale (943109); Species: Plasmodium simiovale (35085); Species: P. vivax (Pv) (malaria parasite P. vivax) (5855); Subspecies: Pv Brazil 1(1033975), Pv India VII (1077284), Pv IQ07 (882766), Pv Mauritania 1 (1035515), Pv North Korean (1035514), Pv Sal-1 (126793), Pv Belem (31273); Species: P. cf. vivax (943110); Species: P. cf. vivax EKgor1179_SGA2.9 (1318701); Species: P. cf. vivax EKgor514_SGA2.6 (1318700); Species: P. cf. vivax FP-2013 (1329927); Species: P. vivax-like sp. (27990)

In preferred embodiments, the antigenic protein of the first aspect may be derived from Plasmodium falciparum (NCBI-ID 5833, or respective subspecies according to List 1), in particular, from Plasmodium falciparum 3D7 (NCBI-ID 36329), or Pf NF54 (5843).

### Suitable Malaria Antigens:

The invention relates to a coding RNA, wherein said coding RNA comprises a coding sequence encoding at least one antigenic protein derived from a Malaria parasite as defined above, or an immunogenic fragment or immunogenic variant of an antigenic protein derived from a Malaria parasite.

Suitably, the at least one antigenic protein may be derived from circumsporozoite protein (CSP), liver stage antigen 1 (LSA1), merozoite surface protein-1 (MSP1), apical membrane antigen 1 (AMA1), thrombospondin related adhesive protein (TRAP), VAR2CSA, Gamete surface antigen (Pfs230), Ookinete surface protein (Pfs28), Sexual stage antigen (pfs25), transmission-blocking target protein (Pfs45/48), reticulocyte-binding protein homologue 5 (RH5), RH5 interacting protein (Ripr), erythrocyte membrane protein 1 (EMP1), sporozoite surface protein 2 (SSP2), or combinations, or immunogenic fragments, or immunogenic variants of any of these.

Suitable antigenic proteins, e.g. AMA1, EMP1, MSP1, SSP2, or TRAP, may be derived from proteins according to Table 3 of WO2017/070624, the content of Table 3 of WO2017/070624, in particular, the NCBI accession NOs disclosed in Table 3 of WO2017/070624 herein included by reference.

In preferred embodiments, the at least one antigenic protein may be derived from circumsporozoite protein (CSP) of a Malaria parasite, or an immunogenic fragment or immunogenic variant thereof.

CSP is a multifunctional protein, forming a dense coat on the surface of the sporozoite of a Malaria parasite. Its overall structure is highly conserved in all Plasmodium species, consisting of a central repeat region flanked by an NH2-terminal domain containing a conserved proteolytic cleavage site, and a C-terminal cell-adhesion domain, the thrombospondin repeat (TSR) domain. It has been proposed in the art that N- and C-terminal regions of CSP have a functional role during egress from oocysts, invasion of salivary glands, exit from the inoculation site, and localization to and invasion of hepatocytes. After their release from oocysts, the N-terminus of CSP mediates adhesion to salivary glands and, in the mammalian host, the region masks the TSR, maintaining the sporozoite in a migratory state. In the liver, a regulated proteolytic cleavage event leads to the removal of the N-terminal third of the protein exposing the TSR, an event that may be critical for efficient invasion of hepatocytes by sporozoites.

As CSP is expressed on the surface of the sporozoite of a Malaria parasite, CSP may represent a main target for antibody mediated immunity. Accordingly, CSP (or fragments, variants thereof) is used as antigen in the context of the invention.

Suitable CSP amino acid sequences may be derived from any CSP provided in List 2 (NCBI Protein Accession numbers).

### List 2: NCBI Protein Accession Numbers of Suitable Malaria Antigens:

XP_001351122.1, BAM84949.1, BAD73956.1, AAA29551.1, AAA29554.1, BAM84958.1, BAN59428.1, ACO49408.1, ACO49420.1, ACO49498.1, ACO49505.1, AAA29576.1, AAA29545.1, AAN87576.1, AAN87620.1, BAN59401.1, BAM85068.1, BAM84914.1, BAM84865.1, ACO49446.1, ACO49503.1, ACO49504.1, ADF48458.1, AAA29543.1, AAA29571.1, AAN87622.1, BAN59407.1, BAM85010.1, BAN59429.1, BAM85085.1, BAM84895.1, ACO49368.1, ACO49378.1, ACO49457.1, ACO49467.1, ACO49541.1, ACO49544.1, AAN87575.1, BAM84946.1, BAM84947.1, BAN59412.1, BAN59422.1, BAN59424.1, BAM84878.1, ACO49384.1, ACO49490.1, ACO49538.1, ACO49540.1, AAA29562.1, BAM84952.1, BAN59425.1, BAM85007.1, BAM85089.1, BAM85102.1, AAA29555.1, BAM84798.1, ADF48375.1, ACO49542.1, ACO49545.1, AAA29574.1, AAW59565.1, AAA29552.1, BAD73952.1, BAM84944.1, BAM84957.1, BAM85032.1, BAM85062.1, BAM85093.1, BAM84896.1, BAM84907.1, BAM84756.1, BAM84758.1, ACO49480.1, ACO49517.1, AAN87591.1, AAN87590.1, BAM84820.1, AAA29550.1, BAM84917.1, BAM84954.1, BAM85044.1, BAM85045.1, BAM84987.1, BAM84993.1, BAM84805.1, BAM84750.1, BAM85260.1, BAM85298.1, AAN87614.1, ACO49328.1, ACO49339.1, ACO49493.1, AGR53780.1, AAN87605.1, AAN87598.1, AAN87611.1, AAN87589.1, BAM85131.1, BAM85145.1, AAN87606.1, BAM84921.1, BAM84929.1, BAM84935.1, BAM85103.1, BAM84801.1, BAM84804.1, BAM84806.1, BAM84753.1, BAM84757.1, BAM84759.1, BAM84763.1, BAM84764.1, BAM84768.1, BAM84770.1, BAM84775.1, BAM84778.1, BAM84779.1, BAM84781.1, BAM84789.1, BAM84792.1, BAM84797.1, AAN87602.1, AAN87587.1, ACO49330.1, AGR53782.1, AAN87609.1, AAN87613.1, BAM85120.1, BAM84840.1, BAM84799.1, BAM84800.1, BAM84765.1, BAM84771.1, BAM84773.1, BAM84783.1, BAM84785.1, BAM84790.1, ACO49332.1, BAM84829.1, BAM84956.1, BAM84795.1, AAN87588.1, ADF48384.1, BAM84833.1, BAM84831.1, AAN87582.1, BAM84782.1, AAN87583.1, AAN87593.1, AAN87594.1, AAN87592.1, AGR53781.1, BAM84819.1, BAM84821.1, BAM84808.1, BAM84814.1, BAM84838.1, BAM84839.1, BAM84802.1, BAM84803.1, AAN87579.1, AAN87578.1, AAN87607.1, AAN87608.1, AAN87595.1, AAN87618.1, AAN87585.1, AAN87577.1, BAM84815.1, BAM84816.1, BAM84812.1, BAM84834.1, BAM84835.1, BAM84822.1, BAM84823.1, BAM84824.1, BAM84825.1, BAM84826.1, BAM84832.1, BAD73957.1, BAM84752.1, BAM84762.1, BAM84774.1, BAM84777.1, BAM84786.1, BAM84796.1, AAN87599.1, AAN87581.1, BAM84810.1, BAM84811.1, BAM84836.1, BAM84827.1, BAM84748.1, BAM84749.1, BAM84751.1, BAM84761.1, BAM84776.1, BAM84794.1, AAN87580.1, AAN87610.1, AAN87619.1, AAN87617.1, AAN87616.1, AAN87600.1, AAN87597.1, AAN87596.1, AAN87615.1, AHF20622.1, XP_002259002.1, AFD97213.1, AFG25469.1, AFD97209.1, AFD97205.1, AFD97206.1, AFD97212.1, AFD97214.1, AFD97210.1, AFD97211.1, AHF20628.1, ADX31295.1, AHF20648.1, AHF20646.1, AFD97208.1, ADN94497.1, ADN94542.1, AHF20661.1, AHF20662.1, AHF20644.1, AFG25481.1, AHF20657.1, ADN94498.1, AHF20637.1, AFD97227.1, AHF20638.1, AHF20666.1, AHF20664.1, AHF20669.1, ADN94489.1, AHF20658.1, AHF20656.1, AFD97232.1, AFD97225.1, ADN94493.1, AHF20651.1, AHF20655.1, ADN94538.1, ADN94539.1, AFG25472.1, AFG25474.1, AFG25479.1, ADN94494.1, ADN94507.1, ADN94522.1, AHF20649.1, AHF20650.1, AHF20668.1, AHF20617.1, AHF20670.1, AHF20615.1, AFG25470.1, AHF20639.1, ADN94495.1, AFG25463.1, AFG25464.1, ADN94508.1, AFG25471.1, AFG25478.1, AHF20663.1, ADN94519.1, AFD97223.1, ADN94525.1, ADN94503.1, AHF20634.1, AHF20635.1, AHF20630.1, AFG25480.1, AHH02601.1, AHH02602.1, ADN94540.1, AHF20652.1, AHF20665.1, AHF20620.1, AHF20621.1, AHF20623.1, ACD86467.1, ADN94491.1, ADN94521.1, ADN94529.1, ADN94527.1, AFG25482.1, ADN94501.1, AHF20624.1, AHF20625.1, AFG25467.1, ADN94515.1, AFG25476.1, AFD97238.1, AFD97243.1, AHF20645.1, ADN94510.1, AFD97233.1, AFD97240.1, AFG25477.1, ADN94524.1, ADN94485.1, ADN94487.1, ADN94523.1, ADN94520.1, AFG25473.1, AHF20667.1, AHF20618.1, AHF20619.1, ADN94505.1, ADN94486.1, ADN94511.1, AFG25475.1, AHF20642.1, AHF20641.1, AHF20632.1, AHF20626.1, ADN94509.1, AHF20653.1, AFD97207.1, ADN94514.1, AFD97239.1, ADN94490.1, ADN94528.1, AFD97222.1, ADN94512.1, ADN94502.1, AFD97229.1, ADN94513.1, CAA05623.1, AAA29557.1, SCN12386.1, SCN12386.1, SBT79431.1, SBT00176.1, SBT84923.1, SBS83173.1, SBT35133.1, SBT34702.1, SBT72933.1, ADB92551.1, XP_001613068.1, SGX77278.1, AHL69650.1, AGN05257.1, AGN05240.1, ADB92533.1, ADB92538.1, AHL69649.1, AGN05254.1, ADB92534.1, ADB92542.1, ADB92553.1, ADB92546.1, ADB92547.1, ADB92548.1, ADB92531.1, AHL69647.1, AGN05236.2, AGN05238.1, ANS71607.1, ANS71618.1, ANS71628.1, ADB92539.1, ADB92540.1, ADB92543.1, ADB92544.1, ADB92545.1, AHL69652.1, AGN05250.1, AGN05252.1, AHL69651.1, ADB92550.1, AGN05267.1, ADB92541.1, ADB92554.1, ADB92528.1, AGN05255.1, AGN05273.2, ANS71590.1, AGN05249.1, AGN05241.2, AGN05234.2, AGN05258.1, AGN05271.1, AGN05268.1, ANS71594.1, ANS71602.1, ANS71608.1, ANS71611.1, ANS71617.1, ANS71630.1, AGN05247.1, AGN05260.1, AGN05266.1, ANS71599.1, ANS71604.1, ANS71615.1, ANS71589.1, ADB92552.1, ADB92555.1, XP_022712148.1.

Accordingly, each of the amino acid sequences for CSP encompassed by the accession numbers of List 2, and corresponding variants having greater than 80%, 95%, 90%, 95% identity to each of the amino acid sequences encompassed by the accession numbers of List 2, are herewith included as part of the disclosure. Further, fragments of the amino acid sequences encompassed by the accession numbers of List 2, e.g. corresponding fragments having more than 60%, 70%, 80%, 90% of the length of the amino acid sequences encompassed by the accession numbers of List 2, are herewith included as part of the disclosure.

In various embodiments, each of the amino acid sequences for CSP being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-36, 2081-2120, 2481-2886, 8742-8753, 10080-10085, or an immunogenic fragment or immunogenic variant of any of these sequences may be the “at least one antigenic protein derived from circumsporozoite protein (CSP) of a Malaria parasite” of the invention. Additional information regarding each of these suitable amino acid sequences encoding proteins derived from Malaria parasites may also be derived from the sequence listing, in particular from the details provided therein under identifier <223> as explained in the following.

It has to be noted that where reference is made to amino acid (aa) residues and their position in a CSP, any numbering used herein—unless stated otherwise—relates to the position of the respective amino acid residue in a corresponding CSP of Plasmodium falciparum 3D7 (Strain NCBI-ID 36329) according to SEQ ID NO: 1. Respective amino acid positions are, throughout the disclosure, exemplarily indicated for CSP of Plasmodium falciparum 3D7 (XP_001351122.1, XM_001351086.1; abbreviated herein as “Pf(3D7)”). The person skilled in the art will of course be able to adapt the teaching relating to CSP of Pf(3D7) to each and every CSP as provided herein, in particular to each and every CSP as provided in List 2, preferably to each and every CSP fragment as provided in the sequence listing (e.g., SEQ ID NOs: 1-36, 2081-2120, 2481-2886, 8742-8753, 10080-10085).

Full length CSP of Plasmodium falciparum 3D7 consists of 397 amino acids, comprising the following elements or regions (indicated by amino acid position) (further information can be found in Doud, Michael B., et al. “Unexpected fold in the circumsporozoite protein target of malaria vaccines.” Proceedings of the National Academy of Sciences 109.20 (2012): 7817-7822):

E1) Full length CSP: amino acid 1-397;
E2) Secretory signal sequence/signal peptide (SP): amino acid 1-18;
E3) RI region+NANP repeat region: amino acid 93-272;
E4) Central repeat region: amino acid 105-272;
E5) NANP repeat region: amino acid 98-272
E6) EcCSP fragment: amino acid 27-384;
E7) PpCSP fragment: amino acid 74-383;
E8) RI region: amino acid 93-97;
E9) RTS,S fragment: amino acid 207-395;
E10) RIII region, Th2R epitope region v1: amino acid 310-327;
E11) RIII region+TSR (RII+) region: amino acid 310-374;
E12) TSR region v3: amino acid 319-375;
E13) TSR region v1: amino acid 326-374;
E14) TSR region v2: amino acid 328-374;
E15) RII+region v1: amino acid 330-347;
E16) RII+region v2: amino acid 330-351;
E17) Glycosylphosphatidylinositol (GPI) anchor: amino acid 375-397;
E18) CSP-delSP-delTSR(v2)-delGPI: amino acid 19-325;
E19) CSP-delTSR(v2)-delGPI: amino acid 1-325;
E20) CSP-delSP-delGPI: amino acid 19-374;
E21) CSP-delSP: amino acid 19-397;
E22) CSP_delGPI: amino acid 1-374;
E23) RIII region, Th2R epitope region v2: amino acid 309-327;
E24) Th3R epitope region v2: amino acid 346-366;
E25) Th3R+CS.T3 epitope region v2: amino acid 346-376;
E26) Th3R epitope region v2: amino acid 346-365;
E27) Th3R+CS.T3 epitope region v2: amino acid 346-375.

Preferably, whenever reference is made to a CSP protein in the context of the invention, it has to be understood that at least one of the above elements E1 to E27, or at least one fragment of the above elements E1 to E27 is present. Accordingly, the coding RNA of the invention encodes at least one of the elements E1 to E27 as described above, or an immunogenic fragment, or immunogenic variant thereof.

CSP of Plasmodium falciparum 3D7 comprises several described epitopes, for example 3A1 antibody binding site (amino acid 69-74), 2C3 antibody binding site (amino acid 75-94), 3H10/3B4 antibody binding site (amino acid 95-100) etc. In preferred embodiments, the coding RNA of the invention encodes at least one CSP epitope, e.g. one of the above exemplified epitopes.

Suitable protein fragments derived from CSP of Pf(3D7), and corresponding RNA coding sequences encoding said fragments are provided in Table 3 (column A: description of fragments with indication of the amino acid position in relation to the full length protein; column B: corresponding amino acid sequences). Examples of preferred protein fragments are, but not limited to CSP(1-397), CSP(19-397), CSP(19-384), and CSP(199-377).

In preferred embodiments, the at least one antigenic protein derived from circumsporozoite protein (CSP) of a Malaria parasite comprises an amino acid sequence stretch derived from CSP with a length of more than 180 amino acids, 200 amino acids, 220 amino acids, 240 amino acids, 260 amino acids, 280 amino acids, 300 amino acids, 320 amino acids, 340 amino acids, 360 amino acids, 380 amino acids, 390 amino acids, wherein the amino acid stretch is preferably derived from CSP of Plasmodium falciparum 3D7.

More preferably, the at least one antigenic protein derived from circumsporozoite protein (CSP) of a Malaria parasite comprises an amino acid sequence stretch derived from CSP with a length of 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397 amino acids, wherein the amino acid stretch is preferably derived from CSP of Plasmodium falciparum 3D7.

In preferred embodiments, the at least one antigenic protein derived from circumsporozoite protein (CSP) of a Malaria parasite comprise an amino acid sequence stretch derived from CSP, wherein said stretch corresponds to at least 75% full length CSP, 80% full length CSP, 85% full length CSP, 86% full length CSP, 87% full length CSP, 88% full length CSP, 89% full length CSP, 90% full length CSP, 91% full length CSP, 92% full length CSP, 93% full length CSP, 94% full length CSP, 95% full length CSP, 96% full length CSP, 97% full length CSP, 98% full length CSP, 99% full length CSP, wherein the amino acid stretch is preferably derived from CSP of Plasmodium falciparum 3D7, wherein full length CSP (that is 100% full length) has a length of 397 amino acids.

“Corresponds to” in that context has to be understood as an amino acid sequence being identical, or at least 80%, 85%, 90%, 95%, 96%, 97%, 98%, 99% identical to the an amino acid sequence of CSP, in particular to the an amino acid sequence of CSP derived from Plasmodium falciparum 3D7.

In this context, preferably, a full-length CSP may be used as suitable antigen and may preferably be derived from any NCBI Protein Accession numbers provided in List 2. In preferred embodiments of the invention, the full-length CSP of Plasmodium falciparum 3D7 (SEQ ID NO: 1) is suitably used.

In preferred embodiments, a more full-length CSP may be used as suitable antigen and may preferably be derived from any NCBI Protein Accession numbers provided in List 2 or may be chosen from any one of SEQ ID NO: 1-36, 2481-2886. In preferred embodiments of the invention, a more full-length CSP of Plasmodium falciparum 3D7 derived from SEQ ID NO: 1 is suitably used.

The term “more full-length CSP” has to be understood as a CSP amino acid sequence that is in regard of the length closer to the full length amino acid sequence as compared to an RTS,S fragment (CSP(207-395); SEQ ID NO: 2112). Accordingly, a “more full-length CSP” comprises more than 190 amino acids, 200 amino acids, 220 amino acids, 240 amino acids, 260 amino acids, 280 amino acids, 300 amino acids, 320 amino acids, 340 amino acids, 360 amino acids, 380 amino acids, 390 amino acids derived from CSP, preferably derived from CSP of Plasmodium falciparum 3D7. In other words, a “more full-length CSP” comprise an amino acid sequence stretch derived from CSP, wherein said stretch corresponds to at least 75% full length CSP, 80% full length CSP, 85% full length CSP, 86% full length CSP, 87% full length CSP, 88% full length CSP, 89% full length CSP, 90% full length CSP, 91% full length CSP, 92% full length CSP, 93% full length CSP, 94% full length CSP, 95% full length CSP, 96% full length CSP, 97% full length CSP, 98% full length CSP, 99% full length CSP.

The more full-length CSP as an antigen induces broader humoral and especially cellular antibody responses compared for example to the truncated CSP (e.g. Pf-CSP(199-377)_Linker(PVTN)_HBsAg). The more full-length CSP may provide additional T cell epitopes, leading to increased cellular immunity, which could potentially enhance protection against Malaria (see e.g. Example 6, 7, 8).

SEQ ID NOs: 1-36, 2081-2120, 2481-2886, 8742-8753, 10080-10085 provide suitable CSP proteins derived from Malaria parasites; corresponding RNA coding sequences encoding said CSP proteins are provided in Table A. Additional information regarding each of these suitable amino acid sequences encoding proteins derived from Malaria parasites may also be derived from the sequence listing, in particular from the details provided therein under identifier <223> as explained in the following.

According to another preferred embodiment, the coding RNA of the invention encodes at least one antigenic Malaria peptide or protein derived from CPS as defined above and additionally at least one further heterologous peptide or protein element.

Suitably, the at least one further peptide or protein element may promote secretion of the encoded antigenic peptide or protein of the invention (e.g. via secretory signal sequences), promote anchoring of the encoded antigenic peptide or protein of the invention in the plasma membrane (e.g. via transmembrane elements), promote formation of antigen complexes (e.g. via multimerization domains), promote virus-like particle formation (VLP forming sequence). In addition, the coding RNA may additionally encode peptide linker elements, self-cleaving peptides, immunologic adjuvant sequences or dendritic cell targeting sequences. Suitable multimerization domains may be selected from the list of amino acid sequences according to SEQ ID NOs: 1116-1167 of the patent application WO2017/081082, or fragments or variants of these sequences. Trimerization and tetramerization elements may be selected from e.g. engineered leucine zippers (engineered α-helical coiled coil peptide that adopt a parallel trimeric state), fibritin foldon domain from enterobacteria phage T4, GCN4pll, GCN4-pLl, and p53. In that context, fibritin foldon domain from enterobacteria phage T4, GCN4pll, GCN4-pLl, and p53 are preferred. Suitable transmembrane elements may be selected from the list of amino acid sequences according to SEQ ID NOs: 1228-1343 of the patent application WO2017/081082, or fragments or variants of these sequences. Suitable VLP forming sequences may be selected from the list of amino acid sequences according to SEQ ID NOs: 1168-1227 of the patent application WO2017/081082, or fragments or variants of these sequences. Suitable peptide linkers may be selected from the list of amino acid sequences according to SEQ ID NOs: 1509-1565 of the patent application WO2017/081082, or fragments or variants of these sequences. Suitable self-cleaving peptides may be selected from the list of amino acid sequences according to SEQ ID NOs: 1434-1508 of the patent application WO2017/081082, or fragments or variants of these sequences. Suitable immunologic adjuvant sequences may be selected from the list of amino acid sequences according to SEQ ID NOs: 1360-1421 of the patent application WO2017/081082, or fragments or variants of these sequences. Suitable dendritic cell (DCs) targeting sequences may be selected from the list of amino acid sequences according to SEQ ID NOs: 1344-1359 of the patent application WO2017/081082, or fragments or variants of these sequences. Suitable secretory signal peptides may be selected from the list of amino acid sequences according to SEQ ID NOs: 1-1115 and SEQ ID NO: 1728 of the patent application WO2017/081082, or fragments or variants of these sequences.

Suitably, the at least one coding RNA of the invention encodes at least one antigenic Malaria peptide or protein derived from CPS as defined above and additionally at least one or more heterologous peptide or protein element selected from a heterologous secretory signal peptide, a peptide linker element, a helper epitope, an antigen clustering domain, or a transmembrane domain.

In preferred embodiments, the coding RNA of the invention additionally encodes heterologous secretory signal peptide.

In embodiments where the coding RNA of the invention additionally encodes heterologous secretory signal peptides, it is particularly preferred and suitable to generate a fusion protein comprising a heterologous N-terminal secretory signal sequence and a C-terminal peptide or protein derived from CPS, wherein said C-terminal peptide or protein derived from CPS is preferably lacking the endogenous N-terminal secretory signal peptide. Accordingly, in the context of CSP proteins, it is suitable to remove at least the first 18 amino acids (representing the Secretory signal sequence of CSP) and to fuse a heterologous N-terminal signal sequence to the CSP antigen. Such constructs may ideally improve the secretion of the CSP protein (that is encoded by the RNA of the first aspect).

Suitable secretory signal peptides may be selected from the list of amino acid sequences according to SEQ ID NOs: 1-1115 and SEQ ID NO: 1728 of the patent application WO2017/081082, or fragments or variants of these sequences, wherein said secretory signal peptides N-terminally fused to a CSP protein (or fragment) lacking the endogenous secretory signal sequence.

In some embodiments, the signal peptide is selected from: SEQ ID NOs: 423-427 of patent application WO2017/070624A1 or a fragment or variant of any of these sequences. In this context SEQ ID NOs: 423-427, of patent application WO2017/070624A1, and the disclosure related thereto, are herewith incorporated by reference.

In particularly preferred embodiments, the signal peptide is derived from human SPARC (HsSPARC) according to SEQ ID NO: 6208. In particularly preferred embodiments, the signal peptide is derived from human Insulin isoform 1 (Hslns-isol) according to SEQ ID NO: 6207. In particularly preferred embodiments, the signal peptide is derived from human albumin (HsALB) according to SEQ ID NO: 6205. In particularly preferred embodiments, the signal peptide is derived from IgE according to SEQ ID NO: 6206.

In particularly preferred embodiments the secretory signal peptide is or is derived from HsALB, wherein the amino acid sequences of said heterologous signal peptide is identical or at least 70%, 80%, 85%, 86%, 87%, 88%, 89%, 90%, 91%, 92%, 93%, 94%, 95%, 96%, 97%, 98%, or 99% identical to amino acid sequence SEQ ID NO: 6205, or fragment or variant of any of these.

In embodiments where the coding RNA of the invention additionally encodes heterologous secretory signal peptides, it is particularly preferred and suitable to generate a fusion protein comprising a heterologous N-terminal secretory signal peptide and a C-terminal peptide or protein derived from CSP wherein said C-terminal peptide or protein derived from CSP is preferably lacking an endogenous N-terminal secretory signal peptide (e.g. CSP(1-18) is lacking). Constructs comprising an N-terminal secretory signal peptide may ideally improve the secretion of the Malaria protein, preferably the CSP protein (that is encoded by the coding RNA of the first aspect). Accordingly, improved secretion of the antigen, preferably the CSP protein, upon administration of the coding RNA of the first aspect, may be advantageous for the induction of immune responses against the encoded Plasmodium antigenic protein (see e.g. Example 8, FIG. 13B, group 5 in comparison with group 6).

Accordingly, in various embodiments any CSP fragment defined by SEQ ID NOs: 2081-2120, 10080-10085 may additionally comprise a heterologous secretory signal sequence, preferably a secretory signal sequence as defined above, to generate a CSP antigen that is secreted in vivo. In particular, any CSP fragment defined by SEQ ID NOs: 2081-2120, 10080-10085 may additionally comprise an N-terminal heterologous secretory signal sequence of SEQ ID NOs: 6205-6208.

Examples of CSP constructs comprising a heterologous secretory signal sequence include but are not limited to HsALB_Pf-CSP(19-397), Hslns-iso1_Pf-CSP(19-397), HsSPARC_Pf-CSP(19-397), IgE_Pf-CSP(19-397), HsALB_Pf-CSP(19-152), HsALB_Pf-CSP(19-192), HsALB_Pf-CSP(19-272), HsALB_Pf-CSP(19-272)_Linker(AAY)_Pf-CSP(310-327)_Linker(AAY)_Pf-CSP(346-375), HsALB_Pf-CSP(19-272)_Linker(AAY)_Pf-CSP(346-365)_Linker(AAY)_P2, HsALB_Pf-CSP(19-272)_Linker(AAY)_Pf-CSP(346-365)_Linker(AAY)_PADRE, HsALB_Pf-CSP(19-272)_Linker(G4S)_Pf-CSP(310-327)_Linker(G4S)_Pf-CSP(346-375), HsALB_Pf-CSP(19-272)_Linker(G4S)_Pf-CSP(310-327)_Pf-CSP(346-375), HsALB_Pf-CSP(19-325), HsALB_Pf-CSP(19-384), HsALB_Pf-CSP(19-384)_TM domain HA, HsALB_Pf-CSP(82-397), HsALB_Pf-CSP(93-192), HsALB_Pf-CSP(93-272), HsALB_Pf-CSP(93-397), HsALB_Pf-CSP(98-192), HsALB_Pf-CSP(98-272), HsALB_Pf-CSP(98-374), HsALB_Pf-CSP(98-397), HsALB_Pf-CSP(199-377)_Linker(PVTN)_HBsAg, HsALB_Pf-CSP(19-272)_Linker(PVTN)_HBsAg, HsALB_Pf-CSP(19-272)_Linker(AAY)_Pf-CSP(346-365)_Linker(AAY)_PADRE_Linker(PVTN)_HBsAg, HsALB_Pf-CSP(19-384)_Linker(PVTN)_HBsAg, HsALB_Pf-CSP(19-384)_Linker(SGG)_Ferritin, HsALB_Pf-CSP(93-384)_Linker(PVTN)_HBsAg, HsALB_Pf-CSP(19-272)_Linker(AAY)_Pf-CSP(346-375)_Linker(AAY)_Pf-CSP(310-327), HsALB_Pf-CSP(19-272)_Linker(AAY)_Pf-CSP(346-375)_Linker(AAY)_Pf-CSP(310-327)_Linker(AAY)_PADRE, HsALB_Pf-CSP(19-272)_Linker(AAY)_Pf-CSP(310-327)_Linker(AAY)_Pf-CSP(346-375)_Linker(AAY)_PADRE, HsALB_Pf-CSP(19-272)_Linker(AAY)_Pf-CSP(310-327)_Linker(AAY)_Pf-CSP(346-375)_Linker(AAY)_PADRE_Linker(AAY)_P2, HsALB_Pf-CSP(19-272)_Linker(AAY)_Pf-CSP(310-327)_Linker (AAY)_Pf-CSP(346-397), HsALB_Pb-CSP(24-340), Hslns-iso1_Pb-CSP(24-340), HsSPARC_Pb-CSP(24-340), IgE_Pb-CSP(24-340), wherein HsALB_Pf-CSP(19-397), HsALB_Pf-CSP(19-384), HsALB_Pf-CSP(19-384)_TM domain HA, and HsALB_Pf-CSP(199-377)_HBsAg are particularly preferred. The corresponding amino acid sequences for each of the above listed constructs can be found in Table 1.

In preferred embodiments, the coding RNA of the invention additionally encodes a heterologous peptide linker element.

Accordingly, the coding RNA of the invention may comprise at least one CSP protein or fragment as defined above, and, at least one peptide linker element, wherein the peptide linker may be selected from the list of amino acid sequences according to SEQ ID NOs: 1509-1565 of the patent application WO2017/081082, or fragments or variants of these sequences.

In particularly preferred embodiments, the heterologous peptide linker element is selected from SEQ ID NOs: 6241-6244, 10141, 10147.

In suitable embodiments, the coding RNA of the invention encodes partially truncated C-terminal regions. The deletion of partial regions of the C-terminal region may protect against unwanted influences of regions, disturbing proper immune responses. It is preferred, that in these C-terminal truncated CSP proteins, CSP-derived T-cell epitopes remain present. These T-cell epitopes are suitably combined with heterologous linker sequences as described above. Suitable and preferred T-cell epitope regions are e.g. Th2R: CSP(309-327), Th2R: CSP(310-327), Th3R: CSP(346-366), Th3R: CSP(346-365), Th3R+CS.T3: CSP(346-375), Th3R+CS.T3: CSP(346-376). The aTSR domain of CSP contains several T-cell epitopes, one of which, (CS).T3, is responsible for a CD4+ T-cell response that correlates with protection. The other T-cell epitopes, Th2R and Th3R, are polymorphic regions of the aTSR (Doud, Michael B., et al. “Unexpected fold in the circumsporozoite protein target of malaria vaccines.” Proceedings of the National Academy of Sciences 109.20 (2012): 7817-7822). Preferred T-cell helper epitopes derived from C-term CSP are selected from amino acid sequences according to SEQ ID NOs: 2100, 2101, 2102, 2113, 10083, 10084.

The deletion of partial regions of the C-terminal region and the combination of T-cell epitopes with heterologous linkers may enhance the immune responses (see Example 9, FIGS. 14 and 15). Differences in the relative location and in the choice of T-cell epitope influence the type and direction of the immune responses. Some combinations results in increased humoral responses (e.g. constructs with C-terminus AAY-CSP(310-327)-AAY-CSP(346-375) and AAY-CSP(346-365)-AAY-CSP(346-375)), others in improved CD4+ and/or CD8+ T-cell responses (e.g. AAY-CSP(310-327)-AAY-CSP(346-375) and G4S-CSP(310-327)-CSP(346-375). Examples of preferred C-terminal ends include but are not limited to _Linker(AAY)_Pf-CSP(310-327)_Linker(AAY)_Pf-CSP(346-375), _Linker(AAY)_Pf-CSP(346-365)_Linker(AAY)_PADRE, _Linker(AAY)_Pf-CSP(310-327)_Linker(AAY)_Pf-CSP(346-375), _Linker(G4S)_Pf-CSP(310-327)_Pf-CSP(346-375), _Linker(G4S)_Pf-CSP(310-327)_Linker(G4S)_Pf-CSP(346-375).

Examples of CSP constructs comprising a heterologous peptide linker element include but are not limited to Pf-CSP_Linker(G4SG4)_TM domain HA, Pf-CSP(199-377)_Linker(PVTN)_HBsAg, Pf-CSP(199-387)_Linker(PVTN)_HBsAg, HsALB_Pf-CSP(19-272)_Linker(AAY)_Pf-CSP(310-327)_Linker(AAY)_Pf-CSP(346-375), HsALB_Pf-CSP(19-272)_Linker(AAY)_Pf-CSP(346-365)_Linker(AAY)_P2, HsALB_Pf-CSP(19-272)_Linker(AAY)_Pf-CSP(346-365)_Linker(AAY)_PADRE, HsALB_Pf-CSP(19-272)_Linker(G4S)_Pf-CSP(310-327)_Linker(G4S)_Pf-CSP(346-375), HsALB_Pf-CSP(19-272)_Linker(G4S)_Pf-CSP(310-327)_Pf-CSP(346-375), HsALB_Pf-CSP(199-377)_Linker(PVTN)_HBsAg, LumSynt_Linker(GGS4-GGG)_Pf-CSP(19-397), HsALB_Pf-CSP(19-272)_Linker(PVTN)_HBsAg, HsALB_Pf-CSP(19-272)_Linker(AAY)_Pf-CSP(346-365)_Linker(AAY)_PADRE_Linker(PVTN)_HBsAg, HsALB_Pf-CSP(19-384)_Linker(PVTN)_HBsAg, HsALB_Pf-CSP(19-384)_Linker(SGG)_Ferritin, HsALB_Pf-CSP(93-384)_Linker(PVTN)_HBsAg, HsALB_Pf-CSP(19-272)_Linker(AAY)_Pf-CSP(346-375)_Linker(AAY)_Pf-CSP(310-327), HsALB_Pf-CSP(19-272)_Linker(AAY)_Pf-CSP(346-375)_Linker(AAY)_Pf-CSP(310-327)_Linker(AAY)_PADRE, HsALB_Pf-CSP(19-272)_Linker(AAY)_Pf-CSP(310-327)_Linker(AAY)_Pf-CSP(346-375)_Linker(AAY)_PADRE, HsALB_Pf-CSP(19-272)_Linker(AAY)_Pf-CSP(310-327)_Linker(AAY)_Pf-CSP(346-375)_Linker(AAY)_PADRE_Linker(AAY)_P2, HsALB_Pf-CSP(19-272)_Linker(AAY)_Pf-CSP(310-327)_Linker (AAY)_Pf-CSP(346-397), Pb-CSP_Linker(G4SG4)_TM domain HA. The corresponding amino acid sequences for each of the above listed constructs can be found in Table 1.

Examples of CSP constructs comprising at least a T-cell helper epitope derived from CSP are HsALB_Pf-CSP(19-272)_Linker(AAY)_Pf-CSP(310-327)_Linker(AAY)_Pf-CSP(346-375), HsALB_Pf-CSP(19-272)_Linker(AAY)_Pf-CSP(346-365)_Linker(AAY)_P2, HsALB_Pf-CSP(19-272)_Linker(AAY)_Pf-CSP(346-365)_Linker(AAY)_PADRE, HsALB_Pf-CSP(19-272)_Linker(G4S)_Pf-CSP(310-327)_Linker(G4S)_Pf-CSP(346-375), HsALB_Pf-CSP(19-272)_Linker(G4S)_Pf-CSP(310-327)_Pf-CSP(346-375), HsALB_Pf-CSP(19-272)_Linker(AAY)_Pf-CSP(346-365)_Linker(AAY)_PADRE_Linker(PVTN)_HBsAg, HsALB_Pf-CSP(19-272)_Linker(AAY)_Pf-CSP(346-375)_Linker(AAY)_Pf-CSP(310-327), HsALB_Pf-CSP(19-272)_Linker(AAY)_Pf-CSP(346-375)_Linker(AAY)_Pf-CSP(310-327)_Linker(AAY)_PADRE, HsALB_Pf-CSP(19-272)_Linker(AAY)_Pf-CSP(310-327)_Linker(AAY)_Pf-CSP(346-375)_Linker(AAY)_PADRE, HsALB_Pf-CSP(19-272)_Linker(AAY)_Pf-CSP(310-327)_Linker(AAY)_Pf-CSP(346-375)_Linker(AAY)_PADRE_Linker(AAY)_P2, HsALB_Pf-CSP(19-272)_Linker(AAY)_Pf-CSP(310-327)_Linker (AAY)_Pf-CSP(346-397).

In preferred embodiments, the coding RNA of the invention additionally encodes at least one heterologous helper epitope. A helper epitope may enhance the immune response of the RNA encoding CSP.

In particularly preferred embodiments, the heterologous helper epitope is derived from P2 helper peptide according to SEQ ID NO: 6272. In particularly preferred embodiments, the helper epitope is derived from PADRE helper epitope according to SEQ ID NO: 6273. In particularly preferred embodiments, the helper epitope is derived from HBsAg (surface antigen of Hepatitis B virus) according to SEQ ID NO: 6274.

In embodiments, the helper epitope is or is derived from P2 tetanus toxin, from PADRE, or from HBsAg, wherein the amino acid sequences of said helper epitopes is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 6272, 6273, or 6274, or fragment or variant of any of these.

Preferably, said heterologous helper epitope is located at the C-terminus of a CSP antigen as defined above.

Preferably, the amino acid sequence of P2 helper epitope of tetanus toxin according to SEQ ID NO: 6272 (GenBank X04436.1 or NC_004565.1 derived from Kovacs-Nolan et al.; PMID 16978788; P2: aa 830-844) may serve as a basis for advantageous designs of the inventive coding RNA. The inclusion of P2 in antigens has been demonstrated to strongly influence the antibody responses to poorly immunogenic B cell epitopes. The addition of a sequence encoding a P2 helper epitope may be particularly effective in enhancing the immune response in an mRNA-based vaccine approach.

Preferably, the helper epitope is pan HLA DR-binding epitope (PADRE) or a fragment, variant or derivative thereof according to SEQ ID NO: 6273. PADRE is an immunodominant helper CD4 T-cell epitope. CD4+ T-cells play an important role in the generation of CD8+ T effector and memory T-cell immune responses. The

CD4+ T cell immune response, and thus the corresponding antigen-specific CD8+ T cell response, can be enhanced by encoding at least one antigenic protein of Plasmodium as defined herein and additionally at least the heterologous helper epitope pan HLA DR-binding epitope (PADRE). The addition of a sequence encoding a PADRE helper epitope may be particularly effective in enhancing the immune response in an mRNA-based vaccine approach.

Preferably, at least one the helper epitope is derived from HBsAg (surface antigen of Hepatitis B virus) according to SEQ ID NO: 6274 or a fragment, variant or derivative thereof according. HBsAg comprises several CD4 T cell helper epitopes (see e.g. Desombere, Isabelle, et al. “Characterization of the T cell recognition of hepatitis B surface antigen (HBsAg) by good and poor responders to hepatitis B vaccines.”

Clinical & Experimental Immunology 122.3 (2000): 390-399). CD4+ T-cells play an important role in the generation of CD8+ T effector and memory T-cell immune responses. The CD4+ T cell immune response, and thus the corresponding antigen-specific CD8+ T cell response, can be enhanced by encoding at least one antigenic protein of Plasmodium as defined herein and additionally at least one of the helper epitopes derived from HBsAg). The addition of a sequence encoding a helper epitope derived from HBsAg may be particularly effective in enhancing the immune response in an mRNA-based vaccine approach.

Examples of CSP constructs comprising a heterologous helper epitope include but are not limited to HsALB_Pf-CSP(19-272)_Linker(AAY)_Pf-CSP(346-365)_Linker(AAY)_PADRE, HsALB_Pf-CSP(19-272)_Linker(AAY)_Pf-CSP(346-365)_Linker(AAY)_PADRE_Linker(PVTN)_HBsAg, HsALB_Pf-CSP(19-272)_Linker(AAY)_Pf-CSP(346-375)_Linker(AAY)_Pf-CSP(310-327)_Linker(AAY)_PADRE, HsALB_Pf-CSP(19-272)_Linker(AAY)_Pf-CSP(310-327)_Linker(AAY)_Pf-CSP(346-375)_Linker(AAY)_PADRE, HsALB_Pf-CSP(19-272)_Linker(AAY)_Pf-CSP(310-327)_Linker(AAY)_Pf-CSP(346-375)_Linker(AAY)_PADRE_Linker(AAY)_P2, HsALB_Pf-CSP(19-272)_Linker(AAY)_Pf-CSP(346-365)_Linker(AAY)_P2, Pf-CSP(199-377)_Linker(PVTN)_HBsAg, Pf-CSP(199-387)_Linker(PVTN)_HBsAg, HsALB_Pf-CSP(199-377)_Linker(PVTN)_HBsAg, HsALB_Pf-CSP(19-272)_Linker(PVTN)_HBsAg, HsALB_Pf-CSP(19-384)_Linker(PVTN)_HBsAg, HsALB_Pf-CSP(93-384)_Linker(PVTN)_HBsAgcarrier matrix. The corresponding amino acid sequences for each of the above listed constructs can be found in Table 1.

A domain or fragment of HBsAg (surface antigen of hepatitis B virus, e.g. according to SEQ ID NO: 6274) may comprise one or more T helper epitope, but moreover the protein or a fragment thereof may function as a antigen clustering domain or as a multimerization domain.

In further preferred embodiments, the coding RNA of the invention additionally encodes an heterologous antigen clustering domain or multimerization domain.

Suitably, the antigen clustering domain (multimerization domain or scaffold moiety) is or is derived from ferritin, lumazine-synthase (LS), surface antigen of hepatitis B virus (HBsAg) or encapsulin.

Antigen clustering domain of scaffold proteins may e.g. improve the immunogenicity of an antigen, e.g., by altering the structure of the antigen, altering the uptake and processing of the antigen, and/or causing the antigen to bind to a binding partner. In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures.

In preferred embodiments, the antigen clustering domain (multimerization domain) is or is derived from surface antigen of hepatitis B virus (HBsAg), ferritin or lumazine-synthase, wherein the amino acid sequences of said antigen clustering domain is identical or at least 70%, 80%, 85%, 86%, 87%, 88%, 89%, 90%, 91%, 92%, 93%, 94%, 95%, 96%, 97%, 98%, or 99% identical to any one of amino acid sequences according to SEQ ID NOs: 6274, 10153, 10162, or a fragment or variant of any of these.

In embodiments where the coding RNA of the invention additionally encodes heterologous antigen clustering domain, it is particularly preferred and suitable to generate a fusion protein comprising a heterologous antigen clustering domain, optionally a linker element, and a peptide or protein derived from CSP. Constructs comprising an antigen clustering domain may enhance the antigen clustering and may therefore promote immune responses e.g. by multiple binding events that occur simultaneously between the clustered antigens and the host cell receptors (see further details in López-Sagaseta, Jacinto, et al. “Self-assembling protein nanoparticles in the design of vaccines”. Computational and structural biotechnology journal 14 (2016):58-68). Additionally, such constructs may additionally comprise an N-terminal secretory signal sequence (as defined above). For example, in some embodiments, the scaffold moiety is a hepatitis B surface antigen (HBsAg). HBsAg forms spherical particles. The addition of a fragment of the surface antigen of hepatitis B virus (HBsAg) sequence may be particularly effective in enhancing the immune response in an mRNA-based vaccine approach.

In particularly preferred embodiments, HBsAg is used to promote the antigen clustering and may therefore promote immune responses of the RNA encoding the Plasmodium antigen, preferably CSP or a fragment or derivative thereof.

In particularly preferred embodiments, the antigen clustering domain (multimerization domain) is or is derived from surface antigen of hepatitis B virus (HBsAg), wherein the amino acid sequences of said antigen clustering domain is preferably identical or at least 70%, 80%, 85%, 86%, 87%, 88%, 89%, 90%, 91%, 92%, 93%, 94%, 95%, 96%, 97%, 98%, or 99% identical to amino acid sequence according to SEQ ID NO: 6274, a fragment or variant of any of these.

Lumazine synthase (LS, LumSynth) is an enzyme with particle-forming properties, present in a broad variety of organisms and involved in riboflavin biosynthesis. Jardine et al reported their attempts to enhance the immunoreactivity of recombinant gp120 against HIV infection through the inclusion of Lumazine synthase (LS) for the optimization of vaccine candidates (Jardine, Joseph, et al. “Rational HIV immunogen design to target specific germline B cell receptors”. Science 340.6133 (2013):711-716).

In particularly preferred embodiments, Lumazine-synthase is used to promote the antigen clustering and may therefore promote immune responses of the RNA encoding the Plasmodium antigen, preferably CSP or a fragment or derivative thereof.

In particularly preferred embodiments, the antigen clustering domain (multimerization domain) is or is derived from Lumazine-synthase (LS), wherein the amino acid sequences of said antigen clustering domain is preferably identical or at least 70%, 80%, 85%, 86%, 87%, 88%, 89%, 90%, 91%, 92%, 93%, 94%, 95%, 96%, 97%, 98%, or 99% identical to amino acid sequence according to SEQ ID NO: 10153, a fragment or variant of any of these.

Ferritin is a protein whose main function is intracellular iron storage. Almost all living organisms produce ferritin which is made of 24 subunits, each composed of a four-alpha-helix bundle, that self-assemble in a quaternary structure with octahedral symmetry. Its properties to self-assemble into nanoparticles are well-suited to carry and expose antigens.

In particularly preferred embodiments, ferritin is used to promote the antigen clustering and may therefore promote immune responses of the RNA encoding the Plasmodium antigen, preferably CSP or a fragement or variant thereof.

In particularly preferred embodiments, the antigen clustering domain (multimerization domain) is or is derived from ferritin wherein the amino acid sequences of said antigen clustering domain is preferably identical or at least 70%, 80%, 85%, 86%, 87%, 88%, 89%, 90%, 91%, 92%, 93%, 94%, 95%, 96%, 97%, 98%, or 99% identical to amino acid sequence according to SEQ ID NO: 10162), a fragment or variant of any of these.

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers.

Suitable examples of CSP constructs comprising a heterologous antigen clustering domain include but are not limited to HsALB_Pf-CSP(19-384)_Linker(SGG)_Ferritin, LumSynt_Linker(GGS4-GGG)_Pf-CSP(19-397), Pf-CSP(199-377)_Linker(PVTN)_HBsAg, Pf-CSP(199-387)_Linker(PVTN)_HBsAg, HsALB_Pf-CSP(199-377)_Linker(PVTN)_HBsAg, HsALB_Pf-CSP(19-272)_Linker(PVTN)_HBsAg, HsALB_Pf-CSP(19-272)_Linker(AAY)_Pf-CSP(346-365)_Linker(AAY)_PADRE_Linker(PVTN)_HBsAg, HsALB_Pf-CSP(19-384)_Linker(PVTN)_HBsAg, HsALB_Pf-CSP(93-384)_Linker(PVTN)_HBsAg. The corresponding amino acid sequences for each of the above listed constructs can be found in Table 1.

Further suitable multimerization domains/antigen clustering domains may be selected from the list of amino acid sequences according to SEQ ID NOs: 1116-1167 of the patent application WO2017/081082, or fragments or variants of these sequences.

In preferred embodiments, the coding RNA of the invention additionally encodes at least one heterologous transmembrane domain.

In particularly preferred embodiments, the heterologous transmembrane domain is derived from a transmembrane domain of HA according to SEQ ID NOs: 6302.

Preferably, said heterologous transmembrane domain is located at the C-terminus of a CSP antigen as defined above.

Examples of CSP constructs comprising a heterologous transmembrane domain are Pf-CSP_Linker(G4SG4)_TM domain HA and HsALB_Pf-CSP(19-384)_TM domain HA. Example 7 shows that mRNA encoding CSP comprising a heterologous transmembrane domain induces humoral and cellular immune responses (FIGS. 10 and 11). The corresponding amino acid sequences for each of the above listed constructs can be found in Table 1.

Suitable heterologous peptide or protein elements that may be fused to a CSP antigen as defined herein and corresponding RNA coding sequences encoding said elements are provided in Table 4.

Accordingly, as outlined above, at least one antigenic protein derived from CSP of a Malaria parasite may comprise, preferably in N-terminal to C-terminal direction:
a) optionally, one N-terminal heterologous secretory signal sequence selected from SEQ ID NOs: 6205-6208 or fragments or variants thereof, wherein SEQ ID NO: 6205 is particularly preferred and
b) at least one antigenic protein derived from CSP, preferably any one of the amino acid sequences selected from SEQ ID NOs: 1-36, 2081-2120, 2481-2886, 8742-8753, 10080-10085, or fragments or variants thereof, and
c) optionally, at least one heterologous helper epitope selected from SEQ ID NOs: 6272, 6273, or 6274, or fragments or variants thereof, and
d) optionally, at least one heterologous antigen clustering domain selected from SEQ ID NOs: 6274, 10153, 10162, or fragments or variants thereof, and
e) optionally, at least one heterologous transmembrane domain selected from SEQ ID NOs: 6302 or fragments or variants thereof.

Further, a), b), c), d) and/or e) may be connected via at least one peptide linker element selected from SEQ ID NOs: 6241-6244, 10141, 10147.

A detailed description of particularly preferred and suitable CSP protein constructs is provided in Table 1 (for schematic overview see column E of Table 1 and FIG. 26).

In Table 1 all references made to amino acid (aa) residues and their position in an CSP protein relates to the position of the respective aa residue in a corresponding CSP protein of Pf(3D7) (SEQ ID NO: 1). Moreover, the abbreviations used to describe suitable CSP antigen designs of Table 1 are also used throughout the description of the invention (as described above) as well as in the ST25 sequence listing. Column A of Table 1 provides a short description of suitable CSP antigen designs. Column B of Table 1 indicates the amino acid stretch or stretches for each of the respective antigen designs corresponding to the full length CSP reference (SEQ ID NO: 1). Column C of Table 1 indicates the percentage of amino acid sequence that corresponds to the full length CSP reference (SEQ ID NO: 1). Column D of Table 1 provides protein SEQ ID NOs of respective CSP antigen designs derived from Pf(3D7). Notably, the description of the invention explicitly includes the information provided under <223> identifier of the ST25 sequence listing of the present application (“L” is read for “Linker”). Column E of Table 1 links the CSP antigen design to its respective schematic view shown in FIG. 26. Preferred RNA sequences encoding of the constructs of Table 1 are provided in Table 5.

TABLE 1
Preferred CSP antigen designs

| A | B | C | D | E |
|---|---|---|---|---|
| Pf-CSP | aa1-397 | 100.00 | 1 | 1 |
| Pf-CSP(1-374) | aa1-374 | 94.21 | 2 | |
| Pf-CSP(1-325) | aa1-325 | 81.86 | 3 | |
| HsALB_Pf-CSP(19-397) | aa19-397 | 95.47 | 4 | 2 |
| HsIns-iso1_Pf-CSP(19-397) | aa19-397 | 95.47 | 5 | 2 |
| HsSPARC_Pf-CSP(19-397) | aa19-397 | 95.47 | 6 | 2 |
| IgE_Pf-CSP(19-397) | aa19-397 | 95.47 | 7 | 2 |
| Pf-CSP_Linker(G4SG4)_TM domain HA | aa1-397 | 100.00 | 8 | |
| Pf-CSP(199-377)_Linker(PVTN)_HBsAg | aa199-377 | 45.09 | 9 | 4 |
| Pf-CSP(199-387)_Linker(PVTN)_HBsAg | aa199-387 | 47.61 | 10 | |
| HsALB_Pf-CSP(19-152) | aa19-152 | 33.75 | 11 | 12 |
| HsALB_Pf-CSP(19-192) | aa19-192 | 43.83 | 12 | 11 |
| HsALB_Pf-CSP(19-272) | aa19-272 | 63.98 | 13 | 10 |
| HsALB_Pf-CSP(19-272)_Linker(AAY)_Pf-CSP(310- | aa19-272, aa310- | 76.07 | 14 | 16 |
| 327)_Linker(AAY)_Pf-CSP(346-375) | 327, aa346-375 | | | |
| HsALB_Pf-CSP(19-272)_Linker(AAY)_Pf-CSP(346- | aa19-272, aa346- | 69.02 | 15 | 24 |
| 365)_Linker(AAY)_P2 | 365 | | | |
| HsALB_Pf-CSP(19-272)_Linker(AAY)_Pf-CSP(346- | aa19-272, aa346- | 69.02 | 16 | 23 |
| 365)_Linker(AAY)_PADRE | 365 | | | |
| HsALB_Pf-CSP(19-272)_Linker(G4S)_Pf-CSP(310- | aa19-272, aa310- | 76.07 | 17 | 17 |
| 327)_Linker(G4S)_Pf-CSP(346-375) | 327, aa346-375 | | | |
| HsALB_Pf-CSP(19-272)_Linker(G4S)_Pf-CSP(310-327)_ | aa19-272, aa310- | 76.07 | 18 | 18 |
| Pf-CSP(346-375) | 327, aa346-375 | | | |
| HsALB_Pf-CSP(19-325) | aa19-325 | 77.33 | 19 | |
| HsALB_Pf-CSP(19-384) | aa19-384 | 92.19 | 20 | 9 |
| HsALB_Pf-CSP(19-384)_TM domain HA | aa19-384 | 92.19 | 21 | 3 |
| HsALB_Pf-CSP(82-397) | aa82-397 | 79.60 | 22 | 15 |
| HsALB_Pf-CSP(93-192) | aa93-192 | 25.19 | 23 | |
| HsALB_Pf-CSP(93-272) | aa93-272 | 45.34 | 24 | |
| HsALB_Pf-CSP(93-397) | aa93-397 | 76.83 | 25 | 14 |
| HsALB_Pf-CSP(98-192) | aa98-192 | 23.93 | 26 | |
| HsALB_Pf-CSP(98-272) | aa98-272 | 44.08 | 27 | |
| HsALB_Pf-CSP(98-374) | aa98-374 | 69.77 | 28 | |
| HsALB_Pf-CSP(98-397) | aa98-397 | 75.57 | 29 | 13 |
| HsALB_Pf-CSP(199-377)_Linker(PVTN)_HBsAg | aa199-377 | 45.09 | 30 | 5 |
| LumSynt_Linker(GGS4-GGG)_Pf-CSP(19-397) | aa19-397 | 95.47 | 8742 | 28 |
| HsALB_Pf-CSP(19-272)_Linker(PVTN)_HBsAg | aa19-272 | 63.98 | 8743 | 7 |
| HsALB_Pf-CSP(19-272)_Linker(AAY)_Pf-CSP(346- | aa19-272, aa346- | 69.02 | 8744 | 26 |
| 365)_Linker(AAY)_PADRE_Linker(PVTN)_HBsAg | 365 | | | |
| HsALB_Pf-CSP(19-384)_Linker(PVTN)_HBsAg | aa19-384 | 92.19 | 8745 | 6 |
| HsALB_Pf-CSP(19-384)_Linker(SGG)_Ferritin | aa19-384 | 92.19 | 8746 | 27 |
| HsALB_Pf-CSP(93-384)_Linker(PVTN)_HBsAg | aa93-384 | 73.55 | 8747 | 8 |
| HsALB_Pf-CSP(19-272)_Linker(AAY)_Pf-CSP(346- | aa19-272, aa346- | 73.55 | 8749 | 22 |
| 375)_Linker(AAY)_Pf-CSP(310-327) | 375, aa310-327 | | | |
| HsALB_Pf-CSP(19-272)_Linker(AAY)_Pf-CSP(346- | aa19-272, aa346- | 73.55 | 8750 | 25 |
| 375)_Linker(AAY)_Pf-CSP(310-327)_Linker(AAY)_PADRE | 375, aa310-327 | | | |
| HsALB_Pf-CSP(19-272)_Linker(AAY)_Pf-CSP(310- | aa19-272, aa310- | 73.55 | 8751 | 19 |
| 327)_Linker(AAY)_Pf-CSP(346-375)_Linker(AAY)_PADRE | 327, aa346-375 | | | |
| HsALB_Pf-CSP(19-272)_Linker(AAY)Pf-CSP(310- | aa19-272, aa310- | 73.55 | 8752 | 20 |
| 327)_Linker(AAY)_Pf-CSP(346- | 327, aa346-375 | | | |
| 375)_Linker(AAY)_PADRE_Linker(AAY)_P2 | | | | |
| HsALB_Pf-CSP(19-272)_Linker(AAY)_Pf-CSP(310-327)_Linker | aa19-272, aa310- | 73.55 | 8753 | 21 |
| (AAY)_Pf-CSP(346-397) | 327, aa346-397 | | | |
| Pb-CSP | aa1-340 | 85.64 | 31 | |
| Pb-CSP_Linker(G4SG4)_TM domain HA | aa1-340 | 85.64 | 32 | |
| HsALB_Pb-CSP(24-340) | aa24-340 | 79.85 | 33 | |
| HsIns-iso1_Pb-CSP(24-340) | aa24-340 | 79.85 | 34 | |
| HsSPARC_Pb-CSP(24-340) | aa24-340 | 79.85 | 35 | |
| IgE_Pb-CSP(24-340) | aa24-340 | 79.85 | 36 | |

In various embodiments, the RNA of the first aspect comprises at least one coding sequence encoding at least one antigenic peptide or protein comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-36, 2081-2120, 2481-2886, 8742-8753, 10080-10085, or an immunogenic fragment or immunogenic variant of any of these sequences. Additional information regarding each of these suitable amino acid sequences encoding CSP antigens may also be derived from the sequence listing, in particular from the details provided therein under identifier <223> as explained in the following.

In preferred embodiments, the RNA of the first aspect comprises at least one coding sequence encoding at least one antigenic peptide or protein comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1, 31, 2081, 2481-2886, or an immunogenic fragment or immunogenic variant of any of these sequences. Additional information regarding each of these suitable amino acid sequences encoding CSP antigens may also be derived from the sequence listing, in particular from the details provided therein under identifier <223> as explained in the following.

In particularly preferred embodiments, the RNA of the first aspect comprises at least one coding sequence encoding at least one antigenic peptide or protein comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 1-36, 8742-8753 (see e.g. Table 1), or an immunogenic fragment or immunogenic variant of any of these sequences. Additional information regarding each of these suitable amino acid sequences encoding CSP antigens may also be derived from the sequence listing, in particular from the details provided therein under identifier <223> as explained in the following.

In other embodiments, the RNA of the first aspect comprises at least one coding sequence encoding at least one antigenic peptide or protein comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 13-17 of patent application WO2017/070624A1 or a fragment or variant of any of these sequences. In this context SEQ ID NOs: 13-17, of patent application WO2017/070624A1, and the disclosure related thereto, are herewith incorporated by reference.

In various embodiments, the RNA of the first aspect comprises at least one coding sequence encoding at least one antigenic peptide or protein derived from circumsporozoite protein (CSP) of a Malaria parasite, or an immunogenic fragment or immunogenic variant thereof, wherein the amino acid sequence is mutated/substituted to delete at least one predicted or potential glycosylation site.

It may suitable in the context of the invention that glycosylation sites in the encoded amino acid sequence are mutated/substituted which means that encoded amino acids which may be glycosylated, e.g. after translation of the coding RNA upon in vivo administration, are exchanged to a different amino acid. Accordingly, on nucleic acid level, codons encoding asparagine which are predicted to be glycosylated (N glycosylation sites) are substituted with codons encoding glutamine.

By mutation/substitution of the relevant amino acids, glycosylation may be prevented. In this context at least one codon coding for an asparagine, arginine, serine, threonine, tyrosine, lysine, proline or tryptophan is modified in such a way that a different amino acid is encoded thereby deleting at least one predicted or potential glycosylation site. The predicted glycosylation sites may be predicted by using artificial neural networks that examine the sequence for common glycosylation sites, e.g. N-glycosylation sites may be predicted by using the NetNGlyc 1.0 Server.

In preferred embodiments, the at least one antigenic protein derived from circumsporozoite protein (CSP) of a Malaria parasite, or an immunogenic fragment or immunogenic variant thereof, is mutated to delete at least one predicted or potential glycosylation site, e.g. asparagine (N) is substituted by a glutamine (Q). Accordingly, on nucleic acid level, the nucleic acid sequence is modified to encode for Q instead of N at predicted N-glycosylation sites, for example at predicted N-glycosylation sites of the encoded CSP protein, or a fragment, variant or derivative thereof. In this context the term “mutated CSP” means that at least one (predicted) glycosylation site is mutated.

In various embodiments, the amino acid sequences of the at least one antigenic protein derived from circumsporozoite protein (CSP) of a Malaria parasite, or an immunogenic fragment or immunogenic variant thereof is mutated to delete all predicted or potential glycosylation sites.

### Suitable Coding Sequences:

According to preferred embodiments, the coding RNA comprises at least one coding sequence encoding at least one antigenic peptide or protein derived from CSP as defined herein, or fragments and variants thereof. In that context, any coding sequence encoding at least one antigenic peptide or protein derived from CSP, preferably derived from CSP from Pf(3D7), or fragments and variants thereof may be understood as suitable coding sequence and may therefore be comprised in the coding RNA of the first aspect.

In preferred embodiments, the coding RNA of the first aspect may comprise or consist of at least one coding sequence encoding at least one antigenic peptide or protein derived from CSP as defined herein, preferably encoding any one of SEQ ID NOs: 1-36, 2081-2120, 2481-2886, 8742-8753, 10080-10085 or fragments of variants thereof.

It has to be understood that, on nucleic acid level, any nucleic acid sequence, in particular, any RNA sequence which encodes an amino acid sequences being identical to SEQ ID NOs: 1-36, 2081-2120, 2481-2886, 8742-8753, 10080-10085 or fragments or variants thereof, or any nucleic acid sequence (e.g. DNA sequence, RNA sequence) which encodes amino acid sequences being at least 70%, 80%, 85%, 86%, 87%, 88%, 89%, 90%, 91%, 92%, 93%, 94%, 95%, 96%, 97%, 98%, or 99% identical to any one of SEQ ID NOs: 1-36, 2081-2120, 2481-2886, 8742-8753, 10080-10085 or fragments or variants thereof, may be selected and may accordingly be understood as suitable coding sequence and may therefore be comprised in the coding RNA of the first aspect.

In preferred embodiments, the coding RNA of the first aspect may comprise or consist of at least one coding sequence encoding any one of SEQ ID NO: 1-36, 8742-8753 or fragments of variants thereof. It has to be understood that, on nucleic acid level, any nucleic acid sequence, in particular, any RNA sequence which encodes an amino acid sequences being identical to SEQ ID NO: 1-36, 8742-8753 or fragments or variants thereof, or any nucleic acid sequence (e.g. DNA sequence, RNA sequence) which encodes amino acid sequences being at least 70%, 80%, 85%, 86%, 87%, 88%, 89%, 90%, 91%, 92%, 93%, 94%, 95%, 96%, 97%, 98%, or 99% identical to any one of SEQ ID NO: 1-36, 8742-8753 or fragments or variants thereof, may be selected and may accordingly be understood as suitable coding sequence and may therefore be comprised in the coding RNA of the first aspect.

In other embodiments, the coding RNA of the first aspect may comprise or consist of at least one coding sequence encoding any one of SEQ ID NOs: 13-17 of patent application WO2017/070624A1 or fragments of variants thereof. It has to be understood that, on nucleic acid level, any nucleic acid sequence, in particular, any RNA sequence which encodes an amino acid sequences being identical to SEQ ID NOs: 13-17 of patent application WO2017/070624A1 or fragments or variants thereof, or any nucleic acid sequence (e.g. DNA sequence, RNA sequence) which encodes amino acid sequences being at least 70%, 80%, 85%, 86%, 87%, 88%, 89%, 90%, 91%, 92%, 93%, 94%, 95%, 96%, 97%, 98%, or 99% identical to any one of SEQ ID NOs: 13-17 of patent application WO2017/070624A1 or fragments or variants thereof, may be selected and may accordingly be understood as suitable coding sequence and may therefore be comprised in the coding RNA of the first aspect.

In preferred embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 37-328, 2121-2480, 2887-6134, 8754-8855, 10086-10139 or a fragment or a fragment or variant of any of these sequences.

Additional information regarding each of these suitable nucleic acid sequences encoding may also be derived from the sequence listing, in particular from the details provided therein under identifier <223>.

In particularly preferred embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 37-328, 8754-8855, or a fragment or a fragment or variant of any of these sequences. Additional information regarding each of these suitable nucleic acid sequences encoding may also be derived from the sequence listing, in particular from the details provided therein under identifier <223>.

In further preferred embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 37, 40, 41, 71, 77, 107, 113, 143, 149, 179, 185, 215, 221, 251, 257, 287, 293, 323, 2121, 2161, 2201, 2241, 2281, 2321, 2361, 2401, 2441, 2887-6134, or a fragment or a fragment or variant of any of these sequences. Additional information regarding each of these suitable nucleic acid sequences encoding may also be derived from the sequence listing, in particular from the details provided therein under identifier <223>.

In further preferred embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 44, 80, 116, 152, 188, 224, 260, 296, 8755, or a fragment or a fragment or variant of any of these sequences (encoding HsALB_Pf-CSP(19-397)). Additional information regarding each of these suitable nucleic acid sequences encoding may also be derived from the sequence listing, in particular from the details provided therein under identifier <223>.

In preferred embodiments, the coding RNA of the first aspect is an artificial RNA.

The term “artificial RNA” as used herein is intended to refer to an RNA that does not occur naturally. In other words, an artificial RNA may be understood as a non-natural nucleic acid molecule. Such RNA molecules may be non-natural due to its individual sequence (e.g. G/C content modified coding sequence, UTRs) and/or due to other modifications, e.g. structural modifications of nucleotides. Typically, artificial RNA may be designed and/or generated by genetic engineering to correspond to a desired artificial sequence of nucleotides (i.e., heterologous sequence). In this context an artificial RNA is a sequence that may not occur naturally, i.e. it differs from the wild type sequence by at least one nucleotide. The term “artificial RNA” is not restricted to mean “one single molecule” but is understood to comprise an ensemble of essentially identical molecules. Accordingly, it may relate to a plurality of essentially identical RNA molecules. The RNA of the invention is preferably an artificial RNA.

In preferred embodiments, the coding RNA of the first aspect is a modified and/or stabilized artificial RNA.

According to preferred embodiments, the RNA of the present invention may thus be provided as a “stabilized artificial RNA” or “stabilized coding RNA” that is to say an RNA showing improved resistance to in vivo degradation and/or an RNA showing improved stability in vivo, and/or an RNA showing improved translatability in vivo. In the following, specific suitable modifications in this context are described which are suitably to “stabilize” the RNA.

Such stabilization may be effected by providing a “dried RNA” and/or a “purified RNA” as specified herein. Alternatively or in addition to that, such stabilization can be effected, for example, by a modified phosphate backbone of the coding RNA of the present invention. A backbone modification in connection with the present invention is a modification in which phosphates of the backbone of the nucleotides contained in the RNA are chemically modified. Nucleotides that may be preferably used in this connection contain e.g. a phosphorothioate-modified phosphate backbone, preferably at least one of the phosphate oxygens contained in the phosphate backbone being replaced by a sulfur atom. Stabilized RNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without implying any limitation, modifications from the group consisting of methylphosphonates, phosphoramidates and phosphorothioates (e.g. cytidine-5″-O-(1-thiophosphate)).

In the following, suitable modifications are described that are capable of “stabilizing” the RNA of the invention.

According to embodiments, the RNA is a modified RNA, wherein the modification refers to chemical modifications comprising backbone modifications as well as sugar modifications or base modifications.

A modified RNA may comprise nucleotide analogues/modifications, e.g. backbone modifications, sugar modifications or base modifications. A backbone modification in the context of the invention is a modification, in which phosphates of the backbone of the nucleotides of the RNA are chemically modified. A sugar modification in the context of the invention is a chemical modification of the sugar of the nucleotides of the RNA. Furthermore, a base modification in the context of the invention is a chemical modification of the base moiety of the nucleotides of the RNA. In this context, nucleotide analogues or modifications are preferably selected from nucleotide analogues which are applicable for transcription and/or translation.

In particularly preferred embodiments, the nucleotide analogues/modifications which may be incorporated into a modified RNA as described herein are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-undine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

In some embodiments, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In some embodiments, 100% of the uracil in the coding sequence have a chemical modification, preferably a chemical modification is in the 5-position of the uracil.

Particularly preferred in the context of the invention are pseudouridine (ψ), N1-methylpseudouridine (m1ψ), 5-methylcytosine, and 5-methoxyuridine. Accordingly, the RNA of the first aspect may comprise at least one modified nucleotide selected from pseudouridine (ψ), N1-methylpseudouridine (m1ψ), 5-methylcytosine, and 5-methoxyuridine.

In preferred embodiments, the RNA comprises at least one codon modified coding sequence.

In preferred embodiments, the amino acid sequence encoded by the at least one codon modified coding sequence is preferably not being modified compared to the amino acid sequence encoded by the corresponding wild type coding sequence.

The term “codon modified coding sequence” relates to coding sequences that differ in at least one codon (triplets of nucleotides coding for one amino acid) compared to the corresponding wild type coding sequence. Suitably, a codon modified coding sequence in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably (cf. Table 2) to optimize/modify the coding sequence for in vivo applications as outlined above.

In particularly preferred embodiments of the first aspect, the at least one sequence is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof, or any combination thereof.

In preferred embodiments, the RNA may be modified, wherein the C content of the at least one coding sequence may be increased, preferably maximized, compared to the C content of the corresponding wild type coding sequence (herein referred to as “C maximized coding sequence”). The amino acid sequence encoded by the C maximized coding sequence of the RNA is preferably not modified as compared to the amino acid sequence encoded by the respective wild type nucleic acid coding sequence. The generation of a C maximized nucleic acid sequences may suitably be carried out using a modification method according to WO2015/062738. In this context, the disclosure of WO2015/062738 is included herewith by reference. Throughout the description, including the <223> identifier of the sequence listing, C maximized coding sequences are indicated by the abbreviation “opt2”.

In embodiments, the RNA may be modified, wherein the G/C content of the at least one coding sequence may be modified compared to the G/C content of the corresponding wild type coding sequence (herein referred to as “G/C content modified coding sequence”). In this context, the terms “G/C optimization” or “G/C content modification” relate to an RNA that comprises a modified, preferably an increased number of guanosine and/or cytosine nucleotides as compared to the corresponding wild type RNA sequence. Such an increased number may be generated by substitution of codons containing adenosine or thymidine nucleotides by codons containing guanosine or cytosine nucleotides. Advantageously, RNA sequences having an increased G (guanosine)/C (cytosine) content are more stable than sequences having an increased A (adenosine)/U (uracil) content. The amino acid sequence encoded by the G/C content modified coding sequence of the RNA is preferably not modified as compared to the amino acid sequence encoded by the respective wild type sequence. Preferably, the G/C content of the coding sequence of the RNA sequence is increased by at least 10%, 20%, 30%, preferably by at least 40% compared to the G/C content of the coding sequence of the corresponding wild type RNA sequence.

In preferred embodiments, the RNA may be modified, wherein the G/C content of the at least one coding sequence may be optimized compared to the G/C content of the corresponding wild type coding sequence (herein referred to as “G/C content optimized coding sequence”). “Optimized” in that context refers to a coding sequence wherein the G/C content is preferably increased to the essentially highest possible G/C content. The amino acid sequence encoded by the G/C content optimized coding sequence of the RNA is preferably not modified as compared to the amino acid sequence encoded by the respective wild type coding sequence. The generation of a G/C content optimized RNA sequence may be carried out using a G/C content optimization method according to WO2002/098443. In this context, the disclosure of WO2002/098443 is included in its full scope in the present invention. Throughout the description, including the <223> identifier of the sequence listing, G/C optimized coding sequences are indicated by the abbreviations “opt1, opt5, opt6, opt11”.

In embodiments, the RNA may be modified, wherein the codons in the at least one coding sequence may be adapted to human codon usage (herein referred to as “human codon usage adapted coding sequence”). Codons encoding the same amino acid occur at different frequencies in a subject, e.g. a human. Accordingly, the coding sequence of the RNA is preferably modified such that the frequency of the codons encoding the same amino acid corresponds to the naturally occurring frequency of that codon according to the human codon usage e.g. as shown in Table 2. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see Table 2). Accordingly, such a procedure (as exemplified for Ala) is applied for each amino acid encoded by the coding sequence of the RNA to obtain sequences adapted to human codon usage. Throughout the description, including the <223> identifier of the sequence listing, human codon usage adapted coding sequences are indicated by the abbreviation “opt3”.

TABLE 2
Human codon usage table with frequencies indicated for each amino acid

| Amino | | | Amino | | |
|---|---|---|---|---|---|
| acid | codon | frequency | acid | codon | frequency |
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Thr | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0.42 |
| Leu | CTC | 0.20 | Tyr | TAC* | 0.58 |
| Met | ATG* | 1 | Stop | TGA* | 0.61 |
| Asn | AAT | 0.44 | Stop | TAG | 0.17 |
| Asn | AAC* | 0.56 | Stop | TAA | 0.22 |

*most frequent human codon

In embodiments, the RNA may be modified, wherein the codon adaptation index (CAI) may be increased or preferably maximised in the at least one coding sequence (herein referred to as “CAI maximized coding sequence”). Accordingly, it is preferred that all codons of the wild type nucleic acid sequence that are relatively rare in e.g. a human cell are exchanged for a respective codon that is frequent in the e.g. a human cell, wherein the frequent codon encodes the same amino acid as the relatively rare codon. Suitably, the most frequent codons are used for each encoded amino acid (see Table 2, most frequent human codons are marked with asterisks). Suitably, the RNA of the first aspect comprises at least one coding sequence, wherein the codon adaptation index (CAI) of the at least one coding sequence is at least 0.5, at least 0.8, at least 0.9 or at least 0.95. Most preferably, the codon adaptation index (CAI) of the at least one coding sequence is 1. For example, in the case of the amino acid Ala, the wild type coding sequence is adapted in a way that the most frequent human codon “GCC” is always used for said amino acid. Accordingly, such a procedure (as exemplified for Ala) is applied for each amino acid encoded by the coding sequence of the RNA to obtain CAI maximized coding sequences. Throughout the description, including the <223> identifier of the sequence listing, CAI maximized coding sequences are indicated by the abbreviation “opt4”.

In preferred embodiments, the RNA of the first aspect comprises at least one coding sequence comprising a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 41-328, 2161-2480, 3293-6134, 8754-8855, 10092-10139 or a fragment or variant of any of these sequences. Additional information regarding each of these suitable nucleic acid sequences encoding may also be derived from the sequence listing, in particular from the details provided therein under identifier <223>.

In particularly preferred embodiments, the RNA of the first aspect comprises at least one coding sequence comprising a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 41-328, 8754-8855 or a fragment or variant of any of these sequences. Additional information regarding each of these suitable nucleic acid sequences encoding may also be derived from the sequence listing, in particular from the details provided therein under identifier <223>.

In preferred embodiments, the coding RNA of the first aspect comprises at least one coding sequence comprising a codon modified nucleic acid sequence which is identical or at least 50%, 60%, 70%, 80%, 85%, 86%, 87%, 88%, 89%, 90%, 91%, 92%, 93%, 94%, 95%, 96%, 97%, 98%, or 99% identical to any one of the G/C optimized nucleic acid sequence according to the SEQ ID NOs: 41-112, 2161-2240, 3293-3698, 8754-8783, 10092-10103 or a fragment or variant of any of these sequences (“opt1”). Additional information regarding each of these suitable nucleic acid sequences encoding may also be derived from the sequence listing, in particular from the details provided therein under identifier <223>.

In particularly preferred embodiments, the coding RNA of the first aspect comprises at least one coding sequence comprising a codon modified nucleic acid sequence which is identical or at least 50%, 60%, 70%, 80%, 85%, 86%, 87%, 88%, 89%, 90%, 91%, 92%, 93%, 94%, 95%, 96%, 97%, 98%, or 99% identical to any one of the G/C optimized nucleic acid sequence according to the SEQ ID NOs: 41-112, 8754-8783 or a fragment or variant of any of these sequences (“opt1”). Additional information regarding each of these suitable nucleic acid sequences encoding may also be derived from the sequence listing, in particular from the details provided therein under identifier <223>.

In particularly preferred embodiments, the coding RNA of the first aspect comprises at least one coding sequence comprising a codon modified nucleic acid sequence which is identical or at least 50%, 60%, 70%, 80%, 85%, 86%, 87%, 88%, 89%, 90%, 91%, 92%, 93%, 94%, 95%, 96%, 97%, 98%, or 99% identical to any one of the G/C optimized nucleic acid sequence according to the SEQ ID NOs: 44, 80, 8755, or a fragment or variant of any of these sequences (“opt1”). Additional information regarding each of these suitable nucleic acid sequences encoding may also be derived from the sequence listing, in particular from the details provided therein under identifier <223>.

As outlined above, the coding RNA of the first aspect comprises at least one coding sequence comprising a nucleic acid sequence encoding a CSP of a malaria parasite. Preferably said CSP is a more full length CSP as defined herein, more preferably a full length CSP as defined herein. Said CSP is preferably derived from Plasmodium falciparum (Pf). Alternatively, the CSP may be derived from Plasmodium knowlesi (Pk), Plasmodium malariae (Pm), Plasmodium ovale curtisi (Poc), Plasmodium ovale wallikeri (Pow), Plasmodium ovale (Po), Plasmodium vivax (Pv), Plasmodium berghei (Pb).

CSP proteins and coding sequences of malaria parasites are disclosed in Table A. Therein, rows 1-7 corresponds to CSP derived from the indicated malaria parasite species. The respective species is provided in Column A (abbreviations, see e.g. List 1). Column B of Table A provides the SEQ ID NO of the corresponding amino acid sequences, column C of Table A provides the SEQ ID NO of the corresponding wild type RNA coding sequences, and column D-J of Table A provides the SEQ ID NO of the codon modified coding sequences for each fragment (in the following order: “opt1”, “opt2”, “opt3”, “opt4”, “opt5”, “opt6”, “opt11”). Additional information regarding each of these suitable sequences may also be derived from the sequence listing, in particular from the details provided therein under identifier <223>.

TABLE A
CSP antigens and respective coding sequences:

| | A | B | C | D | E | F | G | H | I | J |
|---|---|---|---|---|---|---|---|---|---|---|
| 1 | Pf, Pk, | 1, 31, | 37, 40, | 41, 71, 77, | 113, | 149, | 185, | 221, | 257, | 293, |
| | Pm, Poc, | 2081, | 2121, | 107, 2161, | 143, | 179, | 215, | 251, | 287, | 323, |
| | Pow, Po, | 2481- | 2887- | 2201, 3293- | 2241, | 2281, | 2321, | 2361, | 2401, | 2441, |
| | Pv, Pb | 2886 | 3292 | 3698 | 3699- | 4105- | 4511- | 4917- | 5323- | 5729- |
| | | | | | 4104 | 4510 | 4916 | 5322 | 5728 | 6134 |
| 2 | Pf | 1, | 37, 2121, | 41, 77, 2161, | 113, | 149, | 185, | 221, | 257, | 293, |
| | | 2081, | 2887- | 2201, 3293- | 2241, | 2281, | 2321, | 2361, | 2401, | 2441, |
| | | 2481- | 3094 | 3500 | 3699- | 4105- | 4511- | 4917- | 5323- | 5729- |
| | | 2688 | | | 3906 | 4312 | 4718 | 5124 | 5530 | 5936 |
| 3 | Pk | 2689- | 3095- | 3501-3625 | 3907- | 4313- | 4719- | 5125- | 5531- | 5937- |
| | | 2813 | 3219 | | 4031 | 4437 | 4843 | 5249 | 5655 | 6061 |
| 4 | Pm | 2814- | 3220- | 3626-3630 | 4032- | 4438- | 4844- | 5250- | 5656- | 6062- |
| | | 2818 | 3224 | | 4036 | 4442 | 4848 | 5254 | 5660 | 6066 |
| 5 | Poc, | 2819- | 3225- | 3631-3636 | 4037- | 4443- | 4849- | 5255- | 5661- | 6067- |
| | Pow, Po | 2824 | 3230 | | 4042 | 4448 | 4854 | 5260 | 5666 | 6072 |
| 6 | Pv | 2825- | 3231- | 3637-3697 | 4043- | 4449- | 4855- | 5261- | 5667- | 6073- |
| | | 2885 | 3291 | | 4103 | 4509 | 4915 | 5321 | 5727 | 6133 |
| 7 | Pb | 31, | 40, 3292 | 71, 107, | 143, | 179, | 215, | 251, | 287, | 323, |
| | | 2886 | | 3698 | 4104 | 4510 | 4916 | 5322 | 5728 | 6134 |

CSP fragments, e.g. fragments described above (E1-E27) are disclosed in Table 3. Therein, each row (row 1-46) corresponds to a certain fragment of CSP derived from Pf(3D7), wherein e.g. row 1 represents the full length CSP. All amino acid positions described in the present descriptions are in relation to the amino acid positions of CSP from Pf(3D7) (row 1, column B, SEQ ID NO: 1). Column A Table 3 provides a description of the fragments, with an indication of the amino acid position in relation to the full length protein. For example, sequences provided in row 4 “CSP(19-397) CSP-delSP” relate to a CSP fragment ranging from amino acid position 19 to amino acid position 397, characterized in that the construct lacks the signal peptide (“delSP”). Column B Table 3 provides the SEQ ID NO of the corresponding amino acid sequences, column C Table 3 provides the SEQ ID NO of the corresponding wild type RNA coding sequences, and column D Table 3 provides the SEQ ID NO of the codon modified coding sequences for each fragment (in the following order: “opt1”, “opt1”, “opt2”, “opt3”, “opt4”, “opt5”, “opt6”, “opt11”). Additional information regarding each of these suitable sequences may also be derived from the sequence listing, in particular from the details provided therein under identifier <223>.

TABLE 3
CSP fragments and respective coding sequences:

| | A | B | C | D |
|---|---|---|---|---|
| 1 | CSP | 1 | 2121 | 2161, 2201, 2241, 2281, 2321, 2361, 2401, 2441 |
| 2 | CSP(1-374)_CSP-delGPI | 2082 | 2122 | 2162, 2202, 2242, 2282, 2322, 2362, 2402, 2442 |
| 3 | CSP(1-325)_CSP-delTSR(v2)- | 2083 | 2123 | 2163, 2203, 2243, 2283, 2323, 2363, 2403, 2443 |
| | delGPI | | | |
| 4 | CSP(19-397)_CSP-delSP | 2084 | 2124 | 2164, 2204, 2244, 2284, 2324, 2364, 2404, 2444 |
| 5 | CSP(19-374)_CSP-delSP-delGPI | 2085 | 2125 | 2165, 2205, 2245, 2285, 2325, 2365, 2405, 2445 |
| 6 | CSP(19-325)_CSP-delSP- | 2086 | 2126 | 2166, 2206, 2246, 2286, 2326, 2366, 2406, 2446 |
| | delTSR(v2)-delGPI | | | |
| 7 | CSP(19-135) | 2087 | 2127 | 2167, 2207, 2247, 2287, 2327, 2367, 2407, 2447 |
| 8 | CSP(19-193) | 2088 | 2128 | 2168, 2208, 2248, 2288, 2328, 2368, 2408, 2448 |
| 9 | CSP(19-272) | 2089 | 2129 | 2169, 2209, 2249, 2289, 2329, 2369, 2409, 2449 |
| 10 | CSP(19-384) | 2090 | 2130 | 2170, 2210, 2250, 2290, 2330, 2370, 2410, 2450 |
| 11 | CSP(82-397) | 2091 | 2131 | 2171, 2211, 2251, 2291, 2331, 2371, 2411, 2451 |
| 12 | CSP(93-192) | 2092 | 2132 | 2172, 2212, 2252, 2292, 2332, 2372, 2412, 2452 |
| 13 | CSP(93-272)_RI region_NANP | 2093 | 2133 | 2173, 2213, 2253, 2293, 2333, 2373, 2413, 2453 |
| | repeat region | | | |
| 14 | CSP(93-397) | 2094 | 2134 | 2174, 2214, 2254, 2294, 2334, 2374, 2414, 2454 |
| 15 | CSP(98-192) | 2095 | 2135 | 2175, 2215, 2255, 2295, 2335, 2375, 2415, 2455 |
| 16 | CSP(98-374) | 2096 | 2136 | 2176, 2216, 2256, 2296, 2336, 2376, 2416, 2456 |
| 17 | CSP(98-397) | 2097 | 2137 | 2177, 2217, 2257, 2297, 2337, 2377, 2417, 2457 |
| 18 | CSP(199-377) | 2098 | 2138 | 2178, 2218, 2258, 2298, 2338, 2378, 2418, 2458 |
| 19 | CSP(199-387) | 2099 | 2139 | 2179, 2219, 2259, 2299, 2339, 2379, 2419, 2459 |
| 20 | CSP(309-327)_RIII region, Th2R | 2100 | 2140 | 2180, 2220, 2260, 2300, 2340, 2380, 2420, 2460 |
| | v2 | | | |
| 21 | CSP(346-366)_Th3R v2 | 2101 | 2141 | 2181, 2221, 2261, 2301, 2341, 2381, 2421, 2461 |
| 22 | CSP(346-376)_Th3R_CS.T3 v2 | 2102 | 2142 | 2182, 2222, 2262, 2302, 2342, 2382, 2422, 2462 |
| 23 | CSP(1-18)_signal peptide | 2103 | 2143 | 2183, 2223, 2263, 2303, 2343, 2383, 2423, 2463 |
| 24 | CSP(27-384)_EcCSP fragment | 2104 | 2144 | 2184, 2224, 2264, 2304, 2344, 2384, 2424, 2464 |
| 25 | CSP(69-74)_3A1 antibody binding | 2105 | 2145 | 2185, 2225, 2265, 2305, 2345, 2385, 2425, 2465 |
| | site | | | |
| 26 | CSP(74-383)_PpCSP fragment | 2106 | 2146 | 2186, 2226, 2266, 2306, 2346, 2386, 2426, 2466 |
| 27 | CSP(75-94)_2C3 antibody binding | 2107 | 2147 | 2187, 2227, 2267, 2307, 2347, 2387, 2427, 2467 |
| | site | | | |
| 28 | CSP(93-97)_RI region | 2108 | 2148 | 2188, 2228, 2268, 2308, 2348, 2388, 2428, 2468 |
| 29 | CSP(95-100)_3H10/3B4 antibody | 2109 | 2149 | 2189, 2229, 2269, 2309, 2349, 2389, 2429, 2469 |
| | binding site | | | |
| 30 | CSP(98-272)_NANP repeat region | 2110 | 2150 | 2190, 2230, 2270, 2310, 2350, 2390, 2430, 2470 |
| 31 | CSP(105-272)_central repeat | 2111 | 2151 | 2191, 2231, 2271, 2311, 2351, 2391, 2431, 2471 |
| | region | | | |
| 32 | CSP(207-395)_RTS,S fragment | 2112 | 2152 | 2192, 2232, 2272, 2312, 2352, 2392, 2432, 2472 |
| 33 | CSP(310-327)_RIII region, Th2R | 2113 | 2153 | 2193, 2233, 2273, 2313, 2353, 2393, 2433, 2473 |
| | v1 | | | |
| | | 2108 | 2148 | 2188, 2228, |
| 33 | v1 (310-327) RIII region, Th2R n | 2113 | 2153 | 2193, 2233,2273, 2313, 2353, 2393,2433, 2473 |
| 34 | CSP(310-374)_RIII region_TSR | 2114 | 2154 | 2194, 2234, 2274, 2314, 2354, 2394, 2434, 2474 |
| | (RII+) | | | |
| 35 | CSP(319-375)_TSR region v3 | 2115 | 2155 | 2195, 2235, 2275, 2315, 2355, 2395, 2435, 2475 |
| 36 | CSP(326-374)_TSR region v1 | 2116 | 2156 | 2196, 2236, 2276, 2316, 2356, 2396, 2436, 2476 |
| 37 | CSP(328-374)_TSR region v2 | 2117 | 2157 | 2197, 2237, 2277, 2317, 2357, 2397, 2437, 2477 |
| 38 | CSP(330-347)_RII+ region v1 | 2118 | 2158 | 2198, 2238, 2278, 2318, 2358, 2398, 2438, 2478 |
| 39 | CSP(330-351)_RII+ region v2 | 2119 | 2159 | 2199, 2239, 2279, 2319, 2359, 2399, 2439, 2479 |
| 40 | CSP(375-397)_GPI anchor signal | 2120 | 2160 | 2200, 2240, 2280, 2320, 2360, 2400, 2440, 2480 |
| 41 | CSP(93-384) | 10080 | 10086 | 10092, 10098, 10104, 10110, 10116, 10122, |
| | | | | 10128, 10134 |
| 42 | CSP(19-152) | 10081 | 10087 | 10093, 10099, 10105, 10111, 10117, 10123, |
| | | | | 10129, 10135 |
| 43 | CSP(19-192) | 10082 | 10088 | 10094, 10100, 10106, 10112, 10118, 10124, |
| | | | | 10130, 10136 |
| 44 | CSP(346-365)_Th3R v1 | 10083 | 10089 | 10095, 10101, 10107, 10113, 10119, 10125, |
| | | | | 10131, 10137 |
| 45 | CSP(346-375)_Th3R_CS.T3 v1 | 10084 | 10090 | 10096, 10102, 10108, 10114, 10120, 10126, |
| | | | | 10132, 10138 |
| 46 | CSP(346-397) | 10085 | 10091 | 10097, 10103, 10109, 10115, 10121, 10127, |
| | | | | 10133, 10139 |

Heterologous elements suitable in the context of the invention, e.g. elements described above (secretory signal peptides, helper epitopes, etc.) are disclosed in Table 4. Therein, each row (row 1-16) corresponds to a certain heterologous element. Column A of Table 4 provides a description of the fragments. For example, sequences provided in row 3 “signal peptide_HsALB” relate to a heterologous signal peptide derived from a human albumin protein. Column B of Table 4 provides the SEQ ID NO of the corresponding amino acid sequences, column C of Table 4 provides the SEQ ID NO of the corresponding wild type RNA coding sequences, and column D of Table 4 provides the SEQ ID NO of the codon modified coding sequences for each fragment (“opt1”, “opt1”, “opt2”, “opt3”, “opt4”, “opt5”, “opt6”, “opt11”). Additional information regarding each of these suitable sequences may also be derived from the sequence listing, in particular from the details provided therein under identifier <223>.

TABLE 4
Heterologous elements and respective coding sequences:

| | A | B | C | D |
|---|---|---|---|---|
| 1 | signal peptide_HsALB | 6205 | 6209 | 6213, 10140, 6217, 6221, 6225, 6229, 6233, 6237 |
| 2 | signal peptide_IgE | 6206 | 6210 | 6214, 6218, 6222, 6226, 6230, 6234, 6238 |
| 3 | signal peptide_HsIns-iso1 | 6207 | 6211 | 6215, 6219, 6223, 6227, 6231, 6235, 6239 |
| 4 | signal peptide_HsSPARC | 6208 | 6212 | 6216, 6220, 6224, 6228, 6232, 6236, 6240 |
| 5 | Linker(AAY) | 6241 | | 6245-6251 |
| 6 | Linker(G4S) | 6242 | | 6252-6261 |
| 7 | Linker(PVTN) | 6243 | | 6262-6265 |
| 8 | Linker(G4SG4) | 6244 | | 6266-6271 |
| 9 | Linker(SGG) | 10141 | | 10142-10146 |
| 10 | Linker(GGS4-GGG) | 10147 | | 10148-10152 |
| 11 | P2 | 6272 | 6275 | 6278, 6281, 6284, 6287, 6290, 6293, 6296, 6299 |
| 12 | PADRE | 6273 | 6276 | 6279, 6282, 6285, 6288, 6291, 6294, 6297, 6300 |
| 13 | HBsAg | 6274 | 6277 | 6280, 6283, 6286, 6289, 6292, 6295, 6298, 6301 |
| 14 | TM domain HA | 6302 | 6303 | 6304, 6305, 6306, 6307, 6308, 6309, 6310, 6311 |
| 15 | Lumazine Synthase | 10153 | | 10154, 10155, 10156, 10157, 10158, 10159, 10160, |
| | (LumSynt) | | | 10161 |
| 16 | Ferritin | 10162 | 10163 | 10164, 10165, 10166, 10167, 10168, 10169, 10170, |
| | | | | 10171 |

In various embodiments, the coding RNA of the invention comprises at least one coding sequence comprising a nucleic acid sequence comprising at least one of the following nucleic acid sequences, preferably in 5′ to 3′ direction:
a) optionally, at least one nucleic acid sequences encoding a heterologous secretory signal sequence selected from SEQ ID NOs: 6209-6240, 10140, or fragments or variants thereof, and, optionally,
b) at least one nucleic acid sequences encoding an antigenic protein derived from CSP, preferably any one of the nucleic acid sequences selected from SEQ ID NOs: 37-328, 2121-2480, 2887-6134, 8754-8855, 10086-10139, or fragments or variants thereof, and
c) optionally, at least one nucleic acid sequences encoding a heterologous helper epitope selected from SEQ ID NOs: 6275, 6276, 6277, 6278, 6281, 6284, 6287, 6290, 6293, 6296, 6299, 6279, 6282, 6285, 6288, 6291, 6294, 6297, 6300, 6280, 6283, 6286, 6289, 6292, 6295, 6298, 6301, or fragments or variants thereof,
d) optionally, at least one nucleic acid sequences encoding a heterologous antigen clustering domain selected from SEQ ID NOs: 6277, 6280, 6283, 6286, 6289, 6292, 6295, 6298, 6301, 10154, 10155, 10156, 10157, 10158, 10159, 10160, 10161, 10163, 10164, 10165, 10166, 10167, 10168, 10169, 10170, 10171 or fragments or variants thereof,
e) optionally, at least one nucleic acid sequences encoding a heterologous transmembrane domain selected from SEQ ID NOs: 6303-6311 or fragments or variants thereof.

Further, a), b), c), d), and e) may be connected with linker elements, preferably via at least one nucleic acid sequence encoding a linker element selected from SEQ ID NOs: 6245-6271, 10142-10146, 10148-10152 or fragments or variants thereof.

Particularly preferred and suitable coding sequences of the coding RNA of the first aspect are provided in Table 5. Therein, each row (row 1-41) corresponds to a certain CSP constructs of the invention. Column A of Table 5 provides a description of the CSP constructs (cf. Table 1). Column B of Table 5 provides the SEQ ID NOs of the corresponding amino acid sequences (cf. Table 1). Column C of Table 5 provides the SEQ ID NOs of the corresponding opt1 RNA coding sequences, Column D of Table 5 provides the SEQ ID NOs of the corresponding “opt2” RNA coding sequences, Column E of Table 5 provides the SEQ ID NOs of the corresponding “opt3” RNA coding sequences, Column F of Table 5 provides the SEQ ID NOs of the corresponding “opt4” RNA coding sequences, Column G of Table 5 provides the SEQ ID NOs of the corresponding “opt5” RNA coding sequences, Column G of Table 5 provides the SEQ ID NOs of the corresponding “opt5” RNA coding sequences, Column H of Table 5 provides the SEQ ID NOs of the corresponding “opt6” RNA coding sequences, Column I of Table 5 provides the SEQ ID NOs of the corresponding “opt11” RNA coding sequences. Additional information regarding each of these suitable coding sequences may also be derived from the sequence listing, in particular from the details provided therein under identifier <223>.

TABLE 5
Preferred coding sequences of the invention

| A | B | C | D | E | F | G | H | I |
|---|---|---|---|---|---|---|---|---|
| Pf-CSP | 1 | 41, 77 | 113 | 149 | 185 | 221 | 257 | 293 |
| Pf-CSP(1-374) | 2 | 42, 78 | 114 | 150 | 186 | 222 | 258 | 294 |
| Pf-CSP(1-325) | 3 | 43, 79 | 115 | 151 | 187 | 223 | 259 | 295 |
| HsALB_Pf-CSP(19-397) | 4 | 44, 80, | 116 | 152 | 188 | 224 | 260 | 296 |
| | | 8755 | | | | | | |
| HsIns-isol_Pf-CSP(19-397) | 5 | 45, 81 | 117 | 153 | 189 | 225 | 261 | 297 |
| HsSPARC_Pf-CSP(19-397) | 6 | 46, 82 | 118 | 154 | 190 | 226 | 262 | 298 |
| IgE_Pf-CSP(19-397) | 7 | 47, 83 | 119 | 155 | 191 | 227 | 263 | 299 |
| Pf-CSP_Linker(G4SG4)_TM domain HA | 8 | 48, 84 | 120 | 156 | 192 | 228 | 264 | 300 |
| Pf-CSP(199-377)_Linker(PVTN)_HBsAg | 9 | 49, 85 | 121 | 157 | 193 | 229 | 265 | 301 |
| Pf-CSP(199-387)_Linker(PVTN)_HBsAg | 10 | 50, 86 | 122 | 158 | 194 | 230 | 266 | 302 |
| HsALB_Pf-CSP(19-152) | 11 | 51, 87 | 123 | 159 | 195 | 231 | 267 | 303 |
| HsALB_Pf-CSP(19-192) | 12 | 52, 88 | 124 | 160 | 196 | 232 | 268 | 304 |
| HsALB_Pf-CSP(19-272) | 13 | 53, 89 | 125 | 161 | 197 | 233 | 269 | 305 |
| HsALB_Pf-CSP(19-272)_Linker(AAY)_Pf- | 14 | 54, 90, | 126 | 162 | 198 | 234 | 270 | 306 |
| CSP(310-327)_Linker(AAY)_Pf-CSP(346-375) | | 8766 | | | | | | |
| HsALB_Pf-CSP(19-272)_Linker(AAY)_Pf- | 15 | 55, 91 | 127 | 163 | 199 | 235 | 271 | 307 |
| CSP(346-365)_Linker(AAY)_P2 | | | | | | | | |
| HsALB_Pf-CSP(19-272)_Linker(AAY)_Pf- | 16 | 56, 92 | 128 | 164 | 200 | 236 | 272 | 308 |
| CSP(346-365)_Linker(AAY)_PADRE | | | | | | | | |
| HsALB_Pf-CSP(19-272)_Linker(G4S)_Pf- | 17 | 57, 93 | 129 | 165 | 201 | 237 | 273 | 309 |
| CSP(310-327)_Linker(G4S)_Pf-CSP(346-375) | | | | | | | | |
| HsALB_Pf-CSP(19-272)_Linker(G4S)_Pf- | 18 | 58, 94 | 130 | 166 | 202 | 238 | 274 | 310 |
| CSP(310-327)_Pf-CSP(346-375) | | | | | | | | |
| HsALB_Pf-CSP(19-325) | 19 | 59, 95 | 131 | 167 | 203 | 239 | 275 | 311 |
| HsALB_Pf-CSP(19-384) | 20 | 60, 96 | 132 | 168 | 204 | 240 | 276 | 312 |
| HsALB_Pf-CSP(19-384)_TM domain HA | 21 | 61, 97 | 133 | 169 | 205 | 241 | 277 | 313 |
| HsALB_Pf-CSP(82-397) | 22 | 62, 98 | 134 | 170 | 206 | 242 | 278 | 314 |
| HsALB_Pf-CSP(93-192) | 23 | 63, 99 | 135 | 171 | 207 | 243 | 279 | 315 |
| HsALB_Pf-CSP(93-272) | 24 | 64, 100 | 136 | 172 | 208 | 244 | 280 | 316 |
| HsALB_Pf-CSP(93-397) | 25 | 65, 101 | 137 | 173 | 209 | 245 | 281 | 317 |
| HsALB_Pf-CSP(98-192) | 26 | 66, 102 | 138 | 174 | 210 | 246 | 282 | 318 |
| HsALB_Pf-CSP(98-272) | 27 | 67, 103 | 139 | 175 | 211 | 247 | 283 | 319 |
| HsALB_Pf-CSP(98-374) | 28 | 68, 104 | 140 | 176 | 212 | 248 | 284 | 320 |
| HsALB_Pf-CSP(98-397) | 29 | 69, 105 | 141 | 177 | 213 | 249 | 285 | 321 |
| HsALB_Pf-CSP(199-377)_Linker(PVTN)_HBsAg | 30 | 70, | 142 | 178 | 214 | 250 | 286 | 322 |
| | | 106, | | | | | | |
| | | 8765 | | | | | | |
| LumSynt_Linker(GGS4-GGG)_Pf-CSP(19-397) | 8742 | 8754, | 8784 | 8796 | 8808 | 8820 | 8832 | 8844 |
| | | 8772 | | | | | | |
| HsALB_Pf-CSP(19-272)_Linker(PVTN)_HBsAg | 8743 | 8756, | 8785 | 8797 | 8809 | 8821 | 8833 | 8845 |
| | | 8773 | | | | | | |
| HsALB_Pf-CSP(19-272)_Linker(AAY)_Pf- | 8744 | 8757, | 8786 | 8798 | 8810 | 8822 | 8834 | 8846 |
| CSP(346- | | 8758, | | | | | | |
| 365)_Linker(AAY)_PADRE_Linker(PVTN)_HBsAg | | 8774 | | | | | | |
| HsALB_Pf-CSP(19-384)_Linker(PVTN)_HBsAg | 8745 | 8759, | 8787 | 8799 | 8811 | 8823 | 8835 | 8847 |
| | | 8760, | | | | | | |
| | | 8775 | | | | | | |
| HsALB_Pf-CSP(19-384)_Linker(SGG)Ferritin | 8746 | 8761, | 8788 | 8800 | 8812 | 8824 | 8836 | 8848 |
| | | 8762, | | | | | | |
| | | 8776 | | | | | | |
| HsALB_Pf-CSP(93-384)_Linker(PVTN)_HBsAg | 8747 | 8763, | 8789 | 8801 | 8813 | 8825 | 8837 | 8849 |
| | | 8764, | | | | | | |
| | | 8777 | | | | | | |
| HsALB_Pf-CSP(19-272)_Linker(AAY)_Pf- | 8749 | 8767, | 8791 | 8803 | 8815 | 8827 | 8839 | 8851 |
| CSP(346-375)_Linker(AAY)_Pf-CSP(310-327) | | 8779 | | | | | | |
| HsALB_Pf-CSP(19-272)_Linker(AAY)_Pf- | 8750 | 8768, | 8792 | 8804 | 8816 | 8828 | 8840 | 8852 |
| CSP(346-375)_Linker(AAY)_Pf-CSP(310- | | 8780 | | | | | | |
| 327)_Linker(AAY)_PADRE | | | | | | | | |
| HsALB_Pf-CSP(19-272)_Linker(AAY)_Pf- | 8751 | 8769, | 8793 | 8805 | 8817 | 8829 | 8841 | 8853 |
| CSP(310-327)_Linker(AAY)_Pf-CSP(346- | | 8781 | | | | | | |
| 375)_Linker(AAY)_PADRE | | | | | | | | |
| HsALB_Pf-CSP(19-272)_Linker(AAY)_Pf- | 8752 | 8770, | 8794 | 8806 | 8818 | 8830 | 8842 | 8854 |
| CSP(310-327)_Linker(AAY)_Pf-CSP(346- | | 8782 | | | | | | |
| 375)_Linker(AAY)_PADRE_Linker(AAY)_P2 | | | | | | | | |
| HsALB_Pf-CSP(19-272)_Linker(AAY)_Pf- | 8753 | 8771, | 8795 | 8807 | 8819 | 8831 | 8843 | 8855 |
| CSP(310-327)_Linker (AAY)_Pf-CSP(346-397) | | 8783 | | | | | | |

RNA Elements, mRNA Elements:

In embodiments, the coding RNA of the first aspect may be monocistronic, bicistronic, or multicistronic.

The term “monocistronic” will be recognized and understood by the person of ordinary skill in the art, and is e.g. intended to refer to an RNA that comprises only one coding sequences. The terms “bicistronic”, or “multicistronic” as used herein will be recognized and understood by the person of ordinary skill in the art, and are e.g. intended to refer to an RNA that may have two (bicistronic) or more (multicistronic) coding sequences.

In preferred embodiments, the coding RNA of the first aspect is monocistronic.

In embodiments, the coding RNA is monocistronic and the coding sequence of said RNA encodes at least two different antigenic peptides or proteins derived from a Malaria parasite (e.g. Plasmodium CSP). Accordingly, said coding sequence may encode at least two, three, four, five, six, seven, eight and more antigenic peptides or proteins derived from a Malaria parasite (e.g. Plasmodium CSP), linked with or without an amino acid linker sequence, wherein said linker sequence can comprise rigid linkers, flexible linkers, cleavable linkers, or a combination thereof. Such constructs are herein referred to as “multi-antigen-constructs”.

In embodiments, the coding RNA may be bicistronic or multicistronic and comprises at least two coding sequences, wherein the at least two coding sequences encode two or more different antigenic peptides or proteins derived from a Malaria parasite (e.g. Plasmodium CSP). Accordingly, the coding sequences in a bicistronic or multicistronic RNA suitably encode distinct antigenic proteins or peptides as defined herein or immunogenic fragments or immunogenic variants thereof. Preferably, the coding sequences in said bicistronic or multicistronic constructs may be separated by at least one IRES (internal ribosomal entry site) sequence. Thus, the term “encoding two or more antigenic peptides or proteins” may mean, without being limited thereto, that the bicistronic or multicistronic RNA encodes e.g. at least two, three, four, five, six or more (preferably different) antigenic peptides or proteins of different Malaria parasites. Alternatively, the bicistronic or multicistronic RNA may encode e.g. at least two, three, four, five, six or more (preferably different) antigenic peptides or proteins derived from the same Malaria parasite. In that context, suitable IRES sequences may be selected from the list of nucleic acid sequences according to SEQ ID NOs: 1566-1662 of the patent application WO2017/081082, or fragments or variants of these sequences. In this context, the disclosure of WO2017/081082 relating to IRES sequences is herewith incorporated by reference.

It has to be understood that, in the context of the invention, certain combinations of coding sequences may be generated by any combination of monocistronic, bicistronic and multicistronic RNA constructs and/or multi-antigen-constructs to obtain a composition encoding multiple antigenic peptides or proteins as defined herein.

Preferably, the coding RNA of the first aspect typically comprises about 50 to about 20000 nucleotides, or about 500 to about 10000 nucleotides, or about 1000 to about 10000 nucleotides, or preferably about 1000 to about 5000 nucleotides, or even more preferably about 1000 to about 2500 nucleotides.

According to preferred embodiments, the coding RNA of the first aspect may be an mRNA, a self-replicating RNA, a circular RNA, or a replicon RNA.

In embodiments, the coding RNA of the first aspect is a circular RNA. As used herein, “circular RNA” or “circRNAs” have to be understood as a circular polynucleotide constructs that encode at least one antigenic peptide or protein as defined herein. Accordingly, in preferred embodiments, said circRNA comprises at least one coding sequence encoding at least one antigenic protein derived from a Malaria parasite (e.g., Plasmodium CSP), or an immunogenic fragment or an immunogenic variant thereof. The production of circRNA can be performed using various methods provided in the art. Accordingly, methods for producing circular RNA as provided in U.S. Pat. Nos. 6,210,931, 5,773,244, WO1992/001813, WO2015/034925 and WO2016/011222 are incorporated herewith by reference.

In embodiments, the coding RNA is a replicon RNA. The term “replicon RNA” will be recognized and understood by the person of ordinary skill in the art, and is e.g. intended to be an optimized self-replicating RNA. Such constructs may include replicase elements derived from e.g. alphaviruses (e.g. SFV, SIN, VEE, or RRV) and the substitution of the structural virus proteins with the nucleic acid of interest. Alternatively, the replicase may be provided on an independent coding RNA construct. Downstream of the replicase may be a sub-genomic promoter that controls replication of the replicon RNA.

In preferred embodiments, the coding RNA of the first aspect is an mRNA.

The terms “RNA” and “mRNA” will be recognized and understood by the person of ordinary skill in the art, and are e.g. intended to be a ribonucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. The mRNA (messenger RNA) usually provides the nucleotide sequence that may be translated into an amino-acid sequence of a particular peptide or protein.

In the context of the invention, the coding RNA, preferably the mRNA of the first aspect, may provide at least one coding sequence encoding an antigen derived from a Malaria parasite that is translated into a functional antigen after administration (e.g. after administration to a subject, e.g. a human subject). Accordingly, the coding RNA, preferably the mRNA, is suitable for a vaccine, preferable for a malaria vaccine.

Suitably, the RNA may be modified by the addition of a 5′-cap structure, which preferably stabilizes the RNA and/or enhances expression of the encoded antigen.

The RNA may suitably be modified by the addition of a 5′-cap structure, which preferably stabilizes the RNA as described herein and/or enhances expression of the encoded antigen and/or reduces the stimulation of the innate immune system (after administration to a subject). A 5′-cap structure is of particular importance in embodiments where the RNA is a linear, coding RNA, e.g. an mRNA or a linear coding replicon RNA.

Accordingly, in preferred embodiments, the RNA, in particular the mRNA of the first aspect comprises a 5′-cap structure, preferably m7G (m7G(5′)ppp(5′)G), cap0, cap1, cap2, a modified cap0 or a modified cap1 structure.

In particularly preferred embodiments, the mRNA of the first aspect comprises a cap1.

The term “5′-cap structure” as used herein will be recognized and understood by the person of ordinary skill in the art, and is e.g. intended to refer to a 5′ modified nucleotide, particularly a guanine nucleotide, positioned at the 5′-end of an RNA molecule, e.g. an mRNA molecule. Preferably, the 5′-cap structure is connected via a 5′-5′-triphosphate linkage to the RNA.

5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may be formed in chemical RNA synthesis or RNA in vitro transcription (co-transcriptional capping) using cap analogues.

The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of a nucleic acid molecule, particularly of an RNA molecule, when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008/016473, WO2008/157688, WO2009/149253, WO2011/015347, and WO2013/059475). Further suitable cap analogues in that context are described in WO2017/066793, WO2017/066781, WO2017/066791, WO2017/066789, WO2017/053297, WO2017/066782, WO2018/075827 and WO2017/066797 wherein the disclosures referring to cap analogues are incorporated herewith by reference.

In embodiments, a modified cap1 structure is generated using tri-nucleotide cap analogue as disclosed in WO2017/053297, WO2017/066793, WO2017/066781, WO2017/066791, WO2017/066789, WO2017/066782, WO2018/075827 and WO2017/066797. In particular, any cap structures derivable from the structure disclosed in claim 1-5 of WO2017/053297 may be suitably used to co-transcriptionally generate a modified cap1 structure. Further, any cap structures derivable from the structure defined in claim 1 or claim 21 of WO2018/075827 may be suitably used to co-transcriptionally generate a modified cap1 structure.

In preferred embodiments, the 5′-cap structure may suitably be added co-transcriptionally using tri-nucleotide cap analogue as defined herein in an RNA in vitro transcription reaction as defined herein.

In particularly preferred embodiments, the coding RNA, in particular the mRNA of the first aspect comprises a cap1 structure. As shown in the Example section, the presence of a cap1 structure is of particular importance as the induction of a specific immune response against Malaria CSP could be increased (see Examples 11 and 12).

Preferred cap-analogues are the di-nucleotide cap analogues m7G(5′)ppp(5′)G (m7G) or 3′-O-Me-m7G(5′)ppp(5′)G to co-transcriptionally generate cap0 structures. Further preferred cap-analogues are the tri-nucleotide cap analogues m7G(5′)ppp(5′)(2′OMeA)pG or m7G(5′)ppp(5′)(2′OMeG)pG to co-transcriptionally generate cap1 structures.

In other embodiments, the 5′-cap structure is formed via enzymatic capping using capping enzymes (e.g. vaccinia virus capping enzymes and/or cap-dependent 2′-0 methyltransferases) to generate cap0 or cap1 or cap2 structures. The 5′-cap structure (cap0 or cap1) may be added using immobilized capping enzymes and/or cap-dependent 2′-0 methyltransferases using methods and means disclosed in WO2016/193226.

In preferred embodiments, about 70%, 75%, 80%, 85%, 90%, 95% of the coding RNA (species) comprises a cap1 structure as determined using a capping assay. In preferred embodiments, less than about 20%, 15%, 10%, 5%, 4%, 3%, 2%, 1% of the coding RNA (species) does not comprises a cap1 structure as determined using a capping assay. In preferred embodiments, less than about 20%, 15%, 10%, 5%, 4%, 3%, 2%, 1% of the coding RNA (species) comprises a cap0 structure as determined using a capping assay. In preferred embodiments, less than about 20%, 15%, 10%, 5%, 4%, 3%, 2%, 1% of the coding RNA (species) comprises a cap1 intermediate structure as determined using a capping assay.

The term “coding RNA species” is not restricted to mean “one single molecule” but is understood to comprise an ensemble of essentially identical RNA molecules. Accordingly, it may relate to a plurality of essentially identical coding RNA molecules.

For determining the capping degree or the presence of cap1 intermediates, a capping assays as described in published PCT application WO2015/101416, in particular, as described in claims 27 to 46 of published PCT application WO2015/101416 can be used. Other capping assays that may be used to determine the capping degree of the coding RNA are described in PCT/EP2018/08667, or published PCT applications WO2014/152673 and WO2014/152659.

In preferred embodiments, the coding RNA comprises an m7G(5′)ppp(5′)(2′OMeA) cap structure. In such embodiments, the coding RNA comprises a 5′-terminal m7G cap, and an additional methylation of the ribose of the adjacent nucleotide of m7GpppN, in that case, a 2′O methylated adenosine. Preferably, about 70%, 75%, 80%, 85%, 90%, 95% of the coding RNA (species) comprises such a cap1 structure as determined using a capping assay.

In other preferred embodiments, the coding RNA of the first aspect comprises an m7G(5′)ppp(5′)(2′OMeG) cap structure. In such embodiments, the coding RNA comprises a 5′-terminal m7G cap, and an additional methylation of the ribose of the adjacent nucleotide, in that case, a 2′O methylated guanosine. Preferably, about 70%, 75%, 80%, 85%, 90%, 95% of the coding RNA (species) comprises such a cap1 structure as determined using a capping assay.

In a particularly preferred embodiment, the RNA of the first aspect comprises a cap1 structure, wherein said cap1 structure may be formed enzymatically or co-transcriptionally (e.g. using m7G(5′)ppp(5′)(2′OMeA), or m7G(5′)ppp(5′)(2′OMeG) analogues).

In preferred embodiments, the RNA of the first aspect comprises an m7G(5′)ppp(5′)(2′OMeA) cap structure. In such embodiments, the coding RNA comprises a 5′ terminal m7G cap, and an additional methylation of the ribose of the adjacent nucleotide of m7GpppN, in that case, a 2′O methylated adenosine.

In other preferred embodiments, the RNA of the first aspect comprises an m7G(5′)ppp(5′)(2′OMeG) cap structure. In such embodiments, the coding RNA comprises a 5′ terminal m7G cap, and an additional methylation of the ribose of the adjacent nucleotide, in that case, a 2′O methylated guanosine.

Accordingly, whenever reference is made to suitable RNA or mRNA sequences in the context of the invention, the first nucleotide of said RNA or mRNA sequence, that is, the nucleotide downstream of the m7G(5′)ppp structure, may be a 2′O methylated guanosine or a 2′O methylated adenosine.

In embodiments, the A/U content in the environment of the ribosome binding site of the coding RNA may be increased compared to the A/U content in the environment of the ribosome binding site of its respective wild type nucleic acid. This modification (an increased A/U content around the ribosome binding site) increases the efficiency of ribosome binding to the nucleic acid, preferably the RNA. An effective binding of the ribosomes to the ribosome binding site in turn has the effect of an efficient translation of the RNA.

Accordingly, in a particularly preferred embodiment, the coding RNA comprises a ribosome binding site, also referred to as “Kozak sequence” identical to or at least 80%, 85%, 90%, 95% identical to any one of the sequences SEQ ID NOs: 6175, 6176, or fragments or variants thereof.

In preferred embodiments, the RNA of the invention comprises at least one poly(N) sequence, e.g. at least one poly(A) sequence, at least one poly(U) sequence, at least one poly(C) sequence, or combinations thereof.

In preferred embodiments, the RNA of the invention comprises at least one poly(A) sequence.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of an RNA, of up to about 1000 adenosine nucleotides. Preferably, said poly(A) sequence is essentially homopolymeric, e.g. a poly(A) sequence of e.g. 100 adenosine nucleotides has essentially the length of 100 nucleotides. In other embodiments, the poly(A) sequence may be interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide different from an adenosine nucleotide).

The poly(A) sequence, suitable located downstream of the 3′-UTR as defined herein, may comprise about 10 to about 500 adenosine nucleotides, about 10 to about 200 adenosine nucleotides, about 40 to about 200 adenosine nucleotides, or about 40 to about 150 adenosine nucleotides. Suitably, the length of the poly(A) sequence may be at least about or even more than about 10, 50, 64, 75, 100, 200, 300, 400, or 500 adenosine nucleotides. Suitably, the poly(A) sequence of the RNA of the first aspect may be long enough to bind at least 2, 3, 4, 5 or more monomers of PolyA Binding Proteins. In preferred embodiments, the poly(A) sequence comprises about 50 to about 250 adenosines. In a particularly preferred embodiment, the poly(A) sequence comprises about 64 adenosine nucleotides. In further particularly preferred embodiments, the poly(A) sequence comprises about 75 adenosine nucleotides.

In preferred embodiments, the coding RNA comprises at least one poly(A) sequence comprising about 30 to about 200 adenosine nucleotides. In preferred embodiments, the poly(A) sequence comprises about 64 adenosine nucleotides (A64). In particularly preferred embodiments, the poly(A) sequence comprises about 100 adenosine nucleotides (A100). In preferred embodiments, the poly(A) sequence comprises about 150 adenosine nucleotides.

The poly(A) sequence as defined herein is suitably located at the 3′ terminus of the coding RNA. Accordingly it is preferred that the 3′-terminal nucleotide of the coding RNA (that is the last 3′-terminal nucleotide in the polynucleotide chain) is the 3′-terminal A nucleotide of the at least one poly(A) sequence. The term “located at the 3′ terminus” has to be understood as being located exactly at the 3′ terminus—in other words, the 3′ terminus of the coding RNA consists of a poly(A) sequence terminating with an A nucleotide. Examples of sequences having a 3′ terminus consisting of a poly(A) sequence are e.g. SEQ ID NOs: 8013-8741, 9774-10079. For further examples of sequences having a poly(A) sequence located (exactly) at the 3′ terminus see also Table 9 (column “3′-end” with hSL-A100) or Table 6B, column H. The presence of a poly(A) sequence exactly at the 3′ terminus of the coding RNA encoding a Malaria antigenic protein (e.g. CSP) is advantageous as all mRNA vaccine comprising a 3′-end with hSL-A100 induces very strong humoral and cellular immune responses (see Example 13).

Preferably, the poly(A) sequence of the RNA is obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using commercially available polyadenylation kits and corresponding protocols known in the art, or alternatively, by using immobilized poly(A)polymerases e.g. using a methods and means as described in WO2016/174271.

In embodiments, the RNA may comprise a poly(A) sequence derived from a template DNA and may comprise at least one additional poly(A) sequence generated by enzymatic polyadenylation, e.g. as described in WO2016/091391.

In embodiments where enzymatic polyadenylation of RNA is used, it has to be understood that RNA or mRNA sequences as e.g. provided in the sequence listing, may additionally comprise about 30 to about 500 adenosine nucleotides.

In preferred embodiments, the RNA may comprise at least one poly(C) sequence.

The term “poly(C) sequence” as used herein will be recognized and understood by the person of ordinary skill in the art, and are for example intended to be a sequence of cytosine nucleotides, typically located at the 3′-end of an RNA, of up to about 200 cytosine nucleotides.

In preferred embodiments, the poly(C) sequence, suitable located at the 3′ terminus downstream of the 3′-UTR as defined herein, comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In a particularly preferred embodiment, the poly(C) sequence comprises about 30 cytosine nucleotides.

Preferably, the poly(C) sequence in the RNA sequence of the present invention is derived from a DNA template by RNA in vitro transcription. In other embodiments, the poly(C) sequence is obtained in vitro by common methods of chemical synthesis, or enzymatically, without being necessarily transcribed from a DNA template.

In other embodiments, the RNA of the invention does not comprise a poly(C) sequence as defined herein.

In further embodiments, the coding RNA of the invention does comprise a poly(A) sequence as defined herein, preferably A100 located (exactly) at the 3′ terminus, and does not comprise a poly(C) sequence.

In a particularly preferred embodiment, the coding RNA of the invention comprises a cap1 structure as defined herein and at least one poly(A) sequence as defined in herein. Preferably, said cap1 structure is obtainable by co-transcriptional capping as defined herein, and said poly(A) sequence is preferably (exactly) at the 3′ terminus.

In preferred embodiments, the RNA of the first aspect comprises at least one histone stem-loop (sequence).

The term “histone stem-loop” (abbreviated as “hSL” in e.g. the sequence listing) as used herein will be recognized and understood by the person of ordinary skill in the art, and are for example intended to refer to nucleic acid sequences that are predominantly found in histone mRNAs. Exemplary histone stem-loop sequences are described in Lopez et al. (Davila Lopez et al, (2008), RNA, 14(1)).

Histone stem-loop sequences/structures may suitably be selected from histone stem-loop sequences as disclosed in WO2012/019780, the disclosure relating to histone stem-loop sequences/histone stem-loop structures incorporated herewith by reference. A histone stem-loop sequence that may be used within the present invention may preferably be derived from formulae (I) or (II) of WO2012/019780. According to a further preferred embodiment the RNA as defined herein may comprise at least one histone stem-loop sequence derived from at least one of the specific formulae (Ia) or (IIa) of the patent application WO2012/019780.

In particularly preferred embodiment, the RNA of the invention comprises at least one histone stem-loop sequence, wherein said histone stem-loop sequence comprises a nucleic acid sequence identical or at least 70%, 80%, 90%, 91%, 92%, 93%, 94%, 95%, 96%, 97%, 98%, or 99% identical to SEQ ID NOs: 6173 or 6174, or fragments or variants thereof.

In other embodiments, the RNA of the first aspect does not comprise a histone stem-loop as defined herein.

In embodiments, the RNA of the invention comprises a 3″-terminal sequence element. Said 3″-terminal sequence element comprises a poly(A)sequence and a histone-stem-loop sequence, wherein said sequence element is located at the 3′ terminus of the RNA of the invention.

Accordingly, the RNA of the invention may comprise a 3′-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 6179-6200, 10173-10196, or a fragment or variant thereof.

In various embodiments, the RNA may comprise a 5′-terminal sequence element according to SEQ ID NOs: 6177, 6178, or a fragment or variant thereof. Such a 5′-terminal sequence element comprises e.g. a binding site for T7 RNA polymerase. Further, the first nucleotide of said 5′-terminal start sequence may preferably comprise a 2′O methylation, e.g. 2′O methylated guanosine or a 2′O methylated adenosine.

In embodiments, the RNA may comprise a sequence element which represents a cleavage site for a catalytic nucleic acid molecule, wherein the catalytic nucleic acid molecule may be a Ribozyme or a DNAzyme. Said elements may, e.g., allow for the analysis of capping efficiency/quality of the RNA as described in WO2015/101416, or allow for the analysis of poly(N)sequences length/quality of the RNA as described in WO2017/001058. A cleavage site for a catalytic nucleic acid molecule may be located in proximity to the 5′ terminus of the RNA (that is, about 1, 2, 3, 4, 5, 6, 7, 8, 9, 10, 1-30, 1-20, 5-15 nucleotides from the 5′ terminal cap structure). Alternatively, or in addition, a cleavage site for a catalytic nucleic acid molecule as described above may also be positioned in proximity to the 3′ terminus of the RNA (that is, about 50-300, 50-200, 50-150 nucleotides from the 3′ terminus). Said elements may, e.g., allow for the analysis of poly(N)sequences length/quality of the RNA as described in WO2017/001058.

### UTRs:

The RNA of the invention may be composed of a protein-coding region (“coding sequence” or “cds”), and 5′-UTR and/or 3′-UTR. Notably, UTRs may harbor regulatory sequence elements that determine RNA turnover, stability, and localization. Moreover, UTRs may harbor sequence elements that enhance translation. In medical application of RNA, translation of the RNA into at least one peptide or protein is of paramount importance to therapeutic efficacy. Certain combinations of 3′-UTRs and/or 5′-UTRs may enhance the expression of operably linked coding sequences encoding peptides or proteins of the invention. RNA molecules harboring said UTR combinations advantageously enable rapid and transient expression of antigenic peptides or proteins after administration to a subject, preferably after intramuscular administration. Accordingly, the coding RNA comprising certain combinations of 3′-UTRs and/or 5′-UTRs as provided herein is particularly suitable for administration as a vaccine, in particular, suitable for administration into the muscle, the dermis, or the epidermis of a subject.

Suitably, the RNA of the first aspect may comprise at least one heterologous 5′-UTR and/or at least one heterologous 3′-UTR. Said heterologous 5′-UTRs or 3′-UTRs may be derived from naturally occurring genes or may be synthetically engineered. In preferred embodiments, the RNA of the first aspect comprises at least one coding sequence operably linked to at least one (heterologous) 3′-UTR and/or at least one (heterologous) 5′-UTR.

In preferred embodiments, the at least one RNA comprises at least one heterologous 3′-UTR.

The term “3′-untranslated region” or “3′-UTR” or “3′-UTR element” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of an RNA, e.g. an mRNA, located between a cds and a terminal poly(A) sequence. A 3′-UTR may comprise elements for controlling gene expression, also called regulatory elements. Such regulatory elements may be, e.g., ribosomal binding sites, miRNA binding sites etc.

Preferably the RNA comprises a 3′-UTR, which may be derivable from a gene that relates to an RNA with enhanced half-life (i.e. that provides a stable RNA).

In some embodiments, a 3′-UTR comprises one or more of a polyadenylation signal, a binding site for proteins that affect an RNA stability of location in a cell, or one or more miRNA or binding sites for miRNAs.

MicroRNAs (or miRNA) are 19-25 nucleotide long noncoding RNAs that bind to the 3′-UTR of nucleic acid molecules and down-regulate gene expression either by reducing nucleic acid molecule stability or by inhibiting translation. For examples microRNAs are known to regulate RNA, and thereby protein expression, for example in liver (miR-122), heart (miR-Id, miR-149), endothelial cells (miR-17-92, miR-126), adipose tissue (let-7, miR-30c), kidney (miR-192, miR-194, miR-204), myeloid cells (miR-142-3p, miR-142-5p, miR-16, miR-21, miR-223, miR-24, miR-27), muscle (miR-133, miR-206, miR-208), and lung epithelial cells (let-7, miR-133, miR-126). The RNA of the invention may comprise one or more microRNA target sequences, microRNA sequences, or microRNA seeds. Such sequences may e.g. correspond to any known microRNA such as those taught in US Publication US2005/0261218 and US Publication US2005/0059005, the contents of which are incorporated herein by reference in their entirety.

Accordingly, miRNA, or binding sites miRNAs as defined above for may be removed from the 3′-UTR or introduced into the 3′-UTR in order to tailor the expression of the RNA expression to desired cell types or tissues.

In preferred embodiments of the first aspect, the RNA comprises at least one heterologous 3′-UTR, wherein the at least one heterologous 3′-UTR comprises a nucleic acid sequence derived from a 3′-UTR of a gene selected from PSMB3, ALB7, alpha-globin (referred to as “muag”), CASP1, COX6B1, GNAS, NDUFA1 and RPS9, or from a homolog, a fragment or variant of any one of these genes.

Preferred in the context of the invention are nucleic acid sequences derived from a 3′-UTR of an alpha-globin (referred to as “muag”), an ALB7 gene, or a PSMB3 gene, or from a homolog, a fragment or variant of any one of these genes.

In preferred embodiments the 3′-UTR of the coding RNA comprises a nucleic acid sequences derived from a 3′-UTR of a PSMB3 gene.

In embodiments, the RNA may comprise a 3′-UTR derived from a ALB7 gene, wherein said 3′-UTR derived from an ALB7 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6169 or 6170 or a fragment or a variant thereof.

In embodiments, the RNA may comprise a 3′-UTR derived from a alpha-globin gene, wherein said 3′-UTR derived from a alpha-globin gene (“muag”) comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6171 or 6172 or a fragment or a variant thereof.

In preferred embodiments, the RNA may comprise a 3′-UTR derived from a PSMB3 gene, wherein said 3′-UTR derived from a PSMB3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6157 or 6158 or a fragment or a variant thereof.

In embodiments, the RNA may comprise a 3′-UTR derived from a CASP1 gene, wherein said 3′-UTR derived from a CASP1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6159 or 6160 or a fragment or a variant thereof.

In embodiments, the RNA may comprise a 3′-UTR derived from a COX6B1 gene, wherein said 3′-UTR derived from a COX6B1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6161 or 6162 or a fragment or a variant thereof.

In embodiments, the RNA may comprise a 3′-UTR derived from a GNAS gene, wherein said 3′-UTR derived from a GNAS gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6163 or 6164 or a fragment or a variant thereof.

In embodiments, the RNA may comprise a 3′-UTR derived from a NDUFA1 gene, wherein said 3′-UTR derived from a NDUFA1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6165 or 6166 or a fragment or a variant thereof.

In embodiments, the RNA may comprise a 3′-UTR derived from a RPS9 gene, wherein said 3′-UTR derived from a RPS9 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6167 or 6168 or a fragment or a variant thereof.

Accordingly, the coding RNA of the first aspect may suitably comprise at least one 3′-UTR comprising or consisting of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6157 to 6172 or a fragment or a variant thereof.

In other embodiments, the RNA of the first aspect comprises a 3′-UTR as described in WO2016/107877, the disclosure of WO2016/107877 relating to 3′-UTR sequences herewith incorporated by reference. Suitable 3′-UTRs are SEQ ID NOs: 1 to 24 and SEQ ID NOs: 49 to 318 of WO2016/107877, or fragments or variants of these sequences. Accordingly, the 3′-UTRs of the RNA may comprise or consist of a corresponding RNA sequence of the nucleic acid sequence according SEQ ID NOs: 1 to 24 and SEQ ID NOs: 49 to 318 of WO2016/107877. In other embodiments, the RNA of the first aspect comprises a 3′-UTR as described in WO2017/036580, the disclosure of WO2017/036580 relating to 3′-UTR sequences herewith incorporated by reference. Suitable 3′-UTRs are SEQ ID NOs: 152 to 204 of WO2017/036580, or fragments or variants of these sequences. Accordingly, the 3′-UTR of the RNA may comprise or consist of a corresponding RNA sequence of the nucleic acid sequence according SEQ ID NOs: 152 to 204 of WO2017/036580. In other embodiments, the RNA of the first aspect comprises a 3′-UTR as described in WO2016/022914, the disclosure of WO2016/022914 relating to 3′-UTR sequences herewith incorporated by reference. Particularly preferred 3′-UTRs are nucleic acid sequences according to SEQ ID NOs: 20 to 36 of WO2016/022914, or fragments or variants of these sequences. In this context, it is particularly preferred that the 3′-UTR of the RNA comprises or consists of a corresponding RNA sequence of the nucleic acid sequence according to SEQ ID NOs: 20 to 36 of WO2016/022914.

In preferred embodiments, the at least one RNA comprises at least one heterologous 5′-UTR.

The terms “5′-untranslated region” or “5′-UTR” or “5′-UTR element” will be recognized and understood by the person of ordinary skill in the art, and are e.g. intended to refer to a part of a nucleic acid molecule located 5′ (i.e. “upstream”) of a coding sequence and which is not translated into protein. A 5′-UTR may be part of an RNA located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g. by enzymatic or post-transcriptional addition of a 5′-cap structure (as defined above).

Preferably the RNA comprises a 5′-UTR, which may be derivable from a gene that relates to an RNA with enhanced half-life (i.e. that provides a stable RNA).

In some embodiments, a 5′-UTR comprises one or more of a binding site for proteins that affect an RNA stability of location in a cell, or one or more miRNA or binding sites for miRNAs.

Accordingly, miRNA or binding sites miRNAs as defined above for may be removed from the 5′-UTR or introduced into the 5′-UTR in order to tailor the expression of the RNA expression to desired cell types or tissues.

In preferred embodiments of the first aspect, the RNA comprises at least one heterologous 5′-UTR, wherein the at least one heterologous 5′-UTR comprises a nucleic acid sequence derived from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes.

Particularly preferred in the context of the invention are nucleic acid sequences derived from a 5′-UTR of a HSD17B4 gene, a SLC7A3 gene, or a RPL32 gene, or from a homolog, a fragment or variant of any one of these genes.

In preferred embodiments the 5′-UTR of the coding RNA comprises a nucleic acid sequences derived from a 5′-UTR of a SLC7A3 gene.

In particularly preferred embodiments the 5′-UTR of the coding RNA comprises a nucleic acid sequences derived from a 5′-UTR of a HSD17B4 gene.

In embodiments, the RNA may comprise a 5′-UTR derived from a RPL32 gene, wherein said 5′-UTR derived from a RPL32 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6155 or 6156 or a fragment or a variant thereof.

In preferred embodiments, the RNA may comprise a 5′-UTR derived from a HSD17B4 gene, wherein said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6135 or 6136 or a fragment or a variant thereof.

In embodiments, the RNA may comprise a 5′-UTR derived from a ASAH1 gene, wherein said 5′-UTR derived from a ASAH1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6137 or 6138 or a fragment or a variant thereof.

In embodiments, the RNA may comprise a 5′-UTR derived from a ATP5A1 gene, wherein said 5′-UTR derived from a ATP5A1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6139 or 6140 or a fragment or a variant thereof.

In embodiments, the RNA may comprise a 5′-UTR derived from a MP68 gene, wherein said 5′-UTR derived from a MP68 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6141 or 6142 or a fragment or a variant thereof.

In embodiments, the RNA may comprise a 5′-UTR derived from a NDUFA4 gene, wherein said 5′-UTR derived from a NDUFA4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6143 or 6144 or a fragment or a variant thereof.

In embodiments, the RNA may comprise a 5′-UTR derived from a NOSIP gene, wherein said 5′-UTR derived from a NOSIP gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6145 or 6146 or a fragment or a variant thereof.

In embodiments, the RNA may comprise a 5′-UTR derived from a RPL31 gene, wherein said 5′-UTR derived from a RPL31 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6147 or 6148 or a fragment or a variant thereof.

In preferred embodiments, the RNA may comprise a 5′-UTR derived from a SLC7A3 gene, wherein said 5′-UTR derived from a SLC7A3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6149 or 6150 or a fragment or a variant thereof.

In embodiments, the RNA may comprise a 5′-UTR derived from a TUBB4B gene, wherein said 5′-UTR derived from a TUBB4B gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6151 or 6152 or a fragment or a variant thereof.

In embodiments, the RNA may comprise a 5′-UTR derived from a UBQLN2 gene, wherein said 5′-UTR derived from a UBQLN2 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 6153 or 6154 or a fragment or a variant thereof.

Accordingly, the RNA of the first aspect may suitably comprise at least one 5′-UTR comprising or consisting of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6135-6156 or a fragment or a variant thereof.

In other embodiments, the RNA of the first aspect comprises a 5′-UTR as described in WO2013/143700, the disclosure of WO2013/143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013/143700, or fragments or variants of these sequences. In this context, it is preferred that the 5′-UTR of the RNA comprises or consists of a corresponding RNA sequence of the nucleic acid sequence according SEQ ID NOs: 1-1363, SEQ ID NO: 1395, SEQ ID NO: 1421 and SEQ ID NO: 1422 of WO2013/143700. In other embodiments, the RNA of the first aspect comprises a 5′-UTR as described in WO2016/107877, the disclosure of WO2016/107877 relating to 5′-UTR sequences herewith incorporated by reference. Particularly preferred 5′-UTRs are nucleic acid sequences according to SEQ ID NOs: 25 to 30 and SEQ ID NOs: 319 to 382 of WO2016/107877, or fragments or variants of these sequences. In this context, it is particularly preferred that the 5′-UTR of the RNA comprises or consists of a corresponding RNA sequence of the nucleic acid sequence according SEQ ID NOs: 25 to 30 and SEQ ID NOs: 319 to 382 of WO2016/107877. In other embodiments, the RNA of the first aspect comprises a 5′-UTR as described in WO2017/036580, the disclosure of WO2017/036580 relating to 5′-UTR sequences herewith incorporated by reference. Particularly preferred 5′-UTRs are nucleic acid sequences according to SEQ ID NOs: 1 to 151 of WO2017/036580, or fragments or variants of these sequences. In this context, it is particularly preferred that the 5′-UTR of the RNA comprises or consists of a corresponding RNA sequence of the nucleic acid sequence according to SEQ ID NOs: 1 to 151 of WO2017/036580. In other embodiments, the RNA of the first aspect comprises a 5′-UTR as described in WO2016/022914, the disclosure of WO2016/022914 relating to 5′-UTR sequences herewith incorporated by reference. Particularly preferred 5′-UTRs are nucleic acid sequences according to SEQ ID NOs: 3 to 19 of WO2016/022914, or fragments or variants of these sequences. In this context, it is particularly preferred that the 5′-UTR of the RNA comprises or consists of a corresponding RNA sequence of the nucleic acid sequence according to SEQ ID NOs: 3 to 19 of WO2016/022914.

Suitably, in preferred embodiments, the RNA of the first aspect comprises at least one coding sequence encoding at least one peptide or protein derived from a Malaria parasite, operably linked to a 3′-UTR and/or a 5′-UTR selected from the following 5′UTR/3′UTR combinations (“also referred to mRNA designs”): a-1 (HSD17B4/PSMB3), a-2 (NDUFA4/PSMB3), a-3 (SLC7A3/PSMB3), a-4 (NOSIP/PSMB3), a-5 (MP68/PSMB3), b-1 (UBQLN2/RPS9), b-2 (ASAH1/RPS9), b-3 (HSD17B4/RPS9), b-4 (HSD17B4/CASP1), b-5 (NOSIP/COX6B1), c-1 (NDUFA4/RPS9), c-2 (NOSIP/NDUFA1), c-3 (NDUFA4/COX6B1), c-4 (NDUFA4/NDUFA1), c-5 (ATP5A1/PSMB3), d-1 (RpI31/PSMB3), d-2 (ATP5A1/CASP1), d-3 (SLC7A3/GNAS), d-4 (HSD17B4/NDUFA1), d-5 (Slc7a3/Ndufa1), e-1 (TUBB4B/RPS9), e-2 (RPL31/RPS9), e-3 (MP68/RPS9), e-4 (NOSIP/RPS9), e-5 (ATP5A1/RPS9), e-6 (ATP5A1/COX6B1), f-1 (ATP5A1/GNAS), f-2 (ATP5A1/NDUFA1), f-3 (HSD17B4/COX6B1), f-4 (HSD17B4/GNAS), f-5 (MP68/COX6B1), g-1 (MP68/NDUFA1), g-2 (NDUFA4/CASP1), g-3 (NDUFA4/GNAS), g-4 (NOSIP/CASP1), g-5 (RPL31/CASP1), h-1 (RPL31/COX6B1), h-2 (RPL31/GNAS), h-3 (RPL31/NDUFA1), h-4 (Slc7a3/CASP1), h-5 (SLC7A3/COX6B1), i-1 (SLC7A3/RPS9), i-2 (RPL32/ALB7), i-2 (RPL32/ALB7), or i-3 (α-globin gene).

In particularly preferred embodiments of the first aspect, the RNA comprises at least one coding sequence as specified herein encoding at least one peptide or protein derived from a Malaria parasite, wherein said coding sequence is operably linked to a 5′-UTR selected from HSD17B4 and a 3′-UTR selected from PSMB3 (mRNA design a-1 (HSD17B4/PSMB3)).

In preferred embodiments of the first aspect, the RNA comprises at least one coding sequence as specified herein encoding at least one peptide or protein derived from a Malaria parasite, wherein said coding sequence is operably linked to a 5′-UTR selected from SLC7A3 and a 3′-UTR selected from PSMB3 (mRNA design a-3 (SLC7A3/PSMB3)).

Accordingly, the RNA of the first aspect comprises at least one coding sequence encoding at least one peptide or protein as defined herein, wherein administration of said RNA results in expression and/or activity of the encoded peptide or protein in the subject, wherein said coding sequence as defined herein is operably linked to a 5′-UTR and/or 3′-UTR, wherein suitably
said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6135 or 6136 or a fragment or a variant thereof;
said 5′-UTR derived from a ASAH1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6137 or 6138 or a fragment or a variant thereof;
said 5′-UTR derived from a ATP5A1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6139 or 6140 or a fragment or a variant thereof;
said 5′-UTR derived from a MP68 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6141 or 6142 or a fragment or a variant thereof;
said 5′-UTR derived from a NDUFA4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6143 or 6144 or a fragment or a variant thereof;
said 5′-UTR derived from a NOSIP gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6145 or 6146 or a fragment or a variant thereof;
said 5′-UTR derived from a RPL31 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6147 or 6148 or a fragment or a variant thereof;
said 5′-UTR derived from a RPL32 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6155 or 6156 or a fragment or a variant thereof;
said 5′-UTR derived from a SLC7A3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6149 or 6150 or a fragment or a variant thereof;
said 5′-UTR derived from a TUBB4B gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6151 or 6152 or a fragment or a variant thereof;
said 5′-UTR derived from a UBQLN2 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 6153 or 6154 or a fragment or a variant thereof;
said 3′-UTR derived from a PSMB3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6157 or 6158 or a fragment or a variant thereof;
said 3′-UTR derived from a CASP1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6159 or 6160 or a fragment or a variant thereof;
said 3′-UTR derived from a COX6B1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6161 or 6162 or a fragment or a variant thereof;
said 3′-UTR derived from a GNAS gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6163 or 6164 or a fragment or a variant thereof;
said 3′-UTR derived from a NDUFA1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6165 or 6166 or a fragment or a variant thereof;
said 3′-UTR derived from a RPS9 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6167 or 6168 or a fragment or a variant thereof;
said 3′-UTR derived from a ALB7 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6169 or 6170 or a fragment or a variant thereof;
said 3′-UTR derived from a alpha-globin gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 6171 or 6172 or a fragment or a variant thereof.
   Suitable mRNA for a Malaria Vaccine:

In various embodiments of the first aspect, the coding RNA, preferably mRNA comprises, preferably in 5′- to 3′-direction, the following elements:
A) 5′-cap structure, preferably as specified herein;
B) 5′-terminal start element, preferably as specified herein;
C) optionally, a cleavage site for a catalytic nucleic acid molecule, preferably as specified herein;
D) optionally, 5′-UTR, preferably as specified herein;
F) a ribosome binding site, preferably as specified herein;
E) at least one coding sequence, preferably as specified herein;
F) 3′-UTR, preferably as specified herein;
G) optionally, poly(A) sequence, preferably as specified herein;
H) optionally, poly(C) sequence, preferably as specified herein;
I) optionally, histone stem-loop preferably as specified herein;
J) optionally, 3″-terminal sequence element, preferably as specified herein.

In preferred embodiments of the first aspect, the coding RNA, preferably mRNA comprises the following elements preferably in 5′- to 3′-direction:
A) 5′-cap structure selected from m7G(5′), m7G(5′)ppp(5′)(2′OMeA), or m7G(5′)ppp(5′)(2′OMeG);
B) 5′-terminal start element selected from SEQ ID NOs: 6177 or 6178 or fragments or variants thereof;
C) optionally, a cleavage site for a catalytic nucleic acid molecule, preferably as specified herein;
D) optionally, 5′-UTR selected from SEQ ID NOs: 6135-6156 or fragments or variants thereof;
F) a ribosome binding site selected from SEQ ID NOs: 6175, 6176 or fragments or variants thereof;
E) at least one coding sequence selected from SEQ ID NOs: 37-328, 2121-2480, 2887-6134, 8754-8855, 10086-10139 or fragments or variants thereof;
F) 3′-UTR selected from SEQ ID NOs: 6157 to 6172;
G) optionally, poly(A) sequence comprising about 50 to about 500 adenosines;
H) optionally, poly(C) sequence comprising about 10 to about 100 cytosines;
I) optionally, histone stem-loop selected from SEQ ID NOs: 6173 or 6174;
J) optionally, 3″-terminal sequence element SEQ ID NOs: 6179-6200, 10173-10196.

In further preferred embodiments of the first aspect, the coding RNA, preferably mRNA comprises the following elements:
A) 5′-cap structure selected from m7G(5′), m7G(5′)ppp(5′)(2′OMeA), or m7G(5′)ppp(5′)(2′OMeG);
B) 5′-terminal start element selected from SEQ ID NOs: 6177 or 6178 or fragments or variants thereof;
C) 3′-UTR and/or 5′-UTR element according to a-1, a-2, a-3, a-4, a-5, b-1, b-2, b-3, b-4, b-5, c-1, c-2, c-3, c-4, c-5, d-1, d-2, d-3, d-4, d-5, e-1, e-2, e-3, e-4, e-5, e-6, f-1, f-2, f-3, f-4, f-5, g-1, g-2, g-3, g- 4, g-5, h-1, h-2, h-3, h-4, h-5, i-1, i-2, or i-3, as specified herein, wherein a-1, a-3, i-2, i-3 are preferred;
D) a ribosome binding site selected from SEQ ID NOs: 6175, 6176 or fragments or variants thereof;
E) at least one coding sequence selected from SEQ ID NOs: 37-328, 8754-8855 or fragments or variants thereof;
G) poly(A) sequence comprising about 50 to about 500 adenosines, preferably about 64 or 100 adenosines;
H) optionally, poly(C) sequence comprising about 10 to about 100 cytosines, preferably about 30 cytosines;
I) optionally, histone stem-loop selected from SEQ ID NOs: 6173 or 6174.

In particularly preferred embodiments of the first aspect, the coding RNA, preferably mRNA comprises the following elements:
A) 5′-cap structure selected from m7G(5′), m7G(5′)ppp(5′)(2′OMeA), or m7G(5′)ppp(5′)(2′OMeG);
B) 5′-terminal start element selected from SEQ ID NOs: 6177 or 6178 or fragments or variants thereof;
C) 3′-UTR and/or 5′-UTR element according to a-1, a-3, i-2, i-3;
D) a ribosome binding site selected from SEQ ID NOs: 6175, 6176 or fragments or variants thereof;
E) at least one coding sequence selected from SEQ ID NOs: 44, 80, 116, 152, 188, 224, 260, 296, 8755 (HsALB_Pf-CSP(19-397)) or fragments or variants thereof;
G) poly(A) sequence comprising about 50 to about 500 adenosines, preferably about 64 or 100 adenosines;
H) optionally, poly(C) sequence comprising about 10 to about 100 cytosines, preferably about 30 cytosines;
I) optionally, histone stem-loop selected from SEQ ID NOs: 6173 or 6174.

Preferred amino acid sequences, coding sequences, and mRNA sequences of the invention are provided in Table 6A and 6B. Therein, each row represents a specific suitable CSP construct of the invention, wherein the description of the CSP construct is indicated in column A, the SEQ ID NOs of the amino acid sequence is provided in column B. The respective accession number(s), and further information is provided under <223> identifier of the respective SEQ ID NOs in the sequence listing.

The corresponding SEQ ID NOs of the coding sequences encoding the respective CSP constructs are provided in column C of Table 6A and 6B (wild type cds) and D (opt1, opt2, opt3, opt4, opt5, opt11 cds). Further information is provided under <223> identifier of the respective SEQ ID NO in the sequence listing.

For Table 6A, the corresponding RNA sequences comprising preferred coding sequences are provided in columns E to H, wherein column E (“a-1”) provides RNA sequences with advantageous UTR combination “a-1” as defined herein, and wherein column F (“i-2”) provides RNA sequences with advantageous UTR combination “i-2” as defined herein, and wherein column G (1-3″) provides RNA sequences with advantageous UTR combination “i-3” as defined herein, and wherein column H (“a-3”) provides RNA sequences with advantageous UTR combination “a-3” as defined herein.

TABLE 6A
Preferred mRNA constructs encoding CSP

| A | B | C | D | E | F | G | H |
|---|---|---|---|---|---|---|---|
| Pf-CSP | 1 | 37 | 41, 77, | 329, 333, 369, 405, | 913, 917, | 1497, 1501, 1537, | 6312, 6315, 6345, |
| | | | 113, | 441, 477, 513, 549, | 953, 989, | 1573, 1609, 1645, | 6375, 6405, 6435, |
| | | | 149, | 585, 621, 625, 661, | 1025, 1061, | 1681, 1717, 1753, | 6465, 6495, 6525, |
| | | | 185, | 697, 733, 769, 805, | 1097, 1133, | 1789, 1793, 1829, | 6798, 6801, 6831, |
| | | | 221, | 841, 877, 6555, | 1169, 1205, | 1865, 1901, 1937, | 6861, 6891, 6921, |
| | | | 257, | 6558, 6588, 6618, | 1209, 1245, | 1973, 2009, 2045, | 6951, 6981, 7011, |
| | | | 293 | 6648, 6678, 6708, | 1281, 1317, | 7041, 7044, 7074, | 7527, 7530, 7560, |
| | | | | 6738, 6768, 7284, | 1353, 1389, | 7104, 7134, 7164, | 7590, 7620, 7650, |
| | | | | 7287, 7317, 7347, | 1425, 1461 | 7194, 7224, 7254, | 7680, 7710, 7740, |
| | | | | 7377, 7407, 7437, | | 7770, 7773, 7803, | 8256, 8259, 8289, |
| | | | | 7467, 7497, 8013, | | 7833, 7863, 7893, | 8319, 8349, 8379, |
| | | | | 8016, 8046, 8076, | | 7923, 7953, 7983, | 8409, 8439, 8469 |
| | | | | 8106, 8136, 8166, | | 8499, 8502, 8532, | |
| | | | | 8196, 8226 | | 8562, 8592, 8622, | |
| | | | | | | 8652, 8682, 8712 | |
| Pf-CSP(1- | 2 | 38 | 42, 78, | 330, 334, 370, 406, | 914, 918, | 1498, 1502, 1538, | 6313, 6316, 6346, |
| 374) | | | 114, | 442, 478, 514, 550, | 954, 990, | 1574, 1610, 1646, | 6376, 6406, 6436, |
| | | | 150, | 586, 622, 626, 662, | 1026, 1062, | 1682, 1718, 1754, | 6466, 6496, 6526, |
| | | | 186, | 698, 734, 770, 806, | 1098, 1134, | 1790, 1794, 1830, | 6799, 6802, 6832, |
| | | | 222, | 842, 878, 6556, | 1170, 1206, | 1866, 1902, 1938, | 6862, 6892, 6922, |
| | | | 258, | 6559, 6589, 6619, | 1210, 1246, | 1974, 2010, 2046, | 6952, 6982, 7012, |
| | | | 294 | 6649, 6679, 6709, | 1282, 1318, | 7042, 7045, 7075, | 7528, 7531, 7561, |
| | | | | 6739, 6769, 7285, | 1354, 1390, | 7105, 7135, 7165, | 7591, 7621, 7651, |
| | | | | 7288, 7318, 7348, | 1426, 1462 | 7195, 7225, 7255, | 7681, 7711, 7741, |
| | | | | 7378, 7408, 7438, | | 7771, 7774, 7804, | 8257, 8260, 8290, |
| | | | | 7468, 7498, 8014, | | 7834, 7864, 7894, | 8320, 8350, 8380, |
| | | | | 8017, 8047, 8077, | | 7924, 7954, 7984, | 8410, 8440, 8470 |
| | | | | 8107, 8137, 8167, | | 8500, 8503, 8533, | |
| | | | | 8197, 8227 | | 8563, 8593, 8623, | |
| | | | | | | 8653, 8683, 8713 | |
| Pf-CSP(1- | 3 | 39 | 43, 79, | 331, 335, 371, 407, | 915, 919, | 1499, 1503, 1539, | 6314, 6317, 6347, |
| 325) | | | 115, | 443, 479, 515, 551, | 955, 991, | 1575, 1611, 1647, | 6377, 6407, 6437, |
| | | | 151, | 587, 623, 627, 663, | 1027, 1063, | 1683, 1719, 1755, | 6467, 6497, 6527, |
| | | | 187, | 699, 735, 771, 807, | 1099, 1135, | 1791, 1795, 1831, | 6800, 6803, 6833, |
| | | | 223, | 843, 879, 6557, | 1171, 1207, | 1867, 1903, 1939, | 6863, 6893, 6923, |
| | | | 259, | 6560, 6590, 6620, | 1211, 1247, | 1975, 2011, 2047, | 6953, 6983, 7013, |
| | | | 295 | 6650, 6680, 6710, | 1283, 1319, | 7043, 7046, 7076, | 7529, 7532, 7562, |
| | | | | 6740, 6770, 7286, | 1355, 1391, | 7106, 7136, 7166, | 7592, 7622, 7652, |
| | | | | 7289, 7319, 7349, | 1427, 1463 | 7196, 7226, 7256, | 7682, 7712, 7742, |
| | | | | 7379, 7409, 7439, | | 7772, 7775, 7805, | 8258, 8261, 8291, |
| | | | | 7469, 7499, 8015, | | 7835, 7865, 7895, | 8321, 8351, 8381, |
| | | | | 8018, 8048, 8078, | | 7925, 7955, 7985, | 8411, 8441, 8471 |
| | | | | 8108, 8138, 8168, | | 8501, 8504, 8534, | |
| | | | | 8198, 8228 | | 8564, 8594, 8624, | |
| | | | | | | 8654, 8684, 8714 | |
| HsALB_Pf | 4 | | 44, 80, | 336, 372, 408, 444, | 920, 956, | 1504, 1540, 1576, | 6318, 6348, 6378, |
| 4DSP(19- | | | 8755, | 480, 516, 552, 588, | 992, 1028, | 1612, 1648, 1684, | 6408, 6438, 6468, |
| 397) | | | 116, | 628, 664, 700, 736, | 1064, 1100, | 1720, 1756, 1796, | 6498, 6528, 8959, |
| | | | 152, | 772, 808, 844, 880, | 1136, 1172, | 1832, 1868, 1904, | 6804, 6834, 6864, |
| | | | 188, | 8857, 6561, 6591, | 1212, 1248, | 1940, 1976, 2012, | 6894, 6924, 6954, |
| | | | 224, | 6621, 6651, 6681, | 1284, 1320, | 2048, 9061, 7047, | 6984, 7014, 9265, |
| | | | 260, | 6711, 6741, 6771, | 1356, 1392, | 7077, 7107, 7137, | 7533, 7563, 7593, |
| | | | 296 | 9163, 7290, 7320, | 1428, 1464 | 7167, 7197, 7227, | 7623, 7653, 7683, |
| | | | | 7350, 7380, 7410, | | 7257, 9367, 7776, | 7713, 7743, 9571, |
| | | | | 7440, 7470, 7500, | | 7806, 7836, 7866, | 8262, 8292, 8322, |
| | | | | 9469, 8019, 8049, | | 7896, 7926, 7956, | 8352, 8382, 8412, |
| | | | | 8079, 8109, 8139, | | 7986, 9673, 8505, | 8442, 8472, 9877 |
| | | | | 8169, 8199, 8229, | | 8535, 8565, 8595, | |
| | | | | 9775 | | 8625, 8655, 8685, | |
| | | | | | | 8715, 9979 | |
| Hsins- | 5 | | 45, 81, | 337, 373, 409, 445, | 921, 957, | 1505, 1541, 1577, | 6319, 6349, 6379, |
| isol_Pf- | | | 117, | 481, 517, 553, 589, | 993, 1029, | 1613, 1649, 1685, | 6409, 6439, 6469, |
| CSP(19- | | | 153, | 629, 665, 701, 737, | 1065, 1101, | 1721, 1757, 1797, | 6499, 6529, 6805, |
| 397) | | | 189, | 773, 809, 845, 881, | 1137, 1173, | 1833, 1869, 1905, | 6835, 6865, 6895, |
| | | | 225, | 6562, 6592, 6622, | 1213, 1249, | 1941, 1977, 2013, | 6925, 6955, 6985, |
| | | | 261, | 6652, 6682, 6712, | 1285, 1321, | 2049, 7048, 7078, | 7015, 7534, 7564, |
| | | | 297 | 6742, 6772, 7291, | 1357, 1393, | 7108, 7138, 7168, | 7594, 7624, 7654, |
| | | | | 7321, 7351, 7381, | 1429, 1465 | 7198, 7228, 7258, | 7684, 7714, 7744, |
| | | | | 7411, 7441, 7471, | | 7777, 7807, 7837, | 8263, 8293, 8323, |
| | | | | 7501, 8020, 8050, | | 7867, 7897, 7927, | 8353, 8383, 8413, |
| | | | | 8080, 8110, 8140, | | 7957, 7987, 8506, | 8443, 8473 |
| | | | | 8170, 8200, 8230 | | 8536, 8566, 8596, | |
| | | | | | | 8626, 8656, 8686, | |
| | | | | | | 8716 | |
| HsSPARC | 6 | | 46, 82, | 338, 374, 410, 446, | 922, 958, | 1506, 1542, 1578, | 6320, 6350, 6380, |
| Pf- | | | 118, | 482, 518, 554, 590, | 994, 1030, | 1614, 1650, 1686, | 6410, 6440, 6470, |
| CSP(19- | | | 154, | 630, 666, 702, 738, | 1066, 1102, | 1722, 1758, 1798, | 6500, 6530, 6806, |
| 397) | | | 190, | 774, 810, 846, 882, | 1138, 1174, | 1834, 1870, 1906, | 6836, 6866, 6896, |
| | | | 226, | 6563, 6593, 6623, | 1214, 1250, | 1942, 1978, 2014, | 6926, 6956, 6986, |
| | | | 262, | 6653, 6683, 6713, | 1286, 1322, | 2050, 7049, 7079, | 7016, 7535, 7565, |
| | | | 298 | 6743, 6773, 7292, | 1358, 1394, | 7109, 7139, 7169, | 7595, 7625, 7655, |
| | | | | 7322, 7352, 7382, | 1430, 1466 | 7199, 7229, 7259, | 7685, 7715, 7745, |
| | | | | 7412, 7442, 7472, | | 7778, 7808, 7838, | 8264, 8294, 8324, |
| | | | | 7502, 8021, 8051, | | 7868, 7898, 7928, | 8354, 8384, 8414, |
| | | | | 8081, 8111, 8141, | | 7958, 7988, 8507, | 8444, 8474 |
| | | | | 8171, 8201, 8231 | | 8537, 8567, 8597, | |
| | | | | | | 8627, 8657, 8687, | |
| | | | | | | 8717 | |
| IgE_Pf- | 7 | | 47, 83, | 339, 375, 411, 447, | 923, 959, | 1507, 1543, 1579, | 6321, 6351, 6381, |
| CSP(19- | | | 119, | 483, 519, 555, 591, | 995, 1031, | 1615, 1651, 1687, | 6411, 6441, 6471, |
| 397) | | | 155, | 631, 667, 703, 739, | 1067, 1103, | 1723, 1759, 1799, | 6501, 6531, 6807, |
| | | | 191, | 775, 811, 847, 883, | 1139, 1175, | 1835, 1871, 1907, | 6837, 6867, 6897, |
| | | | 227, | 6564, 6594, 6624, | 1215, 1251, | 1943, 1979, 2015, | 6927, 6957, 6987, |
| | | | 263, | 6654, 6684, 6714, | 1287, 1323, | 2051, 7050, 7080, | 7017, 7536, 7566, |
| | | | 299 | 6744, 6774, 7293, | 1359, 1395, | 7110, 7140, 7170, | 7596, 7626, 7656, |
| | | | | 7323, 7353, 7383, | 1431, 1467 | 7200, 7230, 7260, | 7686, 7716, 7746, |
| | | | | 7413, 7443, 7473, | | 7779, 7809, 7839, | 8265, 8295, 8325, |
| | | | | 7503, 8022, 8052, | | 7869, 7899, 7929, | 8355, 8385, 8415, |
| | | | | 8082, 8112, 8142, | | 7959, 7989, 8508, | 8445, 8475 |
| | | | | 8172, 8202, 8232 | | 8538, 8568, 8598, | |
| | | | | | | 8628, 8658, 8688, | |
| | | | | | | 8718 | |
| Pf- | 8 | | 48, 84, | 340, 376, 412, 448, | 924, 960, | 1508, 1544, 1580, | 6322, 6352, 6382, |
| CSP_Link- | | | 120, | 484, 520, 556, 592, | 996, 1032, | 1616, 1652, 1688, | 6412, 6442, 6472, |
| er(G4S | | | 156, | 632, 668, 704, 740, | 1068, 1104, | 1724, 1760, 1800, | 6502, 6532, 6808, |
| G4)_TIm | | | 192, | 776, 812, 848, 884, | 1140, 1176, | 1836, 1872, 1908, | 6838, 6868, 6898, |
| domain | | | 228, | 6565, 6595, 6625, | 1216, 1252, | 1944, 1980, 2016, | 6928, 6958, 6988, |
| HA | | | 264, | 6655, 6685, 6715, | 1288, 1324, | 2052, 7051, 7081, | 7018, 7537, 7567, |
| | | | 300 | 6745, 6775, 7294, | 1360, 1396, | 7111, 7141, 7171, | 7597, 7627, 7657, |
| | | | | 7324, 7354, 7384, | 1432, 1468 | 7201, 7231, 7261, | 7687, 7717, 7747, |
| | | | | 7414, 7444, 7474, | | 7780, 7810, 7840, | 8266, 8296, 8326, |
| | | | | 7504, 8023, 8053, | | 7870, 7900, 7930, | 8356, 8386, 8416, |
| | | | | 8083, 8113, 8143, | | 7960, 7990, 8509, | 8446, 8476 |
| | | | | 8173, 8203, 8233 | | 8539, 8569, 8599, | |
| | | | | | | 8629, 8659, 8689, | |
| | | | | | | 8719 | |
| Pf- | 9 | | 49, 85, | 341, 377, 413, 449, | 925, 961, | 1509, 1545, 1581, | 6323, 6353, 6383, |
| CSP(199- | | | 121, | 485, 521, 557, 593, | 997, 1033, | 1617, 1653, 1689, | 6413, 6443, 6473, |
| 377)_Link- | | | 157, | 633, 669, 705, 741, | 1069, 1105, | 1725, 1761, 1801, | 6503, 6533, 6809, |
| er(PVTN) | | | 193, | 777, 813, 849, 885, | 1141, 1177, | 1837, 1873, 1909, | 6839, 6869, 6899, |
| HBsAg | | | 229, | 6566, 6596, 6626, | 1217, 1253, | 1945, 1981, 2017, | 6929, 6959, 6989, |
| | | | 265, | 6656, 6686, 6716, | 1289, 1325, | 2053, 7052, 7082, | 7019, 7538, 7568, |
| | | | 301 | 6746, 6776, 7295, | 1361, 1397, | 7112, 7142, 7172, | 7598, 7628, 7658, |
| | | | | 7325, 7355, 7385, | 1433, 1469 | 7202, 7232, 7262, | 7688, 7718, 7748, |
| | | | | 7415, 7445, 7475, | | 7781, 7811, 7841, | 8267, 8297, 8327, |
| | | | | 7505, 8024, 8054, | | 7871, 7901, 7931, | 8357, 8387, 8417, |
| | | | | 8084, 8114, 8144, | | 7961, 7991, 8510, | 8447, 8477 |
| | | | | 8174, 8204, 8234 | | 8540, 8570, 8600, | |
| | | | | | | 8630, 8660, 8690, | |
| | | | | | | 8720 | |
| Pf- | 10 | | 50, 86, | 342, 378, 414, 450, | 926, 962, | 1510, 1546, 1582, | 6324, 6354, 6384, |
| CSP(199- | | | 122, | 486, 522, 558, 594, | 998, 1034, | 1618, 1654, 1690, | 6414, 6444, 6474, |
| 387)_Link- | | | 158, | 634, 670, 706, 742, | 1070, 1106, | 1726, 1762, 1802, | 6504, 6534, 6810, |
| er(PVTN) | | | 194, | 778, 814, 850, 886, | 1142, 1178, | 1838, 1874, 1910, | 6840, 6870, 6900, |
| HBsAg | | | 230, | 6567, 6597, 6627, | 1218, 1254, | 1946, 1982, 2018, | 6930, 6960, 6990, |
| | | | 266, | 6657, 6687, 6717, | 1290, 1326, | 2054, 7053, 7083, | 7020, 7539, 7569, |
| | | | 302 | 6747, 6777, 7296, | 1362, 1398, | 7113, 7143, 7173, | 7599, 7629, 7659, |
| | | | | 7326, 7356, 7386, | 1434, 1470 | 7203, 7233, 7263, | 7689, 7719, 7749, |
| | | | | 7416, 7446, 7476, | | 7782, 7812, 7842, | 8268, 8298, 8328, |
| | | | | 7506, 8025, 8055, | | 7872, 7902, 7932, | 8358, 8388, 8418, |
| | | | | 8085, 8115, 8145, | | 7962, 7992, 8511, | 8448, 8478 |
| | | | | 8175, 8205, 8235 | | 8541, 8571, 8601, | |
| | | | | | | 8631, 8661, 8691, | |
| | | | | | | 8721 | |
| HsALB_Pf- | 11 | | 51, 87, | 343, 379, 415, 451, | 927, 963, | 1511, 1547, 1583, | 6325, 6355, 6385, |
| CSP(19- | | | 123, | 487, 523, 559, 595, | 999, 1035, | 1619, 1655, 1691, | 6415, 6445, 6475, |
| 152) | | | 159, | 635, 671, 707, 743, | 1071, 1107, | 1727, 1763, 1803, | 6505, 6535, 6811, |
| | | | 195, | 779, 815, 851, 887, | 1143, 1179, | 1839, 1875, 1911, | 6841, 6871, 6901, |
| | | | 231, | 6568, 6598, 6628, | 1219, 1255, | 1947, 1983, 2019, | 6931, 6961, 6991, |
| | | | 267, | 6658, 6688, 6718, | 1291, 1327, | 2055, 7054, 7084, | 7021, 7540, 7570, |
| | | | 303 | 6748, 6778, 7297, | 1363, 1399, | 7114, 7144, 7174, | 7600, 7630, 7660, |
| | | | | 7327, 7357, 7387, | 1435, 1471 | 7204, 7234, 7264, | 7690, 7720, 7750, |
| | | | | 7417, 7447, 7477, | | 7783, 7813, 7843, | 8269, 8299, 8329, |
| | | | | 7507, 8026, 8056, | | 7873, 7903, 7933, | 8359, 8389, 8419, |
| | | | | 8086, 8116, 8146, | | 7963, 7993, 8512, | 8449, 8479 |
| | | | | 8176, 8206, 8236 | | 8542, 8572, 8602, | |
| | | | | | | 8632, 8662, 8692, | |
| | | | | | | 8722 | |
| HsALB_Pf- | 12 | | 52, 88, | 344, 380, 416, 452, | 928, 964, | 1512, 1548, 1584, | 6326, 6356, 6386, |
| CSP(19- | | | 124, | 488, 524, 560, 596, | 1000, 1036, | 1620, 1656, 1692, | 6416, 6446, 6476, |
| 192) | | | 160, | 636, 672, 708, 744, | 1072, 1108, | 1728, 1764, 1804, | 6506, 6536, 6812, |
| | | | 196, | 780, 816, 852, 888, | 1144, 1180, | 1840, 1876, 1912, | 6842, 6872, 6902, |
| | | | 232, | 6569, 6599, 6629, | 1220, 1256, | 1948, 1984, 2020, | 6932, 6962, 6992, |
| | | | 268, | 6659, 6689, 6719, | 1292, 1328, | 2056, 7055, 7085, | 7022, 7541, 7571, |
| | | | 304 | 6749, 6779, 7298, | 1364, 1400, | 7115, 7145, 7175, | 7601, 7631, 7661, |
| | | | | 7328, 7358, 7388, | 1436, 1472 | 7205, 7235, 7265, | 7691, 7721, 7751, |
| | | | | 7418, 7448, 7478, | | 7784, 7814, 7844, | 8270, 8300, 8330, |
| | | | | 7508, 8027, 8057, | | 7874, 7904, 7934, | 8360, 8390, 8420, |
| | | | | 8087, 8117, 8147, | | 7964, 7994, 8513, | 8450, 8480 |
| | | | | 8177, 8207, 8237 | | 8543, 8573, 8603, | |
| | | | | | | 8633, 8663, 8693, | |
| | | | | | | 8723 | |
| HsALB_P-f | 13 | | 53, 89, | 345, 381, 417, 453, | 929, 965, | 1513, 1549, 1585, | 6327, 6357, 6387, |
| CSP(19- | | | 125, | 489, 525, 561, 597, | 1001, 1037, | 1621, 1657, 1693, | 6417, 6447, 6477, |
| 272) | | | 161, | 637, 673, 709, 745, | 1073, 1109, | 1729, 1765, 1805, | 6507, 6537, 6813, |
| | | | 197, | 781, 817, 853, 889, | 1145, 1181, | 1841, 1877, 1913, | 6843, 6873, 6903, |
| | | | 233, | 6570, 6600, 6630, | 1221, 1257, | 1949, 1985, 2021, | 6933, 6963, 6993, |
| | | | 269, | 6660, 6690, 6720, | 1293, 1329, | 2057, 7056, 7086, | 7023, 7542, 7572, |
| | | | 305 | 6750, 6780, 7299, | 1365, 1401, | 7116, 7146, 7176, | 7602, 7632, 7662, |
| | | | | 7329, 7359, 7389, | 1437, 1473 | 7206, 7236, 7266, | 7692, 7722, 7752, |
| | | | | 7419, 7449, 7479, | | 7785, 7815, 7845, | 8271, 8301, 8331, |
| | | | | 7509, 8028, 8058, | | 7875, 7905, 7935, | 8361, 8391, 8421, |
| | | | | 8088, 8118, 8148, | | 7965, 7995, 8514, | 8451, 8481 |
| | | | | 8178, 8208, 8238 | | 8544, 8574, 8604, | |
| | | | | | | 8634, 8664, 8694, | |
| | | | | | | 8724 | |
| HsALB_Pf- | 14 | | 54, 90, | 346, 382, 418, 454, | 930, 966, | 1514, 1550, 1586, | 6328, 6358, 6388, |
| CSP(19- | | | 8766, | 490, 526, 562, 598, | 1002, 1038, | 1622, 1658, 1694, | 6418, 6448, 6478, |
| 272)_Link- | | | 126, | 638, 674, 710, 746, | 1074, 1110, | 1730, 1766, 1806, | 6508, 6538, 8970, |
| er(AAY)_ | | | 162, | 782, 818, 854, 890, | 1146, 1182, | 1842, 1878, 1914, | 6814, 6844, 6874, |
| Pf- | | | 198, | 8868, 6571, 6601, | 1222, 1258, | 1950, 1986, 2022, | 6904, 6934, 6964, |
| CSP(310- | | | 234, | 6631, 6661, 6691, | 1294, 1330, | 2058, 9072, 7057, | 6994, 7024, 9276, |
| 327)_Link | | | 270, | 6721, 6751, 6781, | 1366, 1402, | 7087, 7117, 7147, | 7543, 7573, 7603, |
| er(AAY)_ | | | 306 | 9174, 7300, 7330, | 1438, 1474 | 7177, 7207, 7237, | 7633, 7663, 7693, |
| Pf- | | | | 7360, 7390, 7420, | | 7267, 9378, 7786, | 7723, 7753, 9582, |
| CSP(346- | | | | 7450, 7480, 7510, | | 7816, 7846, 7876, | 8272, 8302, 8332, |
| 375) | | | | 9480, 8029, 8059, | | 7906, 7936, 7966, | 8362, 8392, 8422, |
| | | | | 8089, 8119, 8149, | | 7996, 9684, 8515, | 8452, 8482, 9888 |
| | | | | 8179, 8209, 8239, | | 8545, 8575, 8605, | |
| | | | | 9786 | | 8635, 8665, 8695, | |
| | | | | | | 8725, 9990 | |
| HsALB_Pf- | 15 | | 55, 91, | 347, 383, 419, 455, | 931, 967, | 1515, 1551, 1587, | 6329, 6359, 6389, |
| CSP(19- | | | 127, | 491, 527, 563, 599, | 1003, 1039, | 1623, 1659, 1695, | 6419, 6449, 6479, |
| 272)_Link- | | | 163, | 639, 675, 711, 747, | 1075, 1111, | 1731, 1767, 1807, | 6509, 6539, 6815, |
| er(AAY)_ | | | 199, | 783, 819, 855, 891, | 1147, 1183, | 1843, 1879, 1915, | 6845, 6875, 6905, |
| Pf- | | | 235, | 6572, 6602, 6632, | 1223, 1259, | 1951, 1987, 2023, | 6935, 6965, 6995, |
| CSP(346- | | | 271, | 6662, 6692, 6722, | 1295, 1331, | 2059, 7058, 7088, | 7025, 7544, 7574, |
| 365)_Link- | | | 307 | 6752, 6782, 7301, | 1367, 1403, | 7118, 7148, 7178, | 7604, 7634, 7664, |
| er(AAY)_ | | | | 7331, 7361, 7391, | 1439, 1475 | 7208, 7238, 7268, | 7694, 7724, 7754, |
| P2 | | | | 7421, 7451, 7481, | | 7787, 7817, 7847, | 8273, 8303, 8333, |
| | | | | 7511, 8030, 8060, | | 7877, 7907, 7937, | 8363, 8393, 8423, |
| | | | | 8090, 8120, 8150, | | 7967, 7997, 8516, | 8453, 8483 |
| | | | | 8180, 8210, 8240 | | 8546, 8576, 8606, | |
| | | | | | | 8636, 8666, 8696, | |
| | | | | | | 8726 | |
| HsALB_Pf- | 16 | | 56, 92, | 348, 384, 420, 456, | 932, 968, | 1516, 1552, 1588, | 6330, 6360, 6390, |
| CSP(19- | | | 128, | 492, 528, 564, 600, | 1004, 1040, | 1624, 1660, 1696, | 6420, 6450, 6480, |
| 272)_Link- | | | 164, | 640, 676, 712, 748, | 1076, 1112, | 1732, 1768, 1808, | 6510, 6540, 6816, |
| er(AAY)_ | | | 200, | 784, 820, 856, 892, | 1148, 1184, | 1844, 1880, 1916, | 6846, 6876, 6906, |
| Pf- | | | 236, | 6573, 6603, 6633, | 1224, 1260, | 1952, 1988, 2024, | 6936, 6966, 6996, |
| CSP(346- | | | 272, | 6663, 6693, 6723, | 1296, 1332, | 2060, 7059, 7089, | 7026, 7545, 7575, |
| 365)_Link- | | | 308 | 6753, 6783, 7302, | 1368, 1404, | 7119, 7149, 7179, | 7605, 7635, 7665, |
| er(AAY)_ | | | | 7332, 7362, 7392, | 1440, 1476 | 7209, 7239, 7269, | 7695, 7725, 7755, |
| PADRE | | | | 7422, 7452, 7482, | | 7788, 7818, 7848, | 8274, 8304, 8334, |
| | | | | 7512, 8031, 8061, | | 7878, 7908, 7938, | 8364, 8394, 8424, |
| | | | | 8091, 8121, 8151, | | 7968, 7998, 8517, | 8454, 8484 |
| | | | | 8181, 8211, 8241 | | 8547, 8577, 8607, | |
| | | | | | | 8637, 8667, 8697, | |
| | | | | | | 8727 | |
| HsALB_Pf- | 17 | | 57, 93, | 349, 385, 421, 457, | 933, 969, | 1517, 1553, 1589, | 6331, 6361, 6391, |
| CSP(19- | | | 129, | 493, 529, 565, 601, | 1005, 1041, | 1625, 1661, 1697, | 6421, 6451, 6481, |
| 272)_Link- | | | 165, | 641, 677, 713, 749, | 1077, 1113, | 1733, 1769, 1809, | 6511, 6541, 6817, |
| er(G4S)_ | | | 201, | 785, 821, 857, 893, | 1149, 1185, | 1845, 1881, 1917, | 6847, 6877, 6907, |
| Pf- | | | 237, | 6574, 6604, 6634, | 1225, 1261, | 1953, 1989, 2025, | 6937, 6967, 6997, |
| CSP(310- | | | 273, | 6664, 6694, 6724, | 1297, 1333, | 2061, 7060, 7090, | 7027, 7546, 7576, |
| 327)_Link- | | | 309 | 6754, 6784, 7303, | 1369, 1405, | 7120, 7150, 7180, | 7606, 7636, 7666, |
| er(G4S)_ | | | | 7333, 7363, 7393, | 1441, 1477 | 7210, 7240, 7270, | 7696, 7726, 7756, |
| Pf- | | | | 7423, 7453, 7483, | | 7789, 7819, 7849, | 8275, 8305, 8335, |
| CSP(346- | | | | 7513, 8032, 8062, | | 7879, 7909, 7939, | 8365, 8395, 8425, |
| 375) | | | | 8092, 8122, 8152, | | 7969, 7999, 8518, | 8455, 8485 |
| | | | | 8182, 8212, 8242 | | 8548, 8578, 8608, | |
| | | | | | | 8638, 8668, 8698, | |
| | | | | | | 8728 | |
| HsALB_Pf- | 18 | | 58, 94, | 350, 386, 422, 458, | 934, 970, | 1518, 1554, 1590, | 6332, 6362, 6392, |
| CSP(19- | | | 130, | 494, 530, 566, 602, | 1006, 1042, | 1626, 1662, 1698, | 6422, 6452, 6482, |
| 272)_Link- | | | 166, | 642, 678, 714, 750, | 1078, 1114, | 1734, 1770, 1810, | 6512, 6542, 6818, |
| er(G4S)_ | | | 202, | 786, 822, 858, 894, | 1150, 1186, | 1846, 1882, 1918, | 6848, 6878, 6908, |
| Pf- | | | 238, | 6575, 6605, 6635, | 1226, 1262, | 1954, 1990, 2026, | 6938, 6968, 6998, |
| CSP(310- | | | 274, | 6665, 6695, 6725, | 1298, 1334, | 2062, 7061, 7091, | 7028, 7547, 7577, |
| 327)_Pf- | | | 310 | 6755, 6785, 7304, | 1370, 1406, | 7121, 7151, 7181, | 7607, 7637, 7667, |
| CSP(346- | | | | 7334, 7364, 7394, | 1442, 1478 | 7211, 7241, 7271, | 7697, 7727, 7757, |
| 375) | | | | 7424, 7454, 7484, | | 7790, 7820, 7850, | 8276, 8306, 8336, |
| | | | | 7514, 8033, 8063, | | 7880, 7910, 7940, | 8366, 8396, 8426, |
| | | | | 8093, 8123, 8153, | | 7970, 8000, 8519, | 8456, 8486 |
| | | | | 8183, 8213, 8243 | | 8549, 8579, 8609, | |
| | | | | | | 8639, 8669, 8699, | |
| | | | | | | 8729 | |
| HsALB_Pf- | 19 | | 59, 95, | 351, 387, 423, 459, | 935, 971, | 1519, 1555, 1591, | 6333, 6363, 6393, |
| CSP(19- | | | 131, | 495, 531, 567, 603, | 1007, 1043, | 1627, 1663, 1699, | 6423, 6453, 6483, |
| 325) | | | 167, | 643, 679, 715, 751, | 1079, 1115, | 1735, 1771, 1811, | 6513, 6543, 6819, |
| | | | 203, | 787, 823, 859, 895, | 1151, 1187, | 1847, 1883, 1919, | 6849, 6879, 6909, |
| | | | 239, | 6576, 6606, 6636, | 1227, 1263, | 1955, 1991, 2027, | 6939, 6969, 6999, |
| | | | 275, | 6666, 6696, 6726, | 1299, 1335, | 2063, 7062, 7092, | 7029, 7548, 7578, |
| | | | 311 | 6756, 6786, 7305, | 1371, 1407, | 7122, 7152, 7182, | 7608, 7638, 7668, |
| | | | | 7335, 7365, 7395, | 1443, 1479 | 7212, 7242, 7272, | 7698, 7728, 7758, |
| | | | | 7425, 7455, 7485, | | 7791, 7821, 7851, | 8277, 8307, 8337, |
| | | | | 7515, 8034, 8064, | | 7881, 7911, 7941, | 8367, 8397, 8427, |
| | | | | 8094, 8124, 8154, | | 7971, 8001, 8520, | 8457, 8487 |
| | | | | 8184, 8214, 8244 | | 8550, 8580, 8610, | |
| | | | | | | 8640, 8670, 8700, | |
| | | | | | | 8730 | |
| HsALB_Pf- | 20 | | 60, 96, | 352, 388, 424, 460, | 936, 972, | 1520, 1556, 1592, | 6334, 6364, 6394, |
| CSP(19- | | | 132, | 496, 532, 568, 604, | 1008, 1044, | 1628, 1664, 1700, | 6424, 6454, 6484, |
| 384) | | | 168, | 644, 680, 716, 752, | 1080, 1116, | 1736, 1772, 1812, | 6514, 6544, 6820, |
| | | | 204, | 788, 824, 860, 896, | 1152, 1188, | 1848, 1884, 1920, | 6850, 6880, 6910, |
| | | | 240, | 6577, 6607, 6637, | 1228, 1264, | 1956, 1992, 2028, | 6940, 6970, 7000, |
| | | | 276, | 6667, 6697, 6727, | 1300, 1336, | 2064, 7063, 7093, | 7030, 7549, 7579, |
| | | | 312 | 6757, 6787, 7306, | 1372, 1408, | 7123, 7153, 7183, | 7609, 7639, 7669, |
| | | | | 7336, 7366, 7396, | 1444, 1480 | 7213, 7243, 7273, | 7699, 7729, 7759, |
| | | | | 7426, 7456, 7486, | | 7792, 7822, 7852, | 8278, 8308, 8338, |
| | | | | 7516, 8035, 8065, | | 7882, 7912, 7942, | 8368, 8398, 8428, |
| | | | | 8095, 8125, 8155, | | 7972, 8002, 8521, | 8458, 8488 |
| | | | | 8185, 8215, 8245 | | 8551, 8581, 8611, | |
| | | | | | | 8641, 8671, 8701, | |
| | | | | | | 8731 | |
| HsALB_Pf- | 21 | | 61, 97, | 353, 389, 425, 461, | 937, 973, | 1521, 1557, 1593, | 6335, 6365, 6395, |
| CSP(19- | | | 133, | 497, 533, 569, 605, | 1009, 1045, | 1629, 1665, 1701, | 6425, 6455, 6485, |
| 384)_TM | | | 169, | 645, 681, 717, 753, | 1081, 1117, | 1737, 1773, 1813, | 6515, 6545, 6821, |
| domain | | | 205, | 789, 825, 861, 897, | 1153, 1189, | 1849, 1885, 1921, | 6851, 6881, 6911, |
| HA | | | 241, | 6578, 6608, 6638, | 1229, 1265, | 1957, 1993, 2029, | 6941, 6971, 7001, |
| | | | 277, | 6668, 6698, 6728, | 1301, 1337, | 2065, 7064, 7094, | 7031, 7550, 7580, |
| | | | 313 | 6758, 6788, 7307, | 1373, 1409, | 7124, 7154, 7184, | 7610, 7640, 7670, |
| | | | | 7337, 7367, 7397, | 1445, 1481 | 7214, 7244, 7274, | 7700, 7730, 7760, |
| | | | | 7427, 7457, 7487, | | 7793, 7823, 7853, | 8279, 8309, 8339, |
| | | | | 7517, 8036, 8066, | | 7883, 7913, 7943, | 8369, 8399, 8429, |
| | | | | 8096, 8126, 8156, | | 7973, 8003, 8522, | 8459, 8489 |
| | | | | 8186, 8216, 8246 | | 8552, 8582, 8612, | |
| | | | | | | 8642, 8672, 8702, | |
| | | | | | | 8732 | |
| HsALB_Pf- | 22 | | 62, 98, | 354, 390, 426, 462, | 938, 974, | 1522, 1558, 1594, | 6336, 6366, 6396, |
| CSP(82- | | | 134, | 498, 534, 570, 606, | 1010, 1046, | 1630, 1666, 1702, | 6426, 6456, 6486, |
| 397) | | | 170, | 646, 682, 718, 754, | 1082, 1118, | 1738, 1774, 1814, | 6516, 6546, 6822, |
| | | | 206, | 790, 826, 862, 898, | 1154, 1190, | 1850, 1886, 1922, | 6852, 6882, 6912, |
| | | | 242, | 6579, 6609, 6639, | 1230, 1266, | 1958, 1994, 2030, | 6942, 6972, 7002, |
| | | | 278, | 6669, 6699, 6729, | 1302, 1338, | 2066, 7065, 7095, | 7032, 7551, 7581, |
| | | | 314 | 6759, 6789, 7308, | 1374, 1410, | 7125, 7155, 7185, | 7611, 7641, 7671, |
| | | | | 7338, 7368, 7398, | 1446, 1482 | 7215, 7245, 7275, | 7701, 7731, 7761, |
| | | | | 7428, 7458, 7488, | | 7794, 7824, 7854, | 8280, 8310, 8340, |
| | | | | 7518, 8037, 8067, | | 7884, 7914, 7944, | 8370, 8400, 8430, |
| | | | | 8097, 8127, 8157, | | 7974, 8004, 8523, | 8460, 8490 |
| | | | | 8187, 8217, 8247 | | 8553, 8583, 8613, | |
| | | | | | | 8643, 8673, 8703, | |
| | | | | | | 8733 | |
| HsALB_Pf- | 23 | | 63, 99, | 355, 391, 427, 463, | 939, 975, | 1523, 1559, 1595, | 6337, 6367, 6397, |
| CSP(93- | | | 135, | 499, 535, 571, 607, | 1011, 1047, | 1631, 1667, 1703, | 6427, 6457, 6487, |
| 192) | | | 171, | 647, 683, 719, 755, | 1083, 1119, | 1739, 1775, 1815, | 6517, 6547, 6823, |
| | | | 207, | 791, 827, 863, 899, | 1155, 1191, | 1851, 1887, 1923, | 6853, 6883, 6913, |
| | | | 243, | 6580, 6610, 6640, | 1231, 1267, | 1959, 1995, 2031, | 6943, 6973, 7003, |
| | | | 279, | 6670, 6700, 6730, | 1303, 1339, | 2067, 7066, 7096, | 7033, 7552, 7582, |
| | | | 315 | 6760, 6790, 7309, | 1375, 1411, | 7126, 7156, 7186, | 7612, 7642, 7672, |
| | | | | 7339, 7369, 7399, | 1447, 1483 | 7216, 7246, 7276, | 7702, 7732, 7762, |
| | | | | 7429, 7459, 7489, | | 7795, 7825, 7855, | 8281, 8311, 8341, |
| | | | | 7519, 8038, 8068, | | 7885, 7915, 7945, | 8371, 8401, 8431, |
| | | | | 8098, 8128, 8158, | | 7975, 8005, 8524, | 8461, 8491 |
| | | | | 8188, 8218, 8248 | | 8554, 8584, 8614, | |
| | | | | | | 8644, 8674, 8704, | |
| | | | | | | 8734 | |
| HsALB_Pf- | 24 | | 64, | 356, 392, 428, 464, | 940, 976, | 1524, 1560, 1596, | 6338, 6368, 6398, |
| CSP(93- | | | 100, | 500, 536, 572, 608, | 1012, 1048, | 1632, 1668, 1704, | 6428, 6458, 6488, |
| 272) | | | 136, | 648, 684, 720, 756, | 1084, 1120, | 1740, 1776, 1816, | 6518, 6548, 6824, |
| | | | 172, | 792, 828, 864, 900, | 1156, 1192, | 1852, 1888, 1924, | 6854, 6884, 6914, |
| | | | 208, | 6581, 6611, 6641, | 1232, 1268, | 1960, 1996, 2032, | 6944, 6974, 7004, |
| | | | 244, | 6671, 6701, 6731, | 1304, 1340, | 2068, 7067, 7097, | 7034, 7553, 7583, |
| | | | 280, | 6761, 6791, 7310, | 1376, 1412, | 7127, 7157, 7187, | 7613, 7643, 7673, |
| | | | 316 | 7340, 7370, 7400, | 1448, 1484 | 7217, 7247, 7277, | 7703, 7733, 7763, |
| | | | | 7430, 7460, 7490, | | 7796, 7826, 7856, | 8282, 8312, 8342, |
| | | | | 7520, 8039, 8069, | | 7886, 7916, 7946, | 8372, 8402, 8432, |
| | | | | 8099, 8129, 8159, | | 7976, 8006, 8525, | 8462, 8492 |
| | | | | 8189, 8219, 8249 | | 8555, 8585, 8615, | |
| | | | | | | 8645, 8675, 8705, | |
| | | | | | | 8735 | |
| HsALB_Pf- | 25 | | 65, | 357, 393, 429, 465, | 941, 977, | 1525, 1561, 1597, | 6339, 6369, 6399, |
| CSP(93- | | | 101, | 501, 537, 573, 609, | 1013, 1049, | 1633, 1669, 1705, | 6429, 6459, 6489, |
| 397) | | | 137, | 649, 685, 721, 757, | 1085, 1121, | 1741, 1777, 1817, | 6519, 6549, 6825, |
| | | | 173, | 793, 829, 865, 901, | 1157, 1193, | 1853, 1889, 1925, | 6855, 6885, 6915, |
| | | | 209, | 6582, 6612, 6642, | 1233, 1269, | 1961, 1997, 2033, | 6945, 6975, 7005, |
| | | | 245, | 6672, 6702, 6732, | 1305, 1341, | 2069, 7068, 7098, | 7035, 7554, 7584, |
| | | | 281, | 6762, 6792, 7311, | 1377, 1413, | 7128, 7158, 7188, | 7614, 7644, 7674, |
| | | | 317 | 7341, 7371, 7401, | 1449, 1485 | 7218, 7248, 7278, | 7704, 7734, 7764, |
| | | | | 7431, 7461, 7491, | | 7797, 7827, 7857, | 8283, 8313, 8343, |
| | | | | 7521, 8040, 8070, | | 7887, 7917, 7947, | 8373, 8403, 8433, |
| | | | | 8100, 8130, 8160, | | 7977, 8007, 8526, | 8463, 8493 |
| | | | | 8190, 8220, 8250 | | 8556, 8586, 8616, | |
| | | | | | | 8646, 8676, 8706, | |
| | | | | | | 8736 | |
| HsALB_Pf- | 26 | | 66, | 358, 394, 430, 466, | 942, 978, | 1526, 1562, 1598, | 6340, 6370, 6400, |
| CSP(98- | | | 102, | 502, 538, 574, 610, | 1014, 1050, | 1634, 1670, 1706, | 6430, 6460, 6490, |
| 192) | | | 138, | 650, 686, 722, 758, | 1086, 1122, | 1742, 1778, 1818, | 6520, 6550, 6826, |
| | | | 174, | 794, 830, 866, 902, | 1158, 1194, | 1854, 1890, 1926, | 6856, 6886, 6916, |
| | | | 210, | 6583, 6613, 6643, | 1234, 1270, | 1962, 1998, 2034, | 6946, 6976, 7006, |
| | | | 246, | 6673, 6703, 6733, | 1306, 1342, | 2070, 7069, 7099, | 7036, 7555, 7585, |
| | | | 282, | 6763, 6793, 7312, | 1378, 1414, | 7129, 7159, 7189, | 7615, 7645, 7675, |
| | | | 318 | 7342, 7372, 7402, | 1450, 1486 | 7219, 7249, 7279, | 7705, 7735, 7765, |
| | | | | 7432, 7462, 7492, | | 7798, 7828, 7858, | 8284, 8314, 8344, |
| | | | | 7522, 8041, 8071, | | 7888, 7918, 7948, | 8374, 8404, 8434, |
| | | | | 8101, 8131, 8161, | | 7978, 8008, 8527, | 8464, 8494 |
| | | | | 8191, 8221, 8251 | | 8557, 8587, 8617, | |
| | | | | | | 8647, 8677, 8707, | |
| | | | | | | 8737 | |
| HsALB_Pf- | 27 | | 67, | 359, 395, 431, 467, | 943, 979, | 1527, 1563, 1599, | 6341, 6371, 6401, |
| CSP(98- | | | 103, | 503, 539, 575, 611, | 1015, 1051, | 1635, 1671, 1707, | 6431, 6461, 6491, |
| 272) | | | 139, | 651, 687, 723, 759, | 1087, 1123, | 1743, 1779, 1819, | 6521, 6551, 6827, |
| | | | 175, | 795, 831, 867, 903, | 1159, 1195, | 1855, 1891, 1927, | 6857, 6887, 6917, |
| | | | 211, | 6584, 6614, 6644, | 1235, 1271, | 1963, 1999, 2035, | 6947, 6977, 7007, |
| | | | 247, | 6674, 6704, 6734, | 1307, 1343, | 2071, 7070, 7100, | 7037, 7556, 7586, |
| | | | 283, | 6764, 6794, 7313, | 1379, 1415, | 7130, 7160, 7190, | 7616, 7646, 7676, |
| | | | 319 | 7343, 7373, 7403, | 1451, 1487 | 7220, 7250, 7280, | 7706, 7736, 7766, |
| | | | | 7433, 7463, 7493, | | 7799, 7829, 7859, | 8285, 8315, 8345, |
| | | | | 7523, 8042, 8072, | | 7889, 7919, 7949, | 8375, 8405, 8435, |
| | | | | 8102, 8132, 8162, | | 7979, 8009, 8528, | 8465, 8495 |
| | | | | 8192, 8222, 8252 | | 8558, 8588, 8618, | |
| | | | | | | 8648, 8678, 8708, | |
| | | | | | | 8738 | |
| HsALB_Pf- | 28 | | 68, | 360, 396, 432, 468, | 944, 980, | 1528, 1564, 1600, | 6342, 6372, 6402, |
| CSP(98- | | | 104, | 504, 540, 576, 612, | 1016, 1052, | 1636, 1672, 1708, | 6432, 6462, 6492, |
| 374) | | | 140, | 652, 688, 724, 760, | 1088, 1124, | 1744, 1780, 1820, | 6522, 6552, 6828, |
| | | | 176, | 796, 832, 868, 904, | 1160, 1196, | 1856, 1892, 1928, | 6858, 6888, 6918, |
| | | | 212, | 6585, 6615, 6645, | 1236, 1272, | 1964, 2000, 2036, | 6948, 6978, 7008, |
| | | | 248, | 6675, 6705, 6735, | 1308, 1344, | 2072, 7071, 7101, | 7038, 7557, 7587, |
| | | | 284, | 6765, 6795, 7314, | 1380, 1416, | 7131, 7161, 7191, | 7617, 7647, 7677, |
| | | | 320 | 7344, 7374, 7404, | 1452, 1488 | 7221, 7251, 7281, | 7707, 7737, 7767, |
| | | | | 7434, 7464, 7494, | | 7800, 7830, 7860, | 8286, 8316, 8346, |
| | | | | 7524, 8043, 8073, | | 7890, 7920, 7950, | 8376, 8406, 8436, |
| | | | | 8103, 8133, 8163, | | 7980, 8010, 8529, | 8466, 8496 |
| | | | | 8193, 8223, 8253 | | 8559, 8589, 8619, | |
| | | | | | | 8649, 8679, 8709, | |
| | | | | | | 8739 | |
| HsALB_Pf- | 29 | | 69, | 361, 397, 433, 469, | 945, 981, | 1529, 1565, 1601, | 6343, 6373, 6403, |
| CSP(98- | | | 105, | 505, 541, 577, 613, | 1017, 1053, | 1637, 1673, 1709, | 6433, 6463, 6493, |
| 397) | | | 141, | 653, 689, 725, 761, | 1089, 1125, | 1745, 1781, 1821, | 6523, 6553, 6829, |
| | | | 177, | 797, 833, 869, 905, | 1161, 1197, | 1857, 1893, 1929, | 6859, 6889, 6919, |
| | | | 213, | 6586, 6616, 6646, | 1237, 1273, | 1965, 2001, 2037, | 6949, 6979, 7009, |
| | | | 249, | 6676, 6706, 6736, | 1309, 1345, | 2073, 7072, 7102, | 7039, 7558, 7588, |
| | | | 285, | 6766, 6796, 7315, | 1381, 1417, | 7132, 7162, 7192, | 7618, 7648, 7678, |
| | | | 321 | 7345, 7375, 7405, | 1453, 1489 | 7222, 7252, 7282, | 7708, 7738, 7768, |
| | | | | 7435, 7465, 7495, | | 7801, 7831, 7861, | 8287, 8317, 8347, |
| | | | | 7525, 8044, 8074, | | 7891, 7921, 7951, | 8377, 8407, 8437, |
| | | | | 8104, 8134, 8164, | | 7981, 8011, 8530, | 8467, 8497 |
| | | | | 8194, 8224, 8254 | | 8560, 8590, 8620, | |
| | | | | | | 8650, 8680, 8710, | |
| | | | | | | 8740 | |
| HsALB_Pf- | 30 | | 70, | 362, 398, 434, 470, | 946, 982, | 1530, 1566, 1602, | 6344, 6374, 6404, |
| CSP(199- | | | 106, | 506, 542, 578, 614, | 1018, 1054, | 1638, 1674, 1710, | 6434, 6464, 6494, |
| 377)_Link- | | | 8765, | 654, 690, 726, 762, | 1090, 1126, | 1746, 1782, 1822, | 6524, 6554, 8969, |
| er(PVTN) | | | 142, | 798, 834, 870, 906, | 1162, 1198, | 1858, 1894, 1930, | 6830, 6860, 6890, |
| HBsAg | | | 178, | 8867, 6587, 6617, | 1238, 1274, | 1966, 2002, 2038, | 6920, 6950, 6980, |
| | | | 214, | 6647, 6677, 6707, | 1310, 1346, | 2074, 9071, 7073, | 7010, 7040, 9275, |
| | | | 250, | 6737, 6767, 6797, | 1382, 1418, | 7103, 7133, 7163, | 7559, 7589, 7619, |
| | | | 286, | 9173, 7316, 7346, | 1454, 1490 | 7193, 7223, 7253, | 7649, 7679, 7709, |
| | | | 322 | 7376, 7406, 7436, | | 7283, 9377, 7802, | 7739, 7769, 9581, |
| | | | | 7466, 7496, 7526, | | 7832, 7862, 7892, | 8288, 8318, 8348, |
| | | | | 9479, 8045, 8075, | | 7922, 7952, 7982, | 8378, 8408, 8438, |
| | | | | 8105, 8135, 8165, | | 8012, 9683, 8531, | 8468, 8498, 9887 |
| | | | | 8195, 8225, 8255, | | 8561, 8591, 8621, | |
| | | | | 9785 | | 8651, 8681, 8711, | |
| | | | | | | 8741, 9989 | |
| LuntSynt_ | 8742 | | 8754, | 8856, 8874, 8886, | — | 9060, 9078, 9090, | 8958, 8976, 8988, |
| Linker(GG | | | 8772, | 8898, 8910, 8922, | | 9102, 9114, 9126, | 9000, 9012, 9024, |
| S4- | | | 8784, | 8934, 8946, 9162, | | 9138, 9150, 9366, | 9036, 9048, 9264, |
| GGG)_Pf- | | | 8796, | 9180, 9192, 9204, | | 9384, 9396, 9408, | 9282, 9294, 9306, |
| CSP(19- | | | 8808, | 9216, 9228, 9240, | | 9420, 9432, 9444, | 9318, 9330, 9342, |
| 397) | | | 8820, | 9252, 9468, 9486, | | 9456, 9672, 9690, | 9354, 9570, 9588, |
| | | | 8832, | 9498, 9510, 9522, | | 9702, 9714, 9726, | 9600, 9612, 9624, |
| | | | 8844 | 9534, 9546, 9558, | | 9738, 9750, 9762, | 9636, 9648, 9660, |
| | | | | 9774, 9792, 9804, | | 9978, 9996, 10008, | |
| | | | | 9816, 9828, 9840, | | 10020, 10032, | |
| | | | | 9852, 9864 | | 10044, 10056, | |
| | | | | | | 10068 | |
| HsALB_Pf- | 8743 | | 8756, | 8858, 8875, 8887, | — | 9062, 9079, 9091, | 8960, 8977, 8989, |
| CSP(19- | | | 8773, | 8899, 8911, 8923, | | 9103, 9115, 9127, | 9001, 9013, 9025, |
| 272)_Link- | | | 8785, | 8935, 8947, 9164, | | 9139, 9151, 9368, | 9037, 9049, 9266, |
| er(PVTN) | | | 8797, | 9181, 9193, 9205, | | 9385, 9397, 9409, | 9283, 9295, 9307, |
| HBsAg | | | 8809, | 9217, 9229, 9241, | | 9421, 9433, 9445, | 9319, 9331, 9343, |
| | | | 8821, | 9253, 9470, 9487, | | 9457, 9674, 9691, | 9355, 9572, 9589, |
| | | | 8833, | 9499, 9511, 9523, | | 9703, 9715, 9727, | 9601, 9613, 9625, |
| | | | 8845 | 9535, 9547, 9559, | | 9739, 9751, 9763, | 9637, 9649, 9661, |
| | | | | 9776, 9793, 9805, | | 9980, 9997, 10009, | 9878, 9895, 9907, |
| | | | | 9817, 9829, 9841, | | 10021, 10033, | 9919, 9931, 9943, |
| | | | | 9853, 9865 | | 10045, 10057, | 9955, 9967 |
| | | | | | | 10069 | |
| HsALB_Pf- | 8744 | | 8757, | 8859, 8876, 8888, | — | 9063, 9080, 9092, | 8961, 8978, 8990, |
| CSP(19- | | | 8758, | 8900, 8912, 8924, | | 9104, 9116, 9128, | 9002, 9014, 9026, |
| 272)_Link- | | | 8774, | 8936, 8948, 8860, | | 9140, 9152, 9064, | 9038, 9050, 8962, |
| er(AAY)_ | | | 8786, | 9165, 9182, 9194, | | 9369, 9386, 9398, | 9267, 9284, 9296, |
| Pf- | | | 8798, | 9206, 9218, 9230, | | 9410, 9422, 9434, | 9308, 9320, 9332, |
| CSP(346- | | | 8810, | 9242, 9254, 9166, | | 9446, 9458, 9370, | 9344, 9356, 9268, |
| 365)_Link- | | | 8822, | 9471, 9488, 9500, | | 9675, 9692, 9704, | 9573, 9590, 9602, |
| er(AAY)_ | | | 8834, | 9512, 9524, 9536, | | 9716, 9728, 9740, | 9614, 9626, 9638, |
| PADRE_L | | | 8846 | 9548, 9560, 9472, | | 9752, 9764, 9676, | 9650, 9662, 9574, |
| inker(PVT | | | | 9777, 9794, 9806, | | 9981, 9998, 10010, | 9879, 9896, 9908, |
| N)_HBsA | | | | 9818, 9830, 9842, | | 10022, 10034, | 9920, 9932, 9944, |
| g | | | | 9854, 9866, 9778 | | 10046, 10058, | 9956, 9968, 9880 |
| | | | | | | 10070, 9982 | |
| HsALB_Pf- | 8745 | | 8759, | 8861, 8877, 8889, | — | 9065, 9081, 9093, | 8963, 8979, 8991, |
| CSP(19- | | | 8760, | 8901, 8913, 8925, | | 9105, 9117, 9129, | 9003, 9015, 9027, |
| 384)_Link- | | | 8775, | 8937, 8949, 8862, | | 9141, 9153, 9066, | 9039, 9051, 8964, |
| er(PVTN) | | | 8787, | 9167, 9183, 9195, | | 9371, 9387, 9399, | 9269, 9285, 9297, |
| HBsAg | | | 8799, | 9207, 9219, 9231, | | 9411, 9423, 9435, | 9309, 9321, 9333, |
| | | | 8811, | 9243, 9255, 9168, | | 9447, 9459, 9372, | 9345, 9357, 9270, |
| | | | 8823, | 9473, 9489, 9501, | | 9677, 9693, 9705, | 9575, 9591, 9603, |
| | | | 8835, | 9513, 9525, 9537, | | 9717, 9729, 9741, | 9615, 9627, 9639, |
| | | | 8847 | 9549, 9561, 9474, | | 9753, 9765, 9678, | 9651, 9663, 9576, |
| | | | | 9779, 9795, 9807, | | 9983, 9999, 10011, | 9881, 9897, 9909, |
| | | | | 9819, 9831, 9843, | | 10023, 10035, | 9921, 9933, 9945, |
| | | | | 9855, 9867, 9780 | | 10047, 10059, | 9957, 9969, 9882 |
| | | | | | | 10071, 9984 | |
| HsALB_Pf- | 8746 | | 8761, | 8863, 8878, 8890, | — | 9067, 9082, 9094, | 8965, 8980, 8992, |
| CSP(19- | | | 8762, | 8902, 8914, 8926, | | 9106, 9118, 9130, | 9004, 9016, 9028, |
| 384)_Link- | | | 8776, | 8938, 8950, 8864, | | 9142, 9154, 9068, | 9040, 9052, 8966, |
| er(SG(3)_ | | | 8788, | 9169, 9184, 9196, | | 9373, 9388, 9400, | 9271, 9286, 9298, |
| Ferritin | | | 8800, | 9208, 9220, 9232, | | 9412, 9424, 9436, | 9310, 9322, 9334, |
| | | | 8812, | 9244, 9256, 9170, | | 9448, 9460, 9374, | 9346, 9358, 9272, |
| | | | 8824, | 9475, 9490, 9502, | | 9679, 9694, 9706, | 9577, 9592, 9604, |
| | | | 8836, | 9514, 9526, 9538, | | 9718, 9730, 9742, | 9616, 9628, 9640, |
| | | | 8848 | 9550, 9562, 9476, | | 9754, 9766, 9680, | 9652, 9664, 9578, |
| | | | | 9781, 9796, 9808, | | 9985, 10000, | 9883, 9898, 9910, |
| | | | | 9820, 9832, 9844, | | 10012, 10024, | 9922, 9934, 9946, |
| | | | | 9856, 9868, 9782 | | 10036, 10048, | 9958, 9970, 9884 |
| | | | | | | 10060, 10072, | |
| | | | | | | 9986 | |
| HsALB_Pf- | 8747 | | 8763, | 8865, 8879, 8891, | — | 9069, 9083, 9095, | 8967, 8981, 8993, |
| CSP(93- | | | 8764, | 8903, 8915, 8927, | | 9107, 9119, 9131, | 9005, 9017, 9029, |
| 384)_Link- | | | 8777, | 8939, 8951, 8866, | | 9143, 9155, 9070, | 9041, 9053, 8968, |
| er(PVTN) | | | 8789, | 9171, 9185, 9197, | | 9375, 9389, 9401, | 9273, 9287, 9299, |
| HBsAg | | | 8801, | 9209, 9221, 9233, | | 9413, 9425, 9437, | 9311, 9323, 9335, |
| | | | 8813, | 9245, 9257, 9172, | | 9449, 9461, 9376, | 9347, 9359, 9274, |
| | | | 8825, | 9477, 9491, 9503, | | 9681, 9695, 9707, | 9579, 9593, 9605, |
| | | | 8837, | 9515, 9527, 9539, | | 9719, 9731, 9743, | 9617, 9629, 9641, |
| | | | 8849 | 9551, 9563, 9478, | | 9755, 9767, 9682, | 9653, 9665, 9580, |
| | | | | 9783, 9797, 9809, | | 9987, 10001, | 9885, 9899, 9911, |
| | | | | 9821, 9833, 9845, | | 10013, 10025, | 9923, 9935, 9947, |
| | | | | 9857, 9869, 9784 | | 10037, 10049, | 9959, 9971, 9886 |
| | | | | | | 10061, 10073, | |
| | | | | | | 9988 | |
| HsALB_Pf- | 8749 | | 8767, | 8869, 8881, 8893, | — | 9073, 9085, 9097, | 8971, 8983, 8995, |
| CSP(19- | | | 8779, | 8905, 8917, 8929, | | 9109, 9121, 9133, | 9007, 9019, 9031, |
| 272)_Link- | | | 8791, | 8941, 8953, 9175, | | 9145, 9157, 9379, | 9043, 9055, 9277, |
| er(AAY)_ | | | 8803, | 9187, 9199, 9211, | | 9391, 9403, 9415, | 9289, 9301, 9313, |
| Pf- | | | 8815, | 9223, 9235, 9247, | | 9427, 9439, 9451, | 9325, 9337, 9349, |
| CSP(346- | | | 8827, | 9259, 9481, 9493, | | 9463, 9685, 9697, | 9361, 9583, 9595, |
| 375)_Link- | | | 8839, | 9505, 9517, 9529, | | 9709, 9721, 9733, | 9607, 9619, 9631, |
| er(AAY)_ | | | 8851 | 9541, 9553, 9565, | | 9745, 9757, 9769, | 9643, 9655, 9667, |
| Pf- | | | | 9787, 9799, 9811, | | 9991, 10003, | 9889, 9901, 9913, |
| CSP(310- | | | | 9823, 9835, 9847, | | 10015, 10027, | 9925, 9937, 9949, |
| 327) | | | | 9859, 9871 | | 10039, 10051, | 9961, 9973 |
| | | | | | | 10063, 10075 | |
| HsALB_Pf- | 8750 | | 8768, | 8870, 8882, 8894, | — | 9074, 9086, 9098, | 8972, 8984, 8996, |
| CSP(19- | | | 8780, | 8906, 8918, 8930, | | 9110, 9122, 9134, | 9008, 9020, 9032, |
| 272)_Link- | | | 8792, | 8942, 8954, 9176, | | 9146, 9158, 9380, | 9044, 9056, 9278, |
| er(AAY)_ | | | 8804, | 9188, 9200, 9212, | | 9392, 9404, 9416, | 9290, 9302, 9314, |
| Pf- | | | 8816, | 9224, 9236, 9248, | | 9428, 9440, 9452, | 9326, 9338, 9350, |
| CSP(346- | | | 8828, | 9260, 9482, 9494, | | 9464, 9686, 9698, | 9362, 9584, 9596, |
| 375)_Link- | | | 8840, | 9506, 9518, 9530, | | 9710, 9722, 9734, | 9608, 9620, 9632, |
| er(AAY)_ | | | 8852 | 9542, 9554, 9566, | | 9746, 9758, 9770, | 9644, 9656, 9668, |
| Pf- | | | | 9788, 9800, 9812, | | 9992, 10004, | 9890, 9902, 9914, |
| CSP(310- | | | | 9824, 9836, 9848, | | 10016, 10028, | 9926, 9938, 9950, |
| 327)_Link | | | | 9860, 9872 | | 10040, 10052, | 9962, 9974 |
| er(AAY)_ | | | | | | 10064, 10076 | |
| PADRE | | | | | | | |
| HsALB_Pf- | 8751 | | 8769, | 8871, 8883, 8895, | — | 9075, 9087, 9099, | 8973, 8985, 8997, |
| CSP(19- | | | 8781, | 8907, 8919, 8931, | | 9111, 9123, 9135, | 9009, 9021, 9033, |
| 272)_Link- | | | 8793, | 8943, 8955, 9177, | | 9147, 9159, 9381, | 9045, 9057, 9279, |
| er(AAY)_ | | | 8805, | 9189, 9201, 9213, | | 9393, 9405, 9417, | 9291, 9303, 9315, |
| Pf- | | | 8817, | 9225, 9237, 9249, | | 9429, 9441, 9453, | 9327, 9339, 9351, |
| CSP(310- | | | 8829, | 9261, 9483, 9495, | | 9465, 9687, 9699, | 9363, 9585, 9597, |
| 327)_Link- | | | 8841, | 9507, 9519, 9531, | | 9711, 9723, 9735, | 9609, 9621, 9633, |
| er(AAY)_ | | | 8853 | 9543, 9555, 9567, | | 9747, 9759, 9771, | 9645, 9657, 9669, |
| Pf- | | | | 9789, 9801, 9813, | | 9993, 10005, | 9891, 9903, 9915, |
| CSP(346- | | | | 9825, 9837, 9849, | | 10017, 10029, | 9927, 9939, 9951, |
| 375)_Link- | | | | 9861, 9873 | | 10041, 10053, | 9963, 9975 |
| er(AAY)_ | | | | | | 10065, 10077 | |
| PADRE | | | | | | | |
| HsALB_Pf- | 8752 | | 8770, | 8872, 8884, 8896, | — | 9076, 9088, 9100, | 8974, 8986, 8998, |
| CSP(19- | | | 8782, | 8908, 8920, 8932, | | 9112, 9124, 9136, | 9010, 9022, 9034, |
| 272)_Link- | | | 8794, | 8944, 8956, 9178, | | 9148, 9160, 9382, | 9046, 9058, 9280, |
| er(AAY)_ | | | 8806, | 9190, 9202, 9214, | | 9394, 9406, 9418, | 9292, 9304, 9316, |
| Pf- | | | 8818, | 9226, 9238, 9250, | | 9430, 9442, 9454, | 9328, 9340, 9352, |
| CSP(310- | | | 8830, | 9262, 9484, 9496, | | 9466, 9688, 9700, | 9364, 9586, 9598, |
| 327)_Link- | | | 8842, | 9508, 9520, 9532, | | 9712, 9724, 9736, | 9610, 9622, 9634, |
| er(AAY)_ | | | 8854 | 9544, 9556, 9568, | | 9748, 9760, 9772, | 9646, 9658, 9670, |
| Pf- | | | | 9790, 9802, 9814, | | 9994, 10006, | 9892, 9904, 9916, |
| CSP(346- | | | | 9826, 9838, 9850, | | 10018, 10030, | 9928, 9940, 9952, |
| 375)_Link- | | | | 9862, 9874 | | 10042, 10054, | 9964, 9976 |
| er(AAY)_ | | | | | | 10066, 10078 | |
| PADRE_L | | | | | | | |
| inker(AA | | | | | | | |
| Y)_P2 | | | | | | | |
| HsALB_Pf- | 8753 | | 8771, | 8873, 8885, 8897, | — | 9077, 9089, 9101, | 8975, 8987, 8999, |
| CSP(19- | | | 8783, | 8909, 8921, 8933, | | 9113, 9125, 9137, | 9011, 9023, 9035, |
| 272)_Link- | | | 8795, | 8945, 8957, 9179, | | 9149, 9161, 9383, | 9047, 9059, 9281, |
| er(AAY)_ | | | 8807, | 9191, 9203, 9215, | | 9395, 9407, 9419, | 9293, 9305, 9317, |
| Pf- | | | 8819, | 9227, 9239, 9251, | | 9431, 9443, 9455, | 9329, 9341, 9353, |
| CSP(310- | | | 8831, | 9263, 9485, 9497, | | 9467, 9689, 9701, | 9365, 9587, 9599, |
| 327)_Link- | | | 8843, | 9509, 9521, 9533, | | 9713, 9725, 9737, | 9611, 9623, 9635, |
| er | | | 8855 | 9545, 9557, 9569, | | 9749, 9761, 9773, | 9647, 9659, 9671, |
| (AAY)_Pf- | | | | 9791, 9803, 9815, | | 9995, 10007, | 9893, 9905, 9917, |
| CSP(346- | | | | 9827, 9839, 9851, | | 10019, 10031, | 9929, 9941, 9953, |
| 397) | | | | 9863, 9875 | | 10043, 10055, | 9965, 9977 |
| | | | | | | 10067, 10079 | |
| Pb-CSP | 31 | 40 | 71, | 332, 363, 399, 435, | 916, 947, | 1500, 1531, 1567, | — |
| | | | 107, | 471, 507, 543, 579, | 983, 1019, | 1603, 1639, 1675, | |
| | | | 143, | 615, 624, 655, 691, | 1055, 1091, | 1711, 1747, 1783, | |
| | | | 179, | 727, 763, 799, 835, | 1127, 1163, | 1792, 1823, 1859, | |
| | | | 215, | 871, 907 | 1199, 1208, | 1895, 1931, 1967, | |
| | | | 251, | | 1239, 1275, | 2003, 2039, 2075 | |
| | | | 287, | | 1311, 1347, | | |
| | | | 323 | | 1383, 1419, | | |
| | | | | | 1455, 1491 | | |
| Pb- | 32 | | 72, | 364, 400, 436, 472, | 948, 984, | 1532, 1568, 1604, | — |
| CSP_Link- | | | 108, | 508, 544, 580, 616, | 1020, 1056, | 1640, 1676, 1712, | |
| er(G4SG4 | | | 144, | 656, 692, 728, 764, | 1092, 1128, | 1748, 1784, 1824, | |
| )_TIm | | | 180, | 800, 836, 872, 908 | 1164, 1200, | 1860, 1896, 1932, | |
| domain | | | 216, | | 1240, 1276, | 1968, 2004, 2040, | |
| HA | | | 252, | | 1312, 1348, | 2076 | |
| | | | 288, | | 1384, 1420, | | |
| | | | 324 | | 1456, 1492 | | |
| HsALB_Pb- | 33 | | 73, | 365, 401, 437, 473, | 949, 985, | 1533, 1569, 1605, | — |
| CSP(24- | | | 109, | 509, 545, 581, 617, | 1021, 1057, | 1641, 1677, 1713, | |
| 340) | | | 145, | 657, 693, 729, 765, | 1093, 1129, | 1749, 1785, 1825, | |
| | | | 181, | 801, 837, 873, 909 | 1165, 1201, | 1861, 1897, 1933, | |
| | | | 217, | | 1241, 1277, | 1969, 2005, 2041, | |
| | | | 253, | | 1313, 1349, | 2077 | |
| | | | 289, | | 1385, 1421, | | |
| | | | 325 | | 1457, 1493 | | |
| HsIns- | 34 | | 74, | 366, 402, 438, 474, | 950, 986, | 1534, 1570, 1606, | — |
| iso-I_Pb- | | | 110, | 510, 546, 582, 618, | 1022, 1058, | 1642, 1678, 1714, | |
| CSP(24- | | | 146, | 658, 694, 730, 766, | 1094, 1130, | 1750, 1786, 1826, | |
| 340) | | | 182, | 802, 838, 874, 910 | 1166, 1202, | 1862, 1898, 1934, | |
| | | | 218, | | 1242, 1278, | 1970, 2006, 2042, | |
| | | | 254, | | 1314, 1350, | 2078 | |
| | | | 290, | | 1386, 1422, | | |
| | | | 326 | | 1458, 1494 | | |
| HsSPARC | 35 | | 75, | 367, 403, 439, 475, | 951, 987, | 1535, 1571, 1607, | — |
| Pb- | | | 111, | 511, 547, 583, 619, | 1023, 1059, | 1643, 1679, 1715, | |
| CSP(24- | | | 147, | 659, 695, 731, 767, | 1095, 1131, | 1751, 1787, 1827, | |
| 340) | | | 183, | 803, 839, 875, 911 | 1167, 1203, | 1863, 1899, 1935, | |
| | | | 219, | | 1243, 1279, | 1971, 2007, 2043, | |
| | | | 255, | | 1315, 1351, | 2079 | |
| | | | 291, | | 1387, 1423, | | |
| | | | 327 | | 1459, 1495 | | |
| IgE_Pb- | 36 | | 76, | 368, 404, 440, 476, | 952, 988, | 1536, 1572, 1608, | — |
| CSP(24- | | | 112, | 512, 548, 584, 620, | 1024, 1060, | 1644, 1680, 1716, | |
| 340) | | | 148, | 660, 696, 732, 768, | 1096, 1132, | 1752, 1788, 1828, | |
| | | | 184, | 804, 840, 876, 912 | 1168, 1204, | 1864, 1900, 1936, | |
| | | | 220, | | 1244, 1280, | 1972, 2008, 2044, | |
| | | | 256, | | 1316, 1352, | 2080 | |
| | | | 292, | | 1388, 1424, | | |
| | | | 328 | | 1460, 1496 | | |

Preferred amino acid sequences, coding sequences, and mRNA sequences of the invention are provided in Table 6A and 6B. Therein, each row represents a specific suitable CSP construct of the invention, wherein the description of the CSP construct is indicated in column A, the SEQ ID NOs of the amino acid sequence is provided in column B. The respective accession number(s), and further information is provided under <223> identifier of the respective SEQ ID NOs in the sequence listing.

The corresponding SEQ ID NOs of the coding sequences encoding the respective CSP constructs are provided in column C of Table 6A and 6B (wild type cds) and D (opt1, opt2, opt3, opt4, opt5, opt11 cds). Further information is provided under <223> identifier of the respective SEQ ID NO in the sequence listing.

For Table 6B, the corresponding RNA sequences comprising a preferred 3′ end/3′ terminus are provided in columns E to H, wherein column E provides RNA sequences with advantageous 3′ end/3′ terminus “A64-N5-C30-hSL-N5” as defined herein, and wherein column F provides RNA sequences with advantageous 3′ end “hSL-A64-N5” as defined herein, and wherein column G provides RNA sequences with advantageous 3′ end/3′ terminus “hSL-A100-N5” as defined herein, and wherein column H provides RNA sequences with advantageous 3′ end/3′ terminus “hSL-A100” as defined herein.

TABLE 6B
Preferred mRNA constructs encoding CSP

| A | B | C | D | E | F | G | H |
|---|---|---|---|---|---|---|---|
| Pf-CSP | 1 | 37 | 41, 77, | 329, 333, 369, 405, | 6555, 6558, | 7284, 7287, 7317, | 8013, 8016, 8046, |
| | | | 113, | 441, 477, 513, 549, | 6588, 6618, | 7347, 7377, 7407, | 8076, 8106, 8136, |
| | | | 149, | 585, 621, 625, 661, | 6648, 6678, | 7437, 7467, 7497, | 8166, 8196, 8226, |
| | | | 185, | 697, 733, 769, 805, | 6708, 6738, | 7527, 7530, 7560, | 8256, 8259, 8289, |
| | | | 221, | 841, 877, 913, 917, | 6768, 6798, | 7590, 7620, 7650, | 8319, 8349, 8379, |
| | | | 257, | 953, 989, 1025, | 6801, 6831, | 7680, 7710, 7740, | 8409, 8439, 8469, |
| | | | 293 | 1061, 1097, 1133, | 6861, 6891, | 7770, 7773, 7803, | 8499, 8502, 8532, |
| | | | | 1169, 1205, 1209, | 6921, 6951, | 7833, 7863, 7893, | 8562, 8592, 8622, |
| | | | | 1245, 1281, 1317, | 6981, 7011, | 7923, 7953, 7983 | 8652, 8682, 8712 |
| | | | | 1353, 1389, 1425, | 7041, 7044, | | |
| | | | | 1461, 1497, 1501, | 7074, 7104, | | |
| | | | | 1537, 1573, 1609, | 7134, 7164, | | |
| | | | | 1645, 1681, 1717, | 7194, 7224, | | |
| | | | | 1753, 1789, 1793, | 7254 | | |
| | | | | 1829, 1865, 1901, | | | |
| | | | | 1937, 1973, 2009, | | | |
| | | | | 2045, 6312, 6315, | | | |
| | | | | 6345, 6375, 6405, | | | |
| | | | | 6435, 6465, 6495, | | | |
| | | | | 6525 | | | |
| Pf-CSP(1- | 2 | 38 | 42, 78, | 330, 334, 370, 406, | 6556, 6559, | 7285, 7288, 7318, | 8014, 8017, 8047, |
| 374) | | | 114, | 442, 478, 514, 550, | 6589, 6619, | 7348, 7378, 7408, | 8077, 8107, 8137, |
| | | | 150, | 586, 622, 626, 662, | 6649, 6679, | 7438, 7468, 7498, | 8167, 8197, 8227, |
| | | | 186, | 698, 734, 770, 806, | 6709, 6739, | 7528, 7531, 7561, | 8257, 8260, 8290, |
| | | | 222, | 842, 878, 914, 918, | 6769, 6799, | 7591, 7621, 7651, | 8320, 8350, 8380, |
| | | | 258, | 954, 990, 1026, | 6802, 6832, | 7681, 7711, 7741, | 8410, 8440, 8470, |
| | | | 294 | 1062, 1098, 1134, | 6862, 6892, | 7771, 7774, 7804, | 8500, 8503, 8533, |
| | | | | 1170, 1206, 1210, | 6922, 6952, | 7834, 7864, 7894, | 8563, 8593, 8623, |
| | | | | 1246, 1282, 1318, | 6982, 7012, | 7924, 7954, 7984 | 8653, 8683, 8713 |
| | | | | 1354, 1390, 1426, | 7042, 7045, | | |
| | | | | 1462, 1498, 1502, | 7075, 7105, | | |
| | | | | 1538, 1574, 1610, | 7135, 7165, | | |
| | | | | 1646, 1682, 1718, | 7195, 7225, | | |
| | | | | 1754, 1790, 1794, | 7255 | | |
| | | | | 1830, 1866, 1902, | | | |
| | | | | 1938, 1974, 2010, | | | |
| | | | | 2046, 6313, 6316, | | | |
| | | | | 6346, 6376, 6406, | | | |
| | | | | 6436, 6466, 6496, | | | |
| | | | | 6526 | | | |
| Pf-CSP(1- | 3 | 39 | 43, 79, | 331, 335, 371, 407, | 6557, 6560, | 7286, 7289, 7319, | 8015, 8018, 8048, |
| 325) | | | 115, | 443, 479, 515, 551, | 6590, 6620, | 7349, 7379, 7409, | 8078, 8108, 8138, |
| | | | 151, | 587, 623, 627, 663, | 6650, 6680, | 7439, 7469, 7499, | 8168, 8198, 8228, |
| | | | 187, | 699, 735, 771, 807, | 6710, 6740, | 7529, 7532, 7562, | 8258, 8261, 8291, |
| | | | 223, | 843, 879, 915, 919, | 6770, 6800, | 7592, 7622, 7652, | 8321, 8351, 8381, |
| | | | 259, | 955, 991, 1027, | 6803, 6833, | 7682, 7712, 7742, | 8411, 8441, 8471, |
| | | | 295 | 1063, 1099, 1135, | 6863, 6893, | 7772, 7775, 7805, | 8501, 8504, 8534, |
| | | | | 1171, 1207, 1211, | 6923, 6953, | 7835, 7865, 7895, | 8564, 8594, 8624, |
| | | | | 1247, 1283, 1319, | 6983, 7013, | 7925, 7955, 7985 | 8654, 8684, 8714 |
| | | | | 1355, 1391, 1427, | 7043, 7046, | | |
| | | | | 1463, 1499, 1503, | 7076, 7106, | | |
| | | | | 1539, 1575, 1611, | 7136, 7166, | | |
| | | | | 1647, 1683, 1719, | 7196, 7226, | | |
| | | | | 1755, 1791, 1795, | 7256 | | |
| | | | | 1831, 1867, 1903, | | | |
| | | | | 1939, 1975, 2011, | | | |
| | | | | 2047, 6314, 6317, | | | |
| | | | | 6347, 6377, 6407, | | | |
| | | | | 6437, 6467, 6497, | | | |
| | | | | 6527 | | | |
| HsALB_Pf | 4 | | 44, 80, | 336, 372, 408, 444, | 6561, 6591, | 7290, 7320, 7350, | 8019, 8049, 8079, |
| 4DSP(19- | | | 8755, | 480, 516, 552, 588, | 6621, 6651, | 7380, 7410, 7440, | 8109, 8139, 8169, |
| 397) | | | 116, | 628, 664, 700, 736, | 6681, 6711, | 7470, 7500, 9469, | 8199, 8229, 9775, |
| | | | 152, | 772, 808, 844, 880, | 6741, 6771, | 7533, 7563, 7593, | 8262, 8292, 8322, |
| | | | 188, | 8857, 920, 956, | 9163, 6804, | 7623, 7653, 7683, | 8352, 8382, 8412, |
| | | | 224, | 992, 1028, 1064, | 6834, 6864, | 7713, 7743, 9571, | 8442, 8472, 9877, |
| | | | 260, | 1100, 1136, 1172, | 6894, 6924, | 7776, 7806, 7836, | 8505, 8535, 8565, |
| | | | 296 | 1212, 1248, 1284, | 6954, 6984, | 7866, 7896, 7926, | 8595, 8625, 8655, |
| | | | | 1320, 1356, 1392, | 7014, 9265, | 7956, 7986, 9673 | 8685, 8715, 9979 |
| | | | | 1428, 1464, 1504, | 7047, 7077, | | |
| | | | | 1540, 1576, 1612, | 7107, 7137, | | |
| | | | | 1648, 1684, 1720, | 7167, 7197, | | |
| | | | | 1756, 1796, 1832, | 7227, 7257, | | |
| | | | | 1868, 1904, 1940, | 9367 | | |
| | | | | 1976, 2012, 2048, | | | |
| | | | | 9061, 6318, 6348, | | | |
| | | | | 6378, 6408, 6438, | | | |
| | | | | 6468, 6498, 6528, | | | |
| | | | | 8959 | | | |
| Hshis- | 5 | | 45, 81, | 337, 373, 409, 445, | 6562, 6592, | 7291, 7321, 7351, | 8020, 8050, 8080, |
| isol_Pf- | | | 117, | 481, 517, 553, 589, | 6622, 6652, | 7381, 7411, 7441, | 8110, 8140, 8170, |
| CSP(19- | | | 153, | 629, 665, 701, 737, | 6682, 6712, | 7471, 7501, 7534, | 8200, 8230, 8263, |
| 397) | | | 189, | 773, 809, 845, 881, | 6742, 6772, | 7564, 7594, 7624, | 8293, 8323, 8353, |
| | | | 225, | 921, 957, 993, | 6805, 6835, | 7654, 7684, 7714, | 8383, 8413, 8443, |
| | | | 261, | 1029, 1065, 1101, | 6865, 6895, | 7744, 7777, 7807, | 8473, 8506, 8536, |
| | | | 297 | 1137, 1173, 1213, | 6925, 6955, | 7837, 7867, 7897, | 8566, 8596, 8626, |
| | | | | 1249, 1285, 1321, | 6985, 7015, | 7927, 7957, 7987 | 8656, 8686, 8716 |
| | | | | 1357, 1393, 1429, | 7048, 7078, | | |
| | | | | 1465, 1505, 1541, | 7108, 7138, | | |
| | | | | 1577, 1613, 1649, | 7168, 7198, | | |
| | | | | 1685, 1721, 1757, | 7228, 7258 | | |
| | | | | 1797, 1833, 1869, | | | |
| | | | | 1905, 1941, 1977, | | | |
| | | | | 2013, 2049, 6319, | | | |
| | | | | 6349, 6379, 6409, | | | |
| | | | | 6439, 6469, 6499, | | | |
| | | | | 6529 | | | |
| HsSPARC | 6 | | 46, 82, | 338, 374, 410, 446, | 6563, 6593, | 7292, 7322, 7352, | 8021, 8051, 8081, |
| Pf- | | | 118, | 482, 518, 554, 590, | 6623, 6653, | 7382, 7412, 7442, | 8111, 8141, 8171, |
| CSP(19- | | | 154, | 630, 666, 702, 738, | 6683, 6713, | 7472, 7502, 7535, | 8201, 8231, 8264, |
| 397) | | | 190, | 774, 810, 846, 882, | 6743, 6773, | 7565, 7595, 7625, | 8294, 8324, 8354, |
| | | | 226, | 922, 958, 994, | 6806, 6836, | 7655, 7685, 7715, | 8384, 8414, 8444, |
| | | | 262, | 1030, 1066, 1102, | 6866, 6896, | 7745, 7778, 7808, | 8474, 8507, 8537, |
| | | | 298 | 1138, 1174, 1214, | 6926, 6956, | 7838, 7868, 7898, | 8567, 8597, 8627, |
| | | | | 1250, 1286, 1322, | 6986, 7016, | 7928, 7958, 7988 | 8657, 8687, 8717 |
| | | | | 1358, 1394, 1430, | 7049, 7079, | | |
| | | | | 1466, 1506, 1542, | 7109, 7139, | | |
| | | | | 1578, 1614, 1650, | 7169, 7199, | | |
| | | | | 1686, 1722, 1758, | 7229, 7259 | | |
| | | | | 1798, 1834, 1870, | | | |
| | | | | 1906, 1942, 1978, | | | |
| | | | | 2014, 2050, 6320, | | | |
| | | | | 6350, 6380, 6410, | | | |
| | | | | 6440, 6470, 6500, | | | |
| | | | | 6530 | | | |
| IgE_Pf- | 7 | | 47, 83, | 339, 375, 411, 447, | 6564, 6594, | 7293, 7323, 7353, | 8022, 8052, 8082, |
| CSP(19- | | | 119, | 483, 519, 555, 591, | 6624, 6654, | 7383, 7413, 7443, | 8112, 8142, 8172, |
| 397) | | | 155, | 631, 667, 703, 739, | 6684, 6714, | 7473, 7503, 7536, | 8202, 8232, 8265, |
| | | | 191, | 775, 811, 847, 883, | 6744, 6774, | 7566, 7596, 7626, | 8295, 8325, 8355, |
| | | | 227, | 923, 959, 995, | 6807, 6837, | 7656, 7686, 7716, | 8385, 8415, 8445, |
| | | | 263, | 1031, 1067, 1103, | 6867, 6897, | 7746, 7779, 7809, | 8475, 8508, 8538, |
| | | | 299 | 1139, 1175, 1215, | 6927, 6957, | 7839, 7869, 7899, | 8568, 8598, 8628, |
| | | | | 1251, 1287, 1323, | 6987, 7017, | 7929, 7959, 7989 | 8658, 8688, 8718 |
| | | | | 1359, 1395, 1431, | 7050, 7080, | | |
| | | | | 1467, 1507, 1543, | 7110, 7140, | | |
| | | | | 1579, 1615, 1651, | 7170, 7200, | | |
| | | | | 1687, 1723, 1759, | 7230, 7260 | | |
| | | | | 1799, 1835, 1871, | | | |
| | | | | 1907, 1943, 1979, | | | |
| | | | | 2015, 2051, 6321, | | | |
| | | | | 6351, 6381, 6411, | | | |
| | | | | 6441, 6471, 6501, | | | |
| | | | | 6531 | | | |
| Pf- | 8 | | 48, 84, | 340, 376, 412, 448, | 6565, 6595, | 7294, 7324, 7354, | 8023, 8053, 8083, |
| CSP_Link- | | | 120, | 484, 520, 556, 592, | 6625, 6655, | 7384, 7414, 7444, | 8113, 8143, 8173, |
| er(G4S | | | 156, | 632, 668, 704, 740, | 6685, 6715, | 7474, 7504, 7537, | 8203, 8233, 8266, |
| G4)_TIm | | | 192, | 776, 812, 848, 884, | 6745, 6775, | 7567, 7597, 7627, | 8296, 8326, 8356, |
| domain | | | 228, | 924, 960, 996, | 6808, 6838, | 7657, 7687, 7717, | 8386, 8416, 8446, |
| HA | | | 264, | 1032, 1068, 1104, | 6868, 6898, | 7747, 7780, 7810, | 8476, 8509, 8539, |
| | | | 300 | 1140, 1176, 1216, | 6928, 6958, | 7840, 7870, 7900, | 8569, 8599, 8629, |
| | | | | 1252, 1288, 1324, | 6988, 7018, | 7930, 7960, 7990 | 8659, 8689, 8719 |
| | | | | 1360, 1396, 1432, | 7051, 7081, | | |
| | | | | 1468, 1508, 1544, | 7111, 7141, | | |
| | | | | 1580, 1616, 1652, | 7171, 7201, | | |
| | | | | 1688, 1724, 1760, | 7231, 7261 | | |
| | | | | 1800, 1836, 1872, | | | |
| | | | | 1908, 1944, 1980, | | | |
| | | | | 2016, 2052, 6322, | | | |
| | | | | 6352, 6382, 6412, | | | |
| | | | | 6442, 6472, 6502, | | | |
| | | | | 6532 | | | |
| Pf- | 9 | | 49, 85, | 341, 377, 413, 449, | 6566, 6596, | 7295, 7325, 7355, | 8024, 8054, 8084, |
| CSP(199- | | | 121, | 485, 521, 557, 593, | 6626, 6656, | 7385, 7415, 7445, | 8114, 8144, 8174, |
| 377)_Link- | | | 157, | 633, 669, 705, 741, | 6686, 6716, | 7475, 7505, 7538, | 8204, 8234, 8267, |
| er(PVTN) | | | 193, | 777, 813, 849, 885, | 6746, 6776, | 7568, 7598, 7628, | 8297, 8327, 8357, |
| HBsAg | | | 229, | 925, 961, 997, | 6809, 6839, | 7658, 7688, 7718, | 8387, 8417, 8447, |
| | | | 265, | 1033, 1069, 1105, | 6869, 6899, | 7748, 7781, 7811, | 8477, 8510, 8540, |
| | | | 301 | 1141, 1177, 1217, | 6929, 6959, | 7841, 7871, 7901, | 8570, 8600, 8630, |
| | | | | 1253, 1289, 1325, | 6989, 7019, | 7931, 7961, 7991 | 8660, 8690, 8720 |
| | | | | 1361, 1397, 1433, | 7052, 7082, | | |
| | | | | 1469, 1509, 1545, | 7112, 7142, | | |
| | | | | 1581, 1617, 1653, | 7172, 7202, | | |
| | | | | 1689, 1725, 1761, | 7232, 7262 | | |
| | | | | 1801, 1837, 1873, | | | |
| | | | | 1909, 1945, 1981, | | | |
| | | | | 2017, 2053, 6323, | | | |
| | | | | 6353, 6383, 6413, | | | |
| | | | | 6443, 6473, 6503, | | | |
| | | | | 6533 | | | |
| Pf- | 10 | | 50, 86, | 342, 378, 414, 450, | 6567, 6597, | 7296, 7326, 7356, | 8025, 8055, 8085, |
| CSP(199- | | | 122, | 486, 522, 558, 594, | 6627, 6657, | 7386, 7416, 7446, | 8115, 8145, 8175, |
| 387)_Link- | | | 158, | 634, 670, 706, 742, | 6687, 6717, | 7476, 7506, 7539, | 8205, 8235, 8268, |
| er(PVTN) | | | 194, | 778, 814, 850, 886, | 6747, 6777, | 7569, 7599, 7629, | 8298, 8328, 8358, |
| HBsAg | | | 230, | 926, 962, 998, | 6810, 6840, | 7659, 7689, 7719, | 8388, 8418, 8448, |
| | | | 266, | 1034, 1070, 1106, | 6870, 6900, | 7749, 7782, 7812, | 8478, 8511, 8541, |
| | | | 302 | 1142, 1178, 1218, | 6930, 6960, | 7842, 7872, 7902, | 8571, 8601, 8631, |
| | | | | 1254, 1290, 1326, | 6990, 7020, | 7932, 7962, 7992 | 8661, 8691, 8721 |
| | | | | 1362, 1398, 1434, | 7053, 7083, | | |
| | | | | 1470, 1510, 1546, | 7113, 7143, | | |
| | | | | 1582, 1618, 1654, | 7173, 7203, | | |
| | | | | 1690, 1726, 1762, | 7233, 7263 | | |
| | | | | 1802, 1838, 1874, | | | |
| | | | | 1910, 1946, 1982, | | | |
| | | | | 2018, 2054, 6324, | | | |
| | | | | 6354, 6384, 6414, | | | |
| | | | | 6444, 6474, 6504, | | | |
| | | | | 6534 | | | |
| HsALB_Pf- | 11 | | 51, 87, | 343, 379, 415, 451, | 6568, 6598, | 7297, 7327, 7357, | 8026, 8056, 8086, |
| CSP(19- | | | 123, | 487, 523, 559, 595, | 6628, 6658, | 7387, 7417, 7447, | 8116, 8146, 8176, |
| 152) | | | 159, | 635, 671, 707, 743, | 6688, 6718, | 7477, 7507, 7540, | 8206, 8236, 8269, |
| | | | 195, | 779, 815, 851, 887, | 6748, 6778, | 7570, 7600, 7630, | 8299, 8329, 8359, |
| | | | 231, | 927, 963, 999, | 6811, 6841, | 7660, 7690, 7720, | 8389, 8419, 8449, |
| | | | 267, | 1035, 1071, 1107, | 6871, 6901, | 7750, 7783, 7813, | 8479, 8512, 8542, |
| | | | 303 | 1143, 1179, 1219, | 6931, 6961, | 7843, 7873, 7903, | 8572, 8602, 8632, |
| | | | | 1255, 1291, 1327, | 6991, 7021, | 7933, 7963, 7993 | 8662, 8692, 8722 |
| | | | | 1363, 1399, 1435, | 7054, 7084, | | |
| | | | | 1471, 1511, 1547, | 7114, 7144, | | |
| | | | | 1583, 1619, 1655, | 7174, 7204, | | |
| | | | | 1691, 1727, 1763, | 7234, 7264 | | |
| | | | | 1803, 1839, 1875, | | | |
| | | | | 1911, 1947, 1983, | | | |
| | | | | 2019, 2055, 6325, | | | |
| | | | | 6355, 6385, 6415, | | | |
| | | | | 6445, 6475, 6505, | | | |
| | | | | 6535 | | | |
| HsALB_Pf | 12 | | 52, 88, | 344, 380, 416, 452, | 6569, 6599, | 7298, 7328, 7358, | 8027, 8057, 8087, |
| 4DSP(19- | | | 124, | 488, 524, 560, 596, | 6629, 6659, | 7388, 7418, 7448, | 8117, 8147, 8177, |
| 192) | | | 160, | 636, 672, 708, 744, | 6689, 6719, | 7478, 7508, 7541, | 8207, 8237, 8270, |
| | | | 196, | 780, 816, 852, 888, | 6749, 6779, | 7571, 7601, 7631, | 8300, 8330, 8360, |
| | | | 232, | 928, 964, 1000, | 6812, 6842, | 7661, 7691, 7721, | 8390, 8420, 8450, |
| | | | 268, | 1036, 1072, 1108, | 6872, 6902, | 7751, 7784, 7814, | 8480, 8513, 8543, |
| | | | 304 | 1144, 1180, 1220, | 6932, 6962, | 7844, 7874, 7904, | 8573, 8603, 8633, |
| | | | | 1256, 1292, 1328, | 6992, 7022, | 7934, 7964, 7994 | 8663, 8693, 8723 |
| | | | | 1364, 1400, 1436, | 7055, 7085, | | |
| | | | | 1472, 1512, 1548, | 7115, 7145, | | |
| | | | | 1584, 1620, 1656, | 7175, 7205, | | |
| | | | | 1692, 1728, 1764, | 7235, 7265 | | |
| | | | | 1804, 1840, 1876, | | | |
| | | | | 1912, 1948, 1984, | | | |
| | | | | 2020, 2056, 6326, | | | |
| | | | | 6356, 6386, 6416, | | | |
| | | | | 6446, 6476, 6506, | | | |
| | | | | 6536 | | | |
| HsALB_Pf | 13 | | 53, 89, | 345, 381, 417, 453, | 6570, 6600, | 7299, 7329, 7359, | 8028, 8058, 8088, |
| 4DSP(19- | | | 125, | 489, 525, 561, 597, | 6630, 6660, | 7389, 7419, 7449, | 8118, 8148, 8178, |
| 272) | | | 161, | 637, 673, 709, 745, | 6690, 6720, | 7479, 7509, 7542, | 8208, 8238, 8271, |
| | | | 197, | 781, 817, 853, 889, | 6750, 6780, | 7572, 7602, 7632, | 8301, 8331, 8361, |
| | | | 233, | 929, 965, 1001, | 6813, 6843, | 7662, 7692, 7722, | 8391, 8421, 8451, |
| | | | 269, | 1037, 1073, 1109, | 6873, 6903, | 7752, 7785, 7815, | 8481, 8514, 8544, |
| | | | 305 | 1145, 1181, 1221, | 6933, 6963, | 7845, 7875, 7905, | 8574, 8604, 8634, |
| | | | | 1257, 1293, 1329, | 6993, 7023, | 7935, 7965, 7995 | 8664, 8694, 8724 |
| | | | | 1365, 1401, 1437, | 7056, 7086, | | |
| | | | | 1473, 1513, 1549, | 7116, 7146, | | |
| | | | | 1585, 1621, 1657, | 7176, 7206, | | |
| | | | | 1693, 1729, 1765, | 7236, 7266 | | |
| | | | | 1805, 1841, 1877, | | | |
| | | | | 1913, 1949, 1985, | | | |
| | | | | 2021, 2057, 6327, | | | |
| | | | | 6357, 6387, 6417, | | | |
| | | | | 6447, 6477, 6507, | | | |
| | | | | 6537 | | | |
| HsALB_Pf- | 14 | | 54, 90, | 346, 382, 418, 454, | 6571, 6601, | 7300, 7330, 7360, | 8029, 8059, 8089, |
| CSP(19- | | | 8766, | 490, 526, 562, 598, | 6631, 6661, | 7390, 7420, 7450, | 8119, 8149, 8179, |
| 272)_Link- | | | 126, | 638, 674, 710, 746, | 6691, 6721, | 7480, 7510, 9480, | 8209, 8239, 9786, |
| er(AAY)_ | | | 162, | 782, 818, 854, 890, | 6751, 6781, | 7543, 7573, 7603, | 8272, 8302, 8332, |
| Pf- | | | 198, | 8868, 930, 966, | 9174, 6814, | 7633, 7663, 7693, | 8362, 8392, 8422, |
| CSP(310- | | | 234, | 1002, 1038, 1074, | 6844, 6874, | 7723, 7753, 9582, | 8452, 8482, 9888, |
| 327)_Link | | | 270, | 1110, 1146, 1182, | 6904, 6934, | 7786, 7816, 7846, | 8515, 8545, 8575, |
| er(AAY)_ | | | 306 | 1222, 1258, 1294, | 6964, 6994, | 7876, 7906, 7936, | 8605, 8635, 8665, |
| Pf- | | | | 1330, 1366, 1402, | 7024, 9276, | 7966, 7996, 9684 | 8695, 8725, 9990 |
| CSP(346- | | | | 1438, 1474, 1514, | 7057, 7087, | | |
| 375) | | | | 1550, 1586, 1622, | 7117, 7147, | | |
| | | | | 1658, 1694, 1730, | 7177, 7207, | | |
| | | | | 1766, 1806, 1842, | 7237, 7267, | | |
| | | | | 1878, 1914, 1950, | 9378 | | |
| | | | | 1986, 2022, 2058, | | | |
| | | | | 9072, 6328, 6358, | | | |
| | | | | 6388, 6418, 6448, | | | |
| | | | | 6478, 6508, 6538, | | | |
| | | | | 8970 | | | |
| HsALB_Pf- | 15 | | 55, 91, | 347, 383, 419, 455, | 6572, 6602, | 7301, 7331, 7361, | 8030, 8060, 8090, |
| CSP(19- | | | 127, | 491, 527, 563, 599, | 6632, 6662, | 7391, 7421, 7451, | 8120, 8150, 8180, |
| 272)_Link- | | | 163, | 639, 675, 711, 747, | 6692, 6722, | 7481, 7511, 7544, | 8210, 8240, 8273, |
| er(AAY)_ | | | 199, | 783, 819, 855, 891, | 6752, 6782, | 7574, 7604, 7634, | 8303, 8333, 8363, |
| Pf- | | | 235, | 931, 967, 1003, | 6815, 6845, | 7664, 7694, 7724, | 8393, 8423, 8453, |
| CSP(346- | | | 271, | 1039, 1075, 1111, | 6875, 6905, | 7754, 7787, 7817, | 8483, 8516, 8546, |
| 365)_Link- | | | 307 | 1147, 1183, 1223, | 6935, 6965, | 7847, 7877, 7907, | 8576, 8606, 8636, |
| er(AAY)_ | | | | 1259, 1295, 1331, | 6995, 7025, | 7937, 7967, 7997 | 8666, 8696, 8726 |
| P2 | | | | 1367, 1403, 1439, | 7058, 7088, | | |
| | | | | 1475, 1515, 1551, | 7118, 7148, | | |
| | | | | 1587, 1623, 1659, | 7178, 7208, | | |
| | | | | 1695, 1731, 1767, | 7238, 7268 | | |
| | | | | 1807, 1843, 1879, | | | |
| | | | | 1915, 1951, 1987, | | | |
| | | | | 2023, 2059, 6329, | | | |
| | | | | 6359, 6389, 6419, | | | |
| | | | | 6449, 6479, 6509, | | | |
| | | | | 6539 | | | |
| HsALB_Pf- | 16 | | 56, 92, | 348, 384, 420, 456, | 6573, 6603, | 7302, 7332, 7362, | 8031, 8061, 8091, |
| CSP(19- | | | 128, | 492, 528, 564, 600, | 6633, 6663, | 7392, 7422, 7452, | 8121, 8151, 8181, |
| 272)_Link- | | | 164, | 640, 676, 712, 748, | 6693, 6723, | 7482, 7512, 7545, | 8211, 8241, 8274, |
| er(AAY)_ | | | 200, | 784, 820, 856, 892, | 6753, 6783, | 7575, 7605, 7635, | 8304, 8334, 8364, |
| Pf- | | | 236, | 932, 968, 1004, | 6816, 6846, | 7665, 7695, 7725, | 8394, 8424, 8454, |
| CSP(346- | | | 272, | 1040, 1076, 1112, | 6876, 6906, | 7755, 7788, 7818, | 8484, 8517, 8547, |
| 365)_Link- | | | 308 | 1148, 1184, 1224, | 6936, 6966, | 7848, 7878, 7908, | 8577, 8607, 8637, |
| er(AAY)_ | | | | 1260, 1296, 1332, | 6996, 7026, | 7938, 7968, 7998 | 8667, 8697, 8727 |
| PADRE | | | | 1368, 1404, 1440, | 7059, 7089, | | |
| | | | | 1476, 1516, 1552, | 7119, 7149, | | |
| | | | | 1588, 1624, 1660, | 7179, 7209, | | |
| | | | | 1696, 1732, 1768, | 7239, 7269 | | |
| | | | | 1808, 1844, 1880, | | | |
| | | | | 1916, 1952, 1988, | | | |
| | | | | 2024, 2060, 6330, | | | |
| | | | | 6360, 6390, 6420, | | | |
| | | | | 6450, 6480, 6510, | | | |
| | | | | 6540 | | | |
| HsALB_Pf- | 17 | | 57, 93, | 349, 385, 421, 457, | 6574, 6604, | 7303, 7333, 7363, | 8032, 8062, 8092, |
| CSP(19- | | | 129, | 493, 529, 565, 601, | 6634, 6664, | 7393, 7423, 7453, | 8122, 8152, 8182, |
| 272)_Link- | | | 165, | 641, 677, 713, 749, | 6694, 6724, | 7483, 7513, 7546, | 8212, 8242, 8275, |
| er(G4S)_ | | | 201, | 785, 821, 857, 893, | 6754, 6784, | 7576, 7606, 7636, | 8305, 8335, 8365, |
| Pf- | | | 237, | 933, 969, 1005, | 6817, 6847, | 7666, 7696, 7726, | 8395, 8425, 8455, |
| CSP(310- | | | 273, | 1041, 1077, 1113, | 6877, 6907, | 7756, 7789, 7819, | 8485, 8518, 8548, |
| 327)_Link- | | | 309 | 1149, 1185, 1225, | 6937, 6967, | 7849, 7879, 7909, | 8578, 8608, 8638, |
| er(G4S)_ | | | | 1261, 1297, 1333, | 6997, 7027, | 7939, 7969, 7999 | 8668, 8698, 8728 |
| Pf- | | | | 1369, 1405, 1441, | 7060, 7090, | | |
| CSP(346- | | | | 1477, 1517, 1553, | 7120, 7150, | | |
| 375) | | | | 1589, 1625, 1661, | 7180, 7210, | | |
| | | | | 1697, 1733, 1769, | 7240, 7270 | | |
| | | | | 1809, 1845, 1881, | | | |
| | | | | 1917, 1953, 1989, | | | |
| | | | | 2025, 2061, 6331, | | | |
| | | | | 6361, 6391, 6421, | | | |
| | | | | 6451, 6481, 6511, | | | |
| | | | | 6541 | | | |
| HsALB_Pf- | 18 | | 58, 94, | 350, 386, 422, 458, | 6575, 6605, | 7304, 7334, 7364, | 8033, 8063, 8093, |
| CSP(19- | | | 130, | 494, 530, 566, 602, | 6635, 6665, | 7394, 7424, 7454, | 8123, 8153, 8183, |
| 272)_Link- | | | 166, | 642, 678, 714, 750, | 6695, 6725, | 7484, 7514, 7547, | 8213, 8243, 8276, |
| er(G4S)_ | | | 202, | 786, 822, 858, 894, | 6755, 6785, | 7577, 7607, 7637, | 8306, 8336, 8366, |
| Pf- | | | 238, | 934, 970, 1006, | 6818, 6848, | 7667, 7697, 7727, | 8396, 8426, 8456, |
| CSP(310- | | | 274, | 1042, 1078, 1114, | 6878, 6908, | 7757, 7790, 7820, | 8486, 8519, 8549, |
| 327)_Pf- | | | 310 | 1150, 1186, 1226, | 6938, 6968, | 7850, 7880, 7910, | 8579, 8609, 8639, |
| CSP(346- | | | | 1262, 1298, 1334, | 6998, 7028, | 7940, 7970, 8000 | 8669, 8699, 8729 |
| 375) | | | | 1370, 1406, 1442, | 7061, 7091, | | |
| | | | | 1478, 1518, 1554, | 7121, 7151, | | |
| | | | | 1590, 1626, 1662, | 7181, 7211, | | |
| | | | | 1698, 1734, 1770, | 7241, 7271 | | |
| | | | | 1810, 1846, 1882, | | | |
| | | | | 1918, 1954, 1990, | | | |
| | | | | 2026, 2062, 6332, | | | |
| | | | | 6362, 6392, 6422, | | | |
| | | | | 6452, 6482, 6512, | | | |
| | | | | 6542 | | | |
| HsALB_Pf | 19 | | 59, 95, | 351, 387, 423, 459, | 6576, 6606, | 7305, 7335, 7365, | 8034, 8064, 8094, |
| 4DSP(19- | | | 131, | 495, 531, 567, 603, | 6636, 6666, | 7395, 7425, 7455, | 8124, 8154, 8184, |
| 325) | | | 167, | 643, 679, 715, 751, | 6696, 6726, | 7485, 7515, 7548, | 8214, 8244, 8277, |
| | | | 203, | 787, 823, 859, 895, | 6756, 6786, | 7578, 7608, 7638, | 8307, 8337, 8367, |
| | | | 239, | 935, 971, 1007, | 6819, 6849, | 7668, 7698, 7728, | 8397, 8427, 8457, |
| | | | 275, | 1043, 1079, 1115, | 6879, 6909, | 7758, 7791, 7821, | 8487, 8520, 8550, |
| | | | 311 | 1151, 1187, 1227, | 6939, 6969, | 7851, 7881, 7911, | 8580, 8610, 8640, |
| | | | | 1263, 1299, 1335, | 6999, 7029, | 7941, 7971, 8001 | 8670, 8700, 8730 |
| | | | | 1371, 1407, 1443, | 7062, 7092, | | |
| | | | | 1479, 1519, 1555, | 7122, 7152, | | |
| | | | | 1591, 1627, 1663, | 7182, 7212, | | |
| | | | | 1699, 1735, 1771, | 7242, 7272 | | |
| | | | | 1811, 1847, 1883, | | | |
| | | | | 1919, 1955, 1991, | | | |
| | | | | 2027, 2063, 6333, | | | |
| | | | | 6363, 6393, 6423, | | | |
| | | | | 6453, 6483, 6513, | | | |
| | | | | 6543 | | | |
| HsALB_Pf | 20 | | 60, 96, | 352, 388, 424, 460, | 6577, 6607, | 7306, 7336, 7366, | 8035, 8065, 8095, |
| 4DSP(19- | | | 132, | 496, 532, 568, 604, | 6637, 6667, | 7396, 7426, 7456, | 8125, 8155, 8185, |
| 384) | | | 168, | 644, 680, 716, 752, | 6697, 6727, | 7486, 7516, 7549, | 8215, 8245, 8278, |
| | | | 204, | 788, 824, 860, 896, | 6757, 6787, | 7579, 7609, 7639, | 8308, 8338, 8368, |
| | | | 240, | 936, 972, 1008, | 6820, 6850, | 7669, 7699, 7729, | 8398, 8428, 8458, |
| | | | 276, | 1044, 1080, 1116, | 6880, 6910, | 7759, 7792, 7822, | 8488, 8521, 8551, |
| | | | 312 | 1152, 1188, 1228, | 6940, 6970, | 7852, 7882, 7912, | 8581, 8611, 8641, |
| | | | | 1264, 1300, 1336, | 7000, 7030, | 7942, 7972, 8002 | 8671, 8701, 8731 |
| | | | | 1372, 1408, 1444, | 7063, 7093, | | |
| | | | | 1480, 1520, 1556, | 7123, 7153, | | |
| | | | | 1592, 1628, 1664, | 7183, 7213, | | |
| | | | | 1700, 1736, 1772, | 7243, 7273 | | |
| | | | | 1812, 1848, 1884, | | | |
| | | | | 1920, 1956, 1992, | | | |
| | | | | 2028, 2064, 6334, | | | |
| | | | | 6364, 6394, 6424, | | | |
| | | | | 6454, 6484, 6514, | | | |
| | | | | 6544 | | | |
| HsALB_Pf | 21 | | 61, 97, | 353, 389, 425, 461, | 6578, 6608, | 7307, 7337, 7367, | 8036, 8066, 8096, |
| 4DSP(19- | | | 133, | 497, 533, 569, 605, | 6638, 6668, | 7397, 7427, 7457, | 8126, 8156, 8186, |
| 384)_TM | | | 169, | 645, 681, 717, 753, | 6698, 6728, | 7487, 7517, 7550, | 8216, 8246, 8279, |
| domain | | | 205, | 789, 825, 861, 897, | 6758, 6788, | 7580, 7610, 7640, | 8309, 8339, 8369, |
| HA | | | 241, | 937, 973, 1009, | 6821, 6851, | 7670, 7700, 7730, | 8399, 8429, 8459, |
| | | | 277, | 1045, 1081, 1117, | 6881, 6911, | 7760, 7793, 7823, | 8489, 8522, 8552, |
| | | | 313 | 1153, 1189, 1229, | 6941, 6971, | 7853, 7883, 7913, | 8582, 8612, 8642, |
| | | | | 1265, 1301, 1337, | 7001, 7031, | 7943, 7973, 8003 | 8672, 8702, 8732 |
| | | | | 1373, 1409, 1445, | 7064, 7094, | | |
| | | | | 1481, 1521, 1557, | 7124, 7154, | | |
| | | | | 1593, 1629, 1665, | 7184, 7214, | | |
| | | | | 1701, 1737, 1773, | 7244, 7274 | | |
| | | | | 1813, 1849, 1885, | | | |
| | | | | 1921, 1957, 1993, | | | |
| | | | | 2029, 2065, 6335, | | | |
| | | | | 6365, 6395, 6425, | | | |
| | | | | 6455, 6485, 6515, | | | |
| | | | | 6545 | | | |
| HsALB_Pf- | 22 | | 62, 98, | 354, 390, 426, 462, | 6579, 6609, | 7308, 7338, 7368, | 8037, 8067, 8097, |
| CSP(82- | | | 134, | 498, 534, 570, 606, | 6639, 6669, | 7398, 7428, 7458, | 8127, 8157, 8187, |
| 397) | | | 170, | 646, 682, 718, 754, | 6699, 6729, | 7488, 7518, 7551, | 8217, 8247, 8280, |
| | | | 206, | 790, 826, 862, 898, | 6759, 6789, | 7581, 7611, 7641, | 8310, 8340, 8370, |
| | | | 242, | 938, 974, 1010, | 6822, 6852, | 7671, 7701, 7731, | 8400, 8430, 8460, |
| | | | 278, | 1046, 1082, 1118, | 6882, 6912, | 7761, 7794, 7824, | 8490, 8523, 8553, |
| | | | 314 | 1154, 1190, 1230, | 6942, 6972, | 7854, 7884, 7914, | 8583, 8613, 8643, |
| | | | | 1266, 1302, 1338, | 7002, 7032, | 7944, 7974, 8004 | 8673, 8703, 8733 |
| | | | | 1374, 1410, 1446, | 7065, 7095, | | |
| | | | | 1482, 1522, 1558, | 7125, 7155, | | |
| | | | | 1594, 1630, 1666, | 7185, 7215, | | |
| | | | | 1702, 1738, 1774, | 7245, 7275 | | |
| | | | | 1814, 1850, 1886, | | | |
| | | | | 1922, 1958, 1994, | | | |
| | | | | 2030, 2066, 6336, | | | |
| | | | | 6366, 6396, 6426, | | | |
| | | | | 6456, 6486, 6516, | | | |
| | | | | 6546 | | | |
| HsALB_Pf- | 23 | | 63, 99, | 355, 391, 427, 463, | 6580, 6610, | 7309, 7339, 7369, | 8038, 8068, 8098, |
| CSP(93- | | | 135, | 499, 535, 571, 607, | 6640, 6670, | 7399, 7429, 7459, | 8128, 8158, 8188, |
| 192) | | | 171, | 647, 683, 719, 755, | 6700, 6730, | 7489, 7519, 7552, | 8218, 8248, 8281, |
| | | | 207, | 791, 827, 863, 899, | 6760, 6790, | 7582, 7612, 7642, | 8311, 8341, 8371, |
| | | | 243, | 939, 975, 1011, | 6823, 6853, | 7672, 7702, 7732, | 8401, 8431, 8461, |
| | | | 279, | 1047, 1083, 1119, | 6883, 6913, | 7762, 7795, 7825, | 8491, 8524, 8554, |
| | | | 315 | 1155, 1191, 1231, | 6943, 6973, | 7855, 7885, 7915, | 8584, 8614, 8644, |
| | | | | 1267, 1303, 1339, | 7003, 7033, | 7945, 7975, 8005 | 8674, 8704, 8734 |
| | | | | 1375, 1411, 1447, | 7066, 7096, | | |
| | | | | 1483, 1523, 1559, | 7126, 7156, | | |
| | | | | 1595, 1631, 1667, | 7186, 7216, | | |
| | | | | 1703, 1739, 1775, | 7246, 7276 | | |
| | | | | 1815, 1851, 1887, | | | |
| | | | | 1923, 1959, 1995, | | | |
| | | | | 2031, 2067, 6337, | | | |
| | | | | 6367, 6397, 6427, | | | |
| | | | | 6457, 6487, 6517, | | | |
| | | | | 6547 | | | |
| HsALB_Pf | 24 | | 64, | 356, 392, 428, 464, | 6581, 6611, | 7310, 7340, 7370, | 8039, 8069, 8099, |
| 4DSP(93- | | | 100, | 500, 536, 572, 608, | 6641, 6671, | 7400, 7430, 7460, | 8129, 8159, 8189, |
| 272) | | | 136, | 648, 684, 720, 756, | 6701, 6731, | 7490, 7520, 7553, | 8219, 8249, 8282, |
| | | | 172, | 792, 828, 864, 900, | 6761, 6791, | 7583, 7613, 7643, | 8312, 8342, 8372, |
| | | | 208, | 940, 976, 1012, | 6824, 6854, | 7673, 7703, 7733, | 8402, 8432, 8462, |
| | | | 244, | 1048, 1084, 1120, | 6884, 6914, | 7763, 7796, 7826, | 8492, 8525, 8555, |
| | | | 280, | 1156, 1192, 1232, | 6944, 6974, | 7856, 7886, 7916, | 8585, 8615, 8645, |
| | | | 316 | 1268, 1304, 1340, | 7004, 7034, | 7946, 7976, 8006 | 8675, 8705, 8735 |
| | | | | 1376, 1412, 1448, | 7067, 7097, | | |
| | | | | 1484, 1524, 1560, | 7127, 7157, | | |
| | | | | 1596, 1632, 1668, | 7187, 7217, | | |
| | | | | 1704, 1740, 1776, | 7247, 7277 | | |
| | | | | 1816, 1852, 1888, | | | |
| | | | | 1924, 1960, 1996, | | | |
| | | | | 2032, 2068, 6338, | | | |
| | | | | 6368, 6398, 6428, | | | |
| | | | | 6458, 6488, 6518, | | | |
| | | | | 6548 | | | |
| HsALB_Pf | 25 | | 65, | 357, 393, 429, 465, | 6582, 6612, | 7311, 7341, 7371, | 8040, 8070, 8100, |
| 4DSP(93- | | | 101, | 501, 537, 573, 609, | 6642, 6672, | 7401, 7431, 7461, | 8130, 8160, 8190, |
| 397) | | | 137, | 649, 685, 721, 757, | 6702, 6732, | 7491, 7521, 7554, | 8220, 8250, 8283, |
| | | | 173, | 793, 829, 865, 901, | 6762, 6792, | 7584, 7614, 7644, | 8313, 8343, 8373, |
| | | | 209, | 941, 977, 1013, | 6825, 6855, | 7674, 7704, 7734, | 8403, 8433, 8463, |
| | | | 245, | 1049, 1085, 1121, | 6885, 6915, | 7764, 7797, 7827, | 8493, 8526, 8556, |
| | | | 281, | 1157, 1193, 1233, | 6945, 6975, | 7857, 7887, 7917, | 8586, 8616, 8646, |
| | | | 317 | 1269, 1305, 1341, | 7005, 7035, | 7947, 7977, 8007 | 8676, 8706, 8736 |
| | | | | 1377, 1413, 1449, | 7068, 7098, | | |
| | | | | 1485, 1525, 1561, | 7128, 7158, | | |
| | | | | 1597, 1633, 1669, | 7188, 7218, | | |
| | | | | 1705, 1741, 1777, | 7248, 7278 | | |
| | | | | 1817, 1853, 1889, | | | |
| | | | | 1925, 1961, 1997, | | | |
| | | | | 2033, 2069, 6339, | | | |
| | | | | 6369, 6399, 6429, | | | |
| | | | | 6459, 6489, 6519, | | | |
| | | | | 6549 | | | |
| HsALB_Pf | 26 | | 66, | 358, 394, 430, 466, | 6583, 6613, | 7312, 7342, 7372, | 8041, 8071, 8101, |
| 4DSP(98- | | | 102, | 502, 538, 574, 610, | 6643, 6673, | 7402, 7432, 7462, | 8131, 8161, 8191, |
| 192) | | | 138, | 650, 686, 722, 758, | 6703, 6733, | 7492, 7522, 7555, | 8221, 8251, 8284, |
| | | | 174, | 794, 830, 866, 902, | 6763, 6793, | 7585, 7615, 7645, | 8314, 8344, 8374, |
| | | | 210, | 942, 978, 1014, | 6826, 6856, | 7675, 7705, 7735, | 8404, 8434, 8464, |
| | | | 246, | 1050, 1086, 1122, | 6886, 6916, | 7765, 7798, 7828, | 8494, 8527, 8557, |
| | | | 282, | 1158, 1194, 1234, | 6946, 6976, | 7858, 7888, 7918, | 8587, 8617, 8647, |
| | | | 318 | 1270, 1306, 1342, | 7006, 7036, | 7948, 7978, 8008 | 8677, 8707, 8737 |
| | | | | 1378, 1414, 1450, | 7069, 7099, | | |
| | | | | 1486, 1526, 1562, | 7129, 7159, | | |
| | | | | 1598, 1634, 1670, | 7189, 7219, | | |
| | | | | 1706, 1742, 1778, | 7249, 7279 | | |
| | | | | 1818, 1854, 1890, | | | |
| | | | | 1926, 1962, 1998, | | | |
| | | | | 2034, 2070, 6340, | | | |
| | | | | 6370, 6400, 6430, | | | |
| | | | | 6460, 6490, 6520, | | | |
| | | | | 6550 | | | |
| HsALB_Pf- | 27 | | 67, | 359, 395, 431, 467, | 6584, 6614, | 7313, 7343, 7373, | 8042, 8072, 8102, |
| CSP(98- | | | 103, | 503, 539, 575, 611, | 6644, 6674, | 7403, 7433, 7463, | 8132, 8162, 8192, |
| 272) | | | 139, | 651, 687, 723, 759, | 6704, 6734, | 7493, 7523, 7556, | 8222, 8252, 8285, |
| | | | 175, | 795, 831, 867, 903, | 6764, 6794, | 7586, 7616, 7646, | 8315, 8345, 8375, |
| | | | 211, | 943, 979, 1015, | 6827, 6857, | 7676, 7706, 7736, | 8405, 8435, 8465, |
| | | | 247, | 1051, 1087, 1123, | 6887, 6917, | 7766, 7799, 7829, | 8495, 8528, 8558, |
| | | | 283, | 1159, 1195, 1235, | 6947, 6977, | 7859, 7889, 7919, | 8588, 8618, 8648, |
| | | | 319 | 1271, 1307, 1343, | 7007, 7037, | 7949, 7979, 8009 | 8678, 8708, 8738 |
| | | | | 1379, 1415, 1451, | 7070, 7100, | | |
| | | | | 1487, 1527, 1563, | 7130, 7160, | | |
| | | | | 1599, 1635, 1671, | 7190, 7220, | | |
| | | | | 1707, 1743, 1779, | 7250, 7280 | | |
| | | | | 1819, 1855, 1891, | | | |
| | | | | 1927, 1963, 1999, | | | |
| | | | | 2035, 2071, 6341, | | | |
| | | | | 6371, 6401, 6431, | | | |
| | | | | 6461, 6491, 6521, | | | |
| | | | | 6551 | | | |
| HsALB_Pf- | 28 | | 68, | 360, 396, 432, 468, | 6585, 6615, | 7314, 7344, 7374, | 8043, 8073, 8103, |
| CSP(98- | | | 104, | 504, 540, 576, 612, | 6645, 6675, | 7404, 7434, 7464, | 8133, 8163, 8193, |
| 374) | | | 140, | 652, 688, 724, 760, | 6705, 6735, | 7494, 7524, 7557, | 8223, 8253, 8286, |
| | | | 176, | 796, 832, 868, 904, | 6765, 6795, | 7587, 7617, 7647, | 8316, 8346, 8376, |
| | | | 212, | 944, 980, 1016, | 6828, 6858, | 7677, 7707, 7737, | 8406, 8436, 8466, |
| | | | 248, | 1052, 1088, 1124, | 6888, 6918, | 7767, 7800, 7830, | 8496, 8529, 8559, |
| | | | 284, | 1160, 1196, 1236, | 6948, 6978, | 7860, 7890, 7920, | 8589, 8619, 8649, |
| | | | 320 | 1272, 1308, 1344, | 7008, 7038, | 7950, 7980, 8010 | 8679, 8709, 8739 |
| | | | | 1380, 1416, 1452, | 7071, 7101, | | |
| | | | | 1488, 1528, 1564, | 7131, 7161, | | |
| | | | | 1600, 1636, 1672, | 7191, 7221, | | |
| | | | | 1708, 1744, 1780, | 7251, 7281 | | |
| | | | | 1820, 1856, 1892, | | | |
| | | | | 1928, 1964, 2000, | | | |
| | | | | 2036, 2072, 6342, | | | |
| | | | | 6372, 6402, 6432, | | | |
| | | | | 6462, 6492, 6522, | | | |
| | | | | 6552 | | | |
| HsALB_Pf- | 29 | | 69, | 361, 397, 433, 469, | 6586, 6616, | 7315, 7345, 7375, | 8044, 8074, 8104, |
| CSP(98- | | | 105, | 505, 541, 577, 613, | 6646, 6676, | 7405, 7435, 7465, | 8134, 8164, 8194, |
| 397) | | | 141, | 653, 689, 725, 761, | 6706, 6736, | 7495, 7525, 7558, | 8224, 8254, 8287, |
| | | | 177, | 797, 833, 869, 905, | 6766, 6796, | 7588, 7618, 7648, | 8317, 8347, 8377, |
| | | | 213, | 945, 981, 1017, | 6829, 6859, | 7678, 7708, 7738, | 8407, 8437, 8467, |
| | | | 249, | 1053, 1089, 1125, | 6889, 6919, | 7768, 7801, 7831, | 8497, 8530, 8560, |
| | | | 285, | 1161, 1197, 1237, | 6949, 6979, | 7861, 7891, 7921, | 8590, 8620, 8650, |
| | | | 321 | 1273, 1309, 1345, | 7009, 7039, | 7951, 7981, 8011 | 8680, 8710, 8740 |
| | | | | 1381, 1417, 1453, | 7072, 7102, | | |
| | | | | 1489, 1529, 1565, | 7132, 7162, | | |
| | | | | 1601, 1637, 1673, | 7192, 7222, | | |
| | | | | 1709, 1745, 1781, | 7252, 7282 | | |
| | | | | 1821, 1857, 1893, | | | |
| | | | | 1929, 1965, 2001, | | | |
| | | | | 2037, 2073, 6343, | | | |
| | | | | 6373, 6403, 6433, | | | |
| | | | | 6463, 6493, 6523, | | | |
| | | | | 6553 | | | |
| HsALB_Pf- | 30_{T} | | 70, | 362, 398, 434, 470, | 6587, 6617, | 7316, 7346, 7376, | 8045, 8075, 8105, |
| CSP(199- | | | 106, | 506, 542, 578, 614, | 6647, 6677, | 7406, 7436, 7466, | 8135, 8165, 8195, |
| 377)_Link- | | | 8765, | 654, 690, 726, 762, | 6707, 6737, | 7496, 7526, 9479, | 8225, 8255, 9785, |
| er(PVTN) | | | 142, | 798, 834, 870, 906, | 6767, 6797, | 7559, 7589, 7619, | 8288, 8318, 8348, |
| HBsAg | | | 178, | 8867, 946, 982, | 9173, 6830, | 7649, 7679, 7709, | 8378, 8408, 8438, |
| | | | 214, | 1018, 1054, 1090, | 6860, 6890, | 7739, 7769, 9581, | 8468, 8498, 9887, |
| | | | 250, | 1126, 1162, 1198, | 6920, 6950, | 7802, 7832, 7862, | 8531, 8561, 8591, |
| | | | 286, | 1238, 1274, 1310, | 6980, 7010, | 7892, 7922, 7952, | 8621, 8651, 8681, |
| | | | 322 | 1346, 1382, 1418, | 7040, 9275, | 7982, 8012, 9683 | 8711, 8741, 9989 |
| | | | | 1454, 1490, 1530, | 7073, 7103, | | |
| | | | | 1566, 1602, 1638, | 7133, 7163, | | |
| | | | | 1674, 1710, 1746, | 7193, 7223, | | |
| | | | | 1782, 1822, 1858, | 7253, 7283, | | |
| | | | | 1894, 1930, 1966, | 9377 | | |
| | | | | 2002, 2038, 2074, | | | |
| | | | | 9071, 6344, 6374, | | | |
| | | | | 6404, 6434, 6464, | | | |
| | | | | 6494, 6524, 6554, | | | |
| | | | | 8969 | | | |
| LuntSynt_ | 8742 | | 8754, | 8856, 8874, 8886, | 9162, 9180, | 9468, 9486, 9498, | 9774, 9792, 9804, |
| Linker(GG | | | 8772, | 8898, 8910, 8922, | 9192, 9204, | 9510, 9522, 9534, | 9816, 9828, 9840, |
| S4- | | | 8784, | 8934, 8946, , 9060, | 9216, 9228, | 9546, 9558, 9570, | 9852, 9864, 9876, |
| GG(S)jPf- | | | 8796, | 9078, 9090, 9102, | 9240, 9252, | 9588, 9600, 9612, | 9894, 9906, 9918, |
| CSP(19- | | | 8808, | 9114, 9126, 9138, | 9264, 9282, | 9624, 9636, 9648, | 9930, 9942, 9954, |
| 397) | | | 8820, | 9150, 8958, 8976, | 9294, 9306, | 9660, 9672, 9690, | 9966, 9978, 9996, |
| | | | 8832, | 8988, 9000, 9012, | 9318, 9330, | 9702, 9714, 9726, | 10008, 10020, |
| | | | 8844 | 9024, 9036, 9048 | 9342, 9354, | 9738, 9750, 9762 | 10032, 10044, |
| | | | | | 9366, 9384, | | 10056, 10068 |
| | | | | | 9396, 9408, | | |
| | | | | | 9420, 9432, | | |
| | | | | | 9444, 9456 | | |
| HsALB_Pf- | 8743 | | 8756, | 8858, 8875, 8887, | 9164, 9181, | 9470, 9487, 9499, | 9776, 9793, 9805, |
| CSP(19- | | | 8773, | 8899, 8911, 8923, | 9193, 9205, | 9511, 9523, 9535, | 9817, 9829, 9841, |
| 272)_Link- | | | 8785, | 8935, 8947, , 9062, | 9217, 9229, | 9547, 9559, 9572, | 9853, 9865, 9878, |
| er(PVTN) | | | 8797, | 9079, 9091, 9103, | 9241, 9253, | 9589, 9601, 9613, | 9895, 9907, 9919, |
| HBsAg | | | 8809, | 9115, 9127, 9139, | 9266, 9283, | 9625, 9637, 9649, | 9931, 9943, 9955, |
| | | | 8821, | 9151, 8960, 8977, | 9295, 9307, | 9661, 9674, 9691, | 9967, 9980, 9997, |
| | | | 8833, | 8989, 9001, 9013, | 9319, 9331, | 9703, 9715, 9727, | 10009, 10021, |
| | | | 8845 | 9025, 9037, 9049 | 9343, 9355, | 9739, 9751, 9763 | 10033, 10045, |
| | | | | | 9368, 9385, | | 10057, 10069 |
| | | | | | 9397, 9409, | | |
| | | | | | 9421, 9433, | | |
| | | | | | 9445, 9457 | | |
| HsALB_Pf- | 8744 | | 8757, | 8859, 8876, 8888, | 9165, 9182, | 9471, 9488, 9500, | 9777, 9794, 9806, |
| CSP(19- | | | 8758, | 8900, 8912, 8924, | 9194, 9206, | 9512, 9524, 9536, | 9818, 9830, 9842, |
| 272)_Link- | | | 8774, | 8936, 8948, 8860, , | 9218, 9230, | 9548, 9560, 9472, | 9854, 9866, 9778, |
| er(AAY)_ | | | 8786, | 9063, 9080, 9092, | 9242, 9254, | 9573, 9590, 9602, | 9879, 9896, 9908, |
| Pf- | | | 8798, | 9104, 9116, 9128, | 9166, 9267, | 9614, 9626, 9638, | 9920, 9932, 9944, |
| CSP(346- | | | 8810, | 9140, 9152, 9064, | 9284, 9296, | 9650, 9662, 9574, | 9956, 9968, 9880, |
| 365)_Link- | | | 8822, | 8961, 8978, 8990, | 9308, 9320, | 9675, 9692, 9704, | 9981, 9998, 10010, |
| er(AAY)_ | | | 8834, | 9002, 9014, 9026, | 9332, 9344, | 9716, 9728, 9740, | 10022, 10034, |
| PADRE_ | | | 8846 | 9038, 9050, 8962 | 9356, 9268, | 9752, 9764, 9676 | 10046, 10058, |
| Linker(PVT | | | | | 9369, 9386, | | 10070, 9982 |
| N)_HBsA | | | | | 9398, 9410, | | |
| g | | | | | 9422, 9434, | | |
| | | | | | 9446, 9458, | | |
| | | | | | 9370 | | |
| HsALB_Pf- | 8745 | | 8759, | 8861, 8877, 8889, | 9167, 9183, | 9473, 9489, 9501, | 9779, 9795, 9807, |
| CSP(19- | | | 8760, | 8901, 8913, 8925, | 9195, 9207, | 9513, 9525, 9537, | 9819, 9831, 9843, |
| 384)_Link- | | | 8775, | 8937, 8949, 8862, , | 9219, 9231, | 9549, 9561, 9474, | 9855, 9867, 9780, |
| er(PVTN) | | | 8787, | 9065, 9081, 9093, | 9243, 9255, | 9575, 9591, 9603, | 9881, 9897, 9909, |
| HBsAg | | | 8799, | 9105, 9117, 9129, | 9168, 9269, | 9615, 9627, 9639, | 9921, 9933, 9945, |
| | | | 8811, | 9141, 9153, 9066, | 9285, 9297, | 9651, 9663, 9576, | 9957, 9969, 9882, |
| | | | 8823, | 8963, 8979, 8991, | 9309, 9321, | 9677, 9693, 9705, | 9983, 9999, 10011, |
| | | | 8835, | 9003, 9015, 9027, | 9333, 9345, | 9717, 9729, 9741, | 10023, 10035, |
| | | | 8847 | 9039, 9051, 8964 | 9357, 9270, | 9753, 9765, 9678 | 10047, 10059, |
| | | | | | 9371, 9387, | | 10071, 9984 |
| | | | | | 9399, 9411, | | |
| | | | | | 9423, 9435, | | |
| | | | | | 9447, 9459, | | |
| | | | | | 9372 | | |
| HsALB_Pf- | 8746 | | 8761, | 8863, 8878, 8890, | 9169, 9184, | 9475, 9490, 9502, | 9781, 9796, 9808, |
| CSP(19- | | | 8762, | 8902, 8914, 8926, | 9196, 9208, | 9514, 9526, 9538, | 9820, 9832, 9844, |
| 384)_Link- | | | 8776, | 8938, 8950, 8864, , | 9220, 9232, | 9550, 9562, 9476, | 9856, 9868, 9782, |
| er(SD3)_ | | | 8788, | 9067, 9082, 9094, | 9244, 9256, | 9577, 9592, 9604, | 9883, 9898, 9910, |
| Ferritin | | | 8800, | 9106, 9118, 9130, | 9170, 9271, | 9616, 9628, 9640, | 9922, 9934, 9946, |
| | | | 8812, | 9142, 9154, 9068, | 9286, 9298, | 9652, 9664, 9578, | 9958, 9970, 9884, |
| | | | 8824, | 8965, 8980, 8992, | 9310, 9322, | 9679, 9694, 9706, | 9985, 10000, |
| | | | 8836, | 9004, 9016, 9028, | 9334, 9346, | 9718, 9730, 9742, | 10012, 10024, |
| | | | 8848 | 9040, 9052, 8966 | 9358, 9272, | 9754, 9766, 9680 | 10036, 10048, |
| | | | | | 9373, 9388, | | 10060, 10072, |
| | | | | | 9400, 9412, | | 9986 |
| | | | | | 9424, 9436, | | |
| | | | | | 9448, 9460, | | |
| | | | | | 9374 | | |
| HsALB_Pf- | 8747 | | 8763, | 8865, 8879, 8891, | 9171, 9185, | 9477, 9491, 9503, | 9783, 9797, 9809, |
| CSP(93- | | | 8764, | 8903, 8915, 8927, | 9197, 9209, | 9515, 9527, 9539, | 9821, 9833, 9845, |
| 384)_Link- | | | 8777, | 8939, 8951, 8866, , | 9221, 9233, | 9551, 9563, 9478, | 9857, 9869, 9784, |
| er(PVTN) | | | 8789, | 9069, 9083, 9095, | 9245, 9257, | 9579, 9593, 9605, | 9885, 9899, 9911, |
| HBsAg | | | 8801, | 9107, 9119, 9131, | 9172, 9273, | 9617, 9629, 9641, | 9923, 9935, 9947, |
| | | | 8813, | 9143, 9155, 9070, | 9287, 9299, | 9653, 9665, 9580, | 9959, 9971, 9886, |
| | | | 8825, | 8967, 8981, 8993, | 9311, 9323, | 9681, 9695, 9707, | 9987, 10001, |
| | | | 8837, | 9005, 9017, 9029, | 9335, 9347, | 9719, 9731, 9743, | 10013, 10025, |
| | | | 8849 | 9041, 9053, 8968 | 9359, 9274, | 9755, 9767, 9682 | 10037, 10049, |
| | | | | | 9375, 9389, | | 10061, 10073, |
| | | | | | 9401, 9413, | | 9988 |
| | | | | | 9425, 9437, | | |
| | | | | | 9449, 9461, | | |
| | | | | | 9376 | | |
| HsALB_Pf- | 8749 | | 8767, | 8869, 8881, 8893, | 9175, 9187, | 9481, 9493, 9505, | 9787, 9799, 9811, |
| CSP(19- | | | 8779, | 8905, 8917, 8929, | 9199, 9211, | 9517, 9529, 9541, | 9823, 9835, 9847, |
| 272)_Link- | | | 8791, | 8941, 8953, , 9073, | 9223, 9235, | 9553, 9565, 9583, | 9859, 9871, 9889, |
| er(AAY)_ | | | 8803, | 9085, 9097, 9109, | 9247, 9259, | 9595, 9607, 9619, | 9901, 9913, 9925, |
| Pf- | | | 8815, | 9121, 9133, 9145, | 9277, 9289, | 9631, 9643, 9655, | 9937, 9949, 9961, |
| CSP(346- | | | 8827, | 9157, 8971, 8983, | 9301, 9313, | 9667, 9685, 9697, | 9973, 9991, 10003, |
| 375)_Link- | | | 8839, | 8995, 9007, 9019, | 9325, 9337, | 9709, 9721, 9733, | 10015, 10027, |
| er(AAY)_ | | | 8851 | 9031, 9043, 9055 | 9349, 9361, | 9745, 9757, 9769 | 10039, 10051, |
| Pf- | | | | | 9379, 9391, | | 10063, 10075 |
| CSP(310- | | | | | 9403, 9415, | | |
| 327) | | | | | 9427, 9439, | | |
| | | | | | 9451, 9463 | | |
| HsALB_Pf- | 8750 | | 8768, | 8870, 8882, 8894, | 9176, 9188, | 9482, 9494, 9506, | 9788, 9800, 9812, |
| CSP(19- | | | 8780, | 8906, 8918, 8930, | 9200, 9212, | 9518, 9530, 9542, | 9824, 9836, 9848, |
| 272)_Link- | | | 8792, | 8942, 8954, , 9074, | 9224, 9236, | 9554, 9566, 9584, | 9860, 9872, 9890, |
| er(AAY)_ | | | 8804, | 9086, 9098, 9110, | 9248, 9260, | 9596, 9608, 9620, | 9902, 9914, 9926, |
| Pf- | | | 8816, | 9122, 9134, 9146, | 9278, 9290, | 9632, 9644, 9656, | 9938, 9950, 9962, |
| CSP(346- | | | 8828, | 9158, 8972, 8984, | 9302, 9314, | 9668, 9686, 9698, | 9974, 9992, 10004, |
| 375)_Link- | | | 8840, | 8996, 9008, 9020, | 9326, 9338, | 9710, 9722, 9734, | 10016, 10028, |
| er(AAY)_ | | | 8852 | 9032, 9044, 9056 | 9350, 9362, | 9746, 9758, 9770 | 10040, 10052, |
| Pf- | | | | | 9380, 9392, | | 10064, 10076 |
| CSP(310- | | | | | 9404, 9416, | | |
| 327)_Link- | | | | | 9428, 9440, | | |
| er(AAY)_ | | | | | 9452, 9464 | | |
| PADRE | | | | | | | |
| HsALB Pf- | 8751 | | 8769, | 8871, 8883, 8895, | 9177, 9189, | 9483, 9495, 9507, | 9789, 9801, 9813, |
| CSP(19- | | | 8781, | 8907, 8919, 8931, | 9201, 9213, | 9519, 9531, 9543, | 9825, 9837, 9849, |
| 272)_Link- | | | 8793, | 8943, 8955, , 9075, | 9225, 9237, | 9555, 9567, 9585, | 9861, 9873, 9891, |
| er(AAY)_ | | | 8805, | 9087, 9099, 9111, | 9249, 9261, | 9597, 9609, 9621, | 9903, 9915, 9927, |
| Pf- | | | 8817, | 9123, 9135, 9147, | 9279, 9291, | 9633, 9645, 9657, | 9939, 9951, 9963, |
| CSP(310- | | | 8829, | 9159, 8973, 8985, | 9303, 9315, | 9669, 9687, 9699, | 9975, 9993, 10005, |
| 327)_Link- | | | 8841, | 8997, 9009, 9021, | 9327, 9339, | 9711, 9723, 9735, | 10017, 10029, |
| er(AAY)_ | | | 8853 | 9033, 9045, 9057 | 9351, 9363, | 9747, 9759, 9771 | 10041, 10053, |
| Pf- | | | | | 9381, 9393, | | 10065, 10077 |
| CSP(346- | | | | | 9405, 9417, | | |
| 375)_Link- | | | | | 9429, 9441, | | |
| er(AAY)_ | | | | | 9453, 9465 | | |
| PADRE | | | | | | | |
| HsALBPf- | 8752 | | 8770, | 8872, 8884, 8896, | 9178, 9190, | 9484. 9496, 9508, | 9790, 9802, 9814, |
| CSP(19- | | | 8782, | 8908, 8920, 8932, | 9202, 9214, | 9520, 9532, 9544, | 9826, 9838, 9850, |
| 272)_Link- | | | 8794, | 8944, 8956, , 9076, | 9226, 9238, | 9556, 9568, 9586, | 9862, 9874, 9892, |
| er(AAY)_ | | | 8806, | 9088, 9100, 9112, | 9250, 9262, | 9598, 9610, 9622, | 9904, 9916, 9928, |
| Pf- | | | 8818, | 9124, 9136, 9148, | 9280, 9292, | 9634, 9646, 9658, | 9940, 9952, 9964, |
| CSP(310- | | | 8830, | 9160, 8974, 8986, | 9304, 9316, | 9670, 9688, 9700, | 9976, 9994, 10006, |
| 327)_Link- | | | 8842, | 8998, 9010, 9022, | 9328, 9340, | 9712, 9724, 9736, | 10018, 10030, |
| er(AAY)_ | | | 8854 | 9034, 9046, 9058 | 9352, 9364, | 9748, 9760, 9772 | 10042, 10054, |
| Pf- | | | | | 9382, 9394, | | 10066, 10078 |
| CSP(346- | | | | | 9406, 9418, | | |
| 375)_Link- | | | | | 9430, 9442, | | |
| er(AAY)_ | | | | | 9454, 9466 | | |
| PADRE_ | | | | | | | |
| Linker(AA | | | | | | | |
| Y)_P2 | | | | | | | |
| HsALB_Pf- | 8753 | | 8771, | 8873, 8885, 8897, | 9179, 9191, | 9485, 9497, 9509, | 9791, 9803, 9815, |
| CSP(19* | | | 8783, | 8909, 8921, 8933, | 9203, 9215, | 9521, 9533, 9545, | 9827, 9839, 9851, |
| 272)_Link- | | | 8795, | 8945, 8957, , 9077, | 9227, 9239, | 9557, 9569, 9587, | 9863, 9875, 9893, |
| er(AAY)_ | | | 8807, | 9089, 9101, 9113, | 9251, 9263, | 9599, 9611, 9623, | 9905, 9917, 9929, |
| Pf- | | | 8819, | 9125, 9137, 9149, | 9281, 9293, | 9635, 9647, 9659, | 9941, 9953, 9965, |
| CSP(310- | | | 8831, | 9161, 8975, 8987, | 9305, 9317, | 9671, 9689, 9701, | 9977, 9995, 10007, |
| 327)_Link- | | | 8843, | 8999, 9011, 9023, | 9329, 9341, | 9713, 9725, 9737, | 10019, 10031, |
| er | | | 8855 | 9035, 9047, 9059 | 9353, 9365, | 9749, 9761, 9773 | 10043, 10055, |
| (AAY)_Pf- | | | | | 9383, 9395, | | 10067, 10079 |
| CSP(346- | | | | | 9407, 9419, | | |
| 397) | | | | | 9431, 9443, | | |
| | | | | | 9455, 9467 | | |
| Pb-CSP | 31 | 40 | 71, | 332, 363, 399, 435, | — | — | — |
| | | | 107, | 471, 507, 543, 579, | | | |
| | | | 143, | 615, 624, 655, 691, | | | |
| | | | 179, | 727, 763, 799, 835, | | | |
| | | | 215, | 871, 907, 916, 947, | | | |
| | | | 251, | 983, 1019, 1055, | | | |
| | | | 287, | 1091, 1127, 1163, | | | |
| | | | 323 | 1199, 1208, 1239, | | | |
| | | | | 1275, 1311, 1347, | | | |
| | | | | 1383, 1419, 1455, | | | |
| | | | | 1491, 1500, 1531, | | | |
| | | | | 1567, 1603, 1639, | | | |
| | | | | 1675, 1711, 1747, | | | |
| | | | | 1783, 1792, 1823, | | | |
| | | | | 1859, 1895, 1931, | | | |
| | | | | 1967, 2003, 2039, | | | |
| | | | | 2075 | | | |
| Pb- | 32 | | 72, | 364, 400, 436, 472, | — | — | — |
| CSP_Link- | | | 108, | 508, 544, 580, 616, | | | |
| er(G4S | | | 144, | 656, 692, 728, 764, | | | |
| G4)_TIm | | | 180, | 800, 836, 872, 908, | | | |
| domain | | | 216, | 948, 984, 1020, | | | |
| HA | | | 252, | 1056, 1092, 1128, | | | |
| | | | 288, | 1164, 1200, 1240, | | | |
| | | | 324 | 1276, 1312, 1348, | | | |
| | | | | 1384, 1420, 1456, | | | |
| | | | | 1492, 1532, 1568, | | | |
| | | | | 1604, 1640, 1676, | | | |
| | | | | 1712, 1748, 1784, | | | |
| | | | | 1824, 1860, 1896, | | | |
| | | | | 1932, 1968, 2004, | | | |
| | | | | 2040, 2076 | | | |
| HsALB_Pb- | 33 | | 73, | 365, 401, 437, 473, | — | — | — |
| CSP(24- | | | 109, | 509, 545, 581, 617, | | | |
| 340) | | | 145, | 657, 693, 729, 765, | | | |
| | | | 181, | 801, 837, 873, 909, | | | |
| | | | 217, | 949, 985, 1021, | | | |
| | | | 253, | 1057, 1093, 1129, | | | |
| | | | 289, | 1165, 1201, 1241, | | | |
| | | | 325 | 1277, 1313, 1349, | | | |
| | | | | 1385, 1421, 1457, | | | |
| | | | | 1493, 1533, 1569, | | | |
| | | | | 1605, 1641, 1677, | | | |
| | | | | 1713, 1749, 1785, | | | |
| | | | | 1825, 1861, 1897, | | | |
| | | | | 1933, 1969, 2005, | | | |
| | | | | 2041, 2077 | | | - |
| Hshis- | 34 | | 74, | 366, 402, 438, 474, | - | - | |
| isol_Pb- | | | 110, | 510, 546, 582, 618, | | | |
| CSP(24- | | | 146, | 658, 694, 730, 766, | | | |
| 340) | | | 182, | 802, 838, 874, 910, | | | |
| | | | 218, | 950, 986, 1022, | | | |
| | | | 254, | 1058, 1094, 1130, | | | |
| | | | 290, | 1166, 1202, 1242, | | | |
| | | | 326 | 1278, 1314, 1350, | | | |
| | | | | 1386, 1422, 1458, | | | |
| | | | | 1494, 1534, 1570, | | | |
| | | | | 1606, 1642, 1678, | | | |
| | | | | 1714, 1750, 1786, | | | |
| | | | | 1826, 1862, 1898, | | | |
| | | | | 1934, 1970, 2006, | | | |
| | | | | 2042, 2078 | | | |
| HsSPARC | 35 | | 75, | 367, 403, 439, 475, | — | — | — |
| Pb- | | | 111, | 511, 547, 583, 619, | | | |
| CSP(24- | | | 147, | 659, 695, 731, 767, | | | |
| 340) | | | 183, | 803, 839, 875, 911, | | | |
| | | | 219, | 951, 987, 1023, | | | |
| | | | 255, | 1059, 1095, 1131, | | | |
| | | | 291, | 1167, 1203, 1243, | | | |
| | | | 327 | 1279, 1315, 1351, | | | |
| | | | | 1387, 1423, 1459, | | | |
| | | | | 1495, 1535, 1571, | | | |
| | | | | 1607, 1643, 1679, | | | |
| | | | | 1715, 1751, 1787, | | | |
| | | | | 1827, 1863, 1899, | | | |
| | | | | 1935, 1971, 2007, | | | |
| | | | | 2043, 2079 | | | |
| IgE_Pb- | 36 | | 76, | 368, 404, 440, 476, | — | — | — |
| CSP(24- | | | 112, | 512, 548, 584, 620, | | | |
| 340) | | | 148, | 660, 696, 732, 768, | | | |
| | | | 184, | 804, 840, 876, 912, | | | |
| | | | 220, | 952, 988, 1024, | | | |
| | | | 256, | 1060, 1096, 1132, | | | |
| | | | 292, | 1168, 1204, 1244, | | | |
| | | | 328 | 1280, 1316, 1352, | | | |
| | | | | 1388, 1424, 1460, | | | |
| | | | | 1496, 1536, 1572, | | | |
| | | | | 1608, 1644, 1680, | | | |
| | | | | 1716, 1752, 1788, | | | |
| | | | | 1828, 1864, 1900, | | | |
| | | | | 1936, 1972, 2008, | | | |
| | | | | 2044, 2080 | | | |

In preferred embodiments, the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 329-2080, 6312-8741, 8856-10079, or a fragment or variant of any of these sequences. Further information is provided under <223> identifier of the respective SEQ ID NO in the sequence listing.

In particularly preferred embodiments, the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 329-2080, 6312-8741, 8856-10079, or a fragment or variant of any of these sequences. Further information is provided under <223> identifier of the respective SEQ ID NO in the sequence listing.

In preferred embodiments, the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 329, 333, 369, 405, 441, 477, 513, 549, 585, 332, 363, 399, 435, 471, 507, 543, 579, 615, 621, 625, 661, 697, 733, 769, 805, 841, 877, 624, 655, 691, 727, 763, 799, 835, 871, 907, 913, 917, 953, 989, 1025, 1061, 1097, 1133, 1169, 916, 947, 983, 1019, 1055, 1091, 1127, 1163, 1199, 1205, 1209, 1245, 1281, 1317, 1353, 1389, 1425, 1461, 1208, 1239, 1275, 1311, 1347, 1383, 1419, 1455, 1491, 1497, 1501, 1537, 1573, 1609, 1645, 1681, 1717, 1753, 1500, 1531, 1567, 1603, 1639, 1675, 1711, 1747, 1783, 1789, 1793, 1829, 1865, 1901, 1937, 1973, 2009, 2045, 1792, 1823, 1859, 1895, 1931, 1967, 2003, 2039, 2075, 6312, 6315, 6345, 6375, 6405, 6435, 6465, 6495, 6525, 6555, 6558, 6588, 6618, 6648, 6678, 6708, 6738, 6768, 6798, 6801, 6831, 6861, 6891, 6921, 6951, 6981, 7011, 7041, 7044, 7074, 7104, 7134, 7164, 7194, 7224, 7254, 7284, 7287, 7317, 7347, 7377, 7407, 7437, 7467, 7497, 7527, 7530, 7560, 7590, 7620, 7650, 7680, 7710, 7740, 7770, 7773, 7803, 7833, 7863, 7893, 7923, 7953, 7983, 8013, 8016, 8046, 8076, 8106, 8136, 8166, 8196, 8226, 8256, 8259, 8289, 8319, 8349, 8379, 8409, 8439, 8469, 8499, 8502, 8532, 8562, 8592, 8622, 8652, 8682, 8712, or a fragment or variant of any of these sequences. Further information is provided under <223> identifier of the respective SEQ ID NO in the sequence listing.

In preferred embodiments, the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 336, 372, 408, 444, 480, 516, 552, 588, 628, 664, 700, 736, 772, 808, 844, 880, 8857, 6561, 6591, 6621, 6651, 6681, 6711, 6741, 6771, 9163, 7290, 7320, 7350, 7380, 7410, 7440, 7470, 7500, 9469, 8019, 8049, 8079, 8109, 8139, 8169, 8199, 8229, 9775, 920, 956, 992, 1028, 1064, 1100, 1136, 1172, 1212, 1248, 1284, 1320, 1356, 1392, 1428, 1464, 1504, 1540, 1576, 1612, 1648, 1684, 1720, 1756, 1796, 1832, 1868, 1904, 1940, 1976, 2012, 2048, 9061, 7047, 7077, 7107, 7137, 7167, 7197, 7227, 7257, 9367, 7776, 7806, 7836, 7866, 7896, 7926, 7956, 7986, 9673, 8505, 8535, 8565, 8595, 8625, 8655, 8685, 8715, 9979, 6318, 6348, 6378, 6408, 6438, 6468, 6498, 6528, 8959, 6804, 6834, 6864, 6894, 6924, 6954, 6984, 7014, 9265, 7533, 7563, 7593, 7623, 7653, 7683, 7713, 7743, 9571, 8262, 8292, 8322, 8352, 8382, 8412, 8442, 8472, 9877 (encoding HsALB_Pf-CSP(19-397)), or a fragment or variant of any of these sequences. Further information is provided under <223> identifier of the respective SEQ ID NO in the sequence listing.

As outlined throughout the specification, additional information regarding suitable amino acid sequences or nucleic acid sequences (coding sequences, mRNA sequences) may also be derived from the sequence listing, in particular from the details provided therein under identifier <223> as explained in the following.

It has to be noted that throughout the sequence listing, information provided under numeric identifier <223> follows the same structure: “<SEQUENCE_DESCRIPTOR> from <CONSTRUCT_IDENTIFIER>”. The <SEQUENCE_DESCRIPTOR> relates to the type of sequence (e.g., “derived and/or modified protein sequence”, “derived and/or modified CDS” “mRNA product design a-1 comprising derived and/or modified sequence”, or “mRNA product Design i-2 comprising derived and/or modified sequence”, or “mRNA product Design i-3 comprising derived and/or modified sequence”, etc.) and whether the sequence comprises or consists of a wild type sequence (“wt”) or whether the sequence comprises or consists of a sequence-optimized sequence (e.g. “opt1”, “opt2”, “opt3”, “opt4”, “opt5”, “opt6”, “opt11”; sequence optimizations are described in further detail below). The <CONSTRUCT_IDENTIFIER> provided under numeric identifier <223> has the following structures: (“organism construct name”, or “organism accession number construct name”) and is intended to help the person skilled in the art to explicitly derive suitable nucleic acid sequences (e.g., RNA, mRNA) encoding the same CSP protein according to the invention.

### RNA Manufacturing Methods:

The coding RNA, preferably the mRNA of the invention may be prepared using any method known in the art, including chemical synthesis such as e.g. solid phase RNA synthesis, as well as in vitro methods, such as RNA in vitro transcription reactions.

In a preferred embodiment, the coding RNA, preferably the mRNA is obtained by RNA in vitro transcription.

Accordingly, the coding RNA of the invention is preferably an in vitro transcribed RNA.

The terms “RNA in vitro transcription” or “in vitro transcription” relate to a process wherein RNA is synthesized in a cell-free system (in vitro). RNA may be obtained by DNA-dependent in vitro transcription of an appropriate DNA template, which according to the present invention is a linearized plasmid DNA template or a PCR-amplified DNA template. The promoter for controlling RNA in vitro transcription can be any promoter for any DNA-dependent RNA polymerase. Particular examples of DNA-dependent RNA polymerases are the T7, T3, SP6, or Syn5 RNA polymerases. In a preferred embodiment of the present invention the DNA template is linearized with a suitable restriction enzyme, before it is subjected to RNA in vitro transcription.

Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein (e.g. m7G(5′)ppp(5′)G (m7G)); optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCl2, which supplies Mg2+ ions as a co-factor for the polymerase; a buffer (TRIS or HEPES) to maintain a suitable pH value, which can also contain antioxidants (e.g. DTT), and/or polyamines such as spermidine at optimal concentrations, e.g. a buffer system comprising TRIS-Citrate as disclosed in WO2017/109161.

In preferred embodiments, the cap1 structure of the coding RNA of the invention is formed using co-transcriptional capping using tri-nucleotide cap analogues m7G(5′)ppp(5′)(2′OMeA)pG or m7G(5′)ppp(5′)(2′OMeG)pG. A preferred cap1 analogue that may suitably be used in manufacturing the coding RNA of the invention is m7G(5′)ppp(5′)(2′OMeA)pG.

In embodiments, the nucleotide mixture used in RNA in vitro transcription may additionally contain modified nucleotides as defined herein. In that context, preferred modified nucleotides comprise pseudouridine (ψ), Ni-methylpseudouridine (m1ψ), 5-methylcytosine, and 5-methoxyuridine. In particular embodiments, uracil nucleotides in the nucleotide mixture are replaced (either partially or completely) by pseudouridine (ψ) and/or N1-methylpseudouridine (m1ψ) to obtain a modified coding RNA.

In preferred embodiments, the nucleotide mixture (i.e. the fraction of each nucleotide in the mixture) used for RNA in vitro transcription reactions may be optimized for the given RNA sequence, preferably as described WO2015/188933.

In embodiment where more than one different coding RNA as defined herein has to be produced, e.g. where 2, 3, 4, 5, 6, 7, 8, 9, 10 or even more different coding RNAs have to be produced (e.g. encoding different CSP antigens, or e.g. a combination of different antigens; see second aspect), procedures as described in WO2017/109134 may be suitably used.

In the context of RNA vaccine production, it may be required to provide GMP-grade RNA. GMP-grade RNA may be produced using a manufacturing process approved by regulatory authorities. Accordingly, in a particularly preferred embodiment, RNA production is performed under current good manufacturing practice (GMP), implementing various quality control steps on DNA and RNA level, preferably according to WO2016/180430. In preferred embodiments, the RNA of the invention is a GMP-grade RNA, particularly a GMP-grade mRNA. Accordingly, a coding RNA for a vaccine is a GMP grade RNA.

The obtained RNA products are preferably purified using PureMessenger® (CureVac, Tubingen, Germany; RP-HPLC according to WO2008/077592) and/or tangential flow filtration (as described in WO2016/193206).

In a further preferred embodiment, the coding RNA, particularly the purified coding RNA, is lyophilized according to WO2016/165831 or WO2011/069586 to yield a temperature stable dried coding RNA (powder) as defined herein. The RNA of the invention, particularly the purified RNA may also be dried using spray-drying or spray-freeze drying according to WO2016/184575 or WO2016/184576 to yield a temperature stable RNA (powder) as defined herein. Accordingly, in the context of manufacturing and purifying RNA, the disclosures of WO2017/109161, WO2015/188933, WO2016/180430, WO2008/077592, WO2016/193206, WO2016/165831, WO2011/069586, WO2016/184575, and WO2016/184576 are incorporated herewith by reference.

Accordingly, in preferred embodiments, the coding RNA is a dried RNA, particularly a dried mRNA.

The term “dried RNA” as used herein has to be understood as RNA that has been lyophilized, or spray-dried, or spray-freeze dried as defined above to obtain a temperature stable dried RNA (powder).

In preferred embodiments, the coding RNA of the invention is a purified RNA, particularly purified mRNA.

The term “purified RNA” or “purified mRNA” as used herein has to be understood as RNA which has a higher purity after certain purification steps (e.g. HPLC, TFF, Oligo d(T) purification, precipitation steps) than the starting material (e.g. in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA fragments, abortive sequences etc.), free nucleotides (modified nucleotides, conventional NTPs, cap analogue), template DNA fragments, buffer components (HEPES, TRIS, MgCl2) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. It is also desirable for the degree of RNA purity that the amount of full-length RNA transcripts is as close as possible to 100%. Accordingly “purified RNA” as used herein has a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target RNA and the total area of all peaks representing the by-products. Alternatively, the degree of purity may for example be determined by an analytical agarose gel electrophoresis or capillary gel electrophoresis.

It has to be understood that “dried RNA” as defined herein and “purified RNA” as defined herein or “GMP-grade mRNA” as defined herein may have superior stability characteristics (in vitro, in vivo) and improved efficiency (e.g. better translatability of the mRNA in vivo) and are therefore particularly suitable for a medical purpose, e.g. a vaccine. Moreover, “dried RNA” as defined herein and “purified RNA” as defined herein or “GMP-grade mRNA” may be particularly suitable for medical use as defined herein.

Accordingly, in preferred embodiments, the coding RNA for a vaccine of the first aspect may be a GMP-grade coding RNA, a purified coding RNA, and/or a dried coding RNA.

Following co-transcriptional capping as defined herein, and following purification as defined herein, the capping degree of the obtained coding RNA may be determined using capping assays as described in published PCT application WO2015/101416, in particular, as described in claims 27 to 46 of published PCT application WO2015/101416 can be used. Alternatively, a capping assays described in PCT/EP2018/08667 may be used.

### Composition, Pharmaceutical Composition:

A second aspect relates to a composition comprising at least one coding RNA of the first aspect.

Notably, embodiments relating to the composition of the second aspect may likewise be read on and be understood as suitable embodiments of the vaccine of the third aspect. Also, embodiments relating to the vaccine of the third aspect may likewise be read on and be understood as suitable embodiments of the composition of the second aspect (comprising the RNA of the first aspect).

In preferred embodiments of the second aspect, said composition comprises at least one RNA encoding CSP of a Malaria parasite according to the first aspect, or an immunogenic fragment or immunogenic variant thereof, wherein said composition is to be, preferably, administered intramuscularly or intradermal.

Preferably, intramuscular or intradermal administration of said composition results in expression of the encoded CSP antigen in a subject. Preferably, the composition of the second aspect is suitable for a vaccine, in particular, suitable for a Malaria vaccine.

The composition may comprise a safe and effective amount of the RNA as specified herein. As used herein, “safe and effective amount” means an amount of the RNA that is sufficient to results in expression and/or activity of the encoded CSP antigenic protein. At the same time, a “safe and effective amount” is small enough to avoid serious side-effects.

A “safe and effective amount” of the RNA of the composition as defined above will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying medical doctor. Moreover, the “safe and effective amount” of the RNA or the composition as described herein may depend from application route (e.g. intramuscular, intradermal), application device (needle injection, injection device), and/or complexation/formulation (e.g. RNA in association with a polymeric carrier or LNP). Moreover, the “safe and effective amount” of the RNA or the composition may depend from the condition of the treated subject (infant, immunocompromised human subject etc.). Accordingly, the suitable “safe and effective amount” has to be adapted and will be chosen and defined by the skilled person.

In the context of the invention, a “composition” refers to any type of composition in which the specified ingredients (e.g. RNA encoding CSP e.g. in association with a polymeric carrier or LNP), may be incorporated, optionally along with any further constituents, usually with at least one pharmaceutically acceptable carrier or excipient. The composition may be a dry composition such as a powder or granules, or a solid unit such as a lyophilized form. Alternatively, the composition may be in liquid form, and each constituent may be independently incorporated in dissolved or dispersed (e.g. suspended or emulsified) form.

In a preferred embodiment of the second aspect, the composition comprises at least one coding RNA of the first aspect and, optionally, at least one pharmaceutically acceptable carrier or excipient.

In particularly preferred embodiments of the second aspect, the composition comprises at least one coding RNA, wherein the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 37-328, 329-2080, 2121-2480, 2887-6134, 6312-8741, 8754-8855, 8856-10079, 10086-10139, and, optionally, at least one pharmaceutically acceptable carrier or excipient.

The term “pharmaceutically acceptable carrier” or “pharmaceutically acceptable excipient” as used herein preferably includes the liquid or non-liquid basis of the composition for administration. If the composition is provided in liquid form, the carrier may be water, e.g. pyrogen-free water; isotonic saline or buffered (aqueous) solutions, e.g. phosphate, citrate etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to preferred embodiments, the sodium, calcium and, optionally, potassium salts may occur in the form of their halogenides, e.g. chlorides, iodides, or bromides, in the form of their hydroxides, carbonates, hydrogen carbonates, or sulfates, etc. Examples of sodium salts include NaCl, NaI, NaBr, Na₂CO₃, NaHCO₃, Na₂SO₄, examples of the optional potassium salts include KCl, KI, KBr, K₂CO₃, KHCO₃, K₂SO₄, and examples of calcium salts include CaCl₂), CaI₂, CaBr₂, CaCO₃, CaSO₄, Ca(OH)₂.

Furthermore, organic anions of the aforementioned cations may be in the buffer. Accordingly, in embodiments, the RNA composition of the invention may comprise pharmaceutically acceptable carriers or excipients using one or more pharmaceutically acceptable carriers or excipients to e.g. increase stability, increase cell transfection, permit the sustained or delayed, increase the translation of encoded CSP protein in vivo, and/or alter the release profile of encoded CSP protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers or diluents or encapsulating compounds may be used as well, which are suitable for administration to a subject. The term “compatible” as used herein means that the constituents of the composition are capable of being mixed with the at least one RNA and, optionally, a plurality of RNAs of the composition, in such a manner that no interaction occurs, which would substantially reduce the biological activity or the pharmaceutical effectiveness of the composition under typical use conditions (e.g., intramuscular or intradermal administration). Pharmaceutically acceptable carriers or excipients must have sufficiently high purity and sufficiently low toxicity to make them suitable for administration to a subject to be treated. Compounds which may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The at least one pharmaceutically acceptable carrier or excipient of the composition may preferably be selected to be suitable for intramuscular or intradermal delivery of said composition. Accordingly, the composition is preferably a pharmaceutical composition, suitable for intramuscular or intradermal administration.

Subjects to which administration of the compositions, preferably the pharmaceutical composition, is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Pharmaceutical compositions of the present invention may suitably be sterile and/or pyrogen-free.

Furthermore, one or more compatible solid or liquid fillers or diluents or encapsulating compounds may be used as well, which are suitable for administration to a person. The term “compatible” as used herein means that the constituents of the composition are capable of being mixed with the at least one RNA and, optionally, the further coding RNA of the composition, in such a manner that no interaction occurs, which would substantially reduce the biological activity or the pharmaceutical effectiveness of the composition under typical use conditions. Pharmaceutically acceptable carriers, fillers and diluents must have sufficiently high purity and sufficiently low toxicity to make them suitable for administration to a person to be treated. Compounds which may be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

Further additives, which may be included in the composition are emulsifiers, such as, for example, Tween; wetting agents, such as, for example, sodium lauryl sulfate; colouring agents; taste-imparting agents, pharmaceutical carriers; tablet-forming agents; stabilizers; antioxidants; preservatives.

In embodiments, the composition as defined herein may comprise a plurality or at least more than one of the coding RNA species as defined in the context of the first aspect of the invention.

In embodiments, the at least one RNA comprised in the composition is a bi- or multicistronic nucleic acid, particularly a bi- or multicistronic nucleic acid as defined herein, which encodes the at least two, three, four, five, six, seven, eight, nine, ten, eleven or twelve distinct antigenic peptides or protein derived from the same Malaria parasite and/or a Malaria parasite.

In embodiments, the composition as defined herein may comprise a plurality or at least more than one of the coding RNA species as defined in the context of the first aspect of the invention. Preferably, the composition as defined herein may comprise 2, 3, 4, 5, 6, 7, 8, 9, or 10 different coding RNAs each defined in the context of the first aspect.

In embodiment, the composition may comprise at least 2, 3, 4, 5, 6, 7, 8, 9, 10 or even more different coding RNA species as defined in the context of the first aspect, each encoding at least one antigenic peptide or protein derived from genetically the same Malaria parasite, or a fragment or variant thereof. Particularly, said (genetically) same Malaria parasite expresses (essentially) the same repertoire of proteins or peptides, wherein all proteins or peptides have (essentially) the same amino acid sequence. Particularly, said (genetically) same Malaria parasite expresses essentially the same proteins, peptides or polyproteins, wherein these protein, peptide or polyproteins preferably do not differ in their amino acid sequence(s). A non-limiting list of exemplary Malaria parasites is provided in List 1.

In preferred embodiments, the composition comprises at least 2, 3, 4, 5, 6, 7, 8, 9, 10 or even more coding RNA construct species each encoding a different CSP Malaria antigen (constructs) as defined in the first aspect, preferably wherein each of the coding RNA constructs are selected from SEQ ID NOs: 329-2080, 6312-8741, 8856-10079.

In preferred embodiments, the composition of the second aspect comprises

(i) at least one coding RNA encoding at least a more full length CSP, and
(ii) at least one coding RNA encoding at least a shortened CSP fragment with HBsAg.

In preferred embodiments, the composition of the second aspect comprises

(i) at least one coding RNA encoding at least a CSP variant inducing strong humoral immune response, and
(ii) at least one coding RNA encoding at least a CSP fragment inducing strong cellular immune response.

In further preferred embodiments, the composition of the second aspect comprises

(i) at least one coding RNA encoding at least a CSP variant inducing strong B-cell immune response,
(ii) at least one coding RNA encoding at least a CSP fragment inducing strong CD4+ T-cell response; and
(ii) at least one coding RNA encoding at least a CSP fragment inducing strong CD8+ T-cell response.

In embodiments, the composition comprises at least 2, 3, 4, 5, 6, 7, 8, 9, 10 or even more different coding RNA species as defined in the context of the first aspect, each encoding at least one peptide or protein derived from a genetically different Malaria parasite, or a fragment or variant thereof. The terms “different” or “different Malaria parasite” as used throughout the present specification in that, has to be understood as the difference between at least two respective Malaria parasites, wherein the difference is manifested on the genome of the respective different Malaria parasites. Particularly, said (genetically) different Malaria parasites may express at least one different protein, peptide or polyprotein, wherein the at least one different protein, peptide or polyprotein preferably differs in at least one amino acid.

In other embodiments, the composition comprises at least 1, 2, 3, 4, 5, 6, 7, 8, 9, 10 or even more coding RNA construct species each encoding a different Malaria antigen selected from CSP, LSA1, MSP1, AMA1, TRAP, VAR2CSA, Pfs230, Pfs28, pfs25, Pfs45/48, RHS, Ripr, EMP1, SSP2, or combinations, or immunogenic fragments, or immunogenic variants of any of these.

In embodiments, the composition comprises at least one coding RNA encoding CSP as defined in the context of the first aspect and, in addition, one coding RNA species encoding an antigen selected from LSA1, MSP1, AMA1, TRAP, VAR2CSA, Pfs230, Pfs28, pfs25, Pfs45/48, RHS, Ripr, EMP1, SSP2.

In embodiments, the composition comprises at least one coding RNA encoding CSP as defined in the context of the first aspect and, in addition, two coding RNA species each encoding a different antigen selected from LSA1, MSP1, AMA1, TRAP, VAR2CSA, Pfs230, Pfs28, pfs25, Pfs45/48, RHS, Ripr, EMP1, SSP2.

In embodiments, the composition comprises at least one coding RNA encoding CSP as defined in the context of the first aspect and, in addition, three coding RNA species each encoding a different antigen selected from LSA1, MSP1, AMA1, TRAP, VAR2CSA, Pfs230, Pfs28, pfs25, Pfs45/48, RHS, Ripr, EMP1, SSP2.

In embodiments, the composition comprises at least one coding RNA encoding CSP as defined in the context of the first aspect and, in addition, at least one coding RNA encoding VAR2CSA.

In embodiments, the composition comprises at least one coding RNA encoding CSP as defined in the context of the first aspect and, in addition, at least one coding RNA encoding VAR2CSA, and at least one coding RNA encoding Pfs25 and/or Pfs230.

In embodiments, the composition comprises at least one coding RNA encoding CSP as defined in the context of the first aspect and, in addition, at least one coding RNA encoding VAR2CSA, and at least one coding RNA encoding Pfs230 and/or Pfs28, and in addition, at least one coding RNA encoding LSA1, MSP1, AMA1, TRAP, VAR2CSA, pfs25, Pfs45/48, RH5, Ripr, EMP1, SSP2

In embodiments, the composition comprises at least one coding RNA encoding CSP as defined in the context of the first aspect and, in addition, at least one coding RNA encoding AMA1, and at least one coding RNA encoding TRAP, and at least one coding RNA encoding MSP1, and/or at least one coding RNA encoding LSA.

In embodiments, the composition comprises at least one coding RNA encoding CSP as defined in the context of the first aspect and, in addition, at least one coding RNA encoding Vaqr2CSA.

In embodiments, the composition comprises at least one coding RNA encoding CSP as defined in the context of the first aspect and, in addition, at least one coding RNA encoding Pfs230, PFS28, Pfs25, Pfs48/50CyRPA, RH5 and/or RIPR.

In embodiments, the composition comprises at least one coding RNA encoding CSP as defined in the context of the first aspect and, in addition, at least one coding RNA encoding AMA1, TRAP, MSP1, LSA, Vaqr2CSA, Pfs230, PFS28, Pfs25, Pfs48/50CyRPA, RH5 and/or RIPR.

### Complexation:

In a preferred embodiment of the second aspect, the at least one coding RNA, or the plurality of coding RNAs (RNA species), is complexed or associated with to obtain a formulated composition. A formulation in that context may have the function of a transfection agent. A formulation in that context may also have the function of protecting the coding RNA from degradation.

In a preferred embodiment of the second aspect, the at least one coding RNA is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

Notably, embodiments relating to “at least one coding RNA” may likewise be read on and be understood as suitable embodiments of more than one or a plurality, e.g. 2, 3, 4, 5, 6, 7, 8, 9, 10, 11, 12, 13, 14, 15 of the RNAs as specified in the context of the first aspect.

The term “cationic or polycationic compound” as used herein will be recognized and understood by the person of ordinary skill in the art, and are for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

Cationic or polycationic compounds, being particularly preferred in this context may be selected from the following list of cationic or polycationic peptides or proteins of fragments thereof: protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides, pAntp, p151, FGF, Lactoferrin, Transportan, Buforin-2, Bac715-24, SynB, SynB(1), pVEC, hCT-derived peptides, SAP, or histones. More preferably, the nucleic acid as defined herein, preferably the mRNA as defined herein, is complexed with one or more polycations, preferably with protamine or oligofectamine, most preferably with protamine.

In a preferred embodiment of the second aspect, the at least one coding RNA is complexed with protamine

Further preferred cationic or polycationic compounds, which can be used as transfection or complexation agent may include cationic polysaccharides, for example chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-aminoacid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

In this context it is particularly preferred that the at least one coding RNA is complexed or at least partially complexed with a cationic or polycationic compound and/or a polymeric carrier, preferably cationic proteins or peptides. In this context, the disclosure of WO2010/037539 and WO2012/113513 is incorporated herewith by reference. Partially means that only a part of the coding RNA is complexed with a cationic compound and that the rest of the RNA is (comprised in the inventive (pharmaceutical) composition) in uncomplexed form (“free”).

In a preferred embodiment of the second aspect, the composition comprises at least one coding RNA complexed with one or more cationic or polycationic compounds, preferably protamine, and at least one free coding RNA.

In this context it is particularly preferred that at least one coding RNA is complexed, or at least partially complexed with protamine. Preferably, the molar ratio of the nucleic acid, particularly the RNA of the protamine-complexed RNA to the free RNA may be selected from a molar ratio of about 0.001:1 to about 1:0.001, including a ratio of about 1:1. Suitably, the complexed RNA is complexed with protamine by addition of protamine-trehalose solution to the RNA sample at a RNA:protamine weight to weight ratio (w/w) of 2:1.

Further preferred cationic or polycationic proteins or peptides that may be used for complexation can be derived from formula (Arg)I;(Lys)m;(His)n;(Orn)o;(Xaa)x of the patent application WO2009/030481 or WO2011/026641, the disclosure of WO2009/030481 or WO2011/026641 relating thereto incorporated herewith by reference.

In a preferred embodiment of the second aspect, the at least one coding RNA is complexed, or at least partially complexed, with at least one cationic or polycationic proteins or peptides preferably selected from SEQ ID NOs: 6201-6204 or any combinations thereof.

According to embodiments, the composition of the present invention comprises the coding RNA as defined in the context of the first aspect, and a polymeric carrier.

The term “polymeric carrier” as used herein will be recognized and understood by the person of ordinary skill in the art, and are e.g. intended to refer to a compound that facilitates transport and/or complexation of another compound (e.g. cargo RNA). A polymeric carrier is typically a carrier that is formed of a polymer. A polymeric carrier may be associated to its cargo (e.g. coding RNA) by covalent or non-covalent interaction. A polymer may be based on different subunits, such as a copolymer.

Suitable polymeric carriers in that context may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PEGylated PLL and polyethylenimine (PEI), dithiobis(succinimidylpropionate) (DSP), Dimethyl-3,3′-dithiobispropionimidate (DTBP), poly(ethylene imine) biscarbamate (PEIC), poly(L-lysine) (PLL), histidine modified PLL, poly(N-vinylpyrrolidone) (PVP), poly(propylenimine (PPI), poly(amidoamine) (PAMAM), poly(amido ethylenimine) (SS-PAEI), triehtylenetetramine (TETA), poly(β-aminoester), poly(4-hydroxy-L-proine ester) (PHP), poly(allylamine), poly(α-[4-aminobutyl]-L-glycolic acid (PAGA), Poly(D,L-lactic-co-glycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PEI, PLL, TETA, poly(allylamine), Poly(N-ethyl-4-vinylpyridinium bromide), pHPMA and pDMAEMA. In one embodiment, the polymer may be a biodegradable PEI such as, but not limited to, DSP, DTBP and PEIC. In one embodiment, the polymer may be biodegradable such as, but not limited to, histine modified PLL, SS-PAEI, poly(β-aminoester), PHP, PAGA, PLGA, PPZ, PPE, PPA and PPE-EA.

When PEI is present, it may be branched PEI of a molecular weight ranging from 10 to 40 kDA, e.g., 25 kDa branched PEI (Sigma #408727).

In some embodiments, the polymer based nanoparticle comprises PEI. In some embodiments, the PEI is branched PEI. PEI may be a branched PEI of a molecular weight ranging from 10 to 40 kDA, e.g., 25 kDa. In some embodiments, the PEI is linear PEI. In some embodiments, the nanoparticle has a size of or less than about 60 nm (e.g., of or less than about 55 nm, of or less than about 50 nm, of or less than about 45 nm, of or less than about 40 nm, of or less than about 35 nm, of or less than about 30 nm, or of or less than about 25 nm). Suitable nanoparticles may be in the range of 25 nm to 60 nm, e.g. 30 nm to 50 nm.

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used according to the present invention may comprise mixtures of cationic peptides, proteins or polymers and optionally further components as defined herein, which are crosslinked by disulfide bonds (via —SH groups).

In this context, polymeric carriers according to formula {(Arg)I;(Lys)m;(His)n;(Orn)o;(Xaa′)x(Cys)y} and formula Cys,{(Arg)I;(Lys)m;(His)n;(Orn)o;(Xaa)x}Cys₂ of the patent application WO2012/013326 are preferred, the disclosure of WO2012/013326 relating thereto incorporated herewith by reference.

In embodiments, the polymeric carrier used to complex the coding RNA may be derived from a polymeric carrier molecule according formula (L—P¹—S—[S—P²—S]ₙ—S—P³—L) of the patent application WO2011/026641, the disclosure of WO2011/026641 relating thereto incorporated herewith by reference.

In embodiments, the polymeric carrier compound is formed by, or comprises or consists of the peptide elements CysArg12Cys (SEQ ID NO: 6201) or CysArg12 (SEQ ID NO: 6202) or TrpArg12Cys (SEQ ID NO: 6203). In particularly preferred embodiments, the polymeric carrier compound consists of a (R₁₂C)-(R₁₂C) dimer, a (WR₁₂C)-(WR₁₂C) dimer, or a (CR₁₂)-(CR₁₂C)-(CR₁₂) trimer, wherein the individual peptide elements in the dimer (e.g. (WR₁₂C)), or the trimer (e.g. (CR₁₂)), are connected via —SH groups.

In a preferred embodiment of the second aspect, the at least one coding RNA of the first aspect is complexed or associated with a polyethylene glycol/peptide polymer comprising HO-PEG5000-S-(S—CHHHHHHRRRRHHHHHHC-S-)7-S-PEG5000-OH (SEQ ID NO: 6204 as peptide monomer), HO-PEG5000-S-(S—CHHHHHHRRRRHHHHHHC-S-)4-S-PEG5000-OH (SEQ ID NO: 6204 as peptide monomer), HO-PEG5000-S-(S-CGHHHHHRRRRHHHHHGC-S-)7-S-PEG5000-OH (SEQ ID NO: 10172 as peptide monomer) and/or a polyethylene glycol/peptide polymer comprising HO-PEG5000-S-(S-CGHHHHHRRRRHHHHHGC-S-)4-S-PEG5000-OH (SEQ ID NO: 10172 of the peptide monomer).

In other embodiments, the composition comprises at least one coding RNA, wherein the at least one coding RNA is complexed or associated with polymeric carriers and, optionally, with at least one lipid component as described in WO2017/212008A1, WO2017/212006A1, WO2017/212007A1, and WO2017/212009A1. In this context, the disclosures of WO2017/212008A1, WO2017/212006A1, WO2017/212007A1, and WO2017/212009A1 are herewith incorporated by reference.

In a particularly preferred embodiment, the polymeric carrier is a peptide polymer, preferably a polyethylene glycol/peptide polymer as defined above, and a lipid component, preferably a lipidoid component.

In preferred embodiment of the second aspect, the at least one coding RNA of the first aspect is complexed or associated with a polymeric carrier, preferably with a polyethylene glycol/peptide polymer as defined above, and a lipidoid component, wherein the lipidoid component is a compound according to formula A

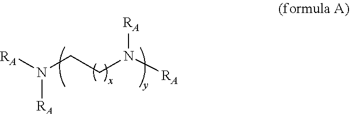

wherein
R_{A} is independently selected for each occurrence an unsubstituted, cyclic or acyclic, branched or unbranched C₁₋₂₀ aliphatic group; a substituted or unsubstituted, cyclic or acyclic, branched or unbranched C₁₋₂₀ heteroaliphatic group; a substituted or unsubstituted aryl; a substituted or unsubstituted heteroaryl;

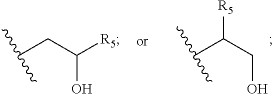

wherein at least one R_{A} is

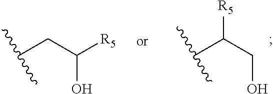

R₅ is independently selected for each occurrence of from an unsubstituted, cyclic or acyclic, branched or unbranched C₀₋₁₆ aliphatic; a substituted or unsubstituted aryl; or a substituted or unsubstituted heteroaryl;
each occurrence of x is an integer from 1 to 10;
each occurrence of y is an integer from 1 to 10; or a pharmaceutically acceptable salt thereof.

In a preferred embodiment, the lipidoid component may be any one selected from the lipidoids of Table 7.

TABLE 7
Lipidoids suitable for the invention:

| | |
|---|---|
| 3-C12-OH | 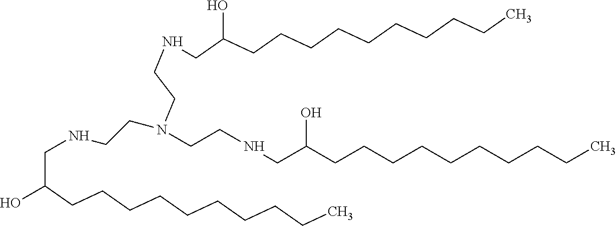 [SMILES: CCCCCCCCCCC(O)CNCCN(CCNCC(O)CCCCCCCCCC)CCNCC(O)CCCCCCCCCC] |
| 3-C12-OH-cat | 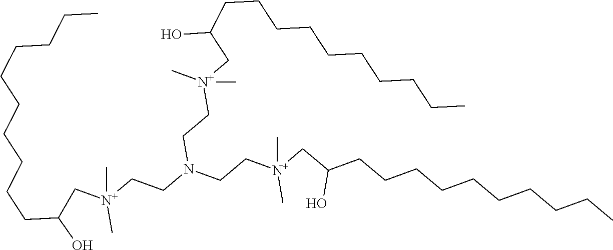 [SMILES: CCCCCCCCCCC(O)C[N+](C)(C)CCN(CC[N+](C)(C)CC(O)CCCCCCCCCC)CC[N+](C)(C)CC(O)CCCCCCCCCC] |
| 3-C12-amide | 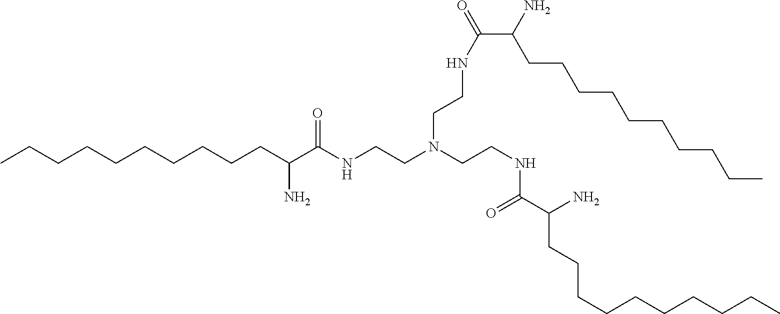 [SMILES: CCCCCCCCCCC(N)C(=O)NCCN(CCNC(=O)C(N)CCCCCCCCCC)CCNC(=O)C(N)CCCCCCCCCC] |
| 3-C12-amide monomethyl | 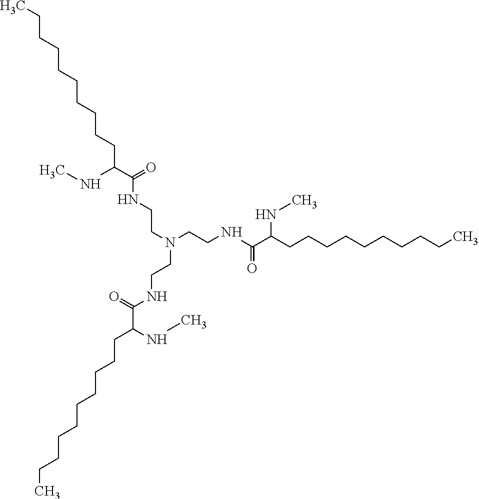 [SMILES: CCCCCCCCCCC(NC)C(=O)NCCN(CCNC(=O)C(CCCCCCCCCC)NC)CCNC(=O)C(CCCCCCCCCC)NC] |
| 3-C12-amide dimethyl | 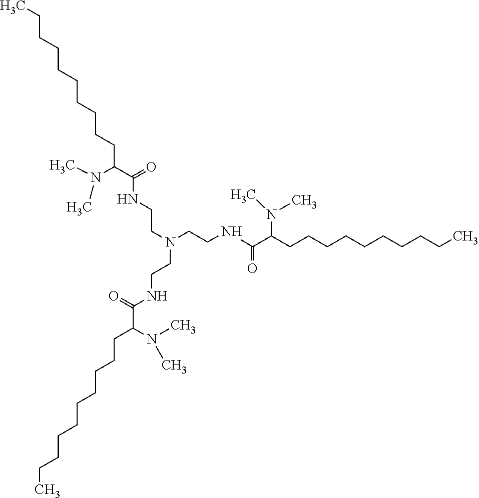 [SMILES: CCCCCCCCCCC(C(=O)NCCN(CCNC(=O)C(CCCCCCCCCC)N(C)C)CCNC(=O)C(CCCCCCCCCC)N(C)C)N(C)C] |
| RevPEG(10)-3- C12-OH | 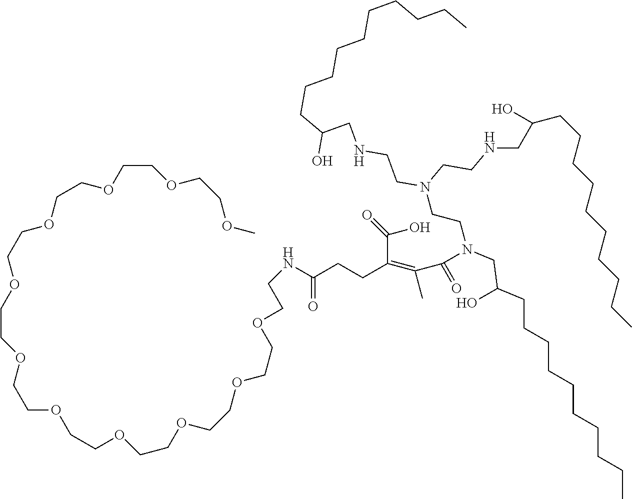 [SMILES: CCCCCCCCCCC(O)CNCCN(CCNCC(O)CCCCCCCCCC)CCN(CC(O)CCCCCCCCCC)C(=O)/C(C)=C(/CCC(=O)NCCOCCOCCOCCOCCOCCOCCOCCOCCOCCOCCOC)C(=O)O] |
| RevPEG(10)- DLin-pAbenzoic | 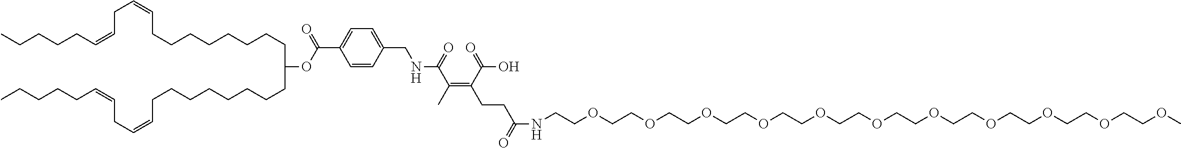 [SMILES: CCCCC/C=C\C/C=C\CCCCCCCCC(CCCCCCCC/C=C\C/C=C\CCCCC)OC(=O)c1ccc(CNC(=O)/C(C)=C(/CCC(=O)NCCOCCOCCOCCOCCOCCOCCOCCOCCOCCOCCOC)C(=O)O)cc1] |
| 3C12amide-TMA cat. | 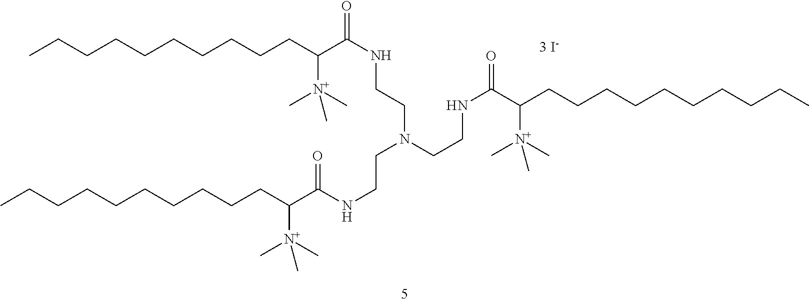 [SMILES: CCCCCCCCCCC(C(=O)NCCN(CCNC(=O)C(CCCCCCCCCC)[N+](C)(C)C)CCNC(=O)C(CCCCCCCCCC)[N+](C)(C)C)[N+](C)(C)C] |
| 3C12amide-DMA | 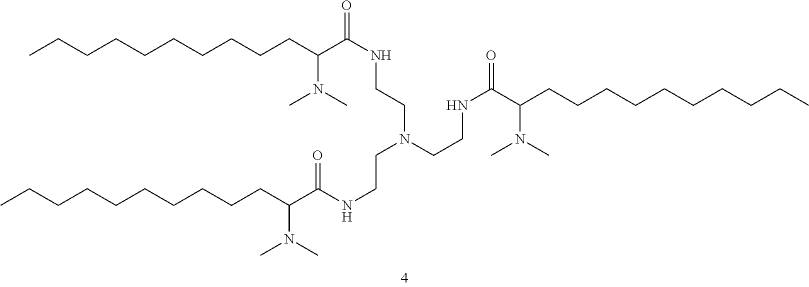 [SMILES: CCCCCCCCCCC(C(=O)NCCN(CCNC(=O)C(CCCCCCCCCC)N(C)C)CCNC(=O)C(CCCCCCCCCC)N(C)C)N(C)C] |
| 3C12amide-NH2 | 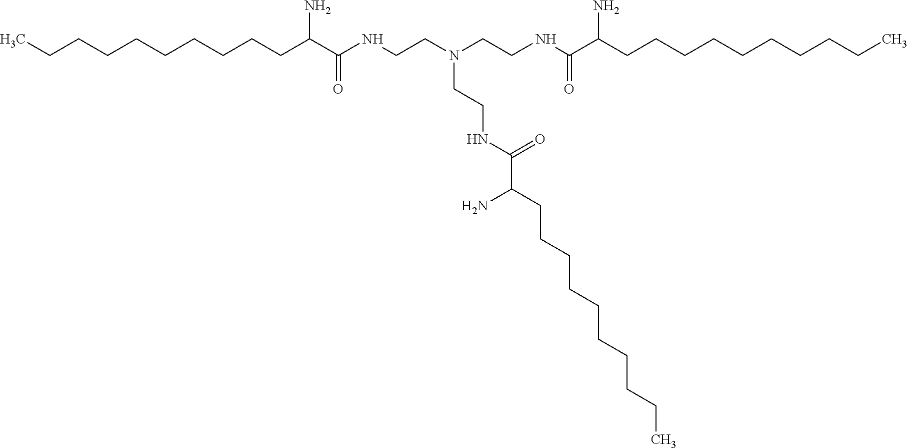 [SMILES: CCCCCCCCCCC(N)C(=O)NCCN(CCNC(=O)C(N)CCCCCCCCCC)CCNC(=O)C(N)CCCCCCCCCC] |
| 3C12amide-OH | 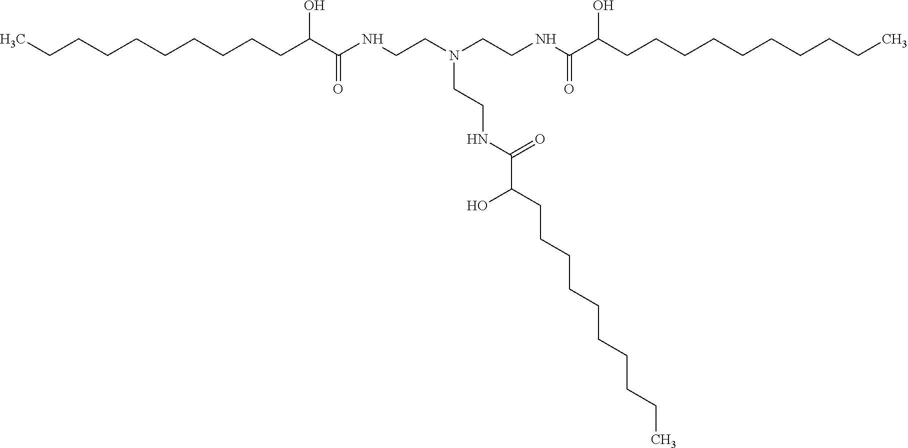 [SMILES: CCCCCCCCCCC(O)C(=O)NCCN(CCNC(=O)C(O)CCCCCCCCCC)CCNC(=O)C(O)CCCCCCCCCC] |
| 3C12Ester-OH | 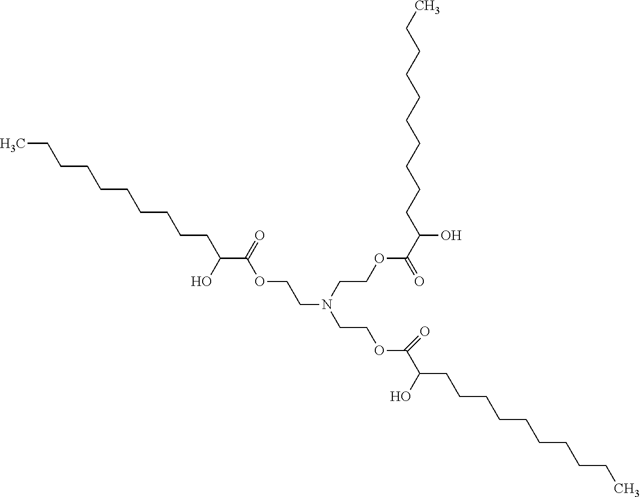 [SMILES: CCCCCCCCCCC(O)C(=O)OCCN(CCOC(=O)C(O)CCCCCCCCCC)CCOC(=O)C(O)CCCCCCCCCC] |
| 3C12 Ester-amin | 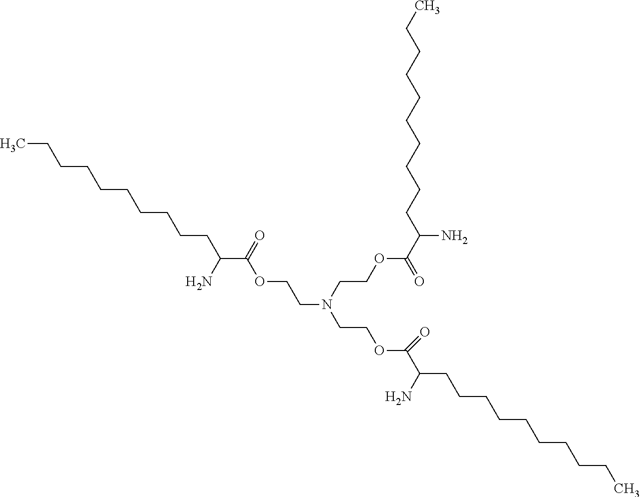 [SMILES: CCCCCCCCCCC(N)C(=O)OCCN(CCOC(=O)C(N)CCCCCCCCCC)CCOC(=O)C(N)CCCCCCCCCC] |
| 3C12Ester-DMA | 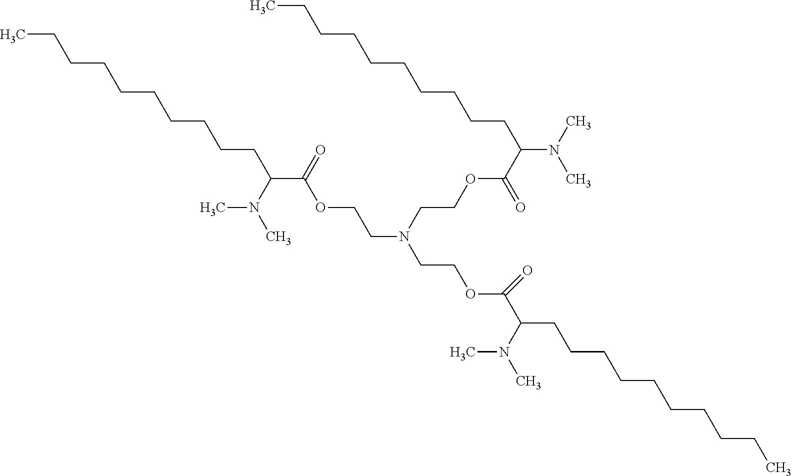 [SMILES: CCCCCCCCCCC(C(=O)OCCN(CCOC(=O)C(CCCCCCCCCC)N(C)C)CCOC(=O)C(CCCCCCCCCC)N(C)C)N(C)C] |
| 2C12Amid-DMA | 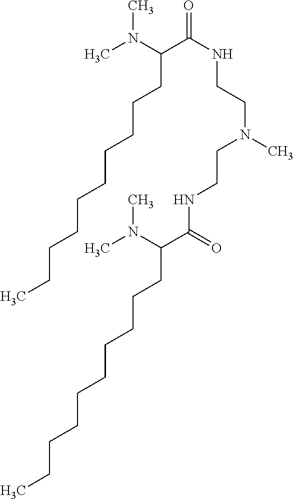 [SMILES: CCCCCCCCCCC(C(=O)NCCN(C)CCNC(=O)C(CCCCCCCCCC)N(C)C)N(C)C] |
| 3C12-lin-amid- DMA | 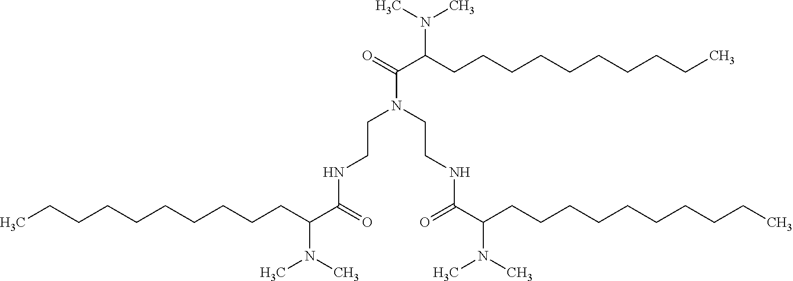 [SMILES: CCCCCCCCCCC(C(=O)NCCN(CCNC(=O)C(CCCCCCCCCC)N(C)C)C(=O)C(CCCCCCCCCC)N(C)C)N(C)C] |
| 2C12-sperm- amid-DMA | 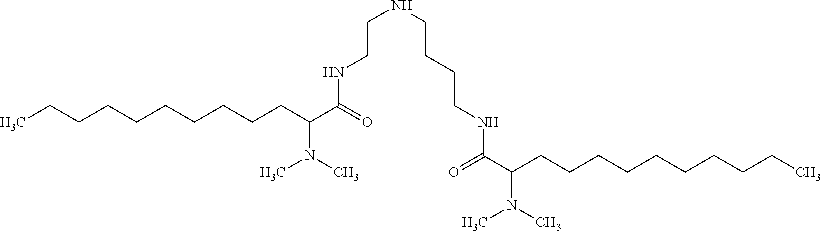 [SMILES: CCCCCCCCCCC(C(=O)NCCCCNCCNC(=O)C(CCCCCCCCCC)N(C)C)N(C)C] |
| 3C12-sperm- amid-DMA | 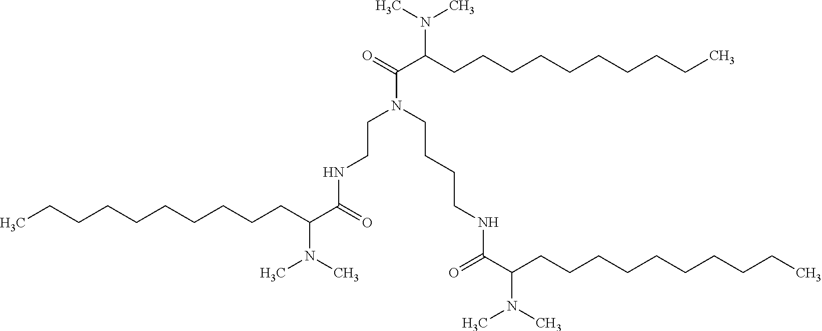 [SMILES: CCCCCCCCCCC(C(=O)NCCCCN(CCNC(=O)C(CCCCCCCCCC)N(C)C)C(=O)C(CCCCCCCCCC)N(C)C)N(C)C] |

According preferred embodiments, the peptide polymer comprises a lipidoid of Table 7, preferably lipidoid 3-C12-OH as specified above, is used to complex the at least one coding RNA of the first aspect to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid. In that context, the disclosure of WO2017/212009A1, in particular claims 1 to 10 of WO2017/212009A1, and the specific disclosure relating thereto is herewith incorporated by reference.

### Encapsulation/Complexation in LNPs:

In preferred embodiments of the second aspect, the at least one coding RNA is complexed, encapsulated, partially encapsulated, or associated with one or more lipids (e.g. cationic lipids and/or neutral lipids), thereby forming liposomes, lipid nanoparticles (LNPs), lipoplexes, and/or nanoliposomes.

The liposomes, lipid nanoparticles (LNPs), lipoplexes, and/or nanoliposomes—incorporated RNA may be completely or partially located in the interior space of the liposomes, lipid nanoparticles (LNPs), lipoplexes, and/or nanoliposomes, within the membrane, or associated with the exterior surface of the membrane. The incorporation of a nucleic acid into liposomes is also referred to herein as “encapsulation” wherein the RNA is entirely contained within the interior space of the liposomes, lipid nanoparticles (LNPs), lipoplexes, and/or nanoliposomes. The purpose of incorporating an RNA into liposomes, lipid nanoparticles (LNPs), lipoplexes, and/or nanoliposomes is to protect the RNA from an environment which may contain enzymes or chemicals that degrade RNA and/or systems or receptors that cause the rapid excretion of the RNA. Moreover, incorporating an RNA into liposomes, lipid nanoparticles (LNPs), lipoplexes, and/or nanoliposomes may promote the uptake of the RNA, and hence, may enhance the therapeutic effect of the RNA encoding antigenic CSP. Accordingly, incorporating an RNA into liposomes, lipid nanoparticles (LNPs), lipoplexes, and/or nanoliposomes may be particularly suitable for a vaccine, e.g. for intramuscular or intradermal administration.

In this context, the terms “complexed” or “associated” refer to the essentially stable combination of coding RNA of the first aspect and with one or more lipids into larger complexes or assemblies without covalent binding.

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of RNA. For example, a liposome, a lipid complex, a lipoplex and the like are within the scope of a lipid nanoparticle (LNP).

Liposomes, lipid nanoparticles (LNPs), lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphophilic polymers and surfactants (e.g., polymerosomes, niosomes, etc.). In the context of the present invention, an LNP typically serves to transport the RNA of the first aspect to a target tissue.

Accordingly, in preferred embodiments of the second aspect, the at least one RNA is complexed with one or more lipids thereby forming lipid nanoparticles (LNP), wherein said LNP is particularly suitable for intramuscular and/or intradermal administration.

LNPs typically comprise a cationic lipid and one or more excipient selected from neutral lipids, charged lipids, steroids and polymer conjugated lipids (e.g. PEGylated lipid). The coding RNA may be encapsulated in the lipid portion of the LNP or an aqueous space enveloped by some or the entire lipid portion of the LNP. The coding RNA or a portion thereof may also be associated and complexed with the LNP. An LNP may comprise any lipid capable of forming a particle to which the nucleic acids are attached, or in which the one or more nucleic acids are encapsulated. Preferably, the LNP comprising nucleic acids comprises one or more cationic lipids, and one or more stabilizing lipids. Stabilizing lipids include neutral lipids and PEGylated lipids.

The cationic lipid of an LNP may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

The LNP may comprise any further cationic or cationisable lipid, i.e. any of a number of lipid species which carry a net positive charge at a selective pH, such as physiological pH.

Such lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyI)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 98N12-5,1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl[1,3]-dioxolane) HGT4003, 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)-N,N-dimethyl-2,2-di((9Z,12Z)-octadeca-9,12-dienyl)tetrahydro-3aH-cyclopenta[d][1,3]dioxol-5-amine)), 1,1″-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)[1,3]-dioxolane (DLin-K-C2-DMA), 2,2-dilinoleyl-4-dimethylaminomethyl[1,3]-dioxolane (DLin-K-DMA), NC98-5 (4,7,13-tris(3-oxo-3-(undecylamino)propyl)-N1,N 16-diundecyl-4,7,10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010/053572 (and particularly, CI 2-200 described at paragraph [00225]) and WO2012/170930, both of which are incorporated herein by reference, HGT4003, HGT5000, HGTS001, HGT5001, HGT5002 (see US20150140070A1).

In some embodiments, the lipid is selected from the group consisting of 98N12-5, C12-200, and ckk-E12.

In one embodiment, the further cationic lipid is an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In one embodiment, the coding RNA of the first aspect may be formulated in an aminoalcohol lipidoid.

Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017/075531A1, hereby incorporated by reference.

In another embodiment, ionizable lipids can also be the compounds as disclosed in WO2015/074085A1 (i.e. ATX-001 to ATX-032 or the compounds as specified in claims 1-26), U.S. Appl. Nos. 61/905,724 and 15/614,499 or U.S. Pat. Nos. 9,593,077 and 9,567,296 hereby incorporated by reference in their entirety.

In that context, any lipid derived from generic formula (X1)

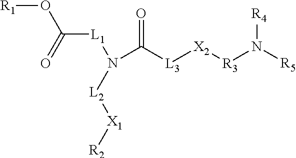

wherein, Ri and R2 are the same or different, each a linear or branched alkyl consisting of 1 to 9 carbons, an alkenyl or alkynyl consisting of 2 to 11carbons, Li and L2 are the same or different, each a linear alkylene or alkenylene consisting of 5 to 18 carbons, or forming a heterocycle with N, Xi is a bond, or is —CO—O— whereby —L2-CO—O—R2 is formed, X2 is S or O, L3 is a bond or a linear or branched alkylene consisting of 1 to 6 carbons, or forming a heterocycle with N, R3 is a linear or branched alkylene consisting of 1 to 6 carbons, and R4 and R 5 are the same or different, each hydrogen or a linear or branched alkyl consisting of 1 to 6 carbons; or a pharmaceutically acceptable salt thereof may be suitably used.

In other embodiments, suitable cationic lipids can also be the compounds as disclosed in WO2017/117530A1 (i.e. lipids 13, 14, 15, 16, 17, 18, 19, 20, or the compounds as specified in the claims), hereby incorporated by reference in its entirety.

In that context, any lipid derived from generic formula (X2)

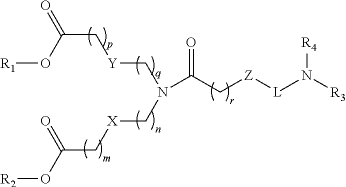

wherein
X is a linear or branched alkylene or alkenylene, monocyclic, bicyclic, or tricyclic arene or heteroarene;
Y is a bond, an ethene, or an unsubstituted or substituted aromatic or heteroaromatic ring; Z is S or 0;
L is a linear or branched alkylene of 1 to 6 carbons;
R-3 and R4 are independently a linear or branched alkyl of 1 to 6 carbons;
Ri and R2 are independently a linear or branched alkyl or alkenyl of 1 to 20 carbons; r is 0 to 6; and
m, n, p, and q are independently 1 to 18;
wherein when n=q, m=p, and Ri=R2, then X and Y differ;
wherein when X=Y, n=q, m=p, then Ri and R2 differ;
wherein when X=Y, n=q, and Ri=R2, then m and p differ; and
wherein when X=Y, m=p, and Ri=R2, then n and q differ;
or a pharmaceutically acceptable salt thereof.

In preferred embodiments, a lipid may be used derived from formula (X2), wherein, X is a bond, linear or branched alkylene, alkenylene, or monocyclic, bicyclic, or tricyclic arene or heteroarene; Y is a monocyclic, bicyclic, or tricyclic arene or heteroarene; Z is S or O; L is a linear or branched alkylene of 1 to 6 carbons; R3 and R4 are independently a linear or branched alkyl of 1 to 6 carbons; Ri and R2 are independently a linear or branched alkyl or alkenyl of 1 to 20 carbons; r is 0 to 6; and m, n, p, and q are independently 1 to 18; or a pharmaceutically acceptable salt thereof may be suitably used.

In preferred embodiments, ionizable lipids may also be selected from the lipids disclosed in WO2018/078053A1 (i.e. lipids derived from formula I, II, and III of WO2018/078053A1, or lipids as specified in claims 1 to 12 of WO2018/078053A1), the disclosure of WO2018/078053A1 hereby incorporated by reference in its entirety. In that context, lipids disclosed in Table 7 of WO2018/078053A1 (e.g. lipids derived from formula I-1 to I-41) and lipids disclosed in Table 8 of WO2018/078053A1 (e.g. lipids derived from formula II-1 to II-36) may be suitably used in the context of the invention. Accordingly, formula I-1 to formula I-41 and formula II-1 to formula II-36 of WO2018/078053A1, and the specific disclosure relating thereto, are herewith incorporated by reference.

In particularly preferred embodiments of the second aspect, a suitable lipid may be a cationic lipid according to formula (III)

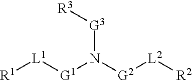

or a pharmaceutically acceptable salt, tautomer, prodrug or stereoisomer thereof, wherein, R1, R2, R3, L1, L2, G1, G2, and G3 are as below.
Formula (III) is further defined in that:
one of L¹ or L² is —O(C═O)—, —(C═O)O—, —C(═O)—, —O—, —S(O)ₓ—, —S—S—, SC(═O)—, —NR^{a}C(═O)—, —C(═O)NR^{a}—, —NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, SC(═O)—, —NR^{a}C(═O)—, —C(═O)NR^{a}—, —NR^{a}C(═O)NR^{a}—, —OC(═O)NR^{a}— or —NR^{a}C(═O)O- or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of formula (III), the lipid has one of the following structures (IIIA) or (IIIB):

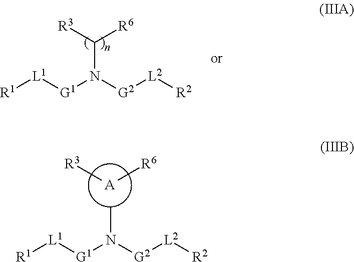

wherein:
A is a 3 to 8-membered cycloalkyl or cycloalkylene ring; R⁶ is, at each occurrence, independently H, OH or C₁-C₂₄ alkyl; n is an integer ranging from 1 to 15.

In some of the foregoing embodiments of formula (III), the lipid has structure (IIIA), and in other embodiments, the lipid has structure (IIIB).

In other embodiments of formula (III), the lipid has one of the following structures (IIIC) or (IIID):

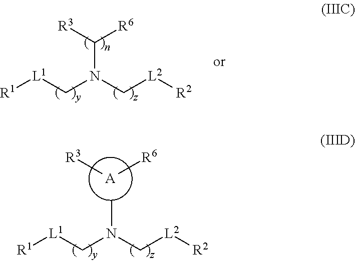

wherein y and z are each independently integers ranging from 1 to 12.

In any of the foregoing embodiments of formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In preferred embodiments of the second aspect, the cationic lipid of the LNP is a compound of formula III,

wherein:
L¹ and L² are each independently —O(C═O)— or (C═O)—O—;
G³ is C₁-C₂₄ alkylene or C₁-C₂₄ alkenylene; and
R³ is H or OR⁵.

In some different embodiments of formula (III), the lipid has one of the following structures (IIIE) or (IIIF):

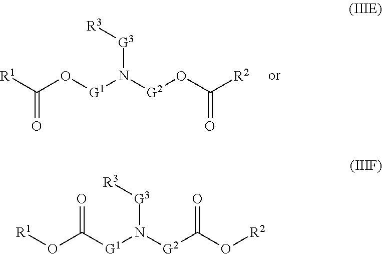

In some of the foregoing embodiments of formula (III), the lipid has one of the following structures (IIIG), (IIIH), (IIII), or (IIIJ):

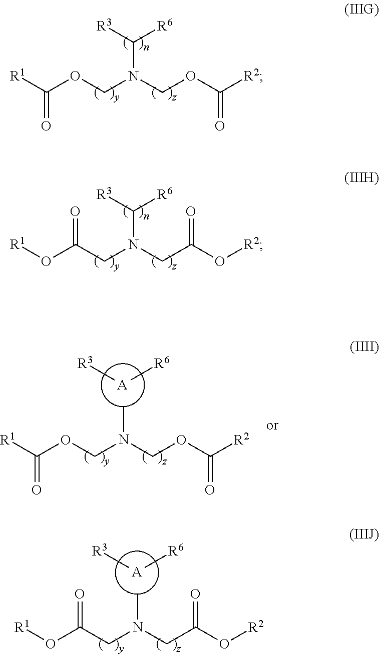

In some of the foregoing embodiments of formula (III), n is an integer ranging from 2 to 12, for example from 2 to 8 or from 2 to 4. In some embodiments, n is 3, 4, 5 or 6. In some embodiments, n is 3. In some embodiments, n is 4. In some embodiments, n is 5. In some embodiments, n is 6. In some other of the foregoing embodiments of formula (III), y and z are each independently an integer ranging from 2 to 10. For example, in some embodiments, y and z are each independently an integer ranging from 4 to 9 or from 4 to 6. In some of the foregoing embodiments of formula (III), R⁶ is H. In other of the foregoing embodiments, R⁶ is C₁-C₂₄ alkyl. In other embodiments, R⁶ is OH. In some embodiments of formula (III), G³ is unsubstituted. In other embodiments, G3 is substituted. In various different embodiments, G³ is linear C₁-C₂₄ alkylene or linear C₁-C₂₄ alkenylene. In some other foregoing embodiments of formula (III), R¹ or R², or both, is C₆-C₂₄ alkenyl. For example, in some embodiments, R¹ and R² each, independently have the following structure:

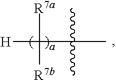

wherein:
R^{7a} and R^{7b} are, at each occurrence, independently H or C₁-C₁₂ alkyl; and a is an integer from 2 to 12, wherein R^{7a}, R^{7b} and a are each selected such that R¹ and R² each independently comprise from 6 to 20 carbon atoms. For example, in some embodiments a is an integer ranging from 5 to 9 or from 8 to 12. In some of the foregoing embodiments of formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of formula (III), R¹ or R², or both, has one of the following structures:

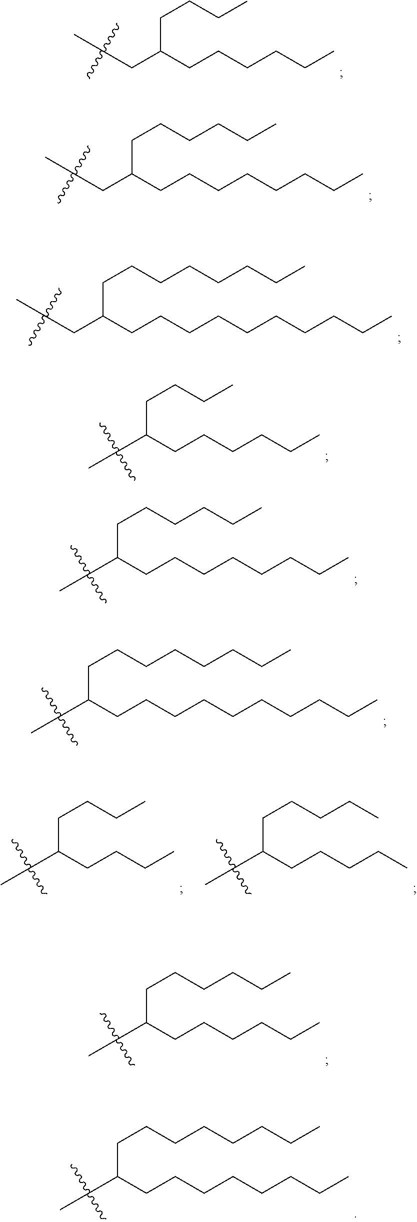

In preferred embodiments of the second aspect, the cationic lipid of the LNP is a compound of formula III, wherein:

L¹ and L² are each independently —O(C═O)— or (C═O)—O—; and
R¹ and R² each independently have one of the following structures:

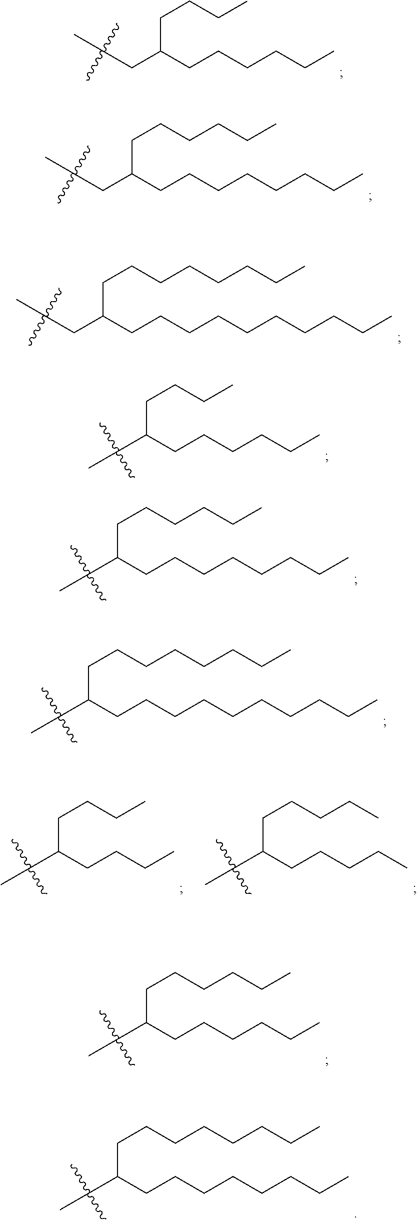

In some of the foregoing embodiments of formula (III), R³ is OH, CN, —C(═O)OR⁴, —OC(═O)R⁴ or —NHC(═O)R⁴. In some embodiments, R⁴ is methyl or ethyl.

In preferred embodiments of the second aspect, the cationic lipid of the LNP is a compound of formula III, wherein R³ is OH.

In particularly preferred embodiment of the second aspect, the coding RNA of the first aspect and is complexed with one or more lipids thereby forming lipid nanoparticles (LNP), wherein the LNP is selected from structures III-1 to III-36 (see Table 8).

TABLE 8
Representative lipid compounds derived from formula (III)

| No. | Structure |
|---|---|
| III-1 | 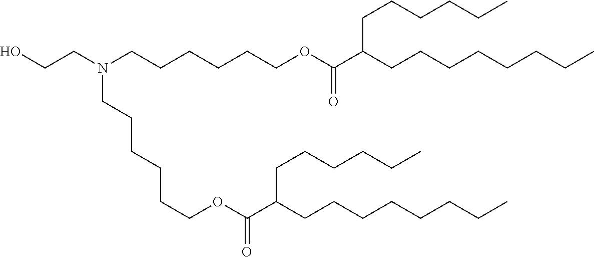 [SMILES: CCCCCCCCC(CCCCCC)C(=O)OCCCCCCN(CCO)CCCCCCOC(=O)C(CCCCCC)CCCCCCCC] |
| III-2 | 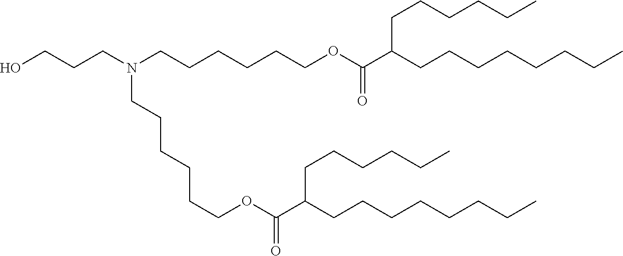 [SMILES: CCCCCCCCC(CCCCCC)C(=O)OCCCCCCN(CCCO)CCCCCCOC(=O)C(CCCCCC)CCCCCCCC] |
| III-3 | 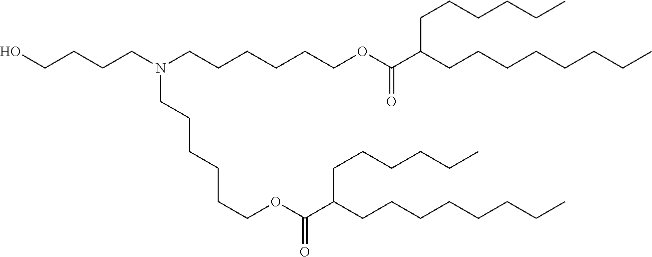 [SMILES: CCCCCCCCC(CCCCCC)C(=O)OCCCCCCN(CCCCO)CCCCCCOC(=O)C(CCCCCC)CCCCCCCC] |
| III-4 | 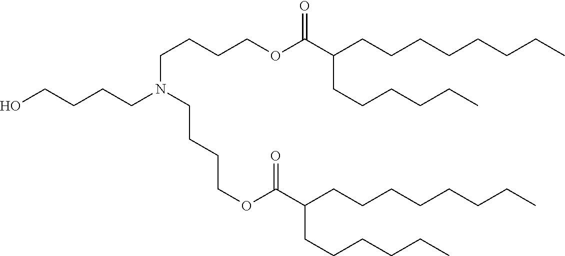 [SMILES: CCCCCCCCC(CCCCCC)C(=O)OCCCCN(CCCCO)CCCCOC(=O)C(CCCCCC)CCCCCCCC] |
| III-5 | 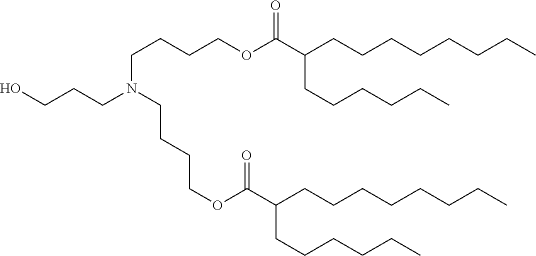 [SMILES: CCCCCCCCC(CCCCCC)C(=O)OCCCCN(CCCO)CCCCOC(=O)C(CCCCCC)CCCCCCCC] |
| III-6 | 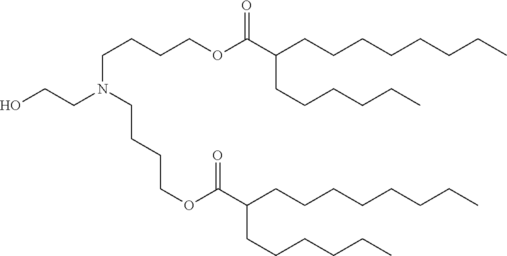 [SMILES: CCCCCCCCC(CCCCCC)C(=O)OCCCCN(CCO)CCCCOC(=O)C(CCCCCC)CCCCCCCC] |
| III-7 | 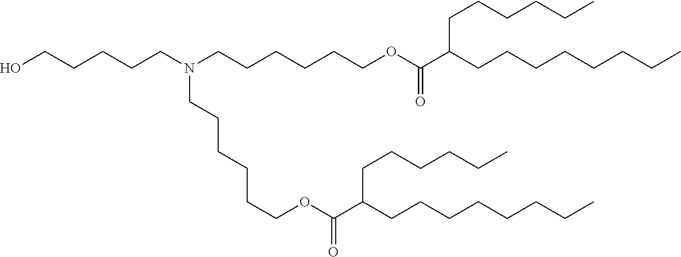 [SMILES: CCCCCCCCC(CCCCCC)C(=O)OCCCCCCN(CCCCCO)CCCCCCOC(=O)C(CCCCCC)CCCCCCCC] |
| III-8 | 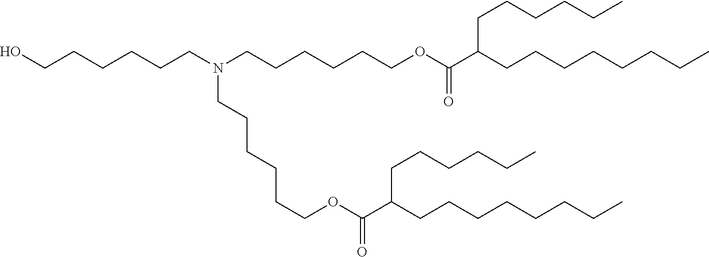 [SMILES: CCCCCCCCC(CCCCCC)C(=O)OCCCCCCN(CCCCCCO)CCCCCCOC(=O)C(CCCCCC)CCCCCCCC] |
| III-9 | 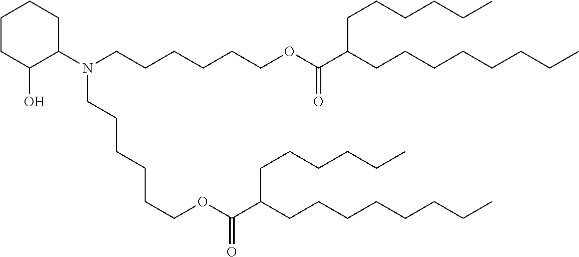 [SMILES: CCCCCCCCC(CCCCCC)C(=O)OCCCCCCN(CCCCCCOC(=O)C(CCCCCC)CCCCCCCC)C1CCCCC1O] |
| III-10 | 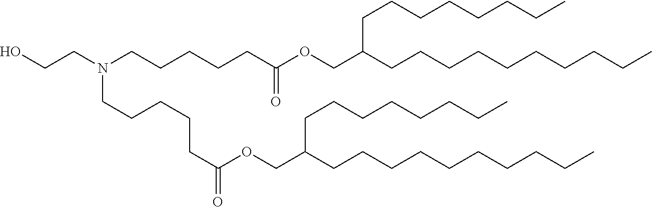 [SMILES: CCCCCCCCCCC(CCCCCCCC)COC(=O)CCCCCN(CCO)CCCCCC(=O)OCC(CCCCCCCC)CCCCCCCCCC] |
| III-11 | 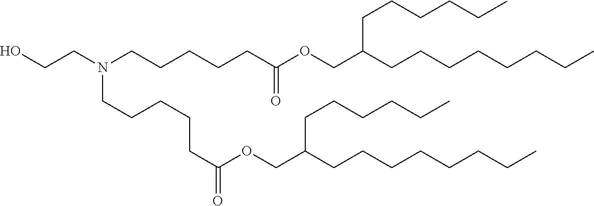 [SMILES: CCCCCCCCC(CCCCCC)COC(=O)CCCCCN(CCO)CCCCCC(=O)OCC(CCCCCC)CCCCCCCC] |
| III-12 | 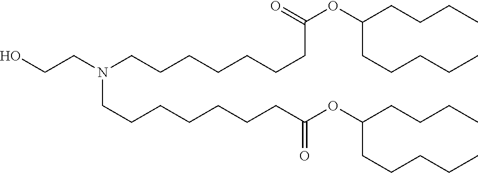 [SMILES: CCCCCC(CCCC)OC(=O)CCCCCCCN(CCO)CCCCCCCC(=O)OC(CCCC)CCCCC] |
| III-13 | 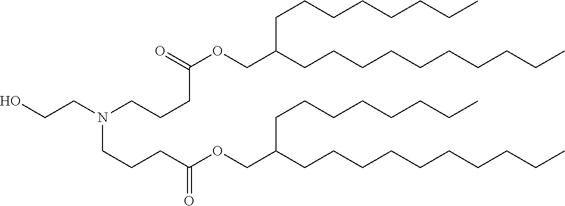 [SMILES: CCCCCCCCCCC(CCCCCCCC)COC(=O)CCCN(CCO)CCCC(=O)OCC(CCCCCCCC)CCCCCCCCCC] |
| III-14 | 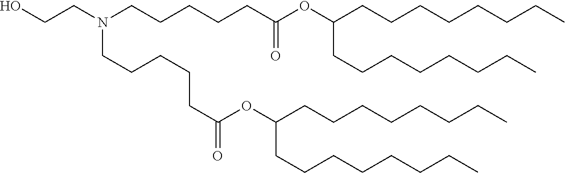 [SMILES: CCCCCCCCC(CCCCCCCC)OC(=O)CCCCCN(CCO)CCCCCC(=O)OC(CCCCCCCC)CCCCCCCC] |
| III-15 | 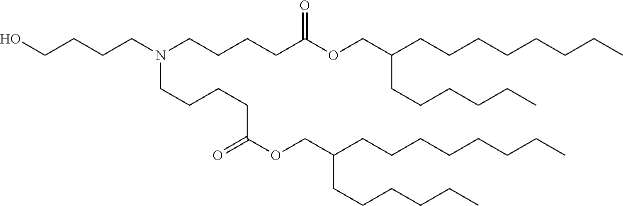 [SMILES: CCCCCCCCC(CCCCCC)COC(=O)CCCCN(CCCCO)CCCCC(=O)OCC(CCCCCC)CCCCCCCC] |
| III-16 | 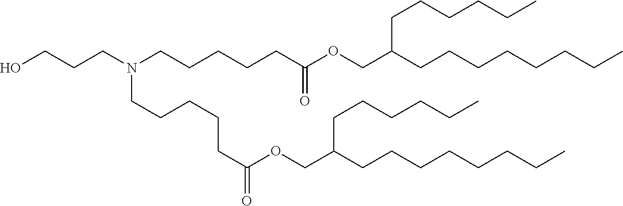 [SMILES: CCCCCCCCC(CCCCCC)COC(=O)CCCCCN(CCCO)CCCCCC(=O)OCC(CCCCCC)CCCCCCCC] |
| III-17 | 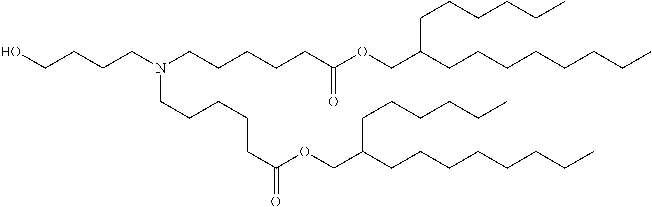 [SMILES: CCCCCCCCC(CCCCCC)COC(=O)CCCCCN(CCCCO)CCCCCC(=O)OCC(CCCCCC)CCCCCCCC] |
| III-18 | 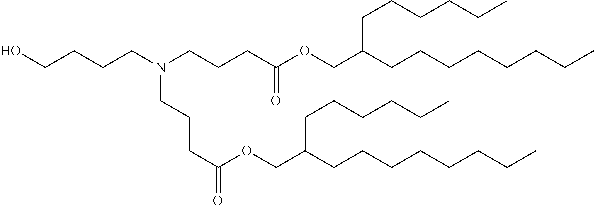 [SMILES: CCCCCCCCC(CCCCCC)COC(=O)CCCN(CCCCO)CCCC(=O)OCC(CCCCCC)CCCCCCCC] |
| III-19 | 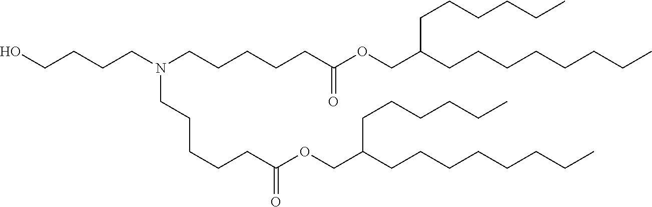 [SMILES: CCCCCCCCC(CCCCCC)COC(=O)CCCCCN(CCCCO)CCCCCC(=O)OCC(CCCCCC)CCCCCCCC] |
| III-20 | 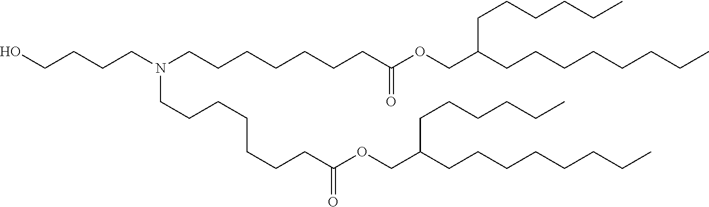 [SMILES: CCCCCCCCC(CCCCCC)COC(=O)CCCCCCCN(CCCCO)CCCCCCCC(=O)OCC(CCCCCC)CCCCCCCC] |
| III-21 | 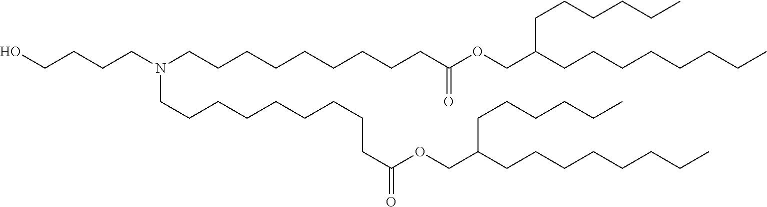 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 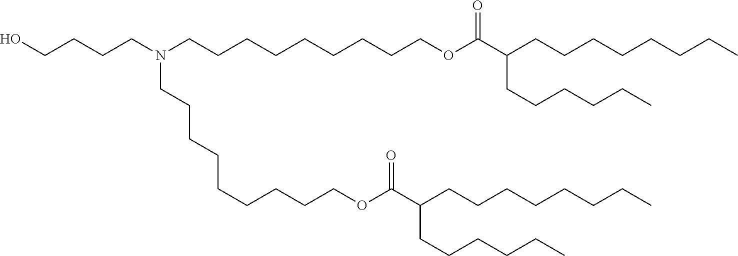 [SMILES: CCCCCCCCC(CCCCCC)C(=O)OCCCCCCCCCN(CCCCO)CCCCCCCCCOC(=O)C(CCCCCC)CCCCCCCC] |
| III-23 | 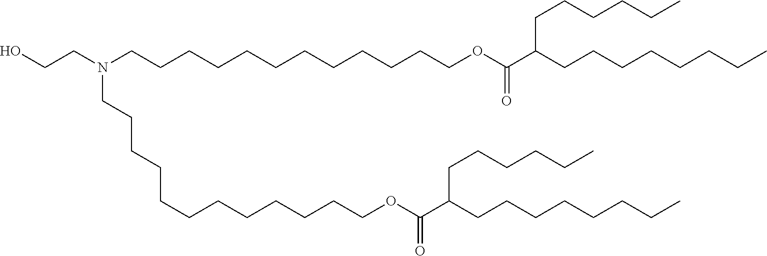 [SMILES: CCCCCCCCC(CCCCCC)C(=O)OCCCCCCCCCCCCN(CCO)CCCCCCCCCCCCOC(=O)C(CCCCCC)CCCCCCCC] |
| III-24 | 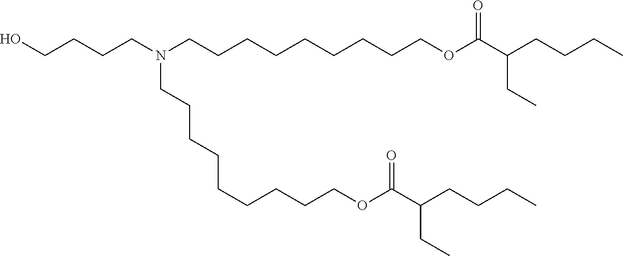 [SMILES: CCCCC(CC)C(=O)OCCCCCCCCCN(CCCCO)CCCCCCCCCOC(=O)C(CC)CCCC] |
| III-25 | 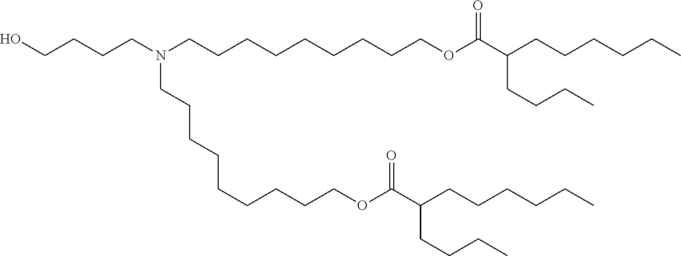 [SMILES: CCCCCCC(CCCC)C(=O)OCCCCCCCCCN(CCCCO)CCCCCCCCCOC(=O)C(CCCC)CCCCCC] |
| III-26 | 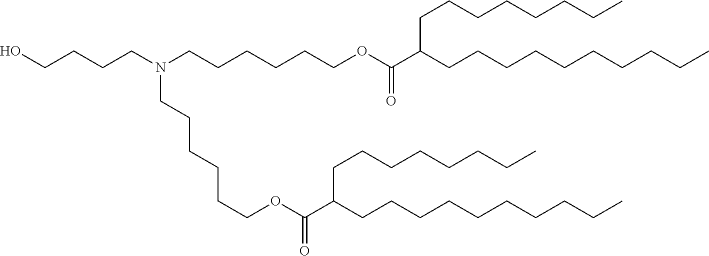 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 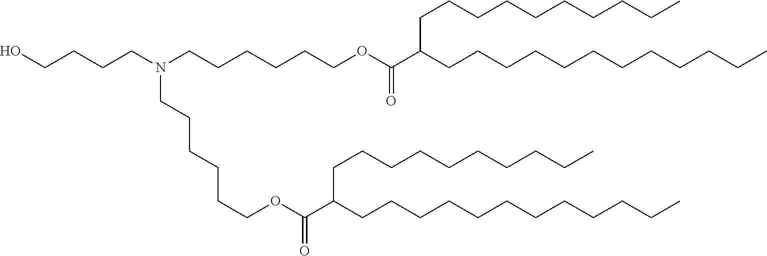 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 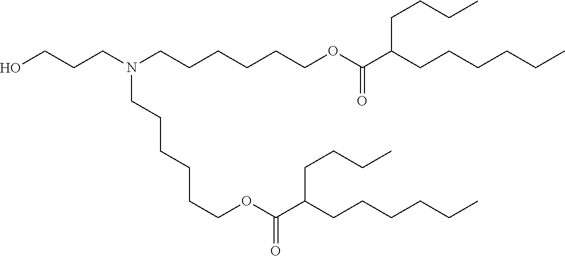 [SMILES: CCCCCCC(CCCC)C(=O)OCCCCCCN(CCCO)CCCCCCOC(=O)C(CCCC)CCCCCC] |
| III-29 | 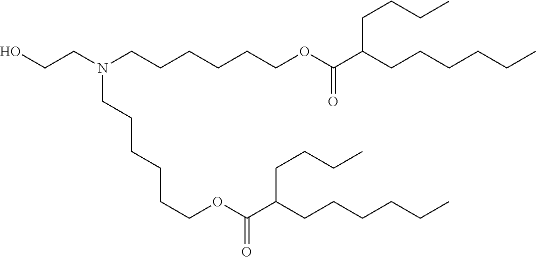 [SMILES: CCCCCCC(CCCC)C(=O)OCCCCCCN(CCO)CCCCCCOC(=O)C(CCCC)CCCCCC] |
| III-30 | 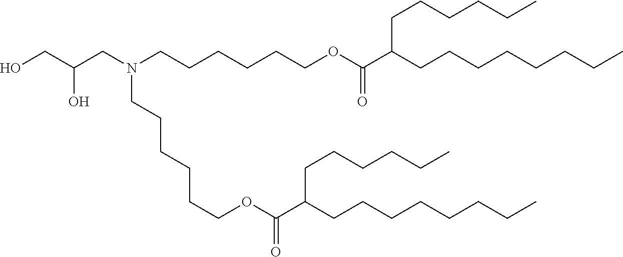 [SMILES: CCCCCCCCC(CCCCCC)C(=O)OCCCCCCN(CCCCCCOC(=O)C(CCCCCC)CCCCCCCC)CC(O)CO] |
| III-31 | 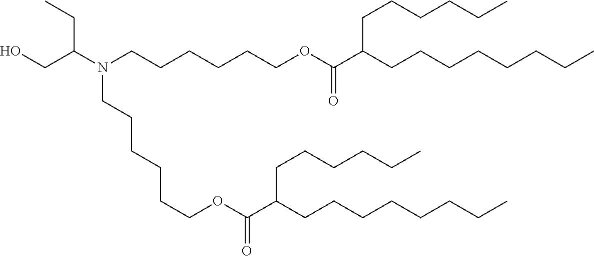 [SMILES: CCCCCCCCC(CCCCCC)C(=O)OCCCCCCN(CCCCCCOC(=O)C(CCCCCC)CCCCCCCC)C(CC)CO] |
| III-32 | 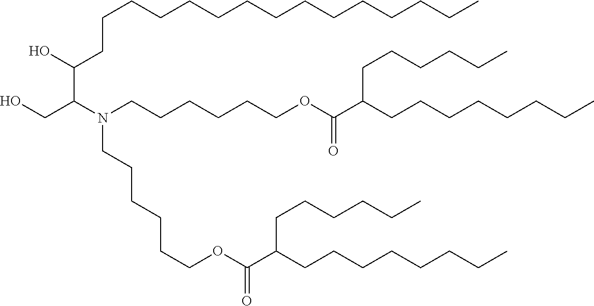 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 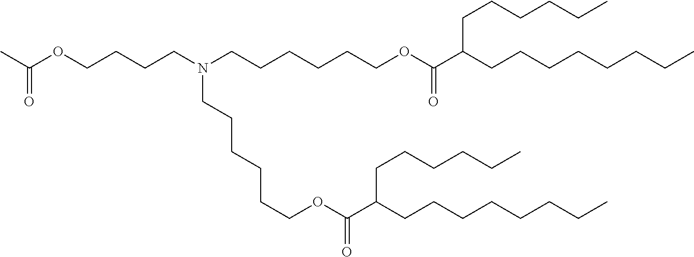 [SMILES: CCCCCCCCC(CCCCCC)C(=O)OCCCCCCN(CCCCCCOC(=O)C(CCCCCC)CCCCCCCC)CCCCOC(C)=O] |
| III-34 | 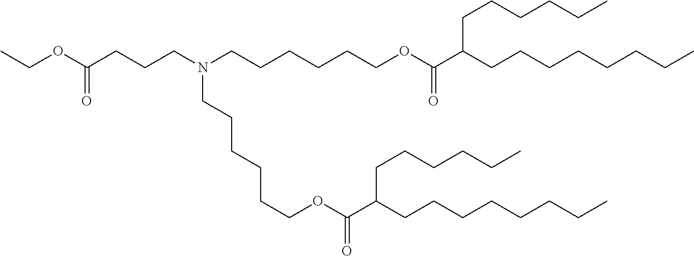 [SMILES: CCCCCCCCC(CCCCCC)C(=O)OCCCCCCN(CCCCCCOC(=O)C(CCCCCC)CCCCCCCC)CCCC(=O)OCC] |
| III-35 | 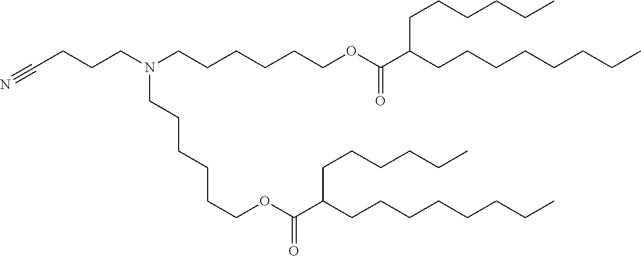 [SMILES: CCCCCCCCC(CCCCCC)C(=O)OCCCCCCN(CCCC#N)CCCCCCOC(=O)C(CCCCCC)CCCCCCCC] |
| III-36 | 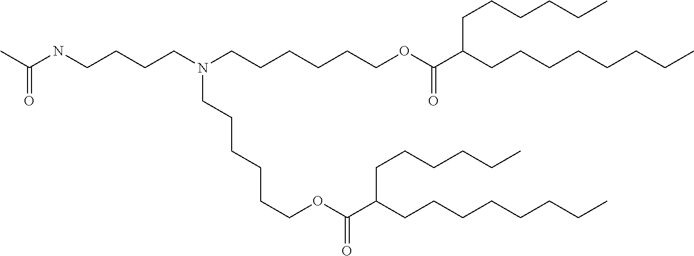 [SMILES: CCCCCCCCC(CCCCCC)C(=O)OCCCCCCN(CCCCCCOC(=O)C(CCCCCC)CCCCCCCC)CCCCNC(C)=O] |

In some embodiments, the LNPs comprise a lipid of formula (III), a coding RNA of the first aspect, and one or more excipient selected from neutral lipids, steroids and PEGylated lipids. In some embodiments the lipid of formula (III) is compound III-3. In some embodiments the lipid of formula (III) is compound III-7.

In preferred embodiments, the LNP comprises a cationic lipid selected from:

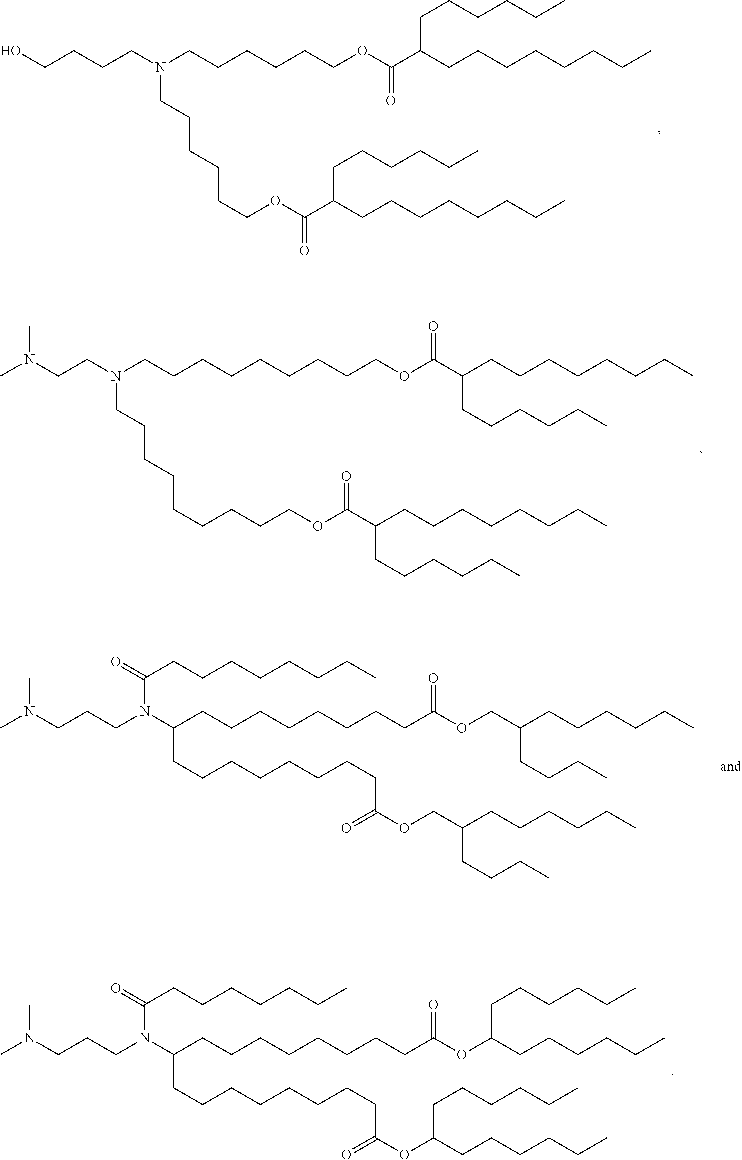

In particularly preferred embodiment of the second aspect, the coding RNA of the first aspect is complexed with one or more lipids thereby forming lipid nanoparticles (LNP), wherein the LNP comprises the following cationic lipid (lipid according to formula III-3 of Table 8):

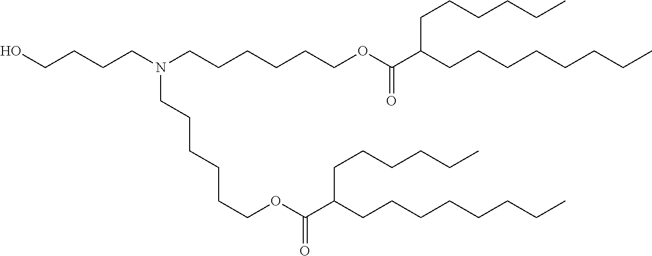 [SMILES: CCCCCCCCC(CCCCCC)C(=O)OCCCCCCN(CCCCO)CCCCCCOC(=O)C(CCCCCC)CCCCCCCC]

In certain embodiments, the cationic lipid as defined herein, preferably as disclosed in Table 8, more preferably cationic lipid compound III-3, is present in the LNP in an amount from about 30 to about 95 mole percent, relative to the total lipid content of the LNP. If more than one cationic lipid is incorporated within the LNP, such percentages apply to the combined cationic lipids.

In one embodiment, the cationic lipid is present in the LNP in an amount from about 30 to about 70 mole percent. In one embodiment, the cationic lipid is present in the LNP in an amount from about 40 to about 60 mole percent, such as about 40, 41, 42, 43, 44, 45, 46, 47, 48, 49, 50, 51, 52, 53, 54, 55, 56, 57, 58, 59 or 60 mole percent, respectively. In embodiments, the cationic lipid is present in the LNP in an amount from about 47 to about 48 mole percent, such as about 47.0, 47.1, 47.2, 47.3, 47.4, 47.5, 47.6, 47.7, 47.8, 47.9, 50.0 mole percent, respectively, wherein 47.7 mole percent are particularly preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to the coding RNA of the first aspect is from about 3 to about 15, such as from about 5 to about 13 or from about 7 to about 11.

In some embodiments of the invention the LNP comprises a combination or mixture of any the lipids described above.

Other suitable (cationic or ionizable) lipids are disclosed in WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US2013/0178541, US2013/0225836, US2014/0039032 and WO2017/112865. In that context, the disclosures of WO2009/086558, WO2009/127060, WO2010/048536, WO2010/054406, WO2010/088537, WO2010/129709, WO2011/153493, WO2013/063468, U52011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US2013/0178541, US2013/0225836 and US2014/0039032 and WO2017/112865 specifically relating to (cationic) lipids suitable for LNPs are incorporated herewith by reference.

In some embodiments, the lipid is selected from the group consisting of 98N12-5, C12-200, and ckk-E12.

In some embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention.

In some embodiments, the protonatable lipids have a pKa of the protonatable group in the range of about 4 to about 11, e.g., a pKa of about 5 to about 7.

LNPs can comprise two or more (different) cationic lipids. The cationic lipids may be selected to contribute different advantageous properties. For example, cationic lipids that differ in properties such as amine pKa, chemical stability, half-life in circulation, half-life in tissue, net accumulation in tissue, or toxicity can be used in the LNP. In particular, the cationic lipids can be chosen so that the properties of the mixed-LNP are more desirable than the properties of a single-LNP of individual lipids.

The amount of the permanently cationic lipid or lipidoid may be selected taking the amount of the RNA cargo into account. In one embodiment, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the RNA which is used as cargo. The N/P ratio may be calculated on the basis that, for example, 1 ug RNA typically contains about 3 nmol phosphate residues, provided that the RNA exhibits a statistical distribution of bases. The “N”-value of the lipid or lipidoid may be calculated on the basis of its molecular weight and the relative content of permanently cationic and—if present—cationisable groups.

LNP in vivo characteristics and behavior can be modified by addition of a hydrophilic polymer coating, e.g. polyethylene glycol (PEG), to the LNP surface to confer steric stabilization. Furthermore, LNPs can be used for specific targeting by attaching ligands (e.g. antibodies, peptides, and carbohydrates) to its surface or to the terminal end of the attached PEG chains (e.g. via PEGylated lipids).

In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

In certain embodiments, the LNP comprises an additional, stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEGylated ceramide (PEG-cer), or a PEG dialkoxypropylcarbamate such as ω-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate.

In preferred embodiments of the second aspect, the coding RNA of the first aspect is complexed with one or more lipids thereby forming lipid nanoparticles (LNP), wherein the LNP additionally comprises a PEGylated lipid with the formula (IV):

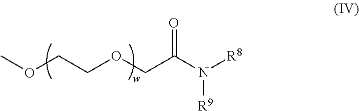

or a pharmaceutically acceptable salt, tautomer or stereoisomer thereof, wherein R⁸ and R⁹ are each independently a straight or branched, saturated or unsaturated alkyl chain containing from 10 to 30 carbon atoms, wherein the alkyl chain is optionally interrupted by one or more ester bonds; and w has mean value ranging from 30 to 60.

In some of the foregoing embodiments of the PEGylated lipid according to formula (IV), R⁸ and R⁹ are not both n-octadecyl when w is 42. In some other embodiments, R⁸ and R⁹ are each independently a straight or branched, saturated or unsaturated alkyl chain containing from 10 to 18 carbon atoms. In some embodiments, R⁸ and R⁹ are each independently a straight or branched, saturated or unsaturated alkyl chain containing from 12 to 16 carbon atoms. In some embodiments, R⁸ and R⁹ are each independently a straight or branched, saturated or unsaturated alkyl chain containing 12 carbon atoms. In some embodiments, R⁸ and R⁹ are each independently a straight or branched, saturated or unsaturated alkyl chain containing 14 carbon atoms. In other embodiments, R⁸ and R⁹ are each independently a straight or branched, saturated or unsaturated alkyl chain containing 16 carbon atoms. In still more embodiments, R⁸ and R⁹ are each independently a straight or branched, saturated or unsaturated alkyl chain containing 18 carbon atoms. In still other embodiments, R⁸ is a straight or branched, saturated or unsaturated alkyl chain containing 12 carbon atoms and R⁹ is a straight or branched, saturated or unsaturated alkyl chain containing 14 carbon atoms.

In various embodiments, w spans a range that is selected such that the PEG portion of the PEGylated lipid according to formula (IV) has an average molecular weight of about 400 to about 6000 g/mol. In some embodiments, the average w is about 50.

In preferred embodiments of the second aspect, R⁸ and R⁹ of the PEGylated lipid according to formula (IV) are saturated alkyl chains.

In a particularly preferred embodiment of the second aspect, the coding RNA of the first aspect is complexed with one or more lipids thereby forming lipid nanoparticles (LNP), wherein the LNP additionally comprises a PEGylated lipid, wherein the PEG lipid is of formula (IVa)

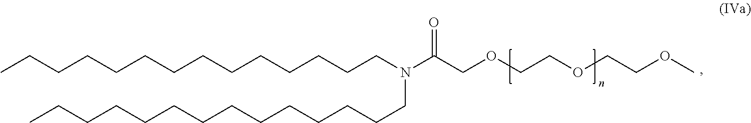

wherein n has a mean value ranging from 30 to 60, such as about 28 to about 32, about 30 to about 34, 32 to about 36, about 34 to about 38, 36 to about 40, about 38 to about 42, 40 to about 44, about 42 to about 46, 44 to about 48, about 46 to about 50, 48 to about 52, about 50 to about 54, 52 to about 56, about 54 to about 58, 56 to about 60, about 58 to about 62. In preferred embodiments, n is about 45, 46, 47, 48, 49, 50, 51, 52, 53, 54. In a most preferred embodiment n has a mean value of 49.

In other embodiments, the PEGylated lipid has one of the following structures:

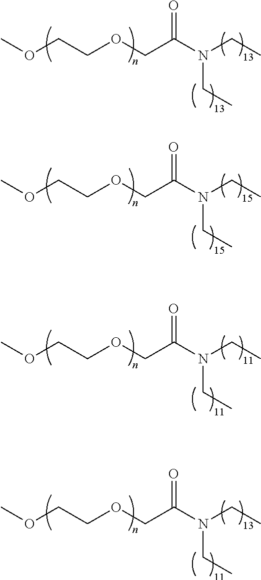

wherein n is an integer selected such that the average molecular weight of the PEGylated lipid is about 2500 g/mol, most preferably n is about 49.

Further examples of PEG-lipids suitable in that context are provided in US2015/0376115A1 and WO2015/199952, each of which is incorporated by reference in its entirety.

In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2.5%, about 2%, about 1.5%, about 1%, about 0.5%, or about 0.3% on a molar basis (based on 100% total moles of lipids in the LNP). In preferred embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the LNP). In various embodiments, the molar ratio of the cationic lipid to the PEGylated lipid ranges from about 100:1 to about 25:1.

In preferred embodiments, the LNP additionally comprises one or more additional lipids which stabilize the formation of particles during their formation (e.g. neutral lipid and/or one or more steroid or steroid analogue).

In preferred embodiments of the second aspect, the coding RNA of the first aspect is complexed with one or more lipids thereby forming lipid nanoparticles (LNP), wherein the LNP additionally comprises one or more neutral lipid and/or one or more steroid or steroid analogue.

Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

In embodiments of the second aspect, the LNP additionally comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In preferred embodiments of the second aspect, the neutral lipid is 1,2-distearoyl-sn-glycero-3-phosphocholine (DSPC). The molar ratio of the cationic lipid to DSPC may be in the range from about 2:1 to 8:1.

In preferred embodiments of the second aspect, the steroid is cholesterol. The molar ratio of the cationic lipid to cholesterol may be in the range from about 2:1 to 1:1.

In some embodiments, the cholesterol may be PEGylated.

The sterol can be about 10 mol % to about 60 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the LNPs include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle).

Preferably, lipid nanoparticles (LNPs) comprise: (a) at least one coding RNA of the first aspect, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In some embodiments, the cationic lipids (as defined above), non-cationic lipids (as defined above), cholesterol (as defined above), and/or PEG-modified lipids (as defined above) may be combined at various relative molar ratios. For example, the ratio of cationic lipid to non-cationic lipid to cholesterol-based lipid to PEGylated lipid may be between about 30-60:20-35:20-30:1-15, or at a ratio of about 40:30:25:5, 50:25:20:5, 50:27:20:3, 40:30:20:10, 40:32:20:8, 40:32:25:3 or 40:33:25:2, or at a ratio of about 50:25:20:5, 50:20:25:5, 50:27:20:3 40:30:20:10, 40:30:25:5 or 40:32:20:8, 40:32:25:3 or 40:33:25:2, respectively.

In some embodiments, the LNPs comprise a lipid of formula (111), at least one coding RNA as defined herein, a neutral lipid, a steroid and a PEGylated lipid. In preferred embodiments, the lipid of formula (111) is lipid compound 111-3, the neutral lipid is DSPC, the steroid is cholesterol, and the PEGylated lipid is the compound of formula (IVa).

In a preferred embodiment of the second aspect, the LNP consists essentially of (i) at least one cationic lipid; (ii) a neutral lipid; (iii) a sterol, e.g., cholesterol; and (iv) a PEG-lipid, e.g. PEG-DMG or PEG-cDMA, in a molar ratio of about 20-60% cationic lipid:5-25% neutral lipid:25-55% sterol; 0.5-15% PEG-lipid.

In particularly preferred embodiments of the second aspect, the coding RNA of the first aspect and is complexed with one or more lipids thereby forming lipid nanoparticles (LNP), wherein the LNP essentially consists of

(i) at least one cationic lipid as defined herein, preferably a lipid of formula (111), more preferably lipid 111-3;
(ii) a neutral lipid as defined herein, preferably 1,2-distearoyl-sn-glycero-3-phosphocholine (DSPC);
(iii) a steroid or steroid analogue as defined herein, preferably cholesterol; and
(iv) a PEG-lipid as defined herein, e.g. PEG-DMG or PEG-cDMA, preferably a PEGylated lipid of formula (IVa), wherein (i) to (iv) are in a molar ratio of about 20-60% cationic lipid: 5-25% neutral lipid: 25-55% sterol; 0.5-15% PEG-lipid.

In one preferred embodiment, the lipid nanoparticle comprises: a cationic lipid with formula (111) and/or PEG lipid with formula (IV), optionally a neutral lipid, preferably 1,2-distearoyl-sn-glycero-3-phosphocholine (DSPC) and optionally a steroid, preferably cholesterol, wherein the molar ratio of the cationic lipid to DSPC is optionally in the range from about 2:1 to 8:1, wherein the molar ratio of the cationic lipid to cholesterol is optionally in the range from about 2:1 to 1:1.

In a particular preferred embodiment, the composition of the second aspect comprising the coding RNA of the first aspect comprises lipid nanoparticles (LNPs, LNP-III-3), which have a molar ratio of approximately 50:10:38.5:1.5, preferably 47.5:10:40.8:1.7 or more preferably 47.4:10:40.9:1.7 (i.e. proportion (mol %) of cationic lipid (preferably lipid 111-3), DSPC, cholesterol and PEG-lipid ((preferably PEG-lipid of formula (IVa) with n=49); solubilized in ethanol).

The total amount of RNA in the lipid nanoparticles may vary and is defined depending on the e.g. RNA to total lipid w/w ratio. In one embodiment of the invention the coding RNA to total lipid ratio is less than 0.06 w/w, preferably between 0.03 w/w and 0.04 w/w.

In some embodiments, the composition of the second aspect comprising the RNA of the first aspect comprises lipid nanoparticles (LNPs), which are composed of only three lipid components, namely imidazole cholesterol ester (ICE), 1,2-dioleoyl-sn-glycero-3-phosphoethanolamine (DOPE), and 1,2-dimyristoyl-sn-glycerol, methoxypolyethylene glycol (DMG-PEG-2K)

In various embodiments, the LNP as defined herein have a mean diameter of from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm and are substantially non-toxic. As used herein, the mean diameter may be represented by the z-average as determined by dynamic light scattering as commonly known in the art.

The polydispersity index (PDI) of the nanoparticles is typically in the range of 0.1 to 0.5. In a particular embodiment, a PDI is below 0.2. Typically, the PDI is determined by dynamic light scattering.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In embodiments where more than one or a plurality, e.g. 2, 3, 4, 5, 6, 7, 8, 9, 10, 11, 12, 13, 14, 15 of the RNAs of the first aspect are comprised in the composition, said more than one or said plurality e.g. 2, 3, 4, 5, 6, 7, 8, 9, 10, 11, 12, 13, 14, 15 of the RNAs may be complexed within one or more lipids thereby forming LNPs comprising more than one or a plurality, e.g. 2, 3, 4, 5, 6, 7, 8, 9, 10, 11, 12, 13, 14, 15 of different RNAs.

In embodiments, the LNPs described herein may be lyophilized in order to improve storage stability of the formulation and/or the RNA. In embodiments, the LNPs described herein may be spray dried in order to improve storage stability of the formulation and/or RNA. Lyoprotectants for lyophilization and or spray driying may be selected from trehalose, sucrose, mannose, dextran and inulin.

In one embodiment, a lipid nanoparticle as used in the invention comprises a cationic lipid, a steroid; a neutral lipid; and a polymer conjugated lipid, preferably a pegylated lipid. Preferably, the polymer conjugated lipid is a pegylated lipid or PEG-lipid. In a specific embodiment, lipid nanoparticles comprise a cationic lipid resembled by the cationic lipid COATSOME® SS-EC (former name: SS-33/4PE-15; NOF Corporation, Tokyo, Japan), in accordance with the following formula

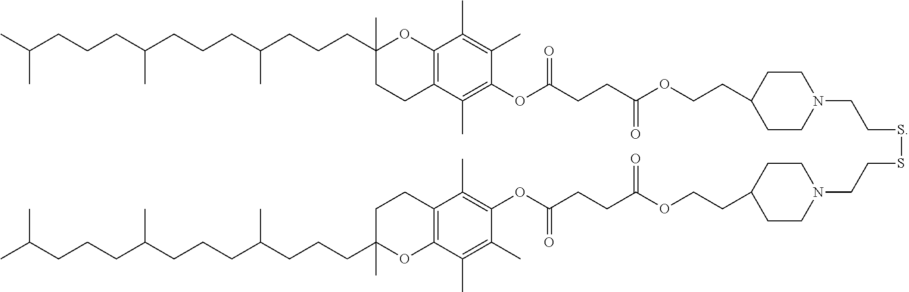 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

As described further below, those lipid nanoparticles are termed “GN01”.

Furthermore, in a specific embodiment, the GN01 lipid nanoparticles comprise a neutral lipid being resembled by the structure 1,2-diphytanoyl-sn-glycero-3-phosphoethanolamine (DPhyPE):

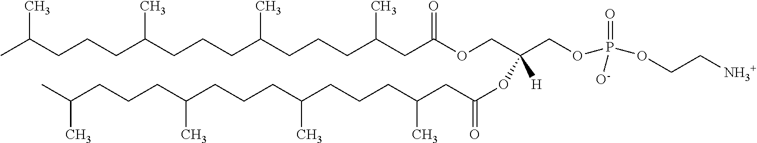 [SMILES: CC(C)CCCC(C)CCCC(C)CCCC(C)CC(=O)OC[C@H](COP(=O)([O-])OCC[NH3+])OC(=O)CC(C)CCCC(C)CCCC(C)CCCC(C)C]

Furthermore, in a specific embodiment, the GN01 lipid nanoparticles comprise a polymer conjugated lipid, preferably a pegylated lipid, being 1,2-dimyristoyl-rac-glycero-3-methoxypolyethylene glycol 2000 (DMG-PEG 2000) having the following structure:

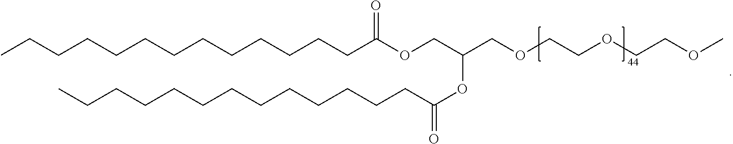

As used in the art, “DMG-PEG 2000” is considered a mixture of 1,2-DMG PEG2000 and 1,3-DMG PEG2000 in ˜97:3 ratio.

Accordingly, GN01 lipid nanoparticles (GN01-LNPs) according to one of the preferred embodiments comprise a SS-EC cationic lipid, neutral lipid DPhyPE, cholesterol, and the polymer conjugated lipid (pegylated lipid) 1,2-dimyristoyl-rac-glycero-3-methoxypolyethylene glycol (PEG-DMG).

In a preferred embodiment, the GN01 LNPs comprise:

(a) cationic lipid SS-EC (former name: SS-33/4PE-15; NOF Corporation, Tokyo, Japan) at an amount of 45-65 mol %;
(b) cholesterol at an amount of 25-45 mol %;
(c) DPhyPE at an amount of 8-12 mol %; and
(d) PEG-DMG 2000 at an amount of 1-3 mol %;
each amount being relative to the total molar amount of all lipidic excipients of the GN01 lipid nanoparticles.

In a further preferred embodiment, the GN01 lipid nanoparticles as described herein comprises 59 mol % cationic lipid, 10 mol % neutral lipid, 29.3 mol % steroid and 1.7 mol % polymer conjugated lipid, preferably pegylated lipid. In a most preferred embodiment, the GN01 lipid nanoparticles as described herein comprise 59 mol % cationic lipid SS-EC, 10 mol % DPhyPE, 29.3 mol % cholesterol and 1.7 mol % DMG-PEG 2000.

The amount of the cationic lipid relative to that of the mRNA compound in the GN01 lipid nanoparticle may also be expressed as a weight ratio (abbreviated f.e. “m/m”). For example, the GN01 lipid nanoparticles comprise the mRNA compound at an amount such as to achieve a lipid to mRNA weight ratio in the range of about 20 to about 60, or about 10 to about 50. In other embodiments, the ratio of cationic lipid to nucleic acid or mRNA is from about 3 to about 15, such as from about 5 to about 13, from about 4 to about 8 or from about 7 to about 11. In a very preferred embodiment of the present invention, the total lipid/mRNA mass ratio is about 40 or 40, i.e. about 40 or 40 times mass excess to ensure mRNA encapsulation. Another preferred RNA/lipid ratio is between about 1 and about 10, about 2 and about 5, about 2 and about 4, or preferably about 3.

Further, the amount of the cationic lipid may be selected taking the amount of the nucleic acid cargo such as the mRNA compound into account. In one embodiment, the N/P ratio can be in the range of about 1 to about 50. In another embodiment, the range is about 1 to about 20, about 1 to about 10, about 1 to about 5. In one preferred embodiment, these amounts are selected such as to result in an N/P ratio of the GN01 lipid nanoparticles or of the composition in the range from about 10 to about 20. In a further very preferred embodiment, the N/P is 14 (i.e. 14 times mol excess of positive charge to ensure mRNA encapsulation).

A particularly preferred embodiment for a GN01 lipid nanoparticle of the present invention is given when 59 mol % cationic lipid COATSOME® SS-EC (former name: SS-33/4PE-15 as apparent from the examples section; NOF Corporation, Tokyo, Japan), 29.3 mol % cholesterol as steroid, 10 mol % DPhyPE as neutral lipid/phospholipid and 1.7 mol % DMG-PEG 2000 as polymer conjugated lipid. Said LNP composition is called herein and in the working examples “GN01”. SS-EC has a positive charge at pH 4 and a neutral charge at pH 7, which is advantageous for the LNPs and formulations/compositions of the present invention. A further inventive advantage connected with the use of DPhyPE is the high capacity for fusogenicity due to its bulky tails, whereby it is able to fuse at a high level with endosomal lipids. For “GN01”, N/P (lipid to mRNA mol ratio) preferably is 14 and total lipid/mRNA mass ratio preferably is 40 (m/m).

### Adjuvants:

According to further embodiments, the composition of the second aspect may comprise at least one adjuvant. Suitably, the adjuvant is preferably added to enhance the immunostimulatory properties of the composition.

The term “adjuvant” as used herein will be recognized and understood by the person of ordinary skill in the art, and is for example intended to refer to a pharmacological and/or immunological agent that may modify, e.g. enhance, the effect of other agents (herein: the effect of the coding RNA) or that may be suitable to support administration and delivery of the composition. The term “adjuvant” refers to a broad spectrum of substances. Typically, these substances are able to increase the immunogenicity of antigens. For example, adjuvants may be recognized by the innate immune systems and, e.g., may elicit an innate immune response (that is, a non-specific immune response). “Adjuvants” typically do not elicit an adaptive immune response. In the context of the invention, adjuvants may enhance the effect of the antigenic peptide or protein provided by the coding RNA. In that context, the at least one adjuvant may be selected from any adjuvant known to a skilled person and suitable for the present case, i.e. supporting the induction of an immune response in a subject, e.g. in a human subject.

Accordingly, the composition of the second aspect may comprise at least one adjuvant, wherein the at least one adjuvant may be suitably selected from any adjuvant provided in WO2016/203025. Adjuvants disclosed in any of the claims 2 to 17 of WO2016/203025, preferably adjuvants disclosed in claim 17 of WO2016/203025 are particularly suitable, the specific content relating thereto herewith incorporated by reference.

The composition of the second aspect may comprise, besides the components specified herein, at least one further component which may be selected from the group consisting of further antigens (e.g. in the form of a peptide or protein) or further antigen-encoding nucleic acids; a further immunotherapeutic agent; one or more auxiliary substances (cytokines, such as monokines, lymphokines, interleukins or chemokines); or any further compound, which is known to be immune stimulating due to its binding affinity (as ligands) to human Toll-like receptors; and/or an adjuvant nucleic acid, preferably an immunostimulatory RNA (isRNA), e.g. CpG-RNA etc.

### Vaccine:

In a third aspect, the present invention provides a Malaria vaccine wherein the vaccine comprises the coding RNA of the first aspect, and, optionally, the composition of the second aspect.

Notably, embodiments relating to the composition of the second aspect may likewise be read on and be understood as suitable embodiments of the vaccine of the third aspect. Also, embodiments relating to the vaccine of the third aspect may likewise be read on and be understood as suitable embodiments of the composition of the second aspect (comprising the RNA of the first aspect).

The term “vaccine” will be recognized and understood by the person of ordinary skill in the art, and is for example intended to be a prophylactic or therapeutic material providing at least one epitope or antigen, preferably an immunogen. In the context of the invention the antigen or antigenic function is provided by the inventive coding RNA of the first aspect (said RNA comprising a coding sequence encoding a antigenic peptide or protein derived from CSP) or the composition of the second aspect (comprising the RNA of the first aspect).

In preferred embodiments of the third aspect, the vaccine comprising the RNA of the first aspect or the composition of the second aspect, elicits an adaptive immune response, preferably an adaptive immune response against a malaria parasite.

In preferred embodiments of the third aspect, the vaccine comprising the RNA of the first aspect or the composition of the second aspect induces strong humoral and cellular immune responses, preferably strong CD4+ and CD8+ T-cell responses.

According to a preferred embodiment of the third aspect, the vaccine as defined herein may further comprise a pharmaceutically acceptable carrier and optionally at least one adjuvant as specified in the context of the second aspect.

Suitable adjuvants in that context may be selected from adjuvants disclosed in claim 17 of WO2016/203025.

In a preferred embodiment, the vaccine is a monovalent vaccine.

In embodiments the vaccine is a polyvalent vaccine comprising a plurality or at least more than one of the coding RNA species as defined in the context of the first aspect. Embodiments relating to a polyvalent composition as disclosed in the context of the second aspect may likewise be read on and be understood as suitable embodiments of the polyvalent vaccine of the third aspect.

The vaccine of the third aspect typically comprises a safe and effective amount of the coding RNA of the first aspect. As used herein, “safe and effective amount” means an amount of the coding RNA that is sufficient to significantly induce a positive modification of a disease or disorder related to an infection with a malaria parasite. At the same time, a “safe and effective amount” is small enough to avoid serious side-effects. In relation to the vaccine or composition of the present invention, the expression “safe and effective amount” preferably means an amount of the coding RNA that is suitable for stimulating the adaptive immune system in such a manner that no excessive or damaging immune reactions are achieved but, preferably, also no such immune reactions below a measurable level.

A “safe and effective amount” of the coding RNA of the composition or vaccine as defined above will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying medical doctor. Moreover, the “safe and effective amount” of the coding RNA, the composition, the vaccine may depend from application route (intradermal, intramuscular), application device (jet injection, needle injection, microneedle patch) and/or complexation (protamine complexation or LNP encapsulation). Moreover, the “safe and effective amount” of the coding RNA, the composition, the vaccine may depend from the condition of the treated subject (infant, pregnant women, immunocompromised human subject etc.). Accordingly, the suitable “safe and effective amount” has to be adapted accordingly and will be chosen and defined by the skilled person.

In some embodiments, the “safe and effective amount” is a dose equivalent to an at least 2-fold, at least 4-fold, at least 10-fold, at least 100-fold, at least 1000-fold reduction in the standard of care dose of a recombinant Malaria protein vaccine, wherein the anti-antigenic polypeptide antibody titer produced in the subject is equivalent to an anti-antigenic polypeptide antibody titer produced in a control subject administered the standard of care dose of a recombinant Malaria protein vaccine, a purified Malaria protein vaccine, a live attenuated Malaria vaccine, an inactivated Malaria vaccine or a Malaria VLP vaccine. In some embodiments, the control is an anti-antigenic polypeptide antibody titer produced in a subject who has been administered a virus-like particle (VLP) vaccine comprising structural proteins of Malaria.

The vaccine can be used according to the invention for human medical purposes and also for veterinary medical purposes (mammals, vertebrates, avian species), as a pharmaceutical composition, or as a vaccine.

Accordingly, the pharmaceutically acceptable carrier as used herein preferably includes the liquid or non-liquid basis of the inventive vaccine. If the inventive vaccine is provided in liquid form, the carrier will be water, typically pyrogen-free water; isotonic saline or buffered (aqueous) solutions, e.g. phosphate, citrate etc. buffered solutions. Preferably, Ringer-Lactate solution is used as a liquid basis for the vaccine or the composition according to the invention as described in WO2006/122828, the disclosure relating to suitable buffered solutions incorporated herewith by reference.

The choice of a pharmaceutically acceptable carrier as defined herein is determined, in principle, by the manner, in which the pharmaceutical composition(s) or vaccine according to the invention is administered. The vaccine is preferably administered locally. Routes for local administration in general include, for example, topical administration routes but also intradermal, transdermal, subcutaneous, or intramuscular injections or intralesional, intracranial, intrapulmonal, intracardial, intraarticular and sublingual injections. More preferably, composition or vaccines according to the present invention may be administered by an intradermal, subcutaneous, or intramuscular route, preferably by injection, which may be needle-free and/or needle injection. Compositions/vaccines are therefore preferably formulated in liquid or solid form. The suitable amount of the vaccine or composition according to the invention to be administered can be determined by routine experiments, e.g. by using animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices.

The inventive vaccine or composition as defined herein can additionally comprise one or more auxiliary substances as defined above in order to further increase the immunogenicity. A synergistic action of the coding RNA contained in the inventive composition/vaccine and of an auxiliary substance, which may be optionally be co-formulated (or separately formulated) with the inventive vaccine or composition as described above, is preferably achieved thereby. Such immunogenicity increasing agents or compounds may be provided separately (not co-formulated with the inventive vaccine or composition) and administered individually.

Further additives which may be included in the inventive vaccine or composition are emulsifiers, such as for example, Tween; wetting agents, such as, for example, sodium lauryl sulfate; colouring agents; taste-imparting agents, pharmaceutical carriers; tablet-forming agents; stabilizers; antioxidants; preservatives.

### Kit or Kit of Parts, Application, Medical Uses, Method of Treatment:

In a fourth aspect, the present invention provides a kit or kit of parts, wherein the kit or kit of parts comprises the coding RNA of the first aspect, the composition of the second aspect (comprising said RNA), and/or the vaccine of the third aspect. In addition, the kit or kit of parts of the fourth aspect may comprise a liquid vehicle for solubilising, and/or technical instructions providing information on administration and dosage of the components.

The kit may further comprise additional components as described in the context of the composition of the second aspect, and/or the vaccine of the third aspect.

The technical instructions of said kit may contain information about administration and dosage and patient groups. Such kits, preferably kits of parts, may be applied e.g. for any of the applications or uses mentioned herein, preferably for the use of the coding RNA of the first aspect, the composition of the second aspect, or the vaccine of the third aspect, for the treatment or prophylaxis of an infection or diseases caused by a Malaria parasite or disorders related thereto. Preferably, the coding RNA of the first aspect, the composition of the second aspect, or the vaccine of the third aspect is provided in a separate part of the kit, wherein the coding RNA of the first aspect, the composition of the second aspect, or the vaccine of the third aspect is preferably lyophilised. The kit may further contain as a part a vehicle (e.g. buffer solution) for solubilising the coding RNA of the first aspect, the composition of the second aspect, or the vaccine of the fifth aspect.

In preferred embodiments, the kit or kit of parts as defined herein comprises Ringer lactate solution.

Any of the above kits may be used in a treatment or prophylaxis as defined herein. More preferably, any of the above kits may be used as a vaccine, preferably a vaccine against infections caused by a Malaria parasite as defined herein.

### Medical Use:

In a further aspect, the present invention relates to the first medical use of the coding RNA of the first aspect, the composition of the second aspect, the vaccine of the third aspect, or the kit or kit of parts of the fourth aspect.

Accordingly, the RNA of the first aspect, the composition of the second aspect, the vaccine of the third aspect, or the kit or kit of parts of the fourth aspect is for use as a medicament.

The present invention furthermore provides several applications and uses of the coding RNA of the first aspect, the composition of the second aspect, the vaccine of the third aspect, or the kit or kit of parts of the fourth aspect.

In particular, said RNA, composition, vaccine, or the kit or kit of parts may be used for human medical purposes and also for veterinary medical purposes, preferably for human medical purposes.

In particular, said RNA, composition, vaccine, or the kit or kit of parts is for use as a medicament for human medical purposes, wherein said RNA, composition, vaccine, or the kit or kit of parts may be particularly suitable for young infants, newborns, immunocompromised recipients, as well as pregnant and breast-feeding women and elderly people.

In yet another aspect, the present invention relates to the second medical use of the coding RNA of the first aspect, the composition of the second aspect, the vaccine of the third aspect, or the kit or kit of parts of the fourth aspect.

In embodiments, the RNA of the first aspect, the composition of the second aspect, the vaccine of the third aspect, or the kit or kit of parts of the fourth aspect is for use in the treatment or prophylaxis of an infection with a pathogen (e.g. a protozoan parasite), in particular with a Malaria parasite, or a disorder related to such an infection.

In embodiments, the RNA of the first aspect, the composition of the second aspect, the vaccine of the third aspect, or the kit or kit of parts of the fourth aspect is for use in the treatment or prophylaxis of an infection with a Malaria parasite, in particular with Plasmodium falciparum (Pf), Plasmodium knowlesi (Pk), Plasmodium ovale (Po), Plasmodium simiovale (Ps), and Plasmodium vivax (Pv), Plasmodium malariae (Pm), Plasmodium ovale curtisi (Poc), Plasmodium ovale wallikeri (Pow), or Plasmodium berghei (Pb).

In preferred embodiments, the RNA of the first aspect, the composition of the second aspect, the vaccine of the third aspect, or the kit or kit of parts of the fourth aspect is for use in the treatment or prophylaxis of an infection with Plasmodium falciparum (Pf).

In particular, the RNA of the first aspect, the composition of the second aspect, the vaccine of the third aspect, or the kit or kit of parts of the fourth aspect may be used in the treatment or prophylaxis of an infection with a Malaria parasite, in particular with Plasmodium falciparum (Pf), Plasmodium knowlesi (Pk), Plasmodium ovale (Po), Plasmodium simiovale (Ps), and Plasmodium vivax (Pv), Plasmodium malariae (Pm), Plasmodium ovale curtisi (Poc), Plasmodium ovale wallikeri (Pow), or Plasmodium berghei (Pb), or a disorder related to such an infection, for human and also for veterinary medical purposes, preferably for human medical purposes.

As used herein, “a disorder related to a Malaria infection” may preferably comprise a typical symptom or a complication of a Malaria infection.

Particularly, the coding RNA of the first aspect, the composition of the second aspect, the vaccine of the third aspect, or the kit or kit of parts of the fourth aspect may be used in a method of prophylactic (pre-exposure prophylaxis or post-exposure prophylaxis) and/or therapeutic treatment of infections caused by a Malaria parasite.

The composition or the vaccine as defined herein may preferably be administered locally. In particular, composition or vaccines may be administered by an intradermal, subcutaneous, intranasal, or intramuscular route. Inventive compositions or vaccines of the invention are therefore preferably formulated in liquid (or sometimes in solid) form. In embodiments, the inventive vaccine may be administered by conventional needle injection or needle-free jet injection. Preferred in that context is the RNA, the composition, the vaccine is administered by intramuscular needle injection.

The term “jet injection”, as used herein, refers to a needle-free injection method, wherein a fluid (vaccine, composition of the invention) containing e.g. at least one RNA of the first aspect is forced through an orifice, thus generating an ultra-fine liquid stream of high pressure that is capable of penetrating mammalian skin and, depending on the injection settings, subcutaneous tissue or muscle tissue. In principle, the liquid stream perforates the skin, through which the liquid stream is pushed into the target tissue. Preferably, jet injection is used for intradermal, subcutaneous or intramuscular injection of the RNA, the compositions, the vaccines disclosed herein.

In embodiments, the RNA as comprised in a composition or vaccine as defined herein is provided in an amount of about 100 ng to about 500 ug, in an amount of about 1 ug to about 200 ug, in an amount of about 1 ug to about 100 ug, in an amount of about 5 ug to about 100 ug, preferably in an amount of about bug to about 50 ug, specifically, in an amount of about 5 ug, 10ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug or 100 ug.

In some embodiments, vaccine comprising the coding RNA is formulated in an effective amount to produce an antigen specific immune response in a subject. In some embodiments, the effective amount is a total dose of 1 ug to 200 ug, 1 ug to 100 ug, or 5 ug to 100 ug.

In some embodiments, the subject is about 5 years old or younger. For example, the subject may be between the ages of about 1 year and about 5 years (e.g., about 1, 2, 3, 4 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject is an adult between the ages of about 20 years and about 50 years (e.g., about 20, 25, 30, 35, 40, 45 or 50 years old). In some embodiments, the subject is an elderly subject about 60 years old, about 70 years old, or older (e.g., about 60, 65, 70, 75, 80, 85 or 90 years old).

In some embodiments, the subject has been exposed to Malaria

Depending from application route (intradermal, intramuscular, intranasal), application device (jet injection, needle injection, microneedle patch) and/or complexation (preferably LNP encapsulation) and the patient group, the suitable amount has to be adapted accordingly and will be chosen and defined by the skilled person.

In one embodiment, the immunization protocol for the treatment or prophylaxis of a subject against Malaria comprises one single doses of the composition or the vaccine.

In some embodiments, the effective amount is a dose of 5 ug administered to the subject in one vaccination. In some embodiments, the effective amount is a dose of bug administered to the subject in one vaccination. In some embodiments, the effective amount is a dose of 20 ug administered to the subject in one vaccination. In some embodiments, the effective amount is a dose of 30 ug administered to the subject in one vaccination. In some embodiments, the effective amount is a dose of 40 ug administered to the subject in one vaccination. In some embodiments, the effective amount is a dose of 50 ug administered to the subject in one vaccination. In some embodiments, the effective amount is a dose of 100 ug administered to the subject in one vaccination. In some embodiments, the effective amount is a dose of 200 ug administered to the subject in one vaccination.

In preferred embodiments, the immunization protocol for the treatment or prophylaxis of an infection as defined herein, i.e. the immunization of a subject against Malaria, typically comprises a series of single doses or dosages of the composition or the vaccine. A single dosage, as used herein, refers to the initial/first dose, a second dose or any further doses, respectively, which are preferably administered in order to “boost” the immune reaction.

In preferred embodiments, the immunization protocol for the treatment or prophylaxis of an infection as defined herein, i.e. the immunization of a subject against Malaria, comprises a prolonged injection interval between the first (prime) immunization and the second (boost) immunization.

The inventors could show that a prolonged interval between prime and boost vaccination may lead to increased humoral immune responses (see e.g. Example 12).

In further preferred embodiments, the immunization protocol for the treatment or prophylaxis of an infection as defined herein, comprises a prolonged injection interval between the first (prime) immunization at day 0 and the second (boost) immunization at day 56.

In some embodiments, the effective amount is a dose of 5 ug administered to the subject a total of two times. In some embodiments, the effective amount is a dose of 10 ug administered to the subject a total of two times. In some embodiments, the effective amount is a dose of 20 ug administered to the subject a total of two times. In some embodiments, the effective amount is a dose of 30 ug administered to the subject a total of two times. In some embodiments, the effective amount is a dose of 40 ug administered to the subject a total of two times. In some embodiments, the effective amount is a dose of 50 ug administered to the subject a total of two times. In some embodiments, the effective amount is a dose of 100 ug administered to the subject a total of two times. In some embodiments, the effective amount is a dose of 200 ug administered to the subject a total of two times.

In preferred embodiments, the vaccine/composition immunizes the subject against Malaria (e.g., P. falciparum, P. vivax, P. malariae and/or P. ovale) for more than 2 years, more than 3 years, more than 4 years, or for 5-10 years.

### Method of Treatment and Use, Diagnostic Method and Use:

In another aspect, the present invention relates to a method of treating or preventing a disorder.

In preferred embodiments, the present invention relates to a method of treating or preventing a disorder, wherein the method comprises applying or administering to a subject in need thereof the RNA of the first aspect, the composition of the second aspect, the vaccine of the third aspect, or the kit or kit of parts of the fourth aspect.

In preferred embodiments, the disorder is an infection with a Malaria parasite, in particular with Plasmodium falciparum (Pf), Plasmodium knowlesi (Pk), Plasmodium ovale (Po), Plasmodium simiovale (Ps), and Plasmodium vivax (Pv), Plasmodium malariae (Pm), Plasmodium ovale curtisi (Poc), Plasmodium ovale wallikeri (Pow), or Plasmodium berghei (Pb), or a disorder related to such infections.

In preferred embodiments, the present invention relates to a method of treating or preventing a disorder as defined above, wherein the method comprises applying or administering to a subject in need thereof the coding RNA of the first aspect, the composition of the second aspect, the vaccine of the third aspect, or the kit or kit of parts of the fourth aspect, wherein the subject in need is preferably a mammalian subject. In particularly preferred embodiments, the mammalian subject is a human subject, particularly an infant, a newborn, a pregnant women, a breast-feeding woman, an elderly, or an immunocompromised human subject.

In particular, such a method may preferably comprise the steps of:
a) providing the coding RNA of the first aspect, the composition of the second aspect, the vaccine of the third aspect, or the kit or kit of parts of the fourth aspect;
b) applying or administering said RNA of the first aspect, composition of the second aspect, vaccine of the third aspect, or kit or kit of parts of the fourth aspect to a tissue or an organism;
c) optionally, administering immunoglobulin (IgGs) against a Malaria parasite;
d) optionally, administering a further substance (adjuvant, auxiliary substance, further antigen, vaccine).

According to a further aspect, the present invention also provides a method for expression of at least one polypeptide comprising at least one peptide or protein derived from a Malaria parasite, or a fragment or variant thereof, wherein the method preferably comprises the following steps:
a) providing the coding RNA of the first aspect or the composition of the second aspect; and
b) applying or administering said coding RNA or composition to an expression system (cells), a tissue, an organism.

The method may be applied for laboratory, for research, for diagnostic, for commercial production of peptides or proteins and/or for therapeutic purposes. The method may furthermore be carried out in the context of the treatment of a specific disease, particularly in the treatment of infectious diseases, particularly Malaria infections.

Likewise, according to another aspect, the present invention also provides the use of the coding RNA of the first aspect, the composition of the second aspect, the vaccine of the third aspect, or the kit or kit of parts of the fourth aspect preferably for diagnostic or therapeutic purposes, e.g. for expression of an encoded Malaria antigenic peptide or protein, e.g. by applying or administering said coding RNA, composition comprising said coding RNA, vaccine comprising said coding RNA, e.g. to a cell-free expression system, a cell (e.g. an expression host cell or a somatic cell), a tissue or an organism. In specific embodiments, applying or administering said coding RNA, composition comprising said coding RNA, vaccine comprising said coding

RNA to a tissue or an organism is followed by e.g. a step of obtaining induced Malaria antibodies e.g. Malaria specific (monoclonal) antibodies.

The use may be applied for a (diagnostic) laboratory, for research, for diagnostics, for commercial production of peptides, proteins, or Malaria antibodies and/or for therapeutic purposes. The use may be carried out in vitro, in vivo or ex vivo. The use may furthermore be carried out in the context of the treatment of a specific disease, particularly in the treatment of a Malaria infection or a related disorder.

### List of Preferred Embodiments/Items

In the following, particularly preferred embodiments (items 1-58) of the invention are provided.

### Items

1. A coding RNA for a vaccine comprising
   a) at least one heterologous 5′ untranslated region (5′-UTR) and/or at least one heterologous 3′ untranslated region (3′-UTR); and
   b) at least one coding sequence operably linked to said 3′-UTR and/or 5′-UTR encoding at least one antigenic protein derived from circumsporozoite protein (CSP) of a Malaria parasite, or an immunogenic fragment or immunogenic variant thereof:
2. Coding RNA of items 1, wherein the Malaria parasite is selected from Plasmodium falciparum (Pf), Plasmodium knowlesi (Pk), Plasmodium ovale (Po), Plasmodium simiovale (Ps), or Plasmodium vivax (Pv).
3. Coding RNA of items 1 or 2, wherein the Malaria parasite is Plasmodium falciparum (Pf), preferably Plasmodium falciparum 3D7.
4. Coding RNA of any one of items 1 to 3, wherein the coding sequence encodes at least one antigenic protein from CSP of a Malaria parasite being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-36, 2081-2120, 2481-2886, 8742-8753, 10080-10085, or of an immunogenic fragment or immunogenic variant thereof.
5. Coding RNA of any one of items 1 to 4, wherein the coding sequence encodes at least a more full-length CSP or an immunogenic fragment or immunogenic variant thereof.
6. Coding RNA of any one of items 1 to 5, wherein the coding sequence additionally encodes at least one heterologous peptide or protein element selected from a heterologous signal peptide, a linker, a helper epitope, an antigen clustering domain, or a transmembrane domain.
7. Coding RNA of item 6, wherein the heterologous signal peptide is derived from SPARC according to SEQ ID NO: 6208, Hslns-iso1 according to SEQ ID NO: 6207, HsALB according to SEQ ID NO: 6205, or IgE according to SEQ ID NO: 6206, or fragment or variant of any of these, wherein HsALB is particularly preferred.
8. Coding RNA of item 6, wherein the linker element is element (L) is selected from SEQ ID NOs: 6241-6244, 10141, 10147.
9. Coding RNA of item 8, wherein the at least one linker element combines at least CSP-derived T-cell epitope preferably selected from sequences according to SEQ ID NOs: 2100, 2101, 2102, 2113, 10083, 10084 with a fragment of CSP and/or wherein the fragment of CSP is preferably combined with a C-terminus according to _Linker(AAY)_Pf-CSP(310-327)_Linker(AAY)_Pf-CSP(346-375), Linker(AAY)_Pf-CSP(346-365)_Linker(AAY)_PADRE, _Linker(AAY)_Pf-CS P(310-327)_Linker(AAY)_Pf-CSP(346-375), _Linker(G4S)_Pf-CSP(310-327)_Pf-CSP(346-375), _Linker(G4S)_Pf-CSP(310-327)_Linker(G4S)_Pf-CSP(346-375).
10. Coding RNA of item 6, wherein the helper epitope is derived from P2 helper peptide according to SEQ ID NO: 6272, PADRE helper epitope according to SEQ ID NO: 6273, HBsAg according to SEQ ID NO: 6274, or fragment or variant of any of these.
11. Coding RNA of item 6, wherein the antigen clustering domain is derived from from ferritin according to SEQ ID NO: 10162, lumazine-synthase (LS) according to SEQ ID NO: 10153, surface antigen of hepatitis B virus (HBsAg) according to SEQ ID NO: 6274, or fragment or variant of any of these.
12. Coding RNA of item 6, wherein the transmembrane domain is derived from a transmembrane domain of HA according to SEQ ID NOs: 6302, or fragment or variant thereof.
13. Coding RNA of any one of the preceding items, wherein the at least one antigenic protein comprises, preferably in N-terminal to C-terminal direction:
   a) optionally, one heterologous secretory signal sequence selected from SEQ ID NOs: 6205-6208;
   b) at least one protein derived from CSP of a Malaria parasite, or fragments or variants thereof;
   c) optionally, at least one heterologous helper epitope selected from SEQ ID NOs: 6272, 6273, or 6274or fragments or variants thereof;
   d) optionally, at least one heterologous antigen clustering domain selected from SEQ ID NOs: 6274, 10153, 10162, or fragments or variants thereof, and
   e) optionally, at least one heterologous transmembrane domain selected from SEQ ID NOs: 6302 or fragments or variants thereof,
   wherein a), b), c), d) and/or e) may be connected preferably via at least one peptide linker element selected from SEQ ID NOs: 6241-6244, 10141, 10147.
14. Coding RNA of any one of the preceding items, wherein the at least one antigenic protein derived from circumsporozoite protein (CSP) of a Malaria parasite, or an immunogenic fragment or immunogenic variant thereof, is mutated to delete at least one predicted or potential glycosylation site.
15. Coding RNA of any one of the preceding items, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-36, 2081-2120, 2481-2886, 8742-8753, 10080, or an immunogenic fragment or immunogenic variant of any of these.
16. Coding RNA of any one of the preceding items, wherein the at least one coding sequence comprises at least one nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 37-328, 2121-2480, 2887-6134, 8754-8855, 10086-10139 or a fragment or variant of any of these sequences.
17. Coding RNA of any one of the preceding items, wherein the least one coding sequence comprises at least one chemical modification or at least one modified nucleotide, preferably selected from pseudouridine (ψ), N1-methylpseudouridine (m1ψ), 5-methylcytosine, and 5-methoxyuridine.
18. Coding RNA of any one of the preceding items, wherein the at least one coding sequence is a codon modified coding sequence, wherein the amino acid sequence encoded by the at least one codon modified coding sequence is preferably not being modified compared to the amino acid sequence encoded by the corresponding wild type coding sequence.
19. Coding RNA according to item 18, wherein the at least one codon modified coding sequence is selected from C maximized coding sequence, CAI maximized coding sequence, human codon usage adapted coding sequence, G/C content modified coding sequence, and G/C optimized coding sequence, or any combination thereof.
20. Coding RNA of items 18 or 19, wherein the at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 41-328, 2161-2480, 3293-6134, 8754-8855, 10092-10139 or a fragment or variant of any of these sequences.
21. Coding RNA of any one of items 18 to 20, wherein the at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 41-328, 8754-8855 or a fragment or variant of any of these sequences.
22. Coding RNA of any one of items 18 to 121, wherein the at least one coding sequence comprises a G/C optimized coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 41-112, 2161-2240, 3293-3698, 8754-8783, 10092-10103 or a fragment or variant of any of these sequences.
23. Coding RNA of any one of the preceding items, wherein the coding RNA is an mRNA, a self-replicating RNA, a circular RNA, or a replicon RNA.
24. Coding RNA of any one of the preceding items, wherein the coding RNA is an mRNA.
25. Coding RNA of any one of the preceding items, wherein the coding RNA comprises a 5′-cap structure, preferably m7G, cap0, cap1, cap2, a modified cap0 or a modified cap1 structure, wherein cap1 is preferred.
26. Coding RNA of any one of the preceding items, wherein the RNA comprises at least one poly(A) sequence, preferably comprising 30 to 150 adenosine nucleotides and/or at least one poly(C) sequence, preferably comprising 10 to 40 cytosine nucleotides, wherein a poly(A) sequences with about 64 adenosine nucleotides (A64) or about 100 adenosine nucleotides (A100) are preferred.
27. Coding RNA of any one of the preceding items, wherein the RNA comprises at least one poly(A) sequence located (exactly) at the 3′ terminus of the coding RNA.
28. Coding RNA of any one of the preceding items, wherein the RNA comprises at least one histone stem-loop, wherein the histone stem-loop preferably comprises a nucleic acid sequence according to SEQ ID NOs: 6173 or 6174 or a fragment or variant thereof.
29. Coding RNA of any one of the preceding items, wherein the at least one heterologous 3′-UTR comprises a nucleic acid sequence derived from a 3′-UTR of a gene selected from PSMB3, ALB7, alpha-globin, CASP1, COX6B1, GNAS, NDUFA1 and RPS9, or from a homolog, a fragment or a variant of any one of these genes.
30. Coding RNA of any one of the preceding items, wherein the at least one heterologous 5′-UTR comprises a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.
31. Coding RNA of any one of the preceding items, comprising
   a-1. at least one 5′-UTR derived from a 5′-UTR of a HSD17B4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
   a-3. at least one 5′-UTR derived from a 5′-UTR of a SLC7A3 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
   i-2. at least one 5′-UTR derived from a 5′-UTR of a RPL32 gene, or from a corresponding RNA sequence, homolog, fragment or variant thereof and at least one 3′-UTR derived from a 3′-UTR of a ALB7 gene, or from a corresponding RNA sequence, homolog, fragment or variant thereof; or
   i-3. at least one 3′-UTR derived from a 3′-UTR of a alpha-globin gene gene, or from a corresponding RNA sequence, homolog, fragment or variant thereof.
32. Coding RNA of any one of the preceding items, wherein the coding RNA comprises the following elements preferably in 5′- to 3′-direction:
   A) 5′-cap structure selected from m7G(5′), m7G(5′)ppp(5′)(2′OMeA), or m7G(5′)ppp(5′)(2′OMeG);
   B) 5′-terminal start element selected from SEQ ID NOs: 6177 or 6178 or fragments or variants thereof;
   C) optionally, a cleavage site for a catalytic nucleic acid molecule, preferably as specified herein;
   D) optionally, 5′-UTR selected from SEQ ID NOs: 6135-6156 or fragments or variants thereof;
   F) a ribosome binding site selected from SEQ ID NOs: 6175, 6176 or fragments or variants thereof;
   E) at least one coding sequence selected from SEQ ID NOs: 37-328, 2121-2480, 2887-6134, 8754-8855, 10086-10139 or fragments or variants thereof;
   F) 3′-UTR selected from SEQ ID NOs: 6157 to 6172;
   G) optionally, poly(A) sequence comprising about 50 to about 500 adenosines;
   H) optionally, poly(C) sequence comprising about 10 to about 100 cytosines;
   I) optionally, histone stem-loop selected from SEQ ID NOs: 6173 or 6174;
   J) optionally, 3″-terminal sequence element SEQ ID NOs: 6179-6200, 10173-10196.
33. Coding RNA of any one of the preceding items, wherein the coding RNA comprises the following elements preferably in 5′- to 3′-direction:
   A) 5′-cap structure selected from m7G(5′), m7G(5′)ppp(5′)(2′OMeA), or m7G(5′)ppp(5′)(2′OMeG);
   B) 5′-terminal start element selected from SEQ ID NOs: 6177 or 6178 or fragments or variants thereof;
   C) 3′-UTR and/or 5′-UTR element according to a-1, a-2, a-3, a-4, a-5, b-1, b-2, b-3, b-4, b-5, c-1, c-2, c-3, c-4, c-5, d-1, d-2, d-3, d-4, d-5, e-1, e-2, e-3, e-4, e-5, e-6, f-1, f-2, f-3, f-4, f-5, g-1, g- 2, g-3, g-4, g-5, h-1, h-2, h-3, h-4, h-5, i-1, i-2, or i-3, as specified herein, wherein a-1, a-3, i-2, i-3 are preferred;
   D) a ribosome binding site selected from SEQ ID NOs: 6175, 6176 or fragments or variants thereof;
   E) at least one coding sequence selected from SEQ ID NOs: 37-328, 8754-8855 or fragments or variants thereof;
   G) poly(A) sequence comprising about 50 to about 500 adenosines, preferably about 64 or 100 adenosines;
   H) optionally, poly(C) sequence comprising about 10 to about 100 cytosines, preferably about 30 cytosines;
   I) optionally, histone stem-loop selected from SEQ ID NOs: 6173 or 6174.
34. Coding RNA of any one of the preceding items, wherein the coding RNA comprises the following elements preferably in 5′- to 3′-direction:
   A) 5′-cap structure selected from m7G(5′), m7G(5′)ppp(5′)(2′OMeA), or m7G(5′)ppp(5′)(2′OMeG);
   B) 5′-terminal start element selected from SEQ ID NOs: 6177 or 6178 or fragments or variants thereof;
   C) 3′-UTR and/or 5′-UTR element according to a-1, a-3, i-2, i-3;
   D) a ribosome binding site selected from SEQ ID NOs: 6175, 6176 or fragments or variants thereof; E) at least one coding sequence selected from SEQ ID NOs: 44, 80, 116, 152, 188, 224, 260, 296, 8755 (HsALB_Pf-CSP(19-397)) or fragments or variants thereof;
   G) poly(A) sequence comprising about 50 to about 500 adenosines, preferably about 64 or 100 adenosines;
   H) optionally, poly(C) sequence comprising about 10 to about 100 cytosines, preferably about 30 cytosines;
   I) optionally, histone stem-loop selected from SEQ ID NOs: 6173 or 6174.
35. Coding RNA of any one of the preceding items, wherein the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 329-2080, 6312-8741, 8856-10079, or a fragment or variant of any of these sequences.
36. A composition comprising at least one coding RNA as defined in any one of items 1 to 35, wherein the composition optionally comprises at least one pharmaceutically acceptable carrier.
37. Composition of item 36, wherein the at least one coding RNA is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.
38. Composition of item 37, wherein the at least one coding RNA is complexed or associated with one or more lipids, thereby forming liposomes, lipid nanoparticles, lipoplexes, and/or nanoliposomes.
39. Composition of item 38, wherein the at least one coding RNA is complexed with one or more lipids thereby forming lipid nanoparticles (LNP).
40. Composition of item 39, wherein the LNP essentially consists of
   (i) at least one cationic lipid;
   (ii) at least one neutral lipid;
   (iii) at least one steroid or steroid analogue; and
   (iv) at least one a PEG-lipid,
   wherein (i) to (iv) are in a molar ratio of about 20-60% cationic lipid, 5-25% neutral lipid, 25-55% sterol, and 0.5-15% PEG-lipid.
41. Composition of item 40, wherein the LNP comprises a cationic lipid according to formula III-3:

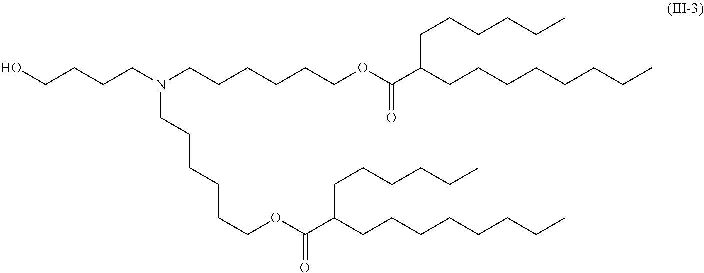 [SMILES: CCCCCCCCC(CCCCCC)C(=O)OCCCCCCN(CCCCO)CCCCCCOC(=O)C(CCCCCC)CCCCCCCC]

42. Composition of any one of items 40 to 41, wherein the LNP comprises a PEG lipid, wherein the PEG-lipid is of formula (IVa):

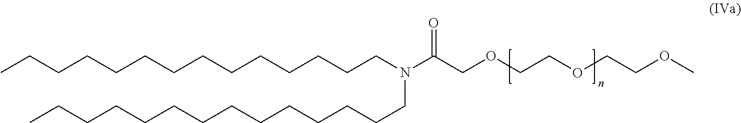

wherein n has a mean value ranging from 30 to 60, preferably wherein n has a mean value of about 45, 46, 47, 48, 49, 50, 51, 52, 53, 54, most preferably wherein n has a mean value of 49.
44. Composition of any one of items 40 to 43, wherein the LNP comprises one or more neutral lipids and/or one or more steroid or steroid analogues.
45. Composition of item 44, wherein the neutral lipid is 1,2-distearoyl-sn-glycero-3-phosphocholine (DSPC), preferably wherein the molar ratio of the cationic lipid to DSPC is in the range from about 2:1 to about 8:1.
46. Composition of item 44, wherein the steroid is cholesterol, preferably wherein the molar ratio of the cationic lipid to cholesterol is in the range from about 2:1 to about 1:1
47. Composition of item 40, wherein the LNP comprises COATSOME® SS-EC.
48. Composition of any one of items 40 and 47, wherein the LNP comprises a PEG lipid, wherein the PEG-lipid is DMG-PEG 2000.
49. Composition of any one of items 40 and 47 to 48, wherein the LNP further comprises 1,2-diphytanoyl-sn-glycero-3-phosphoethanolamine (DPhyPE) and cholesterol.
50. Composition of items 40 to 49, wherein the LNPs are preferably selected from LNP-GN01 or LNP-III-3.
51. A vaccine comprising the coding RNA as defined in any one of items 1 to 35, or the composition as defined in any one of items 36 to 50.
52. Vaccine of item 51, wherein the vaccine elicits an adaptive immune response.
53. A Kit or kit of parts, comprising the coding RNA as defined in any one of items 1 to 35, the composition as defined in any one of items 36 to 50, and/or the vaccine as defined in any one of items 51 to 52, optionally comprising a liquid vehicle for solubilising, and, optionally, technical instructions providing information on administration and dosage of the components.
54. Coding RNA as defined in any one of items 1 to 35, the composition as defined in any one of items 36 to 50, the vaccine as defined in any one of items 51 to 52, or the kit or kit of parts as defined in item 53, for use as a medicament.
55. Coding RNA as defined in any one of items 1 to 35, the composition as defined in any one of items 36 to 50, the vaccine as defined in any one of items 51 to 52, or the kit or kit of parts as defined in item 53, for use in the treatment or prophylaxis of Malaria, or of a disorder related to such an infection.
56. A method of treating or preventing a disorder, wherein the method comprises applying or administering to a subject in need thereof the coding RNA as defined in any one of items 1 to 35, the composition as defined in any one of items 36 to 50, the vaccine as defined in any one of items 51 to 52, or the kit or kit of parts as defined in items 53.
57. Method of item 56, wherein the disorder is an infection with Malaria, or a disorder related to such an infection.
58. Method of items 56 to 57, wherein the subject in need is a mammalian subject, preferably a human subject.

### Brief Description of Lists and Tables

List 1: Malaria parasites/Plasmodium species and subspecies with respective NCBI Taxonomy IDs
List 2: NCBI Protein Accession numbers of suitable Malaria antigens
Table 1: Preferred CSP antigen designs
Table 2: Human codon usage table with frequencies indicated for each amino acid
Table A: CSP antigens and respective coding sequences
Table 3: CSP fragments and respective coding sequences
Table 4: Heterologous elements and respective coding sequences
Table 5: Preferred coding sequences of the invention
Table 6A: Preferred mRNA constructs encoding CSP
Table 6B: Preferred mRNA constructs encoding CSP
Table 7: Lipidoids suitable for the invention
Table 8: Representative lipid compounds derived from formula (III)
Table 9: mRNA constructs encoding CPS used in the present examples (see Examples section)
Table B: Overview of lipid nanoparticle composition of GN01-LNPs formulation
Table 10: Vaccination scheme of Example 2 (see Examples section)
Table C: CSP peptide mix for ICS
Table 11: Vaccination scheme of Example 3 (see Examples section)
Table 12: RNA constructs used for western blot analysis of Example 4 (see Examples section)
Table 13A: Vaccination scheme of Example 6
Table 13B: Vaccination scheme of Example 7
Table 14: Vaccination scheme of Example 8
Table 15: Vaccination scheme of Example 9
Table 16: Vaccination scheme of Example 10
Table 17: Vaccination scheme of Example 11
Table 18: Vaccination scheme of Example 12
Table 19: Vaccination scheme of Example 13
Table 20: RNA constructs used for western blot analysis
Table 21: Overview of mRNA constructs used in Example 15
Table 22: Overview of passive transfer analysis according Example 16.1.
Table 23: Vaccination scheme of Example 16.2.
Table 24: Example of Animal groups and vaccination schedule of Example 17

### BRIEF DESCRIPTION OF THE DRAWINGS

FIG. 1 shows that formulated mRNA encoding CSP induces humoral immune responses in mice, using an ELISA assay. FIG. 1A: IgG1 and IgG2a endpoint titers at day 21 post vaccination; FIG. 1B: IgG1 and IgG2a endpoint titers at day 42 post vaccination; FIG. 1C: IgG1 and IgG2a endpoint titers at 56 days post vaccination. Groups 1 and 2: LNP formulated vaccine; Group 3: NaCl 0.9%; Group 4: Protamine formulated vaccine. Vaccination scheme see Table 10. Further details provided in Example 2.

FIG. 2 shows that formulated mRNA encoding CSP induces binding CSP-specific antibody responses in mice, using a FACS based antibody detection assay. FIG. 2A: Positive cells at day 21 post vaccination; FIG. 2B: Positive cells at day 42 post vaccination; FIG. 2C: Positive cells at day 56 post vaccination. Groups 1 and 2: LNP formulated vaccine; Group 3: NaCl 0.9%; Group 4: Protamine formulated vaccine. Vaccination scheme see Table 10. Further details provided in Example 2.

FIG. 3 shows that formulated mRNA encoding CSP induces cellular immune responses in mice (CD8+ and/or CD4+ T-cell responses), using an intracellular cytokine staining assay (day 56 post vaccination). Groups 1 and 2: LNP formulated vaccine; Group 3: NaCl 0.9%; Group 4: Protamine formulated vaccine. Vaccination scheme see Table 10. Further details provided in Example 2.

FIG. 4 shows that LNP formulated mRNA encoding CSP induces humoral immune responses in mice, using an ELISA assay. IgG1 and IgG2 endpoint titers at day 21 post vaccination are shown. Groups A-C LNP formulated CSP mRNA; Group D LNP formulated RTS,S mRNA; Group E: negative control. Vaccination scheme see Table 11. Further details provided in Example 3.

FIG. 5 shows that LNP formulated mRNA encoding CSP induces humoral immune responses in mice, using an ELISA assay. IgG1 and IgG2 endpoint titers at day 35 post vaccination are shown. Groups A-C LNP formulated CSP mRNA; Group D LNP formulated RTS,S mRNA, Group E: negative control. Vaccination scheme see Table 11. Further details provided in Example 3.

FIG. 6 shows that LNP formulated mRNA encoding CSP induces cellular immune responses in mice (CD8+ and/or CD4+ T-cell responses), using an intracellular cytokine staining assay (day 35 post vaccination). Groups A-C LNP formulated CSP mRNA; Group D LNP formulated RTS,S mRNA, Group E: negative control. Vaccination scheme see Table 11. Further details provided in Example 3.

FIG. 7 shows that mRNA constructs encoding different CSP construct designs are expressed and secreted in mammalian cells using western blot analysis. A: R7111, B: R7641, C: R7642, D: R7643, E: R7647, F: R7649 and G: R7650; M=size ladder (see Table 12). Further details provided in Example 4.

FIG. 8 shows that formulated mRNA encoding CSP variants induces humoral immune responses in mice, using an ELISA assay. FIG. 8A: coating: [NANP]7 peptide; IgG1 and IgG2a endpoint titers at day 21 post vaccination; FIG. 8B: coating: [NANP]7 peptide; IgG1 and IgG2a endpoint titers at day 35 post vaccination; groups 1 and 2: LNP formulated CSP vaccine; group 3: NaCl buffer. Vaccination scheme see Table 13A. Further details provided in Example 6.

FIG. 9 shows that formulated mRNA encoding different CSP variants induces cellular immune responses in mice (CD8+ and/or CD4+ T-cell responses), using an intracellular cytokine staining assay. Groups 1 and 2: LNP formulated CSP vaccine; Group 3: NaCl buffer. Vaccination scheme see Table 13A. Further details provided in Example 6.

FIG. 10 shows that formulated mRNA encoding different CSP variants induces humoral immune responses in mice, using an ELISA assay. FIG. 10A: coating: [NANP]₇ peptide; IgG1 and IgG2a endpoint titers at day 21 post vaccination; FIG. 10B: coating: [NANP]₇ peptide; IgG1 and IgG2a endpoint titers at day 35 post vaccination; FIG. 10C: coating C-term peptide; IgG1 and IgG2a endpoint titers at 21 days post vaccination; FIG. 10D: coating C-term peptide; IgG1 and IgG2a endpoint titers at 35 days post vaccination; FIG. 10E: coating N-term peptide; IgG1 and IgG2a endpoint titers at 21 days post vaccination; FIG. 10F: coating N-term peptide; IgG1 and IgG2a endpoint titers at 35 days post vaccination. Groups 1 to 3: LNP formulated CSP vaccine; Group 4: LNP with irrelevant Poly: C RNA. Vaccination scheme see Table 13B. Further details provided in Example 7.

FIG. 11 shows that formulated mRNA encoding different CSP variants induces cellular immune responses in mice (CD8+ and/or CD4+ T-cell responses), using an intracellular cytokine staining assay (day 35 post vaccination). Groups 1 to 3: LNP formulated CSP vaccine; Group 4: LNP with irrelevant Poly: C RNA. Vaccination scheme see Table 13B. Further details provided in Example 7.

FIG. 12 shows that formulated mRNA encoding CSP with shortened C-terminus induces humoral immune responses in mice, using an ELISA assay. FIG. 12A: coating: [NANP]₇ peptide; IgG1 and IgG2a endpoint titers at day 21 post vaccination; FIG. 12B: coating: [NANP]₇ peptide; IgG1 and IgG2a endpoint titers at day 35 post vaccination; FIG. 12C: coating C-term peptide; IgG1 and IgG2a endpoint titers at 21 days post vaccination; FIG. 12D: coating C-term peptide; IgG1 and IgG2a endpoint titers at 35 days post vaccination. Groups 1 to 6: LNP formulated CSP vaccine (shortened at C-terminus); Group 7: LNP with Poly: C RNA. Vaccination scheme see Table 14. Further details provided in Example 8.

FIG. 13 shows that formulated mRNA encoding CSP with shortened C-terminus induces cellular immune responses in mice (CD8+ and/or CD4+ T-cell responses), using an intracellular cytokine staining assay (day 35 post vaccination). FIG. 13A: all groups. FIG. 13B: only groups 5-7 to show comparison of group 5 and 6. Groups 1 to 6: LNP formulated CSP vaccine (shortened at C-terminus); Group 7: LNP with Poly: C RNA. Vaccination scheme see Table 14. Further details provided in Example 8.

FIG. 14 shows that LNP formulated mRNA encoding CSP with different C-terminus induces humoral immune responses in mice, using an ELISA assay. FIG. 14A: coating: [NANP]₇ peptide; IgG1 and IgG2a endpoint titers at day 21 post vaccination; FIG. 14B: coating: [NANP]₇ peptide; IgG1 and IgG2a endpoint titers at day 35 post vaccination; FIG. 14C: coating C-term peptide; IgG1 and IgG2a endpoint titers at 35 days post vaccination; FIG. 14D: coating N-term peptide; IgG1 and IgG2a endpoint titers at 35 days post vaccination. Groups 1 to 8 LNP formulated CSP vaccine (different C-terminus); Group 9: LNP formulated irrelevant RNA. Vaccination scheme see Table 15. Further details provided in Example 9.

FIG. 15 shows that LNO formulated mRNA encoding CSP with different C-terminus induces cellular immune responses in mice (CD8+ and/or CD4+ T-cell responses), using an intracellular cytokine staining assay (day 35 post vaccination). Groups 1 to 8: LNP formulated CSP vaccine (different C-terminus); Group 9: LNP formulated irrelevant RNA. Vaccination scheme see Table 15. Further details provided in Example 9.

FIG. 16 shows that LNP formulated mRNA encoding CSP vaccine (shortened at N-terminus or NANP repeat region at the C-terminus) induces humoral immune responses in mice, using an ELISA assay. FIG. 16A: coating: [NANP]7 peptide; IgG1 and IgG2a endpoint titers at day 21 post vaccination; FIG. 16B: coating: [NANP]7 peptide; IgG1 and IgG2a endpoint titers at day 35 post vaccination; FIG. 16C: coating C-term peptide; IgG1 and IgG2a endpoint titers at 35 days post vaccination; FIG. 16D: coating N-term peptide; IgG1 and IgG2a endpoint titers at 35 days post vaccination. Groups 1 to 8: LNP formulated CSP vaccine (shortened at N-terminus or NANP repeat region at the C-terminus); Group 9: LNP formulated irrelevant RNA. Vaccination scheme see Table 16. Further details provided in Example 10.

FIG. 17 shows that LNP formulated mRNA encoding CSP vaccine (shortened at N-terminus or NANP repeat region at the C-terminus) induces cellular immune responses in mice (CD8+ and/or CD4+ T-cell responses), using an intracellular cytokine staining assay (day 35 post vaccination). Groups 1 to 8: LNP formulated CSP vaccine (shortened at N-terminus or NANP repeat region at the C-terminus); Group 9: LNP formulated irrelevant RNA. Vaccination scheme see Table 16. Further details provided in Example 10.

FIG. 18 shows that LNP formulated mRNA differently capped encoding CSP vaccine induces humoral immune responses in mice, using an ELISA assay. FIG. 18A: coating: [NANP]₇ peptide; IgG1 and IgG2a endpoint titers at day 21 post vaccination; FIG. 18B: coating: [NANP]7 peptide or C-term peptide or N-term peptide; IgG1 endpoint titers at day 35 post vaccination; FIG. 18C: coating: [NANP]7 peptide or C-term peptide or N-term peptide; IgG2a endpoint titers at day 35 post vaccination. Groups 1 and 2: LNP formulated differently capped mRNA CSP vaccine; Group 3: LNP formulated irrelevant RNA. Vaccination scheme see Table 17. Further details provided in Example 11.

FIG. 19 shows that LNP formulated mRNA differently capped encoding CSP vaccine induces cellular immune responses in mice (CD8+ and/or CD4+ T-cell responses), using an intracellular cytokine staining assay (day 35 post vaccination). Groups 1 and 2: LNP formulated differently capped mRNA CSP vaccine; Group 3: LNP with irrelevant RNA. Vaccination scheme see Table 17. Further details provided in Example 11.

FIG. 20 shows that LNP formulated mRNA (differently capped) encoding CSP vaccine induces humoral immune responses in mice, using an ELISA assay. A prolonged injection interval between prime and boost enhances the humoral immune response. FIG. 20: coating: [NANP]₇ peptide; IgG1 and IgG2a endpoint titers at different days as indicated; Groups 1, 2, 4 and 5: LNP with differently capped CSP vaccine; Group 3 and 6: NaCl buffer. Vaccination scheme see Table 18. Further details provided in Example 12.

FIG. 21 shows that formulated differently capped mRNA encoding CSP vaccine induces cellular immune responses in mice (CD8+ and/or CD4+ T-cell responses), using an intracellular cytokine staining assay. Groups 1, 2, 4 and 5: LNP with differently capped CSP vaccine; Group 3 and 6: NaCl buffer. Vaccination scheme see Table 18. Further details provided in Example 12.

FIG. 22 shows that independently which 3′-end is used mRNA encoding CSP vaccine induces humoral immune responses in mice, using an ELISA assay. FIG. 22A: coating: [NANP]₇ peptide; IgG1 and IgG2a endpoint titers at day 21 post first vaccination; FIG. 22B: coating: [NANP]₇ peptide; IgG1 and IgG2a endpoint titers at day 35 post vaccination; Groups 1-6: mRNA with different 3′-end and unmodified or modified uracil encoding CSP vaccine; Group 7: NaCl buffer. Vaccination scheme see Table 19. Further details provided in Example 13.

FIG. 23 shows that independently which 3′-end is used mRNA encoding CSP vaccine induces cellular immune responses in mice (CD8+ and/or CD4+ T-cell responses), using an intracellular cytokine staining assay. Groups 1-6: mRNA with different 3′-end and unmodified or modified uracil encoding CSP vaccine; Group 7: NaCl buffer. Vaccination scheme see Table 19. Further details provided in Example 13.

FIG. 24 shows that expression is comparable independently which 3′-end is used. The constructs with shortened N-terminus of CSP (group G and H) showed slightly higher expression. The experiment was performed as described in Example 14. For further details see Table 20.

FIG. 25 shows that all mRNA constructs encoding Malaria CSP antigens led to a detectable protein expression using a rabbit reticulocyte lysate system. Further details provided in Example 15 and Table 21.

FIG. 26 shows schematic drawings of preferred CSP constructs/protein designs; CSP: circumsporozoite protein of Plasmodium (fragments are indicated by amino acid positions); SP: heterologous signal peptide; L: Linker; HA-TM: transmembrane domain of Influenza HA; HBsAg: Hepatitis B surface antigen; Tetanus: P2 T cell helper epitope from tetanus toxin; PADRE: pan HLA DR-binding epitope; Ferritin: Iron storage protein ferritin; Lumazine: Lumazine synthase. Further information can be found in Table 1.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Preparation of RNA Constructs, Compositions, and Vaccines

The present Example provides methods of obtaining the coding RNA of the invention, as well as methods of generating a composition or a vaccine of the invention.

1.1. Preparation of DNA and mRNA Constructs:

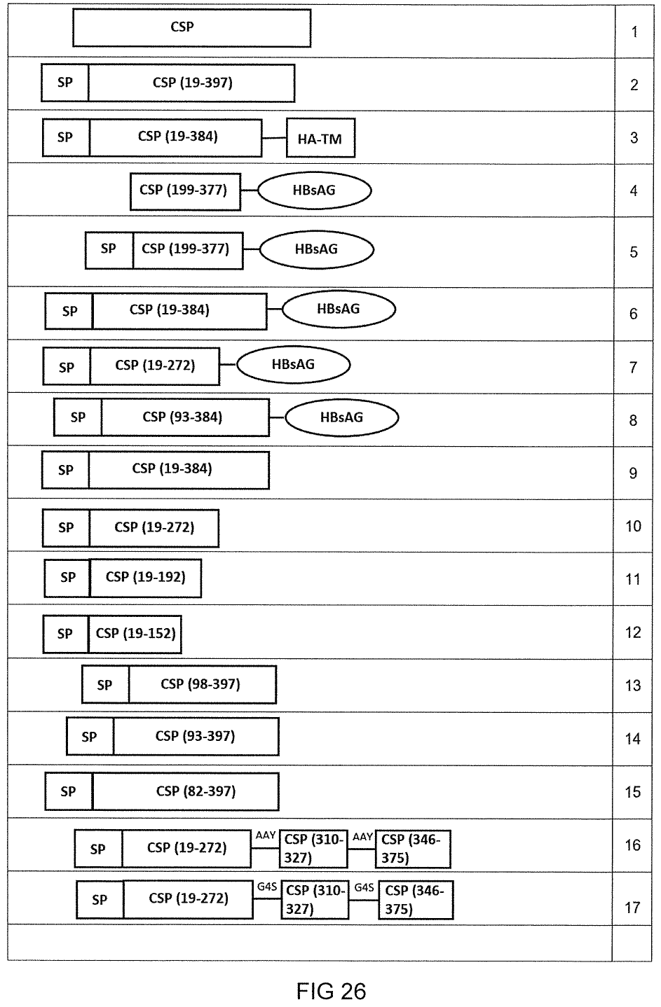

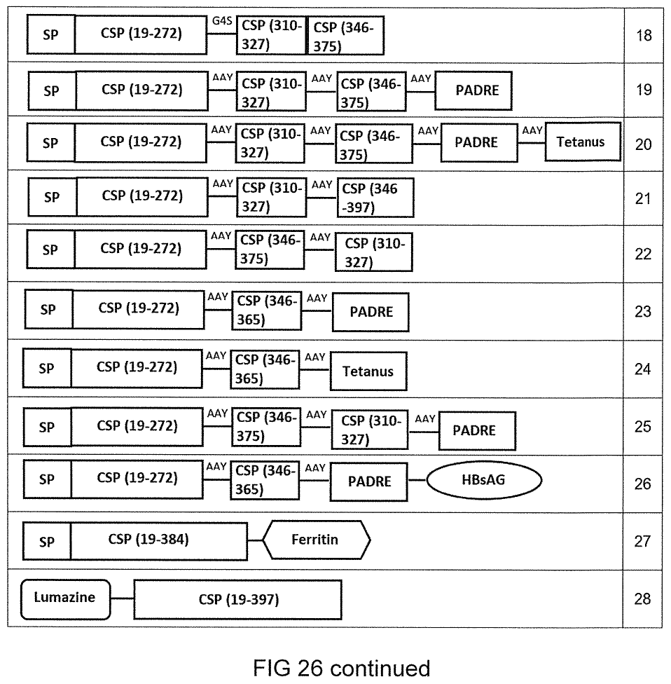

DNA sequences encoding different CSP proteins were prepared and used for subsequent RNA in vitro transcription reactions. Said DNA sequences were prepared by modifying the wild type encoding DNA sequences by introducing a G/C optimized coding sequence (e.g., “cds opt1”) for stabilization and expression optimization. Sequences were introduced into a pUC derived DNA vector to comprise stabilizing 3′-UTR sequences and 5′-UTR sequences, additionally comprising a stretch of adenosines (e.g. 64A or A100), and optionally a histone-stem-loop (hSL) structure, and a stretch of 30 cytosines (e.g. C30) (see Table 9, a (for a schematic overview of CSP construct designs see FIG. 26).

The obtained plasmid DNA constructs were transformed and propagated in bacteria using common protocols known in the art. Eventually, the plasmid DNA constructs were extracted, purified, and used for subsequent RNA in vitro transcription (see section 1.2).

Alternatively, DNA plasmids are used as template for PCR-amplification (see section 1.3).

1.2. RNA In Vitro Transcription from Plasmid DNA Templates:

DNA plasmids prepared according to paragraph 1.1 were linearized using a restriction enzyme and used for DNA dependent RNA in vitro transcription using T7 RNA polymerase in the presence of a nucleotide mixture (ATP/GTP/CTP/UTP) and cap analog (e.g. m7GpppG) under suitable buffer conditions. m7G(5′)ppp(5′)(2′OMeA)pG cap analog was used for preparation of some RNA constructs to generate a cap1 structure (e.g. R8143, R8229, R8233, R8232, R8230, R8231, R8238). Obtained RNA constructs were purified using RP-HPLC (PureMessenger®, CureVac AG, Tubingen, Germany; WO2008/077592) and used for in vitro and in vivo experiments. The generated RNA sequences/constructs are provided in Table 9, with the encoded CSP constructs and the respective UTR elements indicated therein (mRNA design a-1 (HSD17B4/PSMB3), mRNA design a-3 (SLC7A3/PSMB3), mRNA design i-3 (−/muag) and mRNA design i-2 (RPL32/ALB7)). CSP proteins and fragments were derived from Plasmodium falciparum 3D7 (XP_001351122.1, XM_001351086.1; abbreviated herein as “Pf(3D7)”), or Plasmodium berghei ANKA (XP_022712148.1, XM_022858407.1; abbreviated herein as “Pb(ANKA”)).

In addition to the information provided in Table 9, further information relating to specific mRNA construct SEQ-ID NOs may be derived from the information provided under <223> identifier in the ST.25 sequence listing.

To obtain modified mRNA RNA in vitro transcription was performed in the presence of a modified nucleotide mixture (ATP, GTP, CTP, pseudouridine (ψ)) or N(1)-methylpseudouridine (m1ψ-)) and cap analogue (m7GpppG or m7G(5′)ppp(5′)(2′OMeA)pG) under suitable buffer conditions. The obtained ψ-modified mRNAs were purified using RP-HPLC (PureMessenger®, CureVac AG, Tubingen, Germany; WO2008/077592) and used for further experiments.

Some RNA constructs are in vitro transcribed in the absence of a cap analog. The cap-structure (cap1) is added enzymatically using Capping enzymes as commonly known in the art. In short, in vitro transcribed mRNA is capped using an m7G capping kit with 2′-O-methyltransferase to obtain cap1-capped RNA.

RNA for clinical development is produced under current good manufacturing practice e.g. according to WO2016/180430, implementing various quality control steps on DNA and RNA level.

1.3. RNA In Vitro Transcription from PCR Amplified DNA Templates:

Purified PCR amplified DNA templates prepared according to paragraph 1.1 are transcribed in vitro using DNA dependent T7 RNA polymerase in the presence of a nucleotide mixture (ATP/GTP/CTP/UTP) and cap analog (m7GpppG or m7G(5′)ppp(5′)(2′OMeA)pG) under suitable buffer conditions. Alternatively, PCR amplified DNA is transcribed in vitro using DNA dependent T7 RNA polymerase in the presence of a modified nucleotide mixture (ATP, GTP, CTP, N(1)-methylpseudouridine (m14ψ) or pseudouridine (ψ) and cap analog (m7GpppG or m7G(5′)ppp(5′)(2′OMeA)pG) under suitable buffer conditions. Some mRNA constructs are in vitro transcribed in the absence of a cap analog and the cap-structure (cap1) is added enzymatically using capping enzymes as commonly known in the art e.g. using an m7G capping kit with 2′-O-methyltransferase. The obtained mRNAs are purified e.g. using RP-HPLC (PureMessenger®, CureVac AG, Tubingen, Germany; WO2008/077592) and used for in vitro and in vivo experiments.

TABLE 9
mRNA constructs encoding CPS used in the present examples

| | | | | | | | SEQ |
|---|---|---|---|---|---|---|---|
| | | | | | | SEQ | ID |
| | | | | | SEQ | ID | NO: |
| mRNA | | UTR | | 5'-cap | ID NO: | NO: | CDS |
| ID | Protein | Design | 3′-end | structure | mRNA | Protein | opt1 |
| R7112/ | Pf-CSP | a-1 | A64-N5-C30- | cap0 | 333 | 1 | 41 |
| R7111 | | | hSL-N5 | | | | |
| R8143 | Pf-CSP | a-1 | A64-N5-C30- | cap1 | 625 | 1 | 41 |
| | | | hSL-N5 | | | | |
| R6876/ | Pf-CSP | i-2 | A64-N5-C30- | cap0 | 917 | 1 | 41 |
| R5752 | | | hSL-N5 | | | | |
| R8983 | Pf-CSP | a-3 | hSL-A100 | cap1 | 8259 | 1 | 41 |
| R8985 | Pf-CSP | a-3 | hSL-A100-N5 | cap1 | 7530 | 1 | 41 |
| R5980 | Pf-CSP_Linker(G4SG4)_TM domain HA | i-2 | A64-N5-C30- | cap0 | 924 | 8 | 48 |
| | | | hSL-N5 | | | | |
| R7271 | Pf-CSP(199-377)_Linker(PVTN)_HBsAg | a-1 | A64-N5-C30- | cap0 | 341 | 9 | 49 |
| | | | hSL-N5 | | | | |
| R8233 | Pf-CSP(199-377)_Linker(PVTN)_HBsAg | a-1 | A64-N5-C30- | cap1 | 633 | 9 | 49 |
| | | | hSL-N5 | | | | |
| R7216 | Pf-CSP(199-387)_Linker(PVTN)_HBsAg | a-1 | A64-N5-C30- | cap0 | 342 | 10 | 50 |
| | | | hSL-N5 | | | | |
| R8782 | LumSynt_Linker(GGS4-GGG)_Pf- | a-1 | A64-N5-C30- | cap1 | 8856 | 8742 | 8754 |
| | CSP(19-397) | | hSL-N5 | | | | |
| R8783 | LumSynt_Linker(GGS4-GGG)_Pf- | a-1 | hSL-A100 | cap1 | 9774 | 8742 | 8754 |
| | CSP(19-397) | | | | | | |
| R7268 | HsIns-iso1_Pf-CSP(19-397) | a-1 | A64-N5-C30- | cap0 | 337 | 5 | 45 |
| | | | hSL-N5 | | | | |
| R5974 | HsIns-isol_Pf-CSP(19-397) | i-2 | A64-N5-C30- | cap0 | 921 | 5 | 45 |
| | | | hSL-N5 | | | | |
| R7266 | HsSPARC_Pf-CSP(19-397) | a-1 | A64-N5-C30- | cap0 | 338 | 6 | 46 |
| | | | hSL-N5 | | | | |
| R5972 | HsSPARC_Pf-CSP(19-397) | i-2 | A64-N5-C30- | cap0 | 922 | 6 | 46 |
| | | | hSL-N5 | | | | |
| R7265 | IgE_Pf-CSP(19-397) | a-1 | A64-N5-C30- | cap0 | 339 | 7 | 47 |
| | | | hSL-N5 | | | | |
| R5971 | IgE_Pf-CSP(19-397) | i-2 | A64-N5-C30- | cap0 | 923 | 7 | 47 |
| | | | hSL-N5 | | | | |
| R7267 | HsALB_Pf-CSP(19-397) | a-1 | A64-N5-C30- | cap0 | 336 | 4 | 44 |
| | | | hSL-N5 | | | | |
| R8229 | HsALB_Pf-CSP(19-397) | a-1 | A64-N5-C30- | cap1 | 628 | 4 | 44 |
| | | | hSL-N5 | | | | |
| R8524 | HsALB_Pf-CSP(19-397) | a-1 | A64-N5-C30- | cap1 | 628 | 4 | 44 |
| | | | hSL-N5 | | | | |
| R5973 | HsALB_Pf-CSP(19-397) | i-2 | A64-N5-C30- | cap0 | 920 | 4 | 44 |
| | | | hSL-N5 | | | | |
| R8527 | HsALB_Pf-CSP(19-397) | i-3 | A64-N5-C30- | cap1 | 1796 | 4 | 44 |
| | | | hSL-N5 | | | | |
| R8530 | HsALB_Pf-CSP(19-397) | i-3 | A64-N5-C30- | cap1 | 1796 | 4 | 44 |
| | | | hSL-N5 | | | | |
| R8523 | HsALB_Pf-CSP(19-397) | a-1 | hSL-A64-N5 | cap1 | 6561 | 4 | 44 |
| R8525 | HsALB_Pf-CSP(19-397) | a-1 | hSL-A64-N5 | cap1 | 6561 | 4 | 44 |
| R8528 | HsALB_Pf-CSP(19-397) | i-3 | hSL-A64-N5 | cap1 | 7047 | 4 | 44 |
| R8531 | HsALB_Pf-CSP(19-397) | i-3 | hSL-A64-N5 | cap1 | 7047 | 4 | 44 |
| R8987 | HsALB_Pf-CSP(19-397) | a-1 | hSL-A100-N5 | cap1 | 7290 | 4 | 44 |
| R8986 | HsALB_Pf-CSP(19-397) | a-3 | hSL-A100-N5 | cap1 | 7533 | 4 | 44 |
| R8520 | HsALB_Pf-CSP(19-397) | a-1 | hSL-A100 | cap1 | 9775 | 4 | 8755 |
| R8526 | HsALB_Pf-CSP(19-397) | a-1 | hSL-A100 | cap1 | 9775 | 4 | 8755 |
| R8529 | HsALB_Pf-CSP(19-397) | i-3 | hSL-A100 | cap1 | 8505 | 4 | 8755 |
| R8532 | HsALB_Pf-CSP(19-397) | i-3 | hSL-A100 | cap1 | 8505 | 4 | 8755 |
| R8984 | HsALB_Pf-CSP(19-397) | a-3 | hSL-A100 | cap1 | 8262 | 4 | 8755 |
| R8091 | HsALB_Pf-CSP(19-152) | a-1 | A64-N5-C30- | cap0 | 343 | 11 | 51 |
| | | | hSL-N5 | | | | |
| R8090 | HsALB_Pf-CSP(19-192) | a-1 | A64-N5-C30- | cap0 | 344 | 12 | 52 |
| | | | hSL-N5 | | | | |
| R7647 | HsALB_Pf-CSP(19-272) | a-1 | A64-N5-C30- | cap0 | 345 | 13 | 53 |
| | | | hSL-N5 | | | | |
| R8232 | HsALB_Pf-CSP(19-272) | a-1 | A64-N5-C30- | cap1 | 637 | 13 | 53 |
| | | | hSL-N5 | | | | |
| R8567 | HsALB_Pf-CSP(19- | a-1 | A64-N5-C30- | cap1 | 8858 | 8743 | 8756 |
| | 272)_Linker(PVTN)_HBsAg | | hSL-N5 | | | | |
| R8104 | HsALB_Pf-CSP(19-272)_Linker(G4S)_Pf- | a-1 | A64-N5-C30- | cap0 | 350 | 18 | 58 |
| | CSP(310-327)_Pf-CSP(346-375) | | hSL-N5 | | | | |
| R8103 | HsALB_Pf-CSP(19-272)_Linker(G4S)_Pf- | a-1 | A64-N5-C30- | cap0 | 349 | 17 | 57 |
| | CSP(310-327)_Linker(G4S)_Pf-CSP(346- | | hSL-N5 | | | | |
| | 375) | | | | | | |
| R8100 | HsALB_Pf-CSP(19-272)_Linker(AAY)_Pf- | a-1 | A64-N5-C30- | cap0 | 346 | 14 | 54 |
| | CSP(310-327)_Linker(AAY)_Pf-CSP(346- | | hSL-N5 | | | | |
| | 375) | | | | | | |
| R8101 | HsALB_Pf-CSP(19-272)_Linker(AAY)_Pf- | a-1 | A64-N5-C30- | cap0 | 348 | 16 | 56 |
| | CSP(346-365)_Linker(AAY)_PADRE | | hSL-N5 | | | | |
| R8102 | HsALB_Pf-CSP(19-272)_Linker(AAY)_Pf- | a-1 | A64-N5-C30- | cap0 | 347 | 15 | 55 |
| | CSP(346-365)_Linker(AAY)_P2 | | hSL-N5 | | | | |
| R8571 | HsALB_Pf-CSP(19-272)_Linker(AAY)_Pf- | a-1 | A64-N5-C30- | cap1 | 8859 | 8744 | 8757 |
| | CSP(346- | | hSL-N5 | | | | |
| | 365)_Linker(AAY)_PADRE_Linker(PVTN)_ | | | | | | |
| | HBsAg | | | | | | |
| R8572 | HsALB_Pf-CSP(19-272)_Linker(AAY)_Pf- | a-1 | hSL-A100 | cap1 | 9778 | 8744 | 8758 |
| | CSP(346- | | | | | | |
| | 365)_Linker(AAY)_PADRE_Linker(PVTN)_ | | | | | | |
| | HBsAg | | | | | | |
| R7643 | HsALB_Pf-CSP(19-325) | a-1 | A64-N5-C30- | cap0 | 351 | 19 | 59 |
| | | | hSL-N5 | | | | |
| R7642 | HsALB_Pf-CSP(19-384) | a-1 | A64-N5-C30- | cap0 | 352 | 20 | 60 |
| | | | hSL-N5 | | | | |
| R8230 | HsALB_Pf-CSP(19-384) | a-1 | A64-N5-C30- | cap1 | 644 | 20 | 60 |
| | | | hSL-N5 | | | | |
| R7641 | HsALB_Pf-CSP(19-384)_TM domain HA | a-1 | A64-N5-C30- | cap0 | 353 | 21 | 61 |
| | | | hSL-N5 | | | | |
| R8231 | HsALB_Pf-CSP(19-384)_TM domain HA | a-1 | A64-N5-C30- | cap1 | 645 | 21 | 61 |
| | | | hSL-N5 | | | | |
| R8565 | HsALB_Pf-CSP(19- | a-1 | A64-N5-C30- | cap1 | 8861 | 8745 | 8759 |
| | 384)_Linker(PVTN)_HBsAg | | hSL-N5 | | | | |
| R8566 | HsALB_Pf-CSP(19- | a-1 | hSL-A100 | cap1 | 9780 | 8745 | 8760 |
| | 384)_Linker(PVTN)_HBsAg | | | | | | |
| R8780 | HsALB_Pf-CSP(19- | a-1 | A64-N5-C30- | cap1 | 8863 | 8746 | 8761 |
| | 384)_Linker(SGG)_Ferritin | | hSL-N5 | | | | |
| R8781 | HsALB_Pf-CSP(19- | a-1 | hSL-A100 | cap1 | 9782 | 8746 | 8762 |
| | 384)_Linker(SGG)_Ferritin | | | | | | |
| R8092 | HsALB_Pf-CSP(82-397) | a-1 | A64-N5-C30- | cap0 | 354 | 22 | 62 |
| | | | hSL-N5 | | | | |
| R7768 | HsALB_Pf-CSP(93-192) | a-1 | A64-N5-C30- | cap0 | 355 | 23 | 63 |
| | | | hSL-N5 | | | | |
| R7644 | HsALB_Pf-CSP(93-272) | a-1 | A64-N5-C30- | cap0 | 356 | 24 | 64 |
| | | | hSL-N5 | | | | |
| R8573 | HsALB_Pf-CSP(93- | a-1 | A64-N5-C30- | cap1 | 8865 | 8747 | 8763 |
| | 384)_Linker(PVTN)_HBsAg | | hSL-N5 | | | | |
| R8574 | HsALB_Pf-CSP(93- | a-1 | hSL-A100 | cap1 | 9784 | 8747 | 8764 |
| | 384)_Linker(PVTN)_HBsAg | | | | | | |
| R8089 | HsALB_Pf-CSP(93-397) | a-1 | A64-N5-C30- | cap0 | 357 | 25 | 65 |
| | | | hSL-N5 | | | | |
| R7646 | HsALB_Pf-CSP(98-192) | a-1 | A64-N5-C30- | cap0 | 358 | 26 | 66 |
| | | | hSL-N5 | | | | |
| R7645 | HsALB_Pf-CSP(98-272) | a-1 | A64-N5-C30- | cap0 | 359 | 27 | 67 |
| | | | hSL-N5 | | | | |
| R7650 | HsALB_Pf-CSP(98-374) | a-1 | A64-N5-C30- | cap0 | 360 | 28 | 68 |
| | | | hSL-N5 | | | | |
| R7649 | HsALB_Pf-CSP(98-397) | a-1 | A64-N5-C30- | cap0 | 361 | 29 | 69 |
| | | | hSL-N5 | | | | |
| R8031 | HsALB_Pf-CSP(199- | a-1 | A64-N5-C30- | cap0 | 362 | 30 | 70 |
| | 377)_Linker(PVTN)_HBsAg | | hSL-N5 | | | | |
| R8238 | HsALB_Pf-CSP(199- | a-1 | A64-N5-c30- | cap1 | 654 | 30 | 70 |
| | 377)_Linker(PVTN)_HBsAg | | hSL-N5 | | | | |
| R8564 | HsALB_Pf-CSP(199- | a-1 | hSL-A100 | cap1 | 9785 | 30 | 8765 |
| | 377)_Linker(PVTN)_HBsAg | | | | | | |
| R5969 | Pb-CSP | i-2 | A64-N5-C30- | cap0 | 947 | 31 | 71 |
| | | | hSL-N5 | | | | |
| R5975 | IgE_Pb-CSP(24-340) | i-2 | A64-N5-C30- | cap0 | 952 | 36 | 76 |
| | | | hSL-N5 | | | | |
| R5976 | HsSPARC_Pb-CSP(24-340) | i-2 | A64-N5-C30- | cap0 | 951 | 35 | 75 |
| | | | hSL-N5 | | | | |
| R5977 | HsALB_Pb-CSP(24-340) | i-2 | A64-N5-C30- | cap0 | 949 | 33 | 73 |
| | | | hSL-N5 | | | | |
| R5978 | HsIns-iso1_Pb-CSP(24-340) | i-2 | A64-N5-C30- | cap0 | 950 | 34 | 74 |
| | | | hSL-N5 | | | | |
| R5979 | Pb-CSP_Linker(G4SG4)_TM domain HA | i-2 | A64-N5-C30- | cap0 | 948 | 32 | 72 |
| | | | hSL-N5 | | | | |

1.4.1: Preparation of an LNP (LNP-III-3) Formulated mRNA Composition:

LNPs (LNP-III-3) were prepared using cationic lipids, structural lipids, a PEG-lipids, and cholesterol. Lipid solution (in ethanol) was mixed with RNA solution (aqueous buffer) using a microfluidic mixing device. Obtained LNPs were re-buffered in a carbohydrate buffer via dialysis, and up-concentrated to a target concentration using ultracentrifugation tubes. LNP-formulated mRNA was stored at −80° C. prior to use in in vitro or in vivo experiments.

Lipid nanoparticles (LNP), cationic lipids, and polymer conjugated lipids (PEG-lipid) were prepared and tested essentially according to the general procedures described in WO2015/199952, WO2017/004143 and WO2017/075531, the full disclosures of which are incorporated herein by reference. LNP formulated RNA was prepared using an ionizable amino lipid (cationic lipid), phospholipid, cholesterol and a PEGylated lipid. Briefly, cationic lipid compound of formula III-3, DSPC, cholesterol, and PEG-lipid of formula IVa were solubilized in ethanol at a molar ratio (%) of approximately 50:10:38.5:1.5 or 47.4:10:40.9:1.7. LNPs comprising cationic lipid compound of formula III-3 and PEG-lipid compound of formula IVa were prepared at a ratio of RNA to total Lipid of 0.03-0.04 w/w. The RNA was diluted to 0.05 mg/mL to 0.2 mg/mL in 10 mM to 50 mM citrate buffer, pH4. Syringe pumps were used to mix the ethanolic lipid solution with the RNA aqueous solution at a ratio of about 1:5 to 1:3 (vol/vol) with total flow rates above 15 ml/min. The ethanol was then removed and the external buffer replaced with a PBS buffer comprising Sucrose by dialysis. Finally, the lipid nanoparticles were filtered through a 0.2 um pore sterile filter and the LNP-formulated RNA composition was adjusted to about 1 mg/ml total RNA. Lipid nanoparticle particle diameter size was 60-90 nm as determined by quasi-elastic light scattering using a Malvern Zetasizer Nano (Malvern, UK). For other cationic lipid compounds mentioned in the present specification, the formulation process is essentially similar. The obtained LNP-formulated RNA composition (1 mg/ml total RNA) was diluted to the desired target concentration using Saline before in vivo application.

Lipid nanoparticle composition for LNP composition LNP-III-3 are detailed in Table B below.

### Example 1.4.2: Preparation of LNPs (GN01-LNPs) Using the NanoAssemblr™ Microfluidic System

GN01-LNPs were prepared using the NanoAssemblr™ microfluidic system (Precision NanoSystems Inc., Vancouver, BC) according to standard protocols. GN01-LNPs comprising the cationic lipid COATSOME® SS-EC (former name: SS-33/4PE-15; NOF Corporation, Tokyo, Japan).

In the present examples, COATSOME® SS-EC (NOF Corporation, Tokyo, Japan) was used for preparation of lipid nanoparticle compositions. Furthermore, cholesterol (Avanti Polar Lipids; Alabaster, Ala.), neutral lipid/phospholipid DPhyPE (Avanti Polar Lipids; Alabaster, Ala.) and DMG-PEG 2000 (NOF Corporation, Tokyo, Japan) were used.

The lipids were solubilized in alcoholic solution (ethanol) according to standard procedures. The corresponding lipid nanoparticle compositions are detailed in Table B below.

In detail, LNPs were prepared by mixing appropriate volumes of lipid stock solutions in ethanol buffer with an aqueous phase (25 mM sodium acetate, pH 4.0) containing appropriate amounts of mRNA as indicated herein; cholesterol, phospholipid and polymer conjugated lipid: 20 mg/ml in EtOH, cationic lipids, except for GN01: 20 mg/ml in EtOH, GN01-lipid: 30 mg/ml in tert-butanol.

Briefly, the mRNA was diluted to 0.05 to 0.2 mg/ml in 10 to 50 mM acetate buffer, pH 4. Syringe pumps were installed into inlet parts of the NanoAssemblr™ (Precision NanoSystems Inc., Vancouver, BC) and used to mix the ethanolic lipid solution with the mRNA aqueous solution at a ratio of about 1:5 to 1:3 (vol/vol) with total flow rates from about 14 ml/min to about 18 ml/min.

The ethanol was then removed and the external buffer replaced with PBS by dialysis (Slide-A-Lyzer™ Dialysis Cassettes, ThermoFisher). Finally, the lipid nanoparticles were filtered through a 0.2 μm pore sterile filter. Lipid nanoparticle particle diameter size was from about 90 nm to about 140 nm as determined by quasi-elastic light scattering using a Malvern Zetasizer Nano (Malvern Instruments Ltd.; Malvern, UK).

TABLE B
Overview of lipid nanoparticle composition of
GN01-LNPs and LNP III-3 formulation

| | | mol-percentages |
|---|---|---|
| | | for excipients |
| LNP | Excipients | [mol %] |
| formu- | [cationic lipid:steroid:neutral lipid:polymer | |
| lation | conjugated lipid] | |
| GN01 | COATSOME ® | 59:29.3:10:1.7 |
| | SS-EC:Chol:DPhyPE:DMG-PEG 2000 | |
| LNP | III-3:Chol:DSPC:formula (IVa) with | 47.4:40.9:10:1.7 |
| III-3 | n = 49 | |

1.5. Preparation of a Protamine Complexed mRNA Composition:

RNA constructs were complexed with protamine prior to use in in vivo immunization experiments. The RNA formulation consisted of a mixture of 50% free RNA and 50% RNA complexed with protamine at a weight ratio of 2:1. First, mRNA was complexed with protamine by addition of protamine-Ringer's lactate solution to mRNA. After incubation for 10 minutes, when the complexes were stably generated, free mRNA was added, and the final concentration was adjusted with Ringer's lactate solution.

1.6. Expression Analysis of Designed mRNA Constructs:

The mRNA constructs as shown in Table 9 were tested for their expression in cell culture using western blot or FACS as commonly known in the art. An example is described in Example 4.

### Example 2: Vaccination of Mice with Protamine-Formulated and LNP-Formulated mRNA Encoding CSP

The present example shows that Malaria mRNA vaccines encoding CSP induce humoral and cellular immune responses in Balb/c mice.

Malaria mRNA constructs encoding full length CSP were prepared according to Example 1. The mRNA was formulated in lipid nanoparticles (see Example 1.4.2) or with protamine (see Example 1.5). The different mRNA vaccine candidates were applied on days 0, 21, and 42 and administered with doses of RNA, formulations, and administration routes as shown in Table 10. One negative control group 3) received NaCl buffer. Serum samples were taken at day 21, day 42, and day 56 for determination of humoral immune responses.

TABLE 10
Vaccination scheme of Example 2

| | No. of | | | | | |
|---|---|---|---|---|---|---|
| Group | mice | Treatment | mRNA ID | Dose | Route | Volume |
| 1 | 9 | mRNA encoding | R7111 | 10 ug | i.m. | 1 × 25 uL |
| | | CSP LNP | | | | |
| | | formulation 1 | | | | |
| | | (GN01) | | | | |
| 2 | 9 | mRNA encoding | R7111 | 10 ug | i.m. | 1 × 25uL |
| | | CSP LNP | | | | |
| | | formulation 2 | | | | |
| | | (similar | | | | |
| | | composition to | | | | |
| | | GN01) | | | | |
| 3 | 9 | NaCl 0.9% | | | i.m. | 1 × 25 uL |
| 4 | 9 | mRNA encoding | R7112 | 80 ug | i.d. | 2 × 50 uL |
| | | CSP | | | | |
| | | Protamine | | | | |
| | | formulation | | | | |

### 2.1. Determination of Specific Humoral Immune Responses by ELISA:

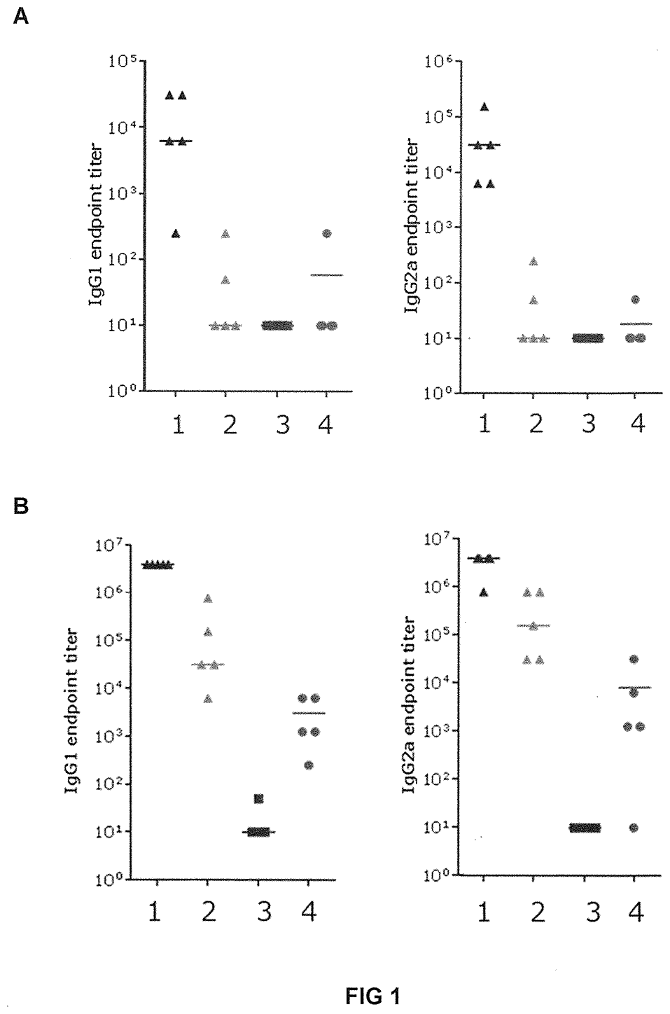

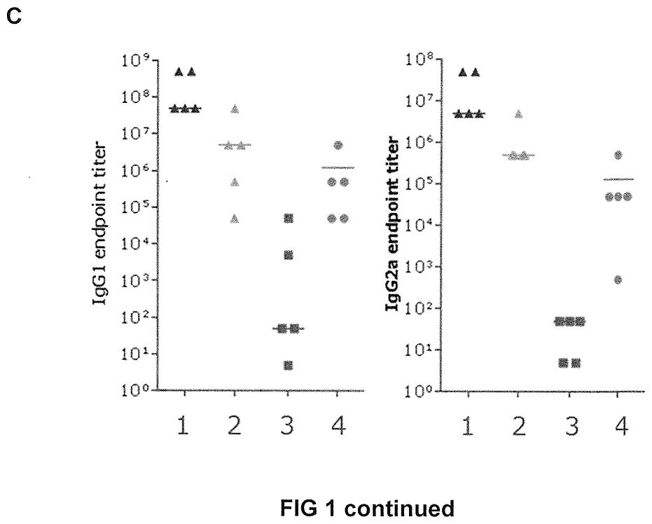

ELISA was performed using malaria [NANP]7 peptide (according to SEQ ID NO: 10209) for coating. Coated plates were incubated using respective serum dilutions, and binding of specific antibodies to the respective malaria [NANP]₇ peptide were detected using biotinylated isotype specific anti-mouse antibodies followed by streptavidin-HRP (horse radish peroxidase) with Amplex as substrate. Endpoint titers of antibodies (IgG1, IgG2a) directed against the malaria [NANP]7 peptide were measured by ELISA on day 21, day 42, and 56 post vaccinations. Results are shown in FIGS. 1 A-C(A: day 21; B: day 42; C: day 56).

### 2.2. Detection of Binding CSP-Specific Immune Responses:

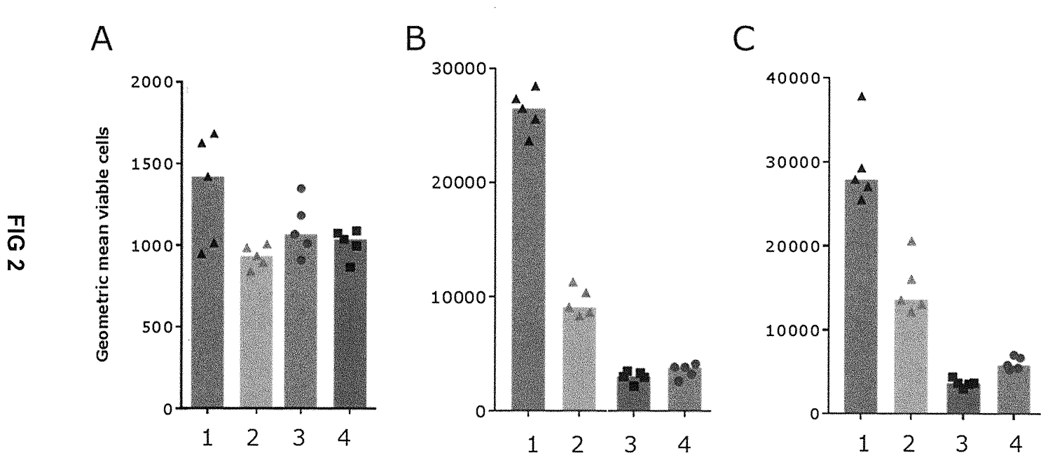

Hela cells were transfected with 2 ug of mRNA encoding CSP (R7111) using lipofectamine. The cells were harvested 20 h post transfection, and seeded at 1×10⁵ per well into a 96 well plate. The cells were incubated with serum samples of vaccinated mice (diluted 1:50) followed by aFITC-conjugated anti-mouse IgG antibody. Cells were acquired on BD FACS Canto II using DIVA software and analyzed by FlowJo. Results are shown in FIGS. 2 A-C (A: day 21; B: day 42; C: day 56).

### 2.3. Intracellular Cytokine Staining (ICS):

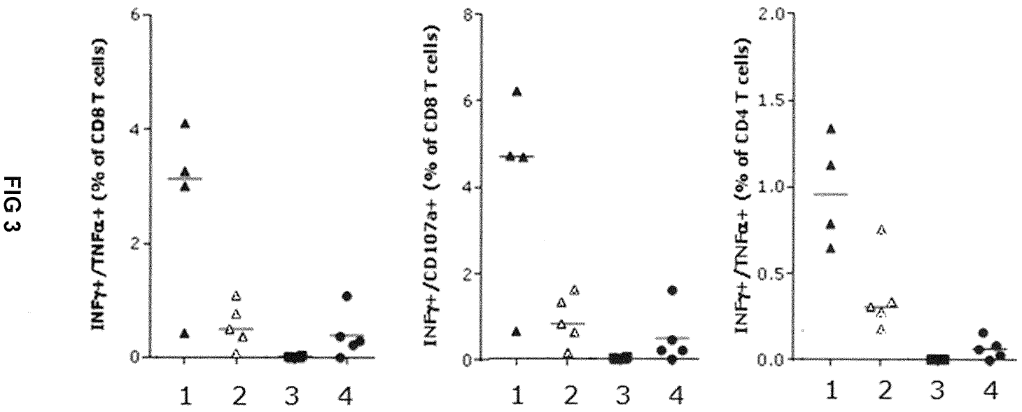

Splenocytes from vaccinated mice were isolated on day 56 according to a standard protocol known in the art. Briefly, isolated spleens were grinded through a cell strainer and washed in PBS/1% FBS followed by red blood cell lysis. After an extensive washing step with PBS/1% FBS, splenocytes were seeded into 96-well plates (2×10⁶ cells per well). Cells were stimulated with a mixture of CSP peptides (1 ug/ml) (see Table C) in the presence of 2.5 ug/ml of an anti-CD28 antibody (BD Biosciences) and a protein transport inhibitor for 6 h at 37° C. After stimulation, cells were washed and stained for intracellular cytokines using the Cytofix/Cytoperm reagent (BD Biosciences) according to the manufacturer's instructions. The following antibodies were used for staining: Thy1.2-FITC (1:100), CD8-PE-Cy7 (1:200), TNF-PE (1:100), IFNγ-APC (1:100) (eBioscience), CD4-BD Horizon V450 (1:200) (BD Biosciences) and incubated with Fcγ-block diluted 1:100. Aqua Dye was used to distinguish live/dead cells (Invitrogen). Cells were acquired using a BD FACS Canto II flow cytometer (Beckton Dickinson). Flow cytometry data was analyzed using FlowJo software (Tree Star, Inc.). Results are shown in FIG. 3.

TABLE C
CSP peptide mix for ICS

| | | | Pf(3D7)-CSP | |
|---|---|---|---|---|
| | SEQ | | Protein Amino | |
| Peptide | ID NO: | Location | Acid Position | Sequence |
| CSP_peptide_1 | 10197 | C-terminal | aa359-368 | DYANDIEKKI |
| CSP_peptide_2 | 10198 | C-terminal | aa377-385 | VFNVVNSSI |
| CSP_peptide_3 | 10199 | N-terminal | aa25-33 | CYGSSSNTR |
| CSP_peptide_4 | 10200 | N-terminal | aa26-34 | YGSSSNTRV |
| CSP_peptide_5 | 10201 | N-terminal | aa39-47 | NYDNAGTNL |
| CSP_peptide_6 | 10202 | N-terminal | aa6-14 | AILSVSSFL |
| CSP_peptide_7 | 10203 | N-terminal | aa30-38 | SNTRVLNEL |
| CSP_peptide_8 | 10204 | C-terminal | aa388-396 | IMVLSFLFL |
| CSP_peptide_9 | 10205 | C-terminal | aa387-395 | LIMVLSFLF |
| CSP_peptide_10 | 10206 | N-terminal | aa57-65 | GKQENWYSL |
| CSP_peptide_11 | 10207 | N-terminal | aa1-15 | MMRKLAILSVSSFLF |
| CSP_peptide_12 | 10208 | C-terminal | aa318-337 | EYLNKIQNSLSTEWSPCSVT |

### Results:

As shown in FIG. 1 and FIG. 2, the LNP formulated CSP mRNA vaccines induced strong, humoral immune responses in mice. Under the tested conditions, the LNP formulated vaccine applied to group 1 (1 ug dose) induced the strongest immune responses.

As shown in FIG. 3, the LNP formulated CSP mRNA vaccines induced cellular immune responses in mice (CD8+ and/or CD4+ T-cell responses). Notably, under the tested conditions, group 1 (1 ug dose) show strong and group 2 (1 ug dose) moderate CD8+ T-cell and CD4+ cell responses in comparison to the protamine formulated mRNA CSP vaccine (10 ug dose, group 4).

As CD8+ T cells may be a major protective immune mechanism against intracellular infections caused by Malaria parasites, an effective Malaria vaccine should induce strong CD8+ T cells responses. Accordingly, these findings highlight one of the advantageous features of the inventive mRNA-based malaria vaccine.

### Example 3: Vaccination of Mice with LNP-Formulated mRNA Encoding CSP

The present example shows that Malaria mRNA vaccines encoding CSP induce strong humoral and cellular immune responses in mice. Notably, the inventive mRNA-based Malaria vaccine induces strong CD8+-T cell responses.

Malaria mRNA vaccine candidates encoding full length CSP were prepared according to Example 1, and the mRNA constructs were formulated in lipid nanoparticles (see Example 1.4.2). The LNP formulations were applied on days 0 and 21 intramuscularly (i.m.; musculus tibialis, Balb/c mice) with doses of RNA, formulations, and control groups as shown in Table 11. One control group (D) received vaccinations with mRNA encoding RTS,S. A negative control group (E) received vaccinations with an irrelevant RNA, formulated in LNPs. Serum samples were taken at day 21 and day 35 for ELISA.

TABLE 11
Vaccination scheme of Example 3

| Group | No. of mice | Treatment | mRNA | Dose | Route | Volume |
|---|---|---|---|---|---|---|
| A | 9 | mRNA encoding CSP | R7111 | 1 ug | i.m. | 1 × 25 uL |
| | | LNP formulation 1 (GN01) | | | | |
| | | full length CSP | | | | |
| B | 9 | mRNA encoding CSP | R7111 | 1 ug | i.m. | 1 × 25 uL |
| | | LNP formulation 2 (similar | | | | |
| | | composition to GN01) | | | | |
| | | full length CSP | | | | |
| C | 9 | mRNA encoding CSP | R7111 | 1 ug | i.m. | 1 × 25 uL |
| | | LNP formulation 3 (similar | | | | |
| | | composition to GN01) | | | | |
| | | full length CSP | | | | |
| D | 9 | mRNA RTS, S | R7271 | 10 ug | i.m. | 1 × 25 uL |
| | | LNP formulation 1 (GN01) | | | | |
| | | Pf-CSP(199- | | | | |
| | | 377)_Linker(PVTN)_HBsAg | | | | |
| E | 5 | irrelevant RNA | Irrelevant RNA | 1 ug | i.m. | 1 × 25 uL |
| | | LNP formulation 1 (GN01) | | | | |

### 3.1. Determination of Specific Humoral Immune Responses by ELISA:

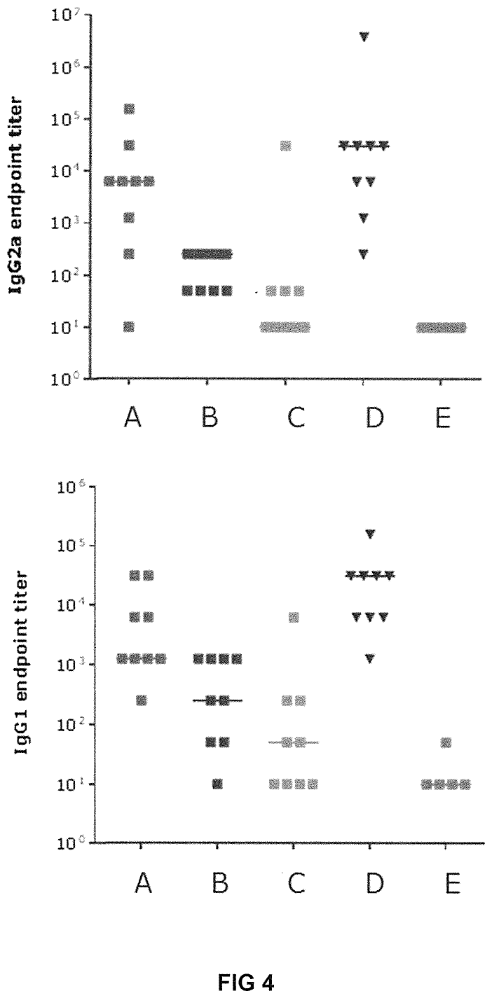

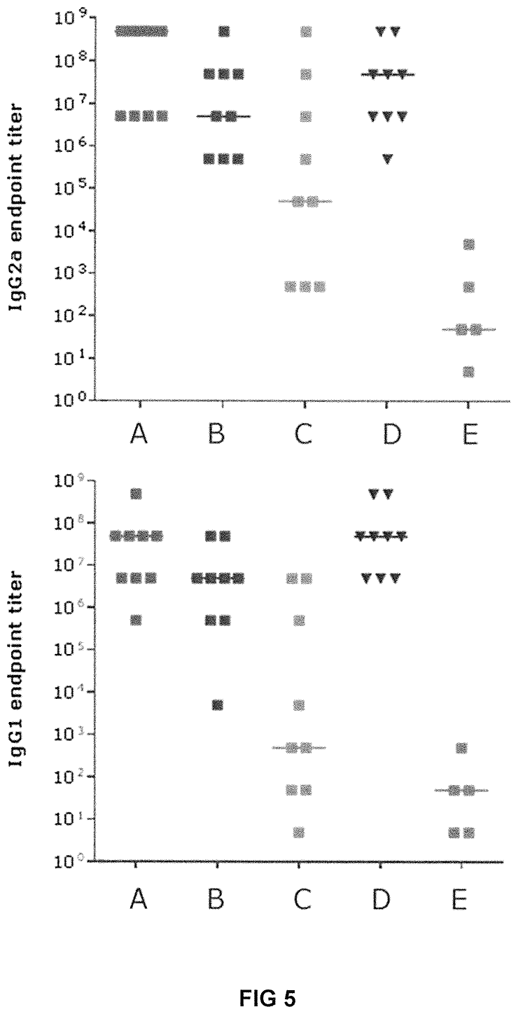

ELISA was performed using malaria peptide [NANP]7 for coating essentially as described in Example 3.1. The results are shown in FIG. 4 and FIG. 5.

### 3.2. Intracellular Cytokine Staining:

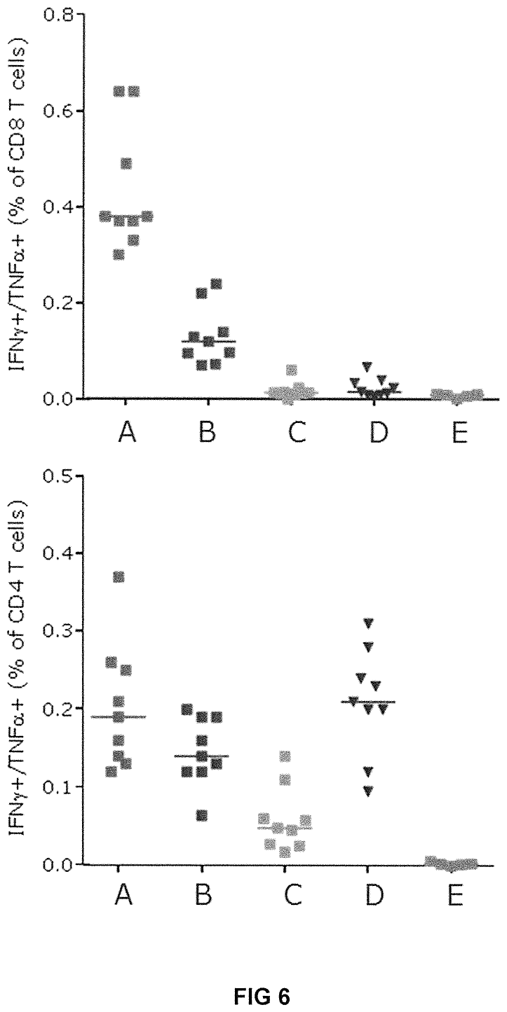

Intracellular cytokine staining was performed essentially as described in Example 3.2. The results are shown in FIG. 6.

### Results:

As shown in FIG. 4 and FIG. 5, the LNP formulated CSP mRNA vaccines induced strong, humoral immune responses in mice. Under the tested conditions, the LNP formulated vaccine applied to group A (1 ug dose) induced the strongest immune responses that were comparable to the RTS,S group (CSP fragment with HBsAg) (Group D; 10 ug dose).

As shown in FIG. 6, the LNP formulated CSP mRNA vaccines induced cellular immune responses in mice (CD8+ and/or CD4+ T-cell responses). Notably, under the tested conditions, group A (1 ug dose) and B (1 ug dose) showed strong CD8+ T-cell and CD4+ cell responses, whereas the RTS,S group (CSP fragment with HBsAg) (Group D; 10 ug dose) only showed CD4+ T-cell responses.

As CD8+ T cells are a major protective immune mechanism against intracellular infections caused by Malaria parasites, an effective Malaria vaccine should induce strong CD8+ T cells responses. Accordingly, these findings highlight one of the advantageous features of the inventive mRNA-based malaria vaccine.

### Example 4: Expression Analysis of Different mRNA Construct Encoding CSP in 293T Cells

The present example shows that RNA constructs encoding modified CSP constructs are expressed and secreted in mammalian cells.

To determine in vitro protein expression of some of the RNA constructs, 293T cells were transiently transfected with mRNA encoding CSP antigen. 24 h prior to transfection, 293T cells were seeded in a 6-well plate at a density of 500,000 cells/well in cell culture medium. Cells were transfected with 1 ug RNA using Lipofectamine 2000 (Invitrogen) as transfection agent. The following mRNA constructs were used in the experiment: R7111, R7641, R7642, R7643, R7647, R7649 and R7650 (see Table 12).

TABLE 12
RNA constructs used for western blot analysis, Example 4

| | | RNA | expected |
|---|---|---|---|
| Group | Construct | ID | protein size |
| A | Pf-CSP | R7111 | approx. 43 kDA |
| B | HsALB_Pf-CSP(19-384)_TM | R7641 | approx. 46 kDA |
| | domain HA | | |
| C | HsALB_Pf-CSP(19-384) | R7642 | approx. 41 kDA |
| D | HsALB_Pf-CSP(19-325) | R7643 | approx. 35 kDA |
| E | HsALB_Pf-CSP(19-272) | R7647 | approx. 29 kDA |
| F | HsALB_Pf-CSP(98-397) | R7649 | approx. 33 kDA |
| G | HsALB_Pf-CSP(98-374) | R7650 | approx. 31 kDA |

| | |
|---|---|
| M | Protein size marker |

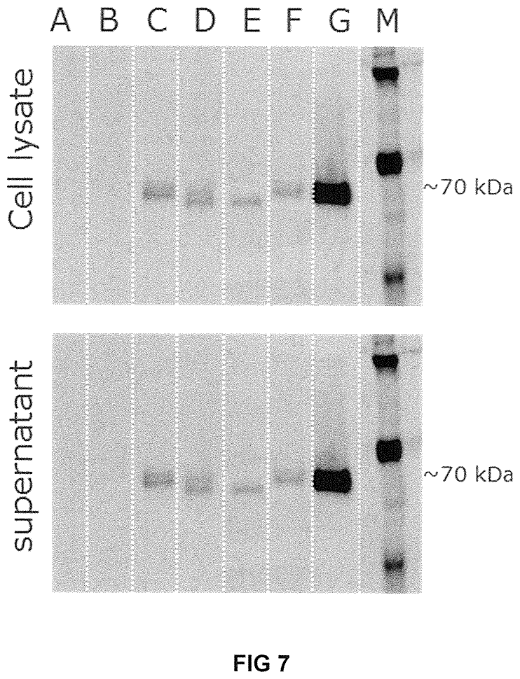

Western Blot analysis was performed as commonly known in the art, using mouse anti-2A10 monoclonal antibody against the repeat region of CSP(1:5000 diluted) as primary antibody in combination with secondary anti-mouse antibody IgG IRDye 800CW (1:10000 diluted) (see FIG. 7A and FIG. 7B).

### Results:

For five of the tested RNA constructs (R7642; R7643; R7647; R7649 and R7650) the encoded CSP protein was detectable in the supernatants of transfected 293T cells (see FIG. 7A). Expression of all seven constructs was demonstrated in the corresponding cell lysates (see FIG. 7B).

### Example 5: Evaluation of the Functional Immunogenicity of mRNA Based Malaria Vaccines (Prophetic)

The aim of the present example is to evaluate the functional immunogenicity of the inventive mRNA based Malaria vaccine with an improved ELISA assay, a passive transfer model (Example 5.1), and a challenge model (Example 5.2).

5.1. Serum Analysis of Mice Vaccinated with Various CSP Based mRNA Vaccines:

Approximately 2 ml serum samples of mice vaccinated with LNP formulated CSP based vaccines are analyzed with an established ELISA model with recombinant CSP and sporozoites as positive controls.

Furthermore, 500 ul mouse sera are analyzed in a passive transfer model: CSP antibodies in serum samples are passively transferred to mice (n=4) that have been infected 2 h or 16 h following injection with Plasmodium berghei-Plasmodium falciparum CSP chimeric sporozoites or by 5 infectious mosquito bites. Mice are then evaluated for reduced parasite burden in the liver.

5.2. Challenge Study of Mice Vaccinated with Various CSP Based mRNA Vaccines:

LNP formulated mRNA-based Malaria vaccines are tested in a challenge model. Mice are vaccinated on days 0 and 21 and then challenged by 5 infectious mosquito bites with the transgenic Plasmodium berghei Malaria parasite (starting day 35 post vaccination). Mice are then evaluated for reduced parasite burden in the liver.

LNP-formulated mRNA vaccines encoding CSP are injected intramuscular on days 0 and 21. Starting on day 35 post vaccination, vaccinated mice are challenged by 5 infectious mosquito bites with the transgenic P. berghei parasite. The transgenic P. berghei parasite strain expresses the full-length P. falciparum CSP protein. These parasites generate highly infectious sporozoites in mice and mosquitoes.

### Example 6: Vaccination of Mice with LNP-Formulated mRNA Encoding CSP

The present example shows that Malaria mRNA vaccines encoding full length CSP induce strong humoral and cellular immune responses in mice.

Malaria mRNA constructs encoding full length CSP (Pf-CSP) or CSP(199-377) fragment with HBsAg (Pf-CSP(199-377)_Linker(PVTN)_HBsAg) were prepared according to Example 1. The mRNA was formulated in lipid nanoparticles GN01-LNPs (see Example 1.4.2). The different mRNA vaccine candidates were applied intramuscularly (i.m.; musculus tibialis, Balb/c mice) on days 0 and 21 and administered with doses of RNA, formulations, and administration routes as shown in Table 13A. One negative control group (3) received NaCl buffer. Serum samples were taken at day 21 and day 35 for determination of humoral immune responses. Splenocytes were taken at day 35 for determination of cellular immune responses.

TABLE 13A
Vaccination scheme of Example 6

| Group | No. of mice | Treatment | mRNA ID | Dose | Route | Volume |
|---|---|---|---|---|---|---|
| 1 | 6 | Pf-CSP | R7111 | 5 ug | i.m. | 1 × 25 uL |
| 2 | 6 | Pf-CSP(199-377)_Linker(PVTN)_HBsAg | R7271 | 5 ug | i.m. | 1 × 25 uL |
| 3 | 6 | NaCl buffer | | | i.m. | 1 × 25 uL |

### 6.1. Determination of Specific Humoral Immune Responses by ELISA:

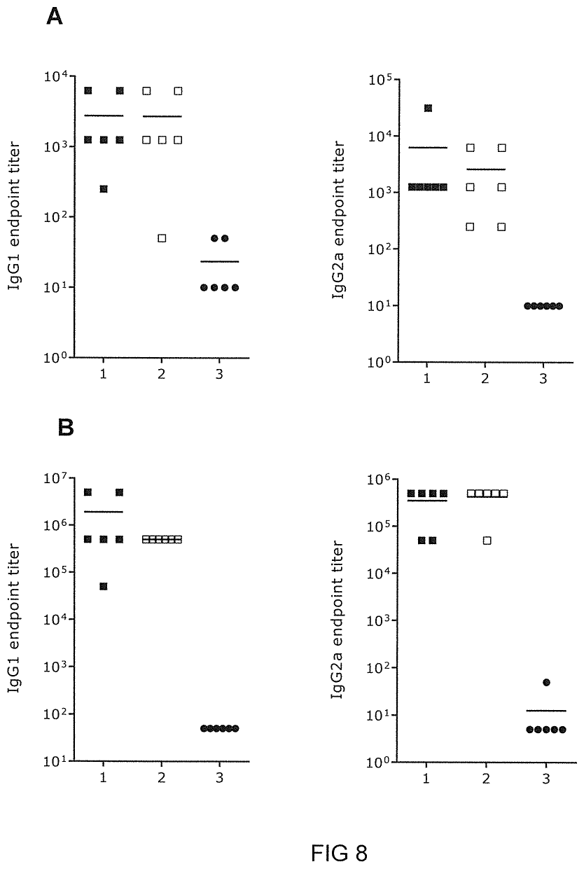

ELISA was performed essentially as described in Example 2.1. Results are shown in FIG. 8.

### 6.2. Intracellular Cytokine Staining:

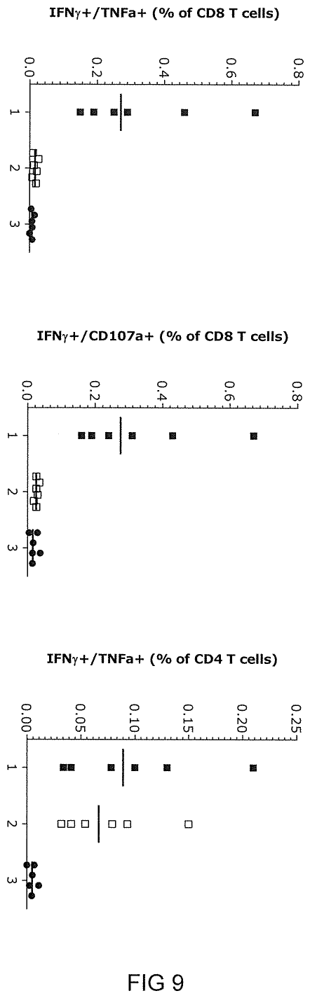

Splenocytes from vaccinated mice were isolated on day 35 according to a standard protocol known in the art. Briefly, isolated spleens were grinded through a cell strainer and washed in PBS/1% FBS followed by red blood cell lysis. After an extensive washing step with PBS/1% FBS, splenocytes were seeded into 96-well plates (2×10⁶ cells per well). Cells were stimulated with CSP peptide library (0.5 ug/ml, ThermoFisher) according to SEQ ID NO: 10212-10276 and one CSP peptide (0.5 ug/ml, CSP_peptide_12 according to SEQ ID NO: 10208, EMC Microcollections GmbH, see Table Cm) in the presence of 2.5 ug/ml of an anti-CD28 antibody (BD Biosciences) and a protein transport inhibitor for 6 h at 37° C. After stimulation, cells were washed and stained for intracellular cytokines using the Cytofix/Cytoperm reagent (BD Biosciences) according to the manufacturer's instructions. The following antibodies were used for staining: Thy1.2-FITC (1:100), CD8-PE-Cy7 (1:200), TNF-PE (1:100), IFNγ-APC (1:100) (eBioscience), CD4-BD Horizon V450 (1:200) (BD Biosciences) and incubated with Fcγ-block diluted 1:100. Aqua Dye was used to distinguish live/dead cells (Invitrogen). Cells were acquired using a BD FACS Canto II flow cytometer (Beckton Dickinson). Flow cytometry data was analyzed using FlowJo software (Tree Star, Inc.). Results are shown in FIG. 9.

### Results:

As shown in FIG. 8, the LNP formulated CSP mRNA vaccines induced strong humoral immune responses in mice. Under the tested conditions, the LNP formulated vaccine applied to group 1 (full length CSP) (5 ug dose) induced slightly increased immune responses in comparison to the LNP formulated mRNA vaccine comprising CSP(199-377) fragment with HBsAg (Group 2; Table 12).

As shown in FIG. 9, the LNP formulated CSP mRNA vaccines induced cellular immune responses in mice (CD8+ and/or CD4+ T-cell responses). Notably, under the tested conditions, group 1 (full length CSP) (5 ug dose) showed strong CD8+ T-cell and CD4+ cell responses, whereas the LNP formulated mRNA vaccine comprising CSP(199-377) fragment with HBsAg (Group 2; Table 12) only showed CD4+ T-cell responses.

As CD8+ T cells may be a major protective immune mechanism against intracellular infections caused by Malaria parasites, an effective Malaria vaccine should induce strong CD8+ T cells responses. Accordingly, these findings highlight one of the advantageous features of the inventive mRNA-based malaria vaccine. The more full-length CSP as an antigen induce broader humoral and especially cellular antibody responses compared to the truncated LNP formulated mRNA vaccine comprising CSP(199-377) fragment with HBsAg. The more full-length CSP may provide additional T cell epitopes, leading to increased cellular immunity, which could potentially enhance protection against Malaria.

### Example 7: Vaccination of Mice with LNP-Formulated mRNA Encoding CSP

The present example shows that the C-terminus of CSP is important for the mRNA Malaria vaccine to induce CD4+-T cell responses.

Malaria mRNA vaccine constructs encoding CSP variants (e.g. comprising a heterologous transmembrane domain (group 1), a deletion mutant of the GPI anchor (group 2), or a C-terminal shortened/deleted CSP variant (group 3)) were prepared according to Example 1. The mRNA was formulated in lipid nanoparticles GN01-LNPs (see Example 1.4.2). The different mRNA vaccine candidates were applied intramuscularly (i.m.; musculus tibialis, Balb/c mice) on days 0 and 21 and administered with doses of RNA, formulations, and administration routes as shown in Table 13B. One negative control group (4) received irrelevant RNA (polycytidylic acid (poly(C) RNA) (Sigma)). Serum samples were taken at day 21, and day 35 for determination of humoral immune responses. Splenocytes were taken at day 35 for determination of cellular immune responses.

TABLE 13B
Vaccination scheme of Example 7

| Group | No. of mice | Treatment | mRNA ID | Dose | Route | Volume |
|---|---|---|---|---|---|---|
| 1 | 8 | HsALB_Pf-CSP(19-384)_TM domain HA | R7641 | 1 ug | i.m. | 1 × 20 uL |
| 2 | 8 | HsALB_Pf-CSP(19-384) (without GPI- | R7642 | 1 ug | i.m. | 1 × 20 uL |
| | | anchor) | | | | |
| 3 | 8 | HsALB_Pf-CSP(19-272) | R7647 | 1 ug | i.m. | 1 × 20 uL |
| 4 | 5 | Poly: C | | 1 ug | i.m. | 1 × 20 uL |

### 7.1. Determination of Specific Humoral Immune Responses by ELISA:

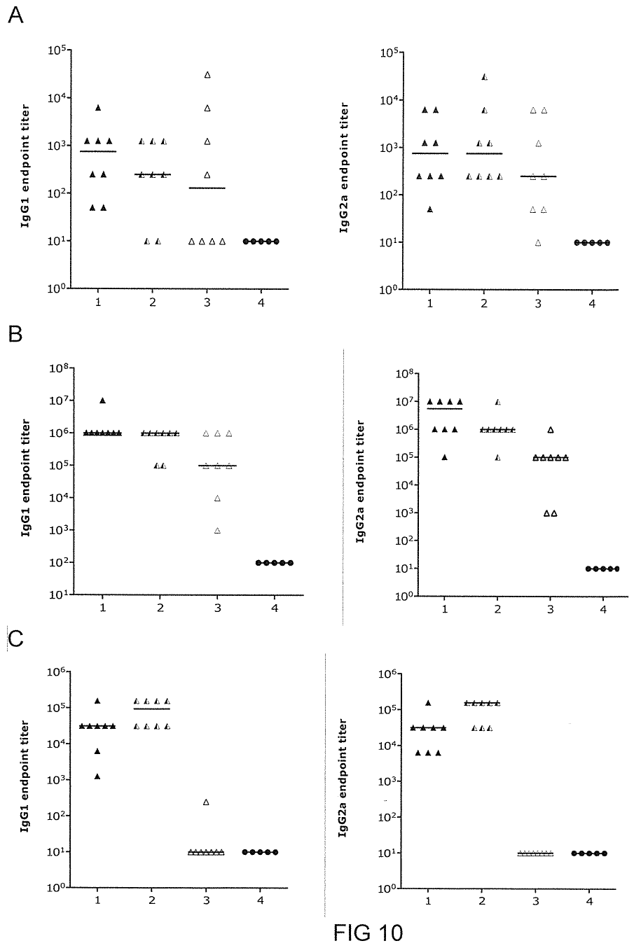

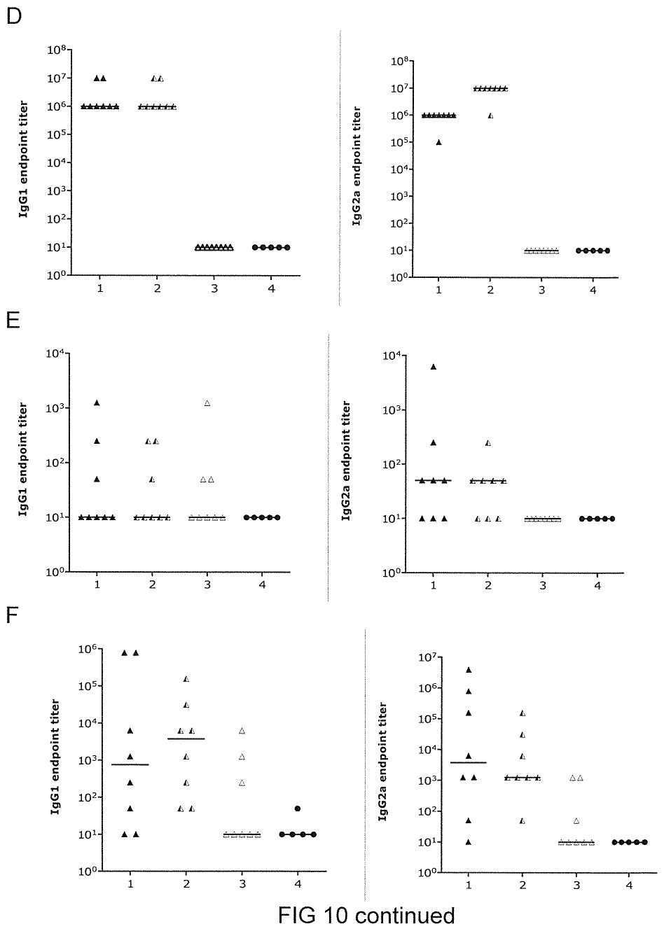

ELISA was performed using malaria [NANP]₇, C-term or N-term peptide for coating (according to SEQ ID NOs: 10209, 10211, 10210 respectively). Coated plates were incubated using respective serum dilutions, and binding of specific antibodies to the malaria [NANP]₇, C-term or N-term peptide, respectively were detected using biotinylated isotype specific anti-mouse antibodies followed by streptavidin-HRP (horse radish peroxidase) with Amplex as substrate. Endpoint titers of antibodies (IgG1, IgG2a) directed against the malaria [NANP]₇, C-term or N-term peptide, respectively were measured by ELISA on day 21 and day 35 post vaccinations. Results are shown in FIGS. 10 A-F.

### 7.2. Intracellular Cytokine Staining:

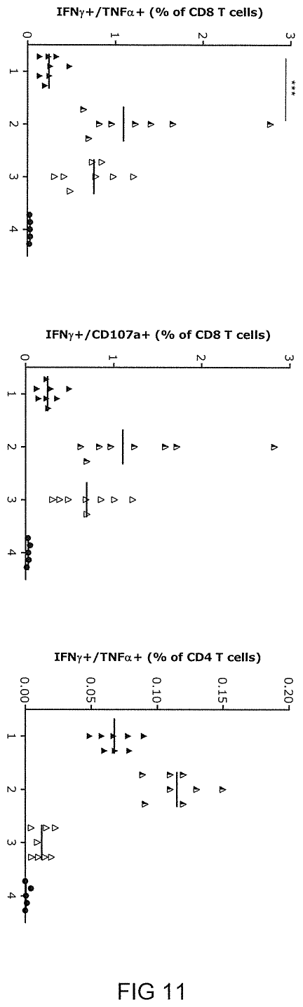

Splenocytes from vaccinated mice were isolated on day 35 according to a standard protocol known in the art. Intracellular cytokine staining was performed essentially as described in Example 2.3. The results are shown in FIG. 11.

### Results:

As shown in FIG. 10, all LNP formulated CSP mRNA vaccines induced strong, humoral immune responses in mice. Under the tested conditions, the “more full length” mRNA vaccines applied to group 1 and 2 (Table 13) induced the strongest immune responses. Accordingly, these findings show that epitopes in C-terminus are important to induce humoral immune response in general, as well against epitopes in the immunodominant NANP-region (see e.g. FIGS. 10 A and 10 B: Elisa analysis with NANP-coating material).

As shown in FIG. 11, all LNP formulated CSP mRNA vaccines induced cellular immune responses in mice (CD8+ and/or CD4+ T-cell responses). Notably, under the tested conditions, the mRNA vaccine without the GPI anchor (group 2, Table 13) showed stronger CD8+ T-cell and CD4+ T-cell responses as group 1 with the transmembrane domain of HA and group 3 the C-terminal shortened CSP variant. These findings again show that epitopes in C-terminus are important to induce CD4+ T-cell responses, as the “more full length” mRNA (applied to group 2) in comparison to the mRNA vaccine encoding a shortened CSP variant (applied to group 3) leads to superior T-cell responses (CD4+ T-cells).

### Example 8: Vaccination of Mice with LNP-Formulated mRNA Encoding CSP

The present example shows that the C-terminus is important for the mRNA Malaria vaccine to induce CD4+-T cell responses.

Malaria mRNA constructs encoding CSP variants (comprising heterologous transmembrane domain (group 1), or a heterologous secretory signal peptide (group 1, 2, 3, 5), CSP constructs with a deletion of the GPI anchor (group 2), or shortened C-terminal CSP (group 3), CSP fragment with heterologous HBsAg (group 4)) were prepared according to Example 1. The mRNA was formulated in lipid nanoparticles (LNP-III-3) (see Example 1.4.1). The different mRNA vaccine candidates were applied intramuscularly (i.m.; musculus tibialis, Balb/c mice) on days 0 and 21 and administered with doses of RNA, formulations, and administration routes as shown in Table 14. One negative control group (7) received irrelevant RNA (polycytidylic acid (poly(C) RNA) (Sigma). Serum samples were taken at day 21, and day 35 for determination of humoral immune responses. Splenocytes were taken at day 35 for determination of cellular immune responses.

TABLE 14
Vaccination scheme of Example 8

| | No. of | | mRNA | | | |
|---|---|---|---|---|---|---|
| Group | mice | Treatment | ID | Dose | Route | Volume |
| 1 | 8 | HsALB_Pf-CSP(19-384)_TM domain HA | R7641 | 1 ug | i.m. | 1 × 20 uL |
| 2 | 8 | HsALB_Pf-CSP(19-384) (without GPI-anchor) | R7642 | 1 ug | i.m. | 1 × 20 uL |
| 3 | 8 | HsALB_Pf-CSP(19-272) | R7647 | 1 ug | i.m. | 1 × 20 uL |
| 4 | 8 | Pf-CSP(199-377)_Linker(PVTN)_HBsAg | R7271 | 1 ug | i.m. | 1 × 20 uL |
| 5 | 8 | HsALB_Pf-CSP(19-397) | R7267 | 1 ug | i.m. | 1 × 20 uL |
| 6 | 8 | Pf-CSP | R7111 | 1 ug | i.m. | 1 × 20 uL |
| 7 | 5 | Poly: C | | 1 ug | i.m. | 1 × 20 uL |

### 8.1. Determination of Specific Humoral Immune Responses by ELISA:

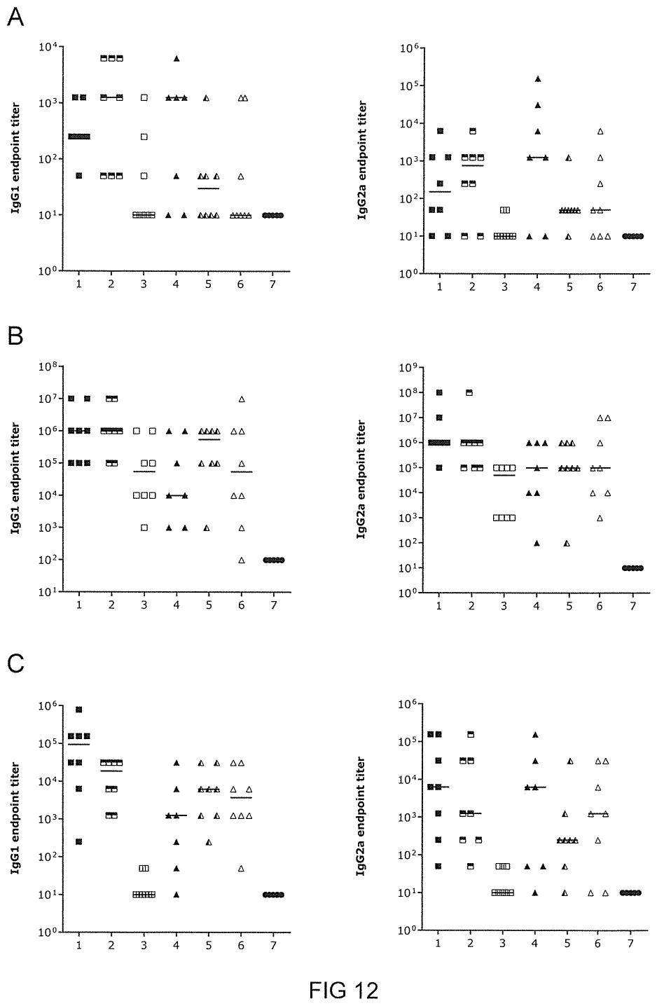

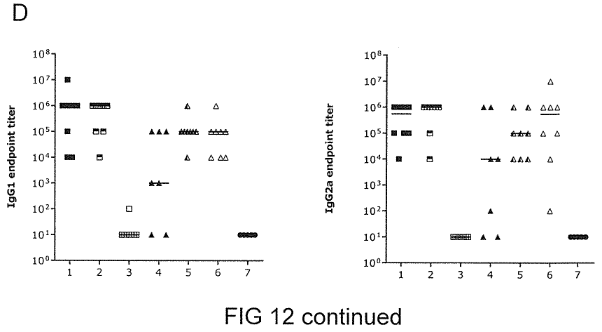

ELISA was performed essentially as described in Example 7.1. Results are shown in FIGS. 12 A-D.

### 8.2. Intracellular Cytokine Staining:

Splenocytes from vaccinated mice were isolated on day 35 according to a standard protocol known in the art.

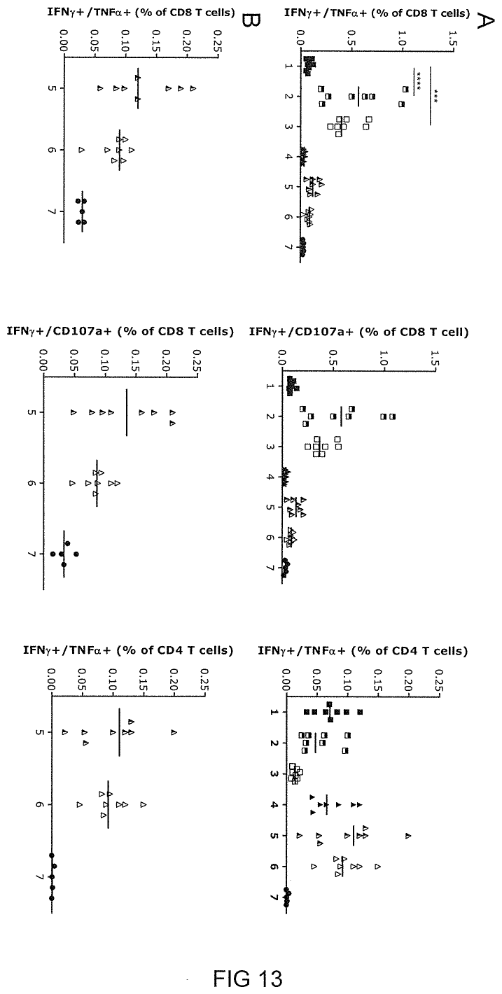

Intracellular cytokine staining was performed essentially as described in Example 2.3. The results are shown in FIG. 13.

### Results:

As shown in FIG. 12, nearly all LNP formulated CSP mRNA vaccines induced strong, humoral immune responses in mice. Under the tested conditions, the more full length” mRNA vaccines applied to group 1, 2 and 5 with the heterologous secretory signal peptide compared to a shortened variant (applied to group 3) or the full length with the natural signal peptide (applied to group 6) (Table 14) induced stronger humoral immune responses. Accordingly, these findings show that epitopes in C-terminus are important to induce humoral immune responses against epitopes in the immunodominant NANP-region (see e.g. FIGS. 12A and 12B: ELISA analysis with [NANP]₇ coating).

As shown in FIG. 13 A, all LNP formulated CSP mRNA vaccines induced cellular immune responses in mice (CD8+ and/or CD4+ T-cell responses). Notably, under the tested conditions, the mRNA vaccine with a deletion of the GPI anchor (group 2, Table 14) showed strongest CD8+ T-cell responses. The mRNA vaccine with the shortest C-terminus (group 3, Table 14) showed low CD4+ T-cell response. These findings again show that epitopes in C-terminus are important to induce especially CD4+ T-cell response, as the “more full length” mRNA (applied to group 1, 2, 5 and 6) in comparison to the mRNA vaccine encoding a shortened CSP variant (applied to group 3) leads to superior CD4+ T-cell responses.

As shown in FIG. 13A, and for better visibility in FIG. 13B, under the tested conditions the mRNA Malaria vaccine encoding with a heterologous secretory signal peptide (human serum albumin signal peptide, group 5, Table 14) showed better CD8+ T-cell and slightly better CD4+ T-cell responses compared to the mRNA Malaria vaccine with the wild type signal peptide (group 6, Table 14).

### Example 9: Vaccination of Mice with LNP-Formulated mRNA Encoding CSP

The present example shows that mRNA vaccines comprising different T cell epitopes at the C-terminus of the CSP induces different pronounced humoral as well as cellular immune responses.

Malaria mRNA constructs encoding CSP with different C-terminus variants were prepared according to Example 1. The mRNA was formulated in lipid nanoparticles (LNP-III-3) (see Example 1.4.1). The different mRNA vaccine candidates were applied intramuscularly (i.m.; musculus tibialis, Balb/c mice) on days 0 and 21 and administered with doses of RNA, formulations, and administration routes as shown in Table 15. One negative control group (9) received irrelevant RNA. Serum samples were taken at day 21, and day 35 for determination of humoral immune responses. Splenocytes were taken at day 35 for determination of cellular immune responses.

TABLE 15
Vaccination scheme of Example 9

| | No. of | | mRNA | | | |
|---|---|---|---|---|---|---|
| Group | mice | Treatment | ID | Dose | Route | Volume |
| 1 | 8 | HsALB_Pf-CSP(19-384) | R7642 | 1 ug | i.m. | 1 × 20 uL |
| 2 | 8 | HsALB_Pf-CSP(19-272)_Linker(AAY)_Pf-CSP(310- | R8100 | 1 ug | i.m. | 1 × 20 uL |
| | | 327)_Linker(AAY)_Pf-CSP(346-375) | | | | |
| 3 | 8 | HsALB_Pf-CSP(19-272)_Linker(AAY)_Pf-CSP(346- | R8101 | 1 ug | i.m. | 1 × 20 uL |
| | | 365)_Linker(AAY)_PADRE | | | | |
| 4 | 8 | HsALB_Pf-CSP(19-272)_Linker(AAY)_Pf-CSP(346- | R8102 | 1 ug | i.m. | 1 × 20 uL |
| | | 365)_Linker(AAY)_P2 | | | | |
| 5 | 8 | HsALB_Pf-CSP(19-272)_Linker(G4S)_Pf-CSP(310- | R8103 | 1 ug | i.m. | 1 × 20 uL |
| | | 327)_Linker(G4S)_Pf-CSP(346-375) | | | | |
| 6 | 8 | HsALB_Pf-CSP(19-272)_Linker(G4S)_Pf-CSP(310- | R8104 | 1 ug | i.m. | 1 × 20 uL |
| | | 327)_Pf-CSP(346-375) | | | | |
| 7 | 8 | HsALB_Pf-CSP(199-377)_Linker(PVTN)_HBsAg | R8031 | 1 ug | i.m. | 1 × 20 uL |
| 8 | 8 | HsALB_Pf-CSP(19-397) | R7267 | 1 ug | i.m. | 1 × 20 uL |
| 9 | 5 | Irrelevant RNA | | 1 ug | i.m. | 1 × 20 uL |

### 9.1. Determination of Specific Humoral Immune Responses by ELISA:

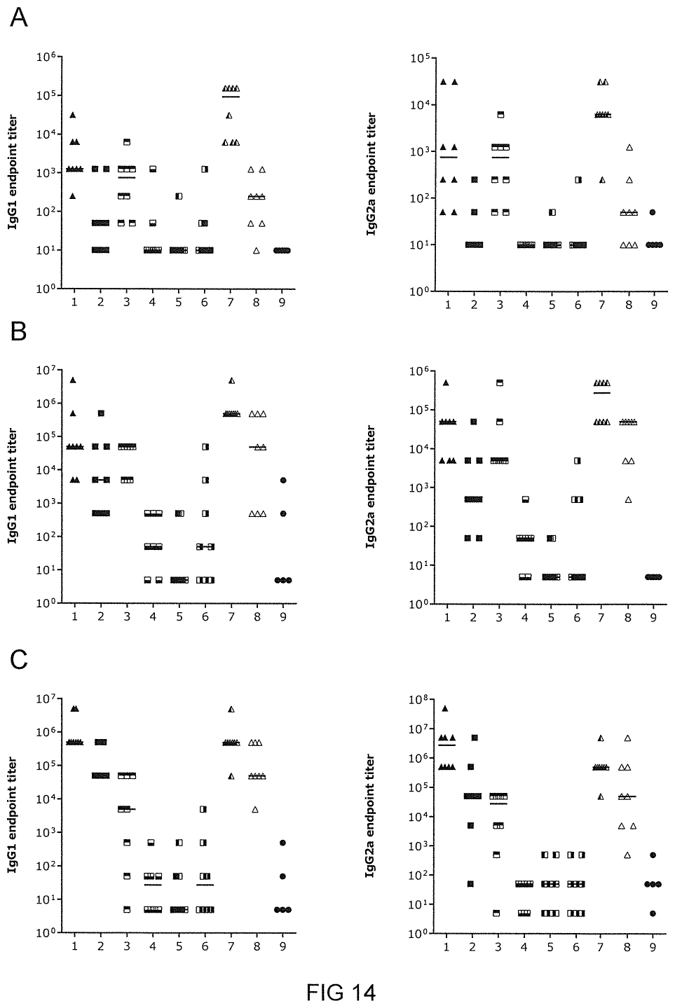

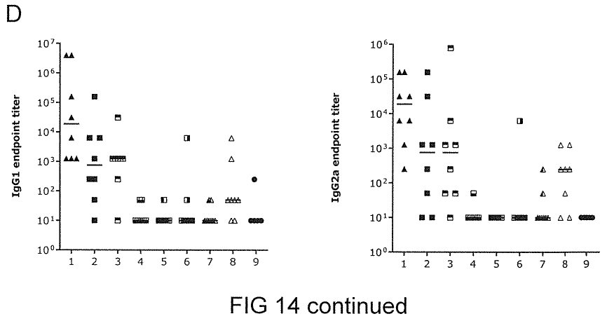

ELISA was performed essentially as described in Example 7.1. Results are shown in FIGS. 14 A-D.

### 9.2. Intracellular Cytokine Staining:

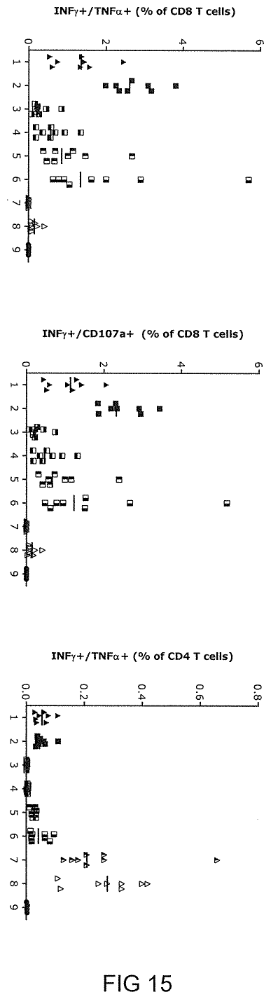

Splenocytes from vaccinated mice were isolated on day 35 according to a standard protocol known in the art. Briefly, isolated spleens were grinded through a cell strainer and washed in PBS/1% FBS followed by red blood cell lysis. After an extensive washing step with PBS/1% FBS, splenocytes were seeded into 96-well plates (2×10⁶ cells per well). Cells were stimulated with a mixture of CSP peptides (1 ug/ml, EMC Microcollections GmbH) (see Table C) for CD4+ T-cell stimulation or with a CSP peptide library (0.5 ug/ml, ThermoFisher) according to SEQ ID NO: 10212-10276 for CD8+ T-cell stimulation in the presence of 2.5 ug/ml of an anti-CD28 antibody (BD Biosciences) and a protein transport inhibitor for 6 h at 37° C. After stimulation, cells were washed and stained for intracellular cytokines using the Cytofix/Cytoperm reagent (BD Biosciences) according to the manufacturer's instructions. The following antibodies were used for staining: Thy1.2-FITC (1:100), CD8-PE-Cy7 (1:200), TNF-PE (1:100), IFNγ-APC (1:100) (eBioscience), CD4-BD Horizon V450 (1:200) (BD Biosciences) and incubated with Fcγ-block diluted 1:100. Aqua Dye was used to distinguish live/dead cells (Invitrogen). Cells were acquired using a BD FACS Canto II flow cytometer (Beckton Dickinson). Flow cytometry data was analyzed using FlowJo software (Tree Star, Inc.). Results are shown in FIG. 15.

### Results:

As shown in FIGS. 14 and 15, nearly all LNP formulated CSP mRNA vaccines induced strong, humoral and/or cellular immune responses in mice.

Under the tested conditions the constructs with C-terminus _Linker(AAY)_Pf-CSP(310-327)_Linker(AAY)_Pf-CSP(346-375) and _Linker(AAY)_Pf-CSP(346-365)_Linker(AAY)_PADRE (R8100 and R8101, group 2 and 3, Table 15) showed strong humoral immune response. Construct with _Linker(AAY)_Pf-CSP(310-327)_Linker(AAY)_Pf-CSP(346-375) (R8100, group 2, Table 15) showed also strong CD8+ T-cell response as well as constructs with _Linker(G4S)_Pf-CSP(310-327)_Pf-CSP(346-375) (R8104, group 6, Table 15) which also showed strong CD8+ T-cell response. These two constructs (R8100 and R8104, group 2 and 6, Table 15) also showed best CD4+ T-cell response of the tested C-terminus constructs.

The relative location between the T-cell epitopes, e.g. reached by the introduction of heterologous linker elements also influenced the induction of humoral or cellular immune responses. The mRNA vaccine comprising e.g. the introduced AAY linker for example (R8100, group 2, Table 15) induced stronger humoral immune response compared to G4S linker and therefore a changed location of the T-cell epitopes (R8103, group 5).

The findings also showed that the direct linking of epitopes like in construct HsALB_Pf-CSP(19-272)_Linker(G4S)_Pf-CSP(310-327)_Pf-CSP(346-375) (R8104, group 6, Table 15) induced stronger CD8+ T-cell response than the separation with an additional linker HsALB_Pf-CSP(19-272)_Linker(G4S)_Pf-CSP(310-327)_Linker(G4S)_Pf-CSP(346-375) (R8103, group 5, Table 15).

Therefore it is very important to test different combinations of linkers and epitopes to induce the best humoral as well as cellular immune response. Each of the tested combination showed its power to induce immune response at different stages. A vaccine composition comprising different mRNAs encoding different CSP protein designs might be a powerful tool to reach balanced and powerful humoral and cellular immune responses.

### Example 10: Vaccination of Mice with LNP-Formulated mRNA Encoding CSP

The present example shows that different length of N-terminus as well as of NANP repeat regions at the C-terminus of the Malaria mRNA vaccine induces different humoral and cellular immune responses.

Malaria mRNA constructs encoding CSP with different NANP repeat region variants at the C-terminus and N-terminus variants were prepared according to Example 1. The mRNA was formulated in lipid nanoparticles (LNP-III-3) (see Example 1.4.1). The different mRNA vaccine candidates were applied intramuscularly (i.m.; musculus tibialis, Balb/c mice) on days 0 and 21 and administered with doses of RNA, formulations, and administration routes as shown in Table 16. One negative control group (9) received irrelevant RNA. Serum samples were taken at day 21, and day 35 for determination of humoral immune responses. Splenocytes were taken at day 35 for determination of cellular immune responses.

TABLE 16
Vaccination scheme of Example 10

| | No. of | | mRNA | | | |
|---|---|---|---|---|---|---|
| Group | mice | Treatment | ID | Dose | Route | Volume |
| 1 | 8 | HsALB_Pf-CSP(19-272) | R7647 | 1 ug | i.m. | 1 × 20 uL |
| 2 | 8 | HsALB_Pf-CSP(19-192) | R8090 | 1 ug | i.m. | 1 × 20 uL |
| 3 | 8 | HsALB_Pf-CSP(19-152) | R8091 | 1 ug | i.m. | 1 × 20 uL |
| 4 | 8 | HsALB_Pf-CSP(82-397) | R8092 | 1 ug | i.m. | 1 × 20 uL |
| 5 | 8 | HsALB_Pf-CSP(93-397) | R8089 | 1 ug | i.m. | 1 × 20 uL |
| 6 | 8 | HsALB_Pf-CSP(98-397) | R7649 | 1 ug | i.m. | 1 × 20 uL |
| 7 | 8 | HsALB_Pf-CSP(199-377)_Linker(PVTN)_HBsAg | R8031 | 1 ug | i.m. | 1 × 20 uL |
| 8 | 8 | HsALB_Pf-CSP(19-397) | R7267 | 1 ug | i.m. | 1 × 20 uL |
| 9 | 5 | Irrelevant RNA | | 1 ug | i.m. | 1 × 20 uL |

### 10.1. Determination of Specific Humoral Immune Responses by ELISA:

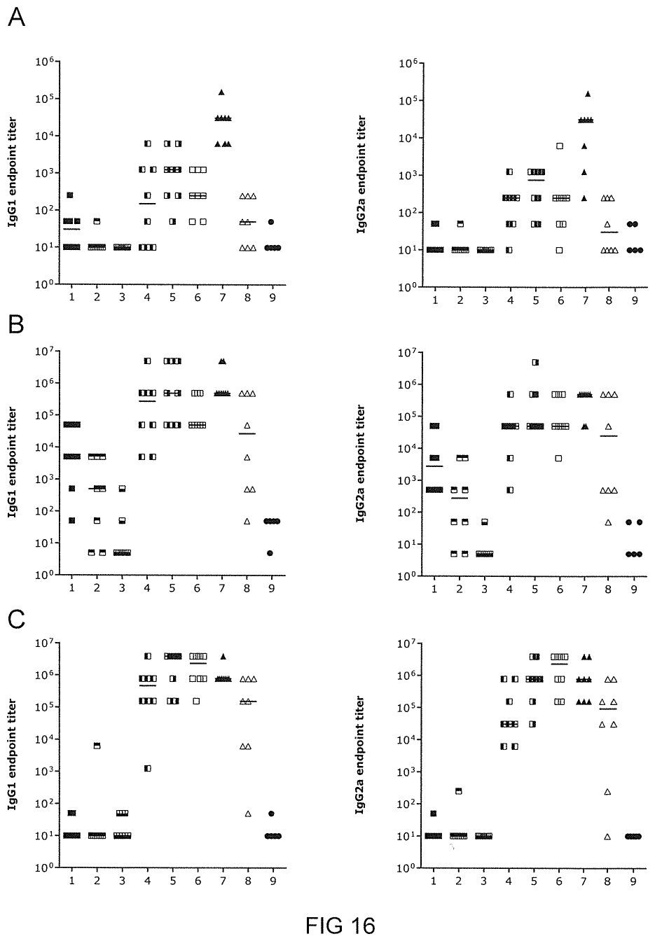

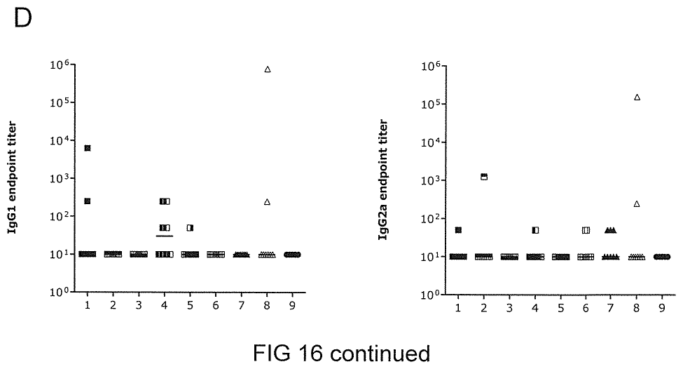

ELISA was performed essentially as described in Example 7.1. Results are shown in FIGS. 16 A-D.

### 10.2. Intracellular Cytokine Staining:

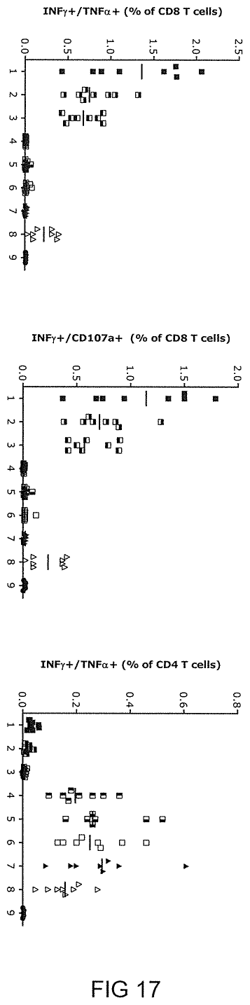

Splenocytes from vaccinated mice were isolated on day 35 according to a standard protocol known in the art. Intracellular cytokine staining was performed essentially as described in Example 9.2. The results are shown in FIG. 17.

### Results:

As shown in FIG. 16, nearly all LNP formulated CSP mRNA vaccines induced humoral immune responses in mice. Under the tested conditions, the mRNA vaccine with shortened NANP repeats at the C-terminus (groups 1-3, Table 16) showed less antibody response compared to mRNA vaccines shortened at the N-terminus (groups 4-6, Table 16). Accordingly, these findings show that epitopes in C-terminus and NANP repeat region are important to induce humoral immune responses.

As shown in FIG. 17, all LNP formulated CSP mRNA vaccines induced cellular immune responses in mice (CD8+ and/or CD4+ T-cell responses). Notably, under the tested conditions, the mRNA vaccine with shortened NANP repeats at the C-terminus (groups 1-3, Table 16) showed strongest CD8+ T-cell response but less CD4+ T-cell response and vice versa mRNA vaccines with shortened N-terminus (groups 4-6, Table 16) showed strongest CD4+ T-cell but less CD8+ T-cell response. Accordingly, these findings show that epitopes in N-terminus are important to induce a CD8+ T-cell response whereas epitopes in the C-terminus are important to induce a CD4+ T-cell immune response.

### Example 11: Vaccination of Mice with LNP-Formulated mRNA Encoding CSP

The present example shows that mRNA Malaria vaccines comprising differently capped mRNA (cap1 or cap0) induce different humoral as well as cellular immune responses.

Malaria mRNA constructs encoding CSP were capped differently and were prepared according to Example 1. The mRNA was formulated in lipid nanoparticles (LNP-III-3) (see Example 1.4.1). The different mRNA vaccine candidates were applied intramuscularly (i.m.; musculus tibialis, Balb/c mice) on days 0 and 21 and administered with doses of RNA, formulations, and administration routes as shown in Table 17. One negative control group (3) received irrelevant RNA. Serum samples were taken at day 21, and day 35 for determination of humoral immune responses. Splenocytes were taken at day 35 for determination of cellular immune responses.

TABLE 17
Vaccination scheme of Example 11

| Group | No. of mice | Treatment | Capping | mRNA ID | Dose | Route | Volume |
|---|---|---|---|---|---|---|---|
| 1 | 8 | HsALB_Pf-CSP(19-384) | cap0 | R7642 | 1 ug | i.m. | 1 × 20 uL |
| 2 | 8 | HsALB_Pf-CSP(19-384) | cap1 | R8230 | 1 ug | i.m. | 1 × 20 uL |
| 3 | 5 | Irrelevant RNA | | | 1 ug | i.m. | 1 × 20 uL |

### 11.1. Determination of Specific Humoral Immune Responses by ELISA:

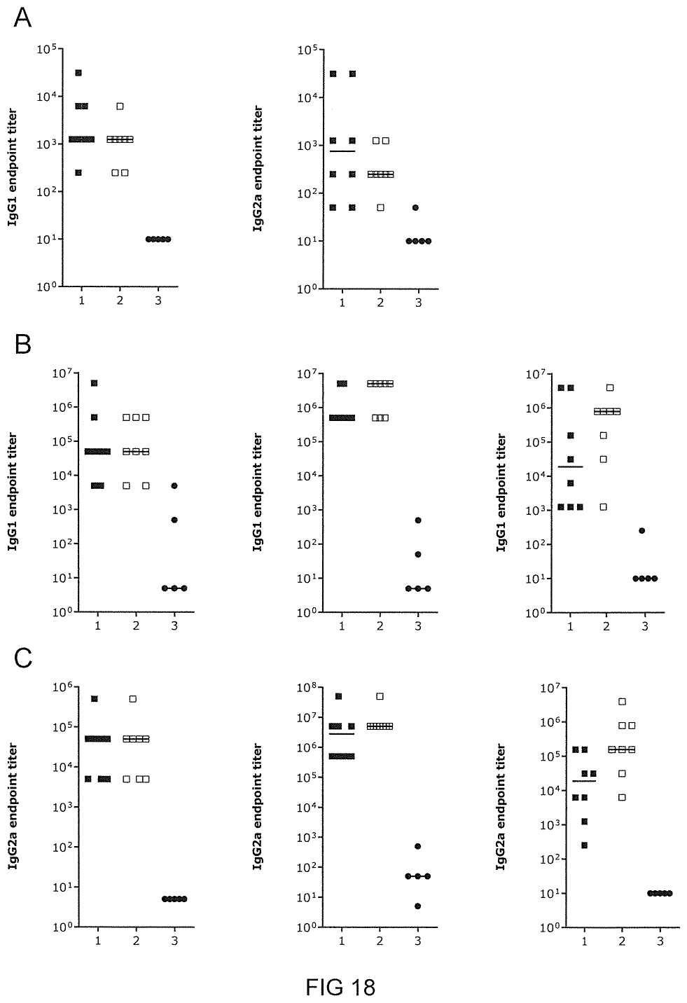

ELISA was performed essentially as described in Example 7.1. Results are shown in FIG. 18.

### 11.2. Intracellular Cytokine Staining:

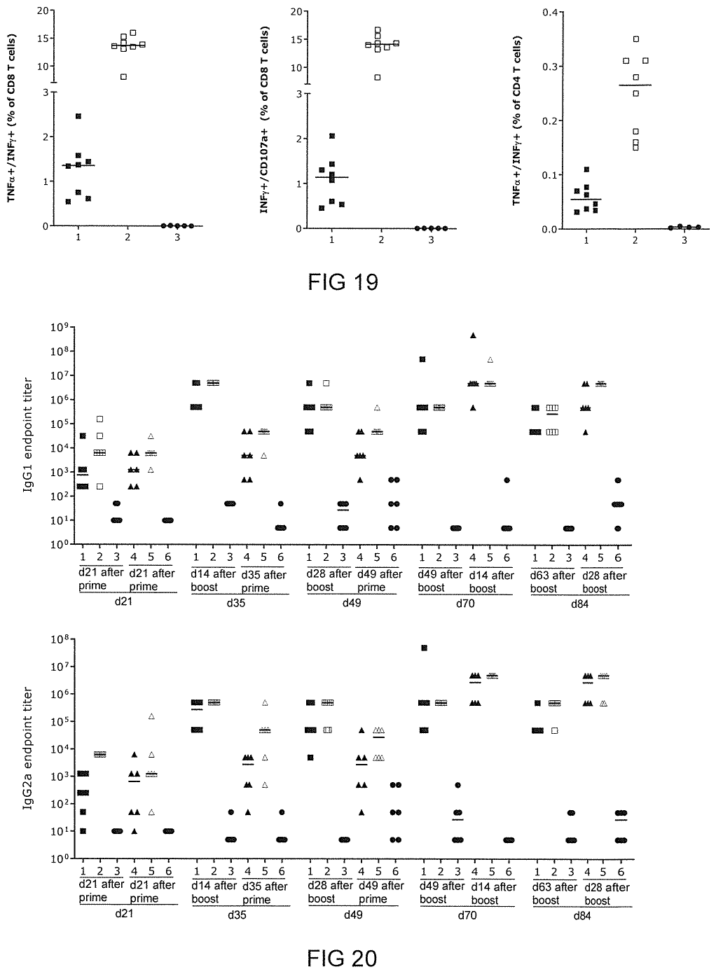

Splenocytes from vaccinated mice were isolated on day 35 according to a standard protocol known in the art. Intracellular cytokine staining was performed essentially as described in Example 9.2. The results are shown in FIG. 19.

### Results:

As shown in FIG. 18, independently which capping is used all LNP formulated CSP mRNA vaccines induced strong humoral immune responses in mice.

As shown in FIG. 19, all LNP formulated CSP mRNA vaccines induced cellular immune responses in mice (CD8+ and/or CD4+ T-cell responses). Notably, under the tested conditions, the mRNA vaccine with cap1 (group 2, Table 18) showed very strong CD8+ T-cell response and also strong CD4+ T-cell response.

### Example 12: Vaccination of Mice with LNP-Formulated mRNA Encoding CSP

The present example shows that differently capped mRNA Malaria vaccine induces different humoral as well as cellular immune response and that a longer interval between prime and boost vaccination can induce stronger immune responses.

Malaria mRNA constructs encoding CSP were capped differently and prepared according to Example 1. The mRNA was formulated in lipid nanoparticles (LNP-III-3) (see Example 1.4.1). The different mRNA vaccine candidates were applied intramuscularly (i.m.; musculus tibialis, Balb/c mice) on day 0 and 21 or 56 and administered with doses of RNA, formulations, and administration routes as shown in Table 18. Two negative control groups (3 and 6) received NaCl buffer. Serum samples were taken at day 21, day 35, day 49, day 70, and day 84 for determination of humoral immune responses. Splenocytes were taken at day 84 for determination of cellular immune responses.

TABLE 18
Vaccination scheme of Example 12

| | No. of | | | mRNA | | | | Vaccination |
|---|---|---|---|---|---|---|---|---|
| Group | mice | Treatment | Capping | ID | Dose | Route | Volume | at day |
| 1 | 6 | HsALB_Pf-CSP(19-397) | cap0 | R7267 | 5 ug | i.m. | 1 × 25 uL | 0 and 21 |
| 2 | 6 | HsALB_Pf-CSP(19-397) | cap1 | R8229 | 5 ug | i.m. | 1 × 25 uL | 0 and 21 |
| 3 | 6 | NaCl buffer | | | | i.m. | 1 × 25 uL | 0 and 21 |
| 4 | 6 | HsALB_Pf-CSP(19-397) | cap0 | R7267 | 5 ug | i.m. | 1 × 25 uL | 0 and 56 |
| 5 | 6 | HsALB_Pf-CSP(19-397) | cap1 | R8229 | 5 ug | i.m. | 1 × 25 uL | 0 and 56 |
| 6 | 6 | NaCl buffer | | | | i.m. | 1 × 25 uL | 0 and 56 |

### 12.1. Determination of Specific Humoral Immune Responses by ELISA:

ELISA was performed essentially as described in Example 7.1. Results are shown in FIG. 20.

### 12.2. Intracellular Cytokine Staining:

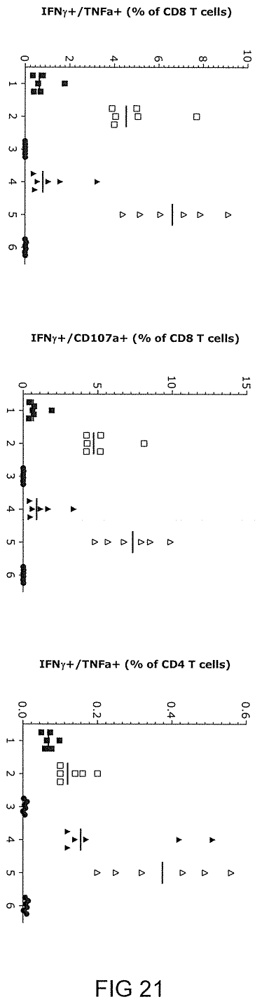

Splenocytes from vaccinated mice were isolated on day 84 according to a standard protocol known in the art. Intracellular cytokine staining was performed essentially as described in Example 6.2. The results are shown in FIG. 21.

### Results:

As shown in FIG. 20, all tested LNP formulated CSP mRNA vaccines induced very strong humoral immune responses in mice at a comparable level. The humoral immune response is enhanced with a later boost at day 56 instead of day 21 of LNP formulated CSP mRNA vaccine. A prolonged interval between prime and boost vaccination leads to increased humoral immune responses (shown for IgG1 and IgG2a endpoint titers (group 5 and 6, day 70 and 84). CSP vaccines comprising cap1 instead of cap0 show in general more pronounced immune responses, especially for the early time points.

As shown in FIG. 21, all LNP formulated CSP mRNA vaccines induced cellular immune responses in mice (CD8+ and/or CD4+ T-cell responses). Notably, under the tested conditions, the mRNA vaccine with cap1 showed very strong CD8+ T-cell response and also strong CD4+ T-cell response.

### Example 13: Vaccination of Mice with LNP-Formulated mRNA Encoding CSP

The present example shows that mRNA Malaria vaccines with mRNA comprising alternative forms of the 3′end (e.g. hSL-A64-N5 or hSL-A100), induce strong humoral as well as cellular immune response in mice. Furthermore, mRNA vaccine comprising mRNA with chemically modified nucleotides (e.g. pseudourinine ψ) induces immune responses.

Malaria mRNA vaccines comprising mRNA constructs with different 3′-ends encoding CSP were prepared according to Example 1. The mRNA was formulated in lipid nanoparticles (LNP-III-3) (see Example 1.4.1). The different mRNA vaccine candidates were applied intramuscularly (i.m.; musculus tibialis, Balb/c mice) on day 0 and day 21 and administered with doses of RNA, formulations, and administration routes as shown in Table 18. One negative control group (7) received NaCl buffer. Serum samples were taken at day 21 and day 35 for determination of humoral immune responses. Splenocytes were taken at day 84 for determination of cellular immune responses.

TABLE 19
Vaccination scheme of Example 13

| | No. of | | Modifi- | | mRNA | | | |
|---|---|---|---|---|---|---|---|---|
| Group | mice | Treatment | cation | 3′-end | ID | Dose | Route | Volume |
| 1 | 8 | HsALB_Pf-CSP(19-397) | non | hSL-A64-N5 | R8523 | 1 ug | i.m. | 1 × 25 uL |
| 2 | 8 | HsALB_Pf-CSP(19-397) | non | hSL-A100 | R8520 | 1 ug | i.m. | 1 × 25 uL |
| 3 | 8 | HsALB_Pf-CSP(19-397) | ψ | hSL-A64-N5 | R8525 | 1 ug | i.m. | 1 × 25 uL |
| 4 | 8 | HsALB_Pf-CSP(19-397) | ψ | hSL-A100 | R8526 | 1 ug | i.m. | 1 × 25 uL |
| 5 | 8 | HsALB_Pf-CSP(199- | non | hSL-A100 | R8564 | 1 ug | i.m. | 1 × 25 uL |
| | | 377)_Linker(PVTN)_HBsAg | | | | | | |
| 6 | 8 | HsALB_Pf-CSP(93- | non | hSL-A100 | R8574 | 1 ug | i.m. | 1 × 25 uL |
| | | 384)_Linker(PVTN)_HBsAg | | | | | | |
| 7 | 8 | NaCl buffer | | | | | | |

### 13.1. Determination of Specific Humoral Immune Responses by ELISA:

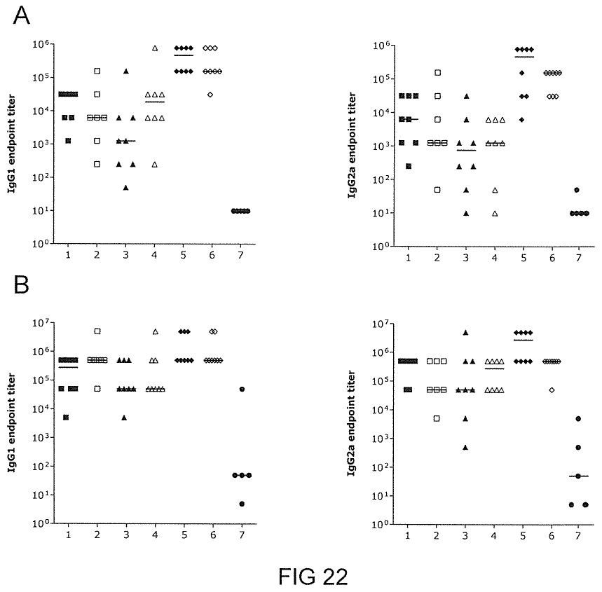

ELISA was performed essentially as described in Example 5.1. Results are shown in FIG. 22. 13.2. Intracellular Cytokine Staining:

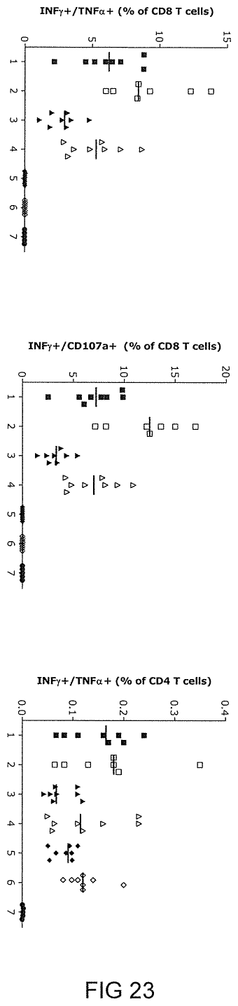

Splenocytes from vaccinated mice were isolated on day 35 according to a standard protocol known in the art. Intracellular cytokine staining was performed essentially as described in Example 6.2. The results are shown in FIG. 23.

### Results:

As shown in FIG. 22, independently which 3′-end is used all LNP formulated CSP mRNA vaccines induced very strong humoral immune responses in mice. Furthermore, mRNA vaccine comprising mRNA with chemically modified nucleotides (e.g. pseudourinine ψ) induces humoral immune responses as well.

As shown in FIG. 23, independently which 3′-end is used all LNP formulated CSP mRNA vaccines induced strong cellular immune responses in mice (CD8+ and/or CD4+ T-cell responses). Notably, under the tested conditions, the mRNA vaccine comprising pseudouridine (ψ) modified nucleotides showed lower CD8+ T-cell and CD4+ T-cell response than mRNA vaccine comprising non-modified nucleotides. Independently which 3′-end is used strong humoral as well as cellular immune responses are generated.

### Example 14: Expression Analysis of Different mRNA Construct Encoding CSP in 293T Cells

The present example shows that mRNA constructs with alternative 3′-ends encoding different CSP constructs are expressed in mammalian cells.

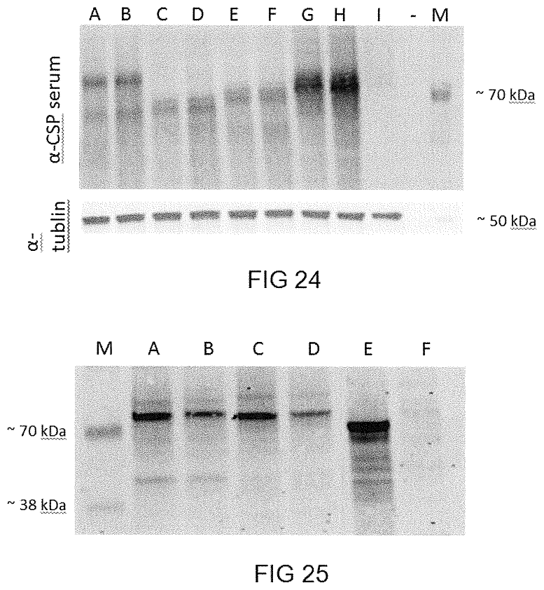

To determine in vitro protein expression of some of the RNA constructs, 293T cells were transiently transfected with mRNA encoding CSP antigen. 24 h prior to transfection, 293T cells were seeded in a 6-well plate at a density of 500,000 cells/well in cell culture medium. Cells were transfected with 1 ug RNA using Lipofectamine 2000 (Invitrogen) as transfection agent. Cell lysates were subjected to SDS-PAGE and Western Blot analysis was performed as commonly known in the art, using rabbit anti-CSP falciparum serum (Alpha Diagnostics) against a part of the repeat region of CSP and the C-terminus (1:1000 diluted) or mouse anti-alpha-tubulin antibody (1:1000; Abcam) as primary antibodies in combination with secondary goat anti-rabbit antibody IgG IRDye 800CW (Li-Cor, 1:10000 diluted) or goat anti-mouse IgG IRDye® 680RD (Li-Cor, 1:100000 diluted) (see FIG. 24). Detection and quantification was performed using a Li-Cor detection system (Odyssey CLx image system) in combination with Image Studio Lite software. Table 19 contains mRNA constructs that were used in the experiment:

TABLE 20
RNA constructs used for western blot analysis

| Group | Treatment | 3′-end | mRNA ID |
|---|---|---|---|
| A | HsALB_Pf-CSP(19-384)_Linker(PVTN)_HBsAg | A64-N5-C30-hSL-N5 | R8565 |
| B | HsALB_Pf-CSP(19-384)_Linker(PVTN)_HBsAg | hSL-A100 | R8566 |
| C | HsALB_Pf-CSP(19-272)_Linker(PVTN)_HBsAg | A64-N5-C30-hSL-N5 | R8567 |
| D | HsALB_Pf-CSP(19-272)_Linker(PVTN)_HBsAg | hSL-A100 | R8568 |
| E | HsALB_Pf-CSP(19-272)_Linker(AAY)_Pf-CSP(346- | A64-N5-C30-hSL-N5 | R8571 |
| | 365)_Linker(AAY)_PADRE_Linker(PVTN)_HBsAg | | |
| F | HsALB_Pf-CSP(19-272)_Linker(AAY)_Pf-CSP(346- | hSL-A100 | R8572 |
| | 365)_Linker(AAY)_PADRE_Linker(PVTN)_HBsAg | | |
| G | HsALB_Pf-CSP(93-384)_Linker(PVTN)_HBsAg | A64-N5-C30-hSL-N5 | R8573 |
| H | HsALB_Pf-CSP(93-384)_Linker(PVTN)_HBsAg | hSL-A100 | R8574 |
| I | Water | | |
| M | Protein size marker | | |

### Results:

As shown in FIG. 24 all tested mRNA constructs expressed detectable CSP proteins, which is a prerequisite for an mRNA-based Malaria vaccine.

### Example 15: Expression Analysis of Different mRNA Construct Encoding CSP

To determine in vitro protein expression of the mRNA constructs, the constructs with different heterologous N-terminus (group A and B, Table 20) or with different heterologous signal peptides (group C and D, Table 20) were mixed with components of Promega Rabbit Reticulocyte Lysate System according to manufacture protocol. The lysate contains the cellular components necessary for protein synthesis (tRNA, ribosomes, amino acids, initiation, elongation and termination factors). As positive control Luciferase RNA from Lysate System Kit was used. The translation result was analyzed by SDS-Page and Western Blot analysis (IRDye 800CW streptavidin antibody (1:2000)). Table 20 summarizes the tested RNA constructs.

TABLE 21
Overview of mRNA constructs used in Example 15

| Group | Treatment | 3′-end | mRNA ID |
|---|---|---|---|
| A | HsALB_Pf-CSP(19-384)_Linker(SGG)_Ferritin | A64-N5-C30-hSL-N5 | R8780 |
| B | HsALB_Pf-CSP(19-384)_Linker(SGG)_Ferritin | hSL-A100 | R8781 |
| C | LumSynt_Linker(GGS4-GGG)_Pf-CSP(19-397) | A64-N5-C30-hSL-N5 | R8782 |
| D | LumSynt_Linker(GGS4-GGG)_Pf-CSP(19-397) | hSL-A100 | R8783 |
| E | control RNA from Lysate System Kit | | |
| F | RNAse free water | | |
| M | Protein size marker | | |

### Results:

As shown in FIG. 25 the used mRNA constructs led to a detectable protein expression, which is a prerequisite for an mRNA-based Malaria vaccine.

### Example 16: Functional Immunogenicity of Malaria mRNA Vaccine in a Challenge

The aim of the present example is to evaluate the functional immunogenicity of the inventive mRNA based Malaria vaccine with an improved ELISA assay, a passive transfer challenge model (Example 16.1), and an active challenge model (Example 16.2).

16.1. Serum Analysis of Mice Vaccinated with Various CSP Based mRNA Vaccines:

Serum samples of mice vaccinated with LNP formulated CSP based vaccines are analyzed with an established ELISA model with recombinant CSP and sporozoites as positive controls.

Furthermore, 400 ul mouse sera are analyzed in a passive transfer model: CSP antibodies in serum samples are passively transferred to mice (n=4) that have been infected 2 h following injection with Plasmodium berghei-Plasmodium falciparum CSP chimeric sporozoites (see Table 22). Mice are then evaluated for reduced parasite burden in the liver.

TABLE 22
Overview of passive transfer analysis
according Example 16.1.

| | No. of | | | | |
|---|---|---|---|---|---|
| Group | mice | Serum | Dose | Route | infection |
| 1 | 4 | mRNA CSP | 400 ul | i.v. | 2 h prior to |
| | | vaccine | | | challenge with |
| 2 | 4 | mAB311 or | 400 ul | i.v. | 2000 sporozites |
| | | 317 | | | |
| 3 | 4 | Na [symbol] ve | 400 ul | i.v. | |
| 4 | 4 | mRNA CSP | 400 ul | i.v. | 2 h prior to |
| | | vaccine | | | challenge with |
| 5 | 4 | mAB311 or | 400 ul | i.v. | 250 sporozites |
| | | 317 | | | |
| 6 | 4 | Na [symbol] ve | 400 ul | i.v. | |

16.2. Active Challenge Study of Mice Vaccinated with Various CSP Based mRNA Vaccines:

LNP formulated mRNA-based Malaria vaccines are tested in an active challenge model. Mice are then evaluated for reduced parasite burden in the liver.

LNP-formulated mRNA vaccines encoding CSP are injected intramuscular on days 0 and 21. Starting on day post vaccination, vaccinated mice are challenged with Plasmodium berghei-Plasmodium falciparum CSP chimeric sporozoites (see Table 23).

TABLE 23
Vaccination scheme of Example 16.2.

| Group | No. of mice | Treatment | Dose | Route | Volume | Challenge/dose |
|---|---|---|---|---|---|---|
| 1 | 5 | m RNA CSP vaccine | 1 ug | i.m. | 1 × 25 uL | On day 35 with 2000 |
| 2 | 5 | m RNA CSP vaccine | 5 ug | i.m. | 1 × 25 uL | sporozoites |
| 3 | 5 | m RNA CSP vaccine | 10 ug | i.m. | 1 × 25 uL | |
| 4 | 5 | m RNA irrelevant control | 10 ug | i.m. | 1 × 25 uL | |
| 5 | 5 | mAB311 or 317 | — | i.m. | 1 × 25 uL | |
| 6 | 5 | Naïve | — | i.m. | 1 × 25 uL | |

### Example 17: Analysis of Polyvalent Malaria mRNA Vaccine

The present example shows a mRNA vaccine composition comprising different CSP constructs according the invention comprising for example, but are not limited to:
at least one mRNA encoding a more full length CSP(I) and at least a second mRNA encoding a shortened CSP fragment with HBsAg (II), or
at least two different mRNA constructs comprising different heterologous or CSP-derived T-cell helper epitopes (I) and (II), or
at least one mRNA encoding a more full length CSP with a heterologous signal peptide (I) and at least a second mRNA encoding a shortened CSP fragment with HBsAg (II).

RNAs encoding different Malaria mRNA vaccine encoding CSP or a fragment or variant thereof (see Table 24) were generated according to Example 1 and formulated in LNPs according to Example 1.4.1 or 1.4.2. Balb/c mice (9 mice per group) are vaccinated on day 0 and day 21 intramuscularly (i.m). Serum is collected at day 21 and 28 to test humoral immune responses. Splenocytes are collected at day 28 to test cellular immune responses via an ICS as described above.

TABLE 24
Example of Animal groups and vaccination
schedule of Example 17

| Group | No. of mice | Vaccine composition |
|---|---|---|
| A | 9 | 0.9% NaCl buffer (negative control) |
| B | 9 | mRNA encoding for CSP (I) |
| C | 9 | mRNA encoding for CSP (II) |
| D | 9 | mRNA encoding for CSP (I) and |
| | | mRNA encoding for CSP (II) |

### Example 18: Safety, Reactogenicity and Immunogenicity of Malaria mRNA Vaccine in Healthy Adults

To demonstrate safety, reactogenicity and immunogenicity of Malaria mRNA vaccine, a phase I clinical trial is initiated.

For clinical development, RNA is used that has been produced under GMP conditions (e.g. using a procedure as described in WO2016/180430).

In this Malaria mRNA vaccine phase I trial different dosages of the candidate Malaria mRNA vaccine will be administered in a one or two-dose schedule to healthy adult subjects. The subjects will be enrolled sequentially into the different trial groups to receive one or two doses of Malaria mRNA vaccine. The subjects in the two-dose groups will be administered a second dose 28 days or preferred 56 days later. An additional group of control subjects will receive a single dose of saline on day 1. Safety information for solicited (days 1-7 post-vaccination) and unsolicited (days 1-28 post-vaccination) adverse events (AEs) will be collected using diary cards. Serious AEs, AEs leading to premature withdrawal from the trial or receipt of the second dose, AEof Special Interest and medically-attended AEs will be collected throughout the trial (Day 1 to Day 365 post last vaccine dose). Specified safety data will be reviewed by an internal safety review team and a DSMB on a pre-defined schedule.

1. A coding RNA for a vaccine comprising
a) at least one heterologous 5′ untranslated region (5′-UTR) and/or at least one heterologous 3′ untranslated region (3′-UTR); and
b) at least one coding sequence operably linked to said 3′-UTR and/or 5′-UTR encoding at least one antigenic protein derived from circumsporozoite protein (CSP) of a Malaria parasite, or an immunogenic fragment or immunogenic variant thereof:

2. Coding RNA of claim 1, wherein the Malaria parasite is selected from Plasmodium falciparum (Pf), Plasmodium knowlesi (Pk), Plasmodium ovale (Po), Plasmodium simiovale (Ps), or Plasmodium vivax (Pv).

3. Coding RNA of claim 1 or 2, wherein the Malaria parasite is Plasmodium falciparum (Pf), preferably Plasmodium falciparum 3D7.

4. Coding RNA of any one of claims 1 to 3, wherein the coding sequence additionally encodes at least one heterologous peptide or protein element selected from a heterologous signal peptide, a linker, a helper epitope, an antigen clustering domain, or a transmembrane domain.

5. Coding RNA of claim 4, wherein the heterologous signal peptide is derived from SPARC according to SEQ ID NO: 6208, Hslns-iso1 according to SEQ ID NO: 6207, HsALB according to SEQ ID NO: 6205, or IgE according to SEQ ID NO: 6206, or fragment or variant of any of these.

6. Coding RNA of claim 4, wherein the helper epitope is derived from P2 helper peptide according to SEQ ID NO: 6272, PADRE helper epitope according to SEQ ID NO: 6273, HBsAg carrier matrix according to SEQ ID NO: 6274, or fragment or variant of any of these.

7. Coding RNA of claim 4, wherein the antigen clustering domain is derived from from ferritin according to SEQ ID NO: 10162, lumazine-synthase (LS) according to SEQ ID NO: 10153, surface antigen of hepatitis B virus (HBsAg) according to SEQ ID NO: 6274, or fragment or variant of any of these.

8. Coding RNA of claim 4, wherein the transmembrane domain is derived from a transmembrane domain of HA according to SEQ ID NOs: 6302, or fragment or variant thereof.

9. Coding RNA of any one of the preceding claims, wherein the at least one antigenic protein comprises, preferably in N-terminal to C-terminal direction:
a) optionally, one heterologous secretory signal sequence selected from SEQ ID NOs: 6205-6208;
b) at least one protein derived from CSP of a Malaria parasite, or fragments or variants thereof;
c) optionally, at least one heterologous helper epitope selected from SEQ ID NOs: 6272, 6273, or 6274or fragments or variants thereof;
d) optionally, at least one heterologous antigen clustering domain selected from SEQ ID NOs: 6274, 10153, 10162, or fragments or variants thereof, and
e) optionally, at least one heterologous transmembrane domain selected from SEQ ID NOs: 6302 or fragments or variants thereof,
wherein a), b), c), d) and/or e) may be connected preferably via at least one peptide linker element selected from SEQ ID NOs: 6241-6244, 10141, 10147.

10. Coding RNA of any one of the preceding claims, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs:
1-36, 2081-2120, 2481-2886, 8742-8753, 10080, or an immunogenic fragment or immunogenic variant of any of these.

11. Coding RNA of any one of the preceding claims, wherein the at least one coding sequence comprises at least one nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 37-328, 2121-2480, 2887-6134, 8754-8855, 10086-10139, or a fragment or variant of any of these sequences.

12. Coding RNA of any one of the preceding claims, wherein the at least one coding sequence is a codon modified coding sequence, wherein the amino acid sequence encoded by the at least one codon modified coding sequence is preferably not being modified compared to the amino acid sequence encoded by the corresponding wild type coding sequence.

13. Coding RNA according to claim 12, wherein the at least one codon modified coding sequence is selected from C maximized coding sequence, CAI maximized coding sequence, human codon usage adapted coding sequence, G/C content modified coding sequence, and G/C optimized coding sequence, or any combination thereof.

14. Coding RNA of claim 11 or 13, wherein the at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 41-328, 2161-2480, 3293-6134, 8754-8855, 10092-10139 or a fragment or variant of any of these sequences.

15. Coding RNA of any one of claims 11 to 14, wherein the at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 41-328, 8754-8855 or a fragment or variant of any of these sequences.

16. Coding RNA of any one of claims 11 to 15, wherein the at least one coding sequence comprises a G/C optimized coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 41-112, 2161-2240, 3293-3698, 8754-8783, 10092-10103 or a fragment or variant of any of these sequences.

17. Coding RNA of any one of the preceding claims, wherein the coding RNA is an mRNA, a self-replicating RNA, a circular RNA, or a replicon RNA.

18. Coding RNA of any one of the preceding claims, wherein the coding RNA is an mRNA.

19. Coding RNA of any one of the preceding claims, wherein the coding RNA comprises a 5′-cap structure, preferably m7G, cap0, cap1, cap2, a modified cap0 or a modified cap1 structure.

20. Coding RNA of any one of the preceding claims, wherein the RNA comprises at least one poly(A) sequence, preferably comprising 30 to 150 adenosine nucleotides and/or at least one poly(C) sequence, preferably comprising 10 to 40 cytosine nucleotides.

21. Coding RNA of any one of the preceding claims, wherein the RNA comprises at least one histone stem-loop, wherein the histone stem-loop preferably comprises a nucleic acid sequence according to SEQ ID NOs: 6173 or 6174 or a fragment or variant thereof.

22. Coding RNA of any one of the preceding claims, wherein the at least one heterologous 3′-UTR comprises a nucleic acid sequence derived from a 3′-UTR of a gene selected from PSMB3, ALB7, alpha-globin, CASP1, COX6B1, GNAS, NDUFA1 and RPS9, or from a homolog, a fragment or a variant of any one of these genes.

23. Coding RNA of any one of the preceding claims, wherein the at least one heterologous 5′-UTR comprises a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

24. Coding RNA of any one of the preceding claims, comprising
a-1. at least one 5′-UTR derived from a 5′-UTR of a HSD17B4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
a-3. at least one 5′-UTR derived from a 5′-UTR of a SLC7A3 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
i-2. at least one 5′-UTR derived from a 5′-UTR of a RPL32 gene, or from a corresponding RNA sequence, homolog, fragment or variant thereof and at least one 3′-UTR derived from a 3′-UTR of a ALB7 gene, or from a corresponding RNA sequence, homolog, fragment or variant thereof; or
i-3. at least one 3′-UTR derived from a 3′-UTR of a alpha-globin gene gene, or from a corresponding RNA sequence, homolog, fragment or variant thereof.

25. Coding RNA of any one of the preceding claims, wherein the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 329-2080, 6312-8741, 8856-10079 or a fragment or variant of any of these sequences.

26. A composition comprising at least one coding RNA as defined in any one of claims 1 to 24, wherein the composition optionally comprises at least one pharmaceutically acceptable carrier.

27. Composition of claim 26, wherein the at least one coding RNA is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

28. Composition of claim 27, wherein the at least one coding RNA is complexed or associated with one or more lipids, thereby forming liposomes, lipid nanoparticles, lipoplexes, and/or nanoliposomes.

29. Composition of claim 28, wherein the at least one coding RNA is complexed with one or more lipids thereby forming lipid nanoparticles (LNP).

30. Composition of claim 29, wherein the LNP essentially consists of
(i) at least one cationic lipid;
(ii) at least one neutral lipid;
(iii) at least one steroid or steroid analogue; and
(iv) at least one a PEG-lipid,
wherein (i) to (iv) are in a molar ratio of about 20-60% cationic lipid, 5-25% neutral lipid, 25-55% sterol, and 0.5-15% PEG-lipid.

31. A vaccine comprising the coding RNA as defined in any one of claims 1 to 25, or the composition as defined in any one of claims 26 to 30.

32. Vaccine of claim 31, wherein the vaccine elicits an adaptive immune response.

33. A Kit or kit of parts, comprising the coding RNA as defined in any one of claims 1 to 25, the composition as defined in any one of claims 26 to 30, and/or the vaccine as defined in any one of claims 31 to 32, optionally comprising a liquid vehicle for solubilising, and, optionally, technical instructions providing information on administration and dosage of the components.

34. Coding RNA as defined in any one of claims 1 to 25, the composition as defined in any one of claims 26 to 30, the vaccine as defined in any one of claims 31 to 32, or the kit or kit of parts as defined in claim 33, for use as a medicament.

35. Coding RNA as defined in any one of claims 1 to 25, the composition as defined in any one of claims 26 to 30, the vaccine as defined in any one of claims 31 to 32, or the kit or kit of parts as defined in claim 33, for use in the treatment or prophylaxis of Malaria, or of a disorder related to such an infection.

36. A method of treating or preventing a disorder, wherein the method comprises applying or administering to a subject in need thereof the coding RNA as defined in any one of claims 1 to 25, the composition as defined in any one of claims 26 to 30, the vaccine as defined in any one of claims 31 to 32, or the kit or kit of parts as defined in claim 33.

37. Method of claim 36, wherein the disorder is an infection with Malaria, or a disorder related to such an infection.

38. Method of claims 36 to 37, wherein the subject in need is a mammalian subject, preferably a human subject.
